(19) United States
(12) Patent Application Publication
(10) Pub. No.: US 2008/0299042 A1
(43) Pub. Date: Dec. 4, 2008

(54) Membrane Associated Molecules

(75) Inventors: Pamela Bechtel, San Diego, CA (US); Mark Daniels, San Diego, CA (US); Karen McLachlan, Del Mar, CA (US); Yufeng Zhai, San Diego, CA (US); Benjamin L. Colson, Carlsbad, CA (US); Nicole W. O'Brien, San Marcos, CA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec MA Inc., Cambridge, MA (US)

(21) Appl. No.: 11/579,106

(22) PCT Filed: May 2, 2005
(86) PCT No.: PCT/US2005/015207

§ 371 Date: May 12, 2008

Related U.S. Application Data
(60) Provisional application No. 60/567,187, filed on Apr. 30, 2004.

(51) Int. Cl. A61K 49/00; C40B 40/08; C40B 50/02; C07H 21/00; A61P 31/00; C07K 14/00; A61K 39/395; G01N 33/574

(52) U.S. Cl. 424/9.1; 506/17; 506/24; 424/138.1; 435/7.23; 536/23.5; 530/350

(57) ABSTRACT

The present invention is directed to novel methods of treating, identifying or diagnosing a hyperproliferative disorder in a patient in need thereof. The methods of the invention include administering to a patient a composition comprising a binding molecule which binds to a cell surface expressed glycoprotein expressed predominantly in tumor or tumor-associated cells. In particular, the therapeutic and diagnostic methods of the present invention include the use of a binding molecule, for example an antibody or immunospecific fragment thereof, which specifically binds to a membrane associated molecule, variant polypeptide or fragment thereof. The present invention is based, at least in part, on the discovery of membrane associated proteins, i.e., nucleic acid molecules which encode membrane proteins and the use of these molecules to generate custom arrays to screen for markers associated with various diseases and disorders, e.g., cancer, e.g., lung, colon, pancreatic and ovarian cancer and autoimmune diseases or disorders. The invention further relates to various methods, reagents and kits for diagnosing, staging, prognosing, monitoring and treating hyperproliferative diseases or disorders such as cancer, e.g., lung, colon, pancreatic and ovarian cancer and autoimmune diseases or disorders.

### BACKGROUND OF THE INVENTION

This application includes a “Sequence Listing,” which is provided as an electronic document on a compact disc (CD-R). This compact disc contains the file “Sequence Listing.txt” (4,348,000 bytes, created on Apr. 29, 2005), which is hereby incorporated by reference in its entirety.

### FIELD OF THE INVENTION

This invention relates to nucleic acid molecules that encode membrane associated proteins, the proteins themselves, as well as methods for identifying membrane associated molecules. The invention further relates to methods of applying these membrane associated molecules on array(s) and utilizing the array(s) to identify markers associated with hyperproliferative diseases or disorders, such as cancer and autoimmune diseases.

The present invention is further directed to novel methods of treating and diagnosing hyperproliferative disorders utilizing binding molecules which bind to polypeptides expressed predominantly in tumor or tumor-associated cells.

### RELATED ART

Cancer afflicts approximately 1.2 million people in the United States each year. About 50% of these cancers are curable with surgery, radiation therapy, and chemotherapy. Despite significant technical advances in these three types of treatments, each year more than 500,000 people will die of cancer in the United States alone. (Jaffee, E. M., Ann. N.Y. Acad. Sci. 886:67-72 (1999)). Because most recurrences are at distant sites such as the liver, brain, bone, and lung, there is an urgent need for improved systemic therapies.

Advances have been made in detection and therapy of cancer, however no vaccine or other universally successful method for prevention or treatment is currently available. One reason for failure of a cancer treatment is often the growth of secondary metastatic lesions in distant organs. Therapy for metastasis currently relies on a combination of early diagnosis and aggressive treatment, which may include radiotherapy, chemotherapy or hormone therapy. However, the toxicity of such treatments limits the use of presently available anticancer agents for treatment of malignant disease. The high mortality rate for many cancers indicates that improvements are needed in metastasis prevention and treatment. The goal of cancer treatment is to develop modalities that specifically target tumor cells, thereby avoiding unnecessary side effects to normal tissue. Immunotherapy has the potential to provide an alternative systemic treatment for most types of cancer. The advantage of immunotherapy over radiation and chemotherapy is that it can act specifically against the tumor without causing normal tissue damage.

The development of less toxic antitumor agents would facilitate the long term treatment of latent or residual disease. Such agents could also be used prophylactically after the removal of a precancerous tumor.

Arrays of binding agents, such as oligonucleotides and polynucleotides, have become an increasingly important tool in the biotechnology industry and related fields. These arrays, in which a plurality of binding agents are deposited onto a solid support surface in the form of an array or pattern, find use in a variety of applications, including drug screening, nucleic acid sequencing, mutation analysis, and the like. One important use of arrays is in the analysis of differential gene expression, where the expression of genes in different cells, normally a cell of interest and a control, is compared and any discrepancies in expression are identified.

A variety of different array technologies have been developed in order to meet the growing need of the biotechnology industry. Despite the wide variety of array technologies currently in preparation or available on the market, many of the commercially available arrays do not contain sequences for all of the predicted genes in the human genome. In addition, a significant number of the sequences on commercial arrays are routinely ineffective. As a result, sequence databases which are generated using the sequence information on commercial arrays are often incomplete. There is therefore a continued need to produce improved arrays containing a more complete of sequences.

The amount of sequence information resulting from the rapid cloning of the genomes of species ranging from yeast to man, as well as access to databases containing cloned nucleotide sequences, provides an initial source of information from which to attempt to identify genes such as transmembrane molecules. Transmembrane molecules such as, G-protein coupled receptors (GPCR's), tyrosine kinases receptors, Fc receptors, killer cell inhibitory receptor and HIV receptors, play an essential role in the intracellular communication during the development of a multi-cellular organism. Such transmembrane molecules participate in a multitude of biological processes including cell growth, cell proliferation, cell differentiation and cell death. These proteins can also act as mediators for the transfer of signals from the external environment in to the cell itself, thereby modulating gene expression. Despite their importance in many biological processes, only a small number of transmembrane molecules have been characterized due to their complex nature and the fact that it is extremely difficult to determine their three-dimensional structures by conventional methods.

Several programs have been utilized to mine known databases for relevant sequences. TMHMM, based on the hidden Markov model (HMM) for membrane protein topology prediction, is one program utilized for mining nucleotide databases for transmembrane helices in transmembrane polypeptides (Sonnhammer et al., 1998). TMHMM specializes in modeling various regions of a membrane protein such as the helix caps, middle of the helix, regions close to the membrane, and globular domains. TMHMM is well suited for the prediction of transmembrane molecules because it can incorporate hydrophobicity, charge bias, helix lengths, and grammatical constraints into one model for which algorithms for parameter estimation and prediction already exist (Durbin et al., 1988).

Accordingly, there exists a need for identifying transmembrane proteins, as well as the nucleotide sequences encoding these proteins which are associated with a disease. Such sequences may be utilized to identify and characterize markers that may be used as potential drug targets, as well as to diagnose diseases and disorders associated with transmembrane molecules. Therefore, a major goal in the design and development of new therapies is the identification and characterization of transmembrane molecules, and applying these molecules on an array to allow for the detection of an amplification of specific DNA sequences that are associated with hyperproliferative diseases or disorders, such as cancer.

Accordingly, there is a need in the art for the development of further methods for detecting, inhibiting, and treating cancer, e.g., metastasis.

### BRIEF SUMMARY OF THE INVENTION

This invention involves the use of membrane associated proteins in the diagnosis and treatment of hyperproliferative diseases or disorders using binding molecules.

In one embodiment, the present invention provides a method for identifying membrane associated molecules which are upregulated in hyperproliferative diseases or disorders comprising: development of a unique expression array to detect membrane associated molecules, obtaining biological samples, contacting samples with the unique array and measuring expression level of the membrane associated molecules.

In another embodiment, the present invention provides a method for treating a hyperproliferative disorder in an animal, comprising administering to an animal in need of treatment a composition comprising a binding molecule which specifically binds to a membrane associated molecule, variant or fragment thereof.

In another embodiment the invention provides a method of detecting abnormal hyperproliferative cell growth in a patient, comprising: obtaining a biological sample from the patient; contacting the sample with a binding molecule which specifically binds to a membrane associated molecule, variant or fragment thereof, and assaying the expression level of the membrane associated molecule in the sample.

Yet another embodiment provides a method of diagnosing a hyperproliferative disease or disorder in a patient, comprising administering to the patient a sufficient amount of a detectably labeled binding molecule which specifically binds to a membrane associated molecule, variant or fragment thereof, waiting for a time interval following the administration to allow the binding molecule to contact the membrane associated molecule, variant or fragment thereof and detecting the amount of binding molecule which is bound to the membrane associated molecule, variant or fragment thereof in the patient.

In various embodiments, binding molecules for use in the above methods include antibodies and antigen-specific fragments thereof, fusion proteins, T-cell receptors, and small molecules.

In the above methods, binding molecules bind to polypeptide variants or fragments thereof which are at least 70% identical to membrane associated molecules selected from the group consisting of SEQ ID NOs:1288, 3446-3452 and 3458-3462. Additionally, the binding molecules of the above methods bind to polypeptide variants or fragments which comprise specific domains of the membrane associated molecules as described in Table 2 or the extracellular domains of the membrane associated molecules described in Table 3.

The invention further involves preparing therapeutic agents such as monoclonal antibodies and fusion proteins bearing extracellular binding domains that bind with high affinity and specificity to proteins that are specifically present in disease- or disorder-associated tissues, e.g., proteins that are useful targets for killing or interfering with the function of cells of the tissue that express the targeted proteins.

In another aspect, the present invention is directed to methods for the identification of nucleic acid molecules that encode membrane associated molecules, i.e., nucleic acid molecules which encode transmembrane proteins or which encode proteins with GPI link, ITIM, ITAM or ITSM motifs. It will be appreciated that one particularly preferred embodiment of the present invention comprises methods of using these identified polynucleotides to generate custom arrays to identify markers associated with selected diseases or disorders including various proliferative afflictions and autoimmune disorders. Those skilled in the art will also appreciate that the present invention further comprises the identified polynucleotides as well as the encoded membrane associated molecules and methods of their use. In another important aspect, the present invention is directed to small molecules, ligands or immunoreactive species (e.g. immunoglobulins) that bind to, or otherwise interact or associate with the membrane associated molecules or their respective polynucleotides. Still other aspects of the instant invention comprise methods of using such membrane associated molecules or compounds that interact with them for the diagnosis, prevention or treatment of various diseases or disorders e.g., cancer or autoimmune diseases or disorders. Yet other embodiments of the instant invention are enumerated below in more detail or will become apparent to the skilled artisan in view of the instant specification and examples.

In one aspect, the present invention provides an array comprising a plurality of nucleic acid molecules selected from the group consisting of the nucleotide sequences set forth in SEQ ID NOs:1-1146, 3439-3445, and 3452-3457, their complements and hybridizing fragments thereof. In another aspect, the present invention provides an array comprising a plurality of nucleic acid molecules selected from the group consisting of nucleic acid molecules having at least 70% identity with the nucleotide sequences set forth in SEQ ID NOs:1-1146, 3439-3445, and 3452-3457, their complements and hybridizing fragments thereof. In still another aspect, the invention provides an array comprising a plurality of nucleic acid molecules selected from the group consisting of the nucleic acid molecules set forth in Table 7, Table 8, Table 9 and/or Table 10, their complements and hybridizing fragments thereof.

In one embodiment, the nucleic acid molecules of the present invention encode polypeptides having at least one transmembrane domain, GPI link, immunoreceptor tyrosine-based inhibitory motif ITIM), immunoreceptor tyrosine-based activatory motif (ITAM) and immunoreceptor tyrosine-based switch motif (ITSM), or a fragment thereof. In another embodiment, the nucleic acid molecules are produced synthetically. In still another embodiment, the nucleic acid molecules of the present invention are associated with a substrate e.g., in a predetermined region. In yet another embodiment, the substrate is selected from the group consisting of glass, plastic, or a filter. In a related embodiment, the nucleic acid molecules of the present invention are associated with the substrate by covalent bonding or by a linker.

In another embodiment, the substrate-associated nucleic acid molecules of the present invention hybridize with one or more nucleic acid molecules derived from a target sample. The target sample nucleic acid molecules may be labeled, e.g., fluorescently labeled. In still another embodiment, the target sample nucleic acid molecules of the present invention originate from a biological source selected from the group consisting of a cell, blood, plasma, lymph, urine, tissue, mucus, sputum and saliva. In a related embodiment, the biological source is derived from a cancerous tissue e.g., lung, colon, ovary, pancreas, prostate and breast tissues. In yet another embodiment, the target sample is obtained from a patient suffering from an autoimmune disorder.

In another aspect, the present invention provides methods of associating a plurality of molecules selected from the group consisting of nucleic acid molecules, protein molecules and fragments thereof comprising the steps of querying a sequence database for the presence of transmembrane molecules using a transmembrane protein topology prediction program to provide a transmembrane selection set; comparing homology of the molecules of the transmembrane selection set with array associated molecules; and excluding those molecules from the transmembrane selection set exhibiting substantial homology with one or more array associated molecules to provide a transmembrane signature set comprising a plurality of molecules.

In one embodiment, the methods of the present invention further comprise the steps of comparing the sequences of one or more molecules excluded from the transmembrane selection set with sequences of molecules set forth in an expression database; and excluding those compared molecules which are associated with substantial intensity values in the expression database to provide an expression signature set.

In another embodiment, the methods of the present invention further comprise the step of combining one or more of the molecules set forth in the transmembrane signature set with one or more of the molecules set forth in the expression signature set to provide a master transmembrane signature set.

In one embodiment, the transmembrane signature set, the expression signature set and the master transmembrane signature set comprise nucleic acid molecules. In another embodiment, the invention provides arrays comprising one or more transmembrane signature set nucleic acid molecules, their complement or hybridizing fragments thereof. In yet another embodiment, the invention provides arrays comprising one or more expression signature set nucleic acids molecules, their complement or hybridizing fragments thereof. In a further embodiment, inventor provides arrays comprising one or more master transmembrane signature set nucleic acid molecules, their complement or hybridizing fragments thereof.

In another aspect, the present invention provides methods of associating a plurality of molecules selected from the group consisting of nucleic acid molecules, protein molecules and fragments thereof comprising the steps of querying said sequence database for the presence of molecules exhibiting a GPI link, ITIM, ITAM or ITSM motif to provide a motif selection set comprising a plurality of molecules; comparing homology of the molecules of the motif selection set with said array associated molecules; and excluding those molecules from the motif selection set exhibiting substantial homology with one or more array associated molecules to provide a motif signature set comprising a plurality of molecules. In one embodiment, the methods further comprise combining at least one of the molecules from the motif signature set with at least one molecule from the master transmembrane signature set to provide a screening signature set comprising a plurality of molecules.

In one embodiment, the screening signature set molecules are nucleic acid molecules. In yet another embodiment, the invention provides arrays comprising one or more motif signature set nucleic acid molecules, their complements or hybridization fragments thereof. In another embodiment, the invention provides arrays comprising one or more screening signature set nucleic acid molecules, their complement or hybridization fragments thereof.

In another aspect, the invention pertains to methods of identifying a membrane associated molecule indicative of a hyperproliferative disease comprising contacting nucleic acid molecules derived from a target sample with an array of the present invention; detecting the nucleic acid molecules exhibiting an altered expression profile; and identifying at least one membrane associated molecule corresponding to the nucleic acid molecules having the altered expression profile.

The present invention also provides markers, e.g., marker proteins having altered expression profiles, where the markers, e.g., marker proteins, are identified in a target sample using an array of the invention. The present invention further provides antibodies or immunoreactive fragments thereof, which react with a marker protein of the invention.

### BRIEF DESCRIPTION OF THE DRAWINGS/FIGURES

FIG. 1 is a diagrammatic representation illustrating preferred methods of the present invention.

FIG. 2 shows a subset of genes identified from the custom chip as upregulated in colon cancer, evaluated by quantitative PCR (QPCR) as described in Example 4. FIG. 2 depicts the results for seven genes; SEQ ID NO:3446, SEQ ID NO:3447, SEQ ID NO:3448, SEQ ID NO:3449, SEQ BD NO:3450, SEQ ID NO:3451 and SEQ ID NO:3452.

### BRIEF DESCRIPTION OF THE INCORPORATED TABLES

Table 1 lists membrane associated molecules of the present invention which were isolated from the cellular membranes of tumors from human patients with colon cancer and were identified via quantitative PCR (QPCR). Table 2 describes exemplary fragments of the membrane associated molecules of Table 1 based on homologies to known domains and the amino acid sequence positions which define the approximate beginning and end of the domains. Table 3 provides portions of membrane associated molecules of Table 1 predicted to be part of the intracellular, extracellular or nontransmembrane regions. Table 4 shows all of the bases which code for an amino acid. Table 5 lists the SEQ ID NOs for the membrane associated molecule polynucleotides identified in Table 1. Table 6 lists each of the nucleotide SEQ ID NOs, corresponding amino acid SEQ ID NOs and corresponding probe SEQ ID NOs identified by the methods described herein. The nucleotide sequences of the genes identified by the methods described herein are set forth as SEQ ID NOs:1-1133. SEQ ID NOs:1134-1146 represent control sequences. One skilled in the art will appreciate the use of these sequences as positive and negative controls. The arrays of the invention may or may not include these control sequences. The amino acid sequences of the proteins corresponding to these genes are set forth as SEQ ID NOs:1147-2292. The probe sequences, set forth in SEQ ID NOs:2293-2438, correspond to a partial complement of the identified nucleotide sequences of the invention. In preferred embodiments, those skilled in the art will appreciate that these probes may be particularly useful for the preparation of arrays as described herein. In addition to providing a listing of sequences identified using the present invention, Table 6 further provides a summary of expression profiles for each of the membrane associated molecules in four different tumor types. More specifically, an “X” next to particular SEQ ID NOs. indicates that the identified molecule exhibits an altered expression profile in either colon, lung, pancreatic or ovarian tumors as compared to normal tissue. The designation represents measurements from a number of tumors in each specific proliferative disorder. One skilled in the art will appreciate that this differential expression is strongly indicative that the subject membrane associated molecule or associated nucleic acid molecule may provide a marker or target for the enumerated disorder.

Markers, e.g., marker proteins or marker nucleic acid molecules, which may be associated with cancer, can be identified using the methods of the present invention and arrays generated from some or all of the molecules comprising the motif signature set, transmembrane signature set, expression signature set, master transmembrane signature set or screening signature set. For example, custom arrays generated using a plurality of molecules from one of the aforementioned associations may be used to screen for molecules which are overexpressed in cancer, e.g., lung, colon, pancreatic and/or ovarian cancer. In exemplary embodiments of the present invention, specific membrane associated molecules (sometimes referred to herein as tumor associated molecules) have been identified which are associated with colon cancer, lung cancer, pancreatic cancer and ovarian cancer. Tables 7 through 10 are subsets of Table 6 and identify, respectively, those membrane associated molecules (by nucleotide SEQ ID NOs.) that exhibit altered expression profiles in colon tumors, lung tumors, pancreatic tumors and ovarian tumors. Put another way, Table 8 is a listing of each of those nucleotide SEQ ID NOs. which have an “X” in the column headed “colon” in Table 6. Similarly, Table 8 is representative of those SEQ ID NOs being designated as having an altered expression profile in colon tumors in Table 6. Tables 9 and 10 are the same for pancreatic and ovarian tumors respectively. It will be appreciated that the plurality of molecules associated in each of these subsets, or their complement or hybridizing fragments thereof, may be used to generate custom arrays useful in screening for particular indications. For example, an array could be generated using some or all of the molecules set forth in Table 7 that would be particularly useful in screening or diagnosing colon cancer.

More particularly, Tables 7-10 comprise membrane associated molecules which correlate to, or are indicative of, colon, lung, pancreatic or ovarian cancers. Using nonparametric methods, the molecules were analyzed according to expression rank (Exp) and intensity order (Order). In each case the malignant samples were compared to normal heart, liver, kidney, colon and lung. For the malignant samples, each molecule is significantly overexpressed compared to the number of normal tissue indicated in the table for the Exp and Order columns. In addition, the molecules were examined using global normalization techniques (Global). Again the malignant samples were compared with normal heart, liver, kidney, colon and lung. The table indicates the number of malignant samples which are overexpressed compared with 0, 1, 2, 3, 4 or 5 normal tissues.

### DETAILED DESCRIPTION OF THE INVENTION

### Definitions

So that the invention may be more readily understood, certain terms are first defined.

It is to be noted that the term “a” or “an” entity, refers to one or more of that entity; for example, “an immunoglobulin molecule,” is understood to represent one or more immunoglobulin molecules. As such, the terms “a” (or “an”), “one or more,” and “at least one” can be used interchangeably herein.

In the present invention, “isolated” refers to material removed from its native environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide.

In the present invention, a “membrane protein” or “membrane polypeptide” is a polypeptide that is present in the membrane of cells through either direct or indirect association with the lipid bilayer, including, in particular, through prenylation of a carboxyl-terminal amino acid motif. Membrane proteins are amphipathic, meaning that the polypeptide has a hydrophobic and a hydrophilic region. Typically the hydrophobic regions interact with the lipid bilayer of the cell and the hydrophilic regions interact with the aqueous interior or exterior of the cell.

Certain membrane proteins are “transmembrane proteins” and have a extracellular domain, which interacts with the external cellular environment, an intracellular domain, which interacts with the internal cellular environment and a transmembrane domain which traverses the cellular lipid bilayer. Certain membrane proteins however do not have extracellular domains and interact with the lipid bilayer through covalently attached fatty acid groups, prenyl groups, oligosaccharides or through protein-protein interacts with other proteins in the cellular membrane. The addition of prenyl groups is known as prenylation and involves the covalent modification of a protein by the addition of either a farnesyl or geranylgeranyl isoprenoid. Prenylation occurs on a cysteine residue located near the carboxyl-terminus of a protein.

As used herein, a “polynucleotide” can contain the nucleotide sequence of the fall length cDNA sequence, including the untranslated 5′ and 3′ sequences, the coding sequences, as well as fragments, eptiopes, domains, and variants of the nucleic acid sequence. The polynucleotide can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotides can be composed of triple-stranded regions comprising RNA or DNA or both RNA and DNA. polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

In the present invention, a “polypeptide” can be composed of amino acids joined to each other by peptide bonds or modified peptide bonds, i.e., peptide isosteres, and may contain amino acids other than the 20 gene-encoded amino acids. The polypeptides of the present invention may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T. E. Creighton, W.H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

In the present invention, a “polypeptide fragment” refers to a short amino acid sequence, for example, short amino acid sequences derived from the polypeptides derived from SEQ ID NOs:1288, 3446-3452 and 3458-3462. Protein fragments may be “free-standing,” or comprised within a larger polypeptide of which the fragment forms a part of region. Representative examples of polypeptide fragments of the invention, include, for example, fragments comprising about 5 amino acids, about 10 amino acids, about 15 amino acids, about 20 amino acids, about 30 amino acids, about 40 amino acids, about 50 amino acids, about 60 amino acids, about 70 amino acids, about 80 amino acids, about 90 amino acids, and about 100 amino acids in length.

As used herein, the term “array” represents an intentionally created collection of molecules which can be prepared either synthetically or biosynthetically. In particular, the term “array” as used herein means an intentionally created collection of peptides, proteins, oligonucleotides or polynucleotides attached to at least a first surface of at least one substrate wherein the identity of each molecule at a given predefined region is known.

As used herein, the term “oligonucleotide” as used herein refers to a nucleic acid molecule comprising from about 2 to about 300 nucleotides or more. Oligonucleotides for use in the present invention are preferably from 20-150 nucleotides in length, more preferably from 30-80 nucleotides in length.

As used herein, the term “solid support,” “support,” and “substrate” refer to a material or group of materials having a rigid or semi-rigid surface or surfaces. In many embodiments, at least one surface of the solid support will be substantially flat, although in some embodiments it may be desirable to create separate regions on the solid support, for example, wells, raised regions, pins, etched trenches, or the like. In one embodiment, the surface is glass, plastic (e.g., polypropylene, nylon), polyacrylamide or a filter, e.g., a nitrocellulose filter. According to other embodiments, the solid support(s) will take the form of beads, resins, gels, microspheres, fibers or other geometric configurations.

As used herein, the term “immobilization” refers to both noncovalent association, such as absorption, or covalent attachment, such as by way of a cross-linking agent.

A “marker” is a gene whose altered level of expression in a tissue or cell from its expression level in normal or healthy tissue or cell is associated with a disease state, such as cancer. A “marker nucleic acid” is a nucleic acid (e.g., mRNA, cDNA) encoded by or corresponding to a marker of the invention. For example, such marker nucleic acid molecules include DNA (e.g., cDNA) comprising the entire or a partial sequence of any of the nucleic acid sequences set forth in Tables 7-10 or the complement or hybridizing fragment of such a sequence. Marker nucleic acid molecules compatible with the instant invention also include RNA comprising the entire or a partial sequence of any of the nucleic acid sequences set forth in Tables 7-10 or the complement of such a sequence, wherein all thymidine residues are replaced with uridine residues. In one embodiment, a “marker” is a gene encoding a membrane associated protein, or a fragment thereof. For example, a “marker protein” is a protein encoded by or corresponding to a marker of the invention. Thus, exemplary marker proteins of the instant invention comprise the entire or a partial sequence of a protein encoded by any of the sequences set forth in Tables 7-10 or a fragment thereof. In one embodiment, a “marker protein” is a membrane associated protein or a fragment thereof. The terms “protein” and “polypeptide” are used interchangeably herein.

The term “probe” refers to any molecule which is capable of selectively binding to a specifically intended target molecule, for example, a nucleotide transcript or protein encoded by or corresponding to a marker. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

The term “target nucleic acid” or “target sequence” refers to a nucleic acid or nucleic acid sequence which is to be analyzed. A target nucleotide can be a nucleic acid to which a probe will hybridize. The probe may or may not be specifically designed to hybridize to the target. It is either the presence or absence of the target nucleic acid that is to be detected, or the amount of the target nucleic acid that is to be quantified. The term target nucleic acid may refer to the specific subsequence of a larger nucleic acid to which the probe is directed or to the overall sequence (e.g., gene or mRNA) whose expression level it is desired to detect. The difference in usage will be apparent from context.

The term “hybridizing fragment,” as used herein, refers to a nucleic acid sequence which is capable of hybridizing to a target sequence.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybrids can contain two DNA strands, two RNA strands, or one DNA and one RNA strand. In one embodiment, a hybridizing fragment hybridizes to a target sequence under stringent conditions. As used herein, the term “hybridizes under stringent conditions” is intended to describe conditions for hybridization and washing under which nucleotide sequences at least 60% (65%, 70%, 75%, 80%, 85%, 90%, preferably 95%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A non-limiting example of stringent hybridization conditions are hybridization in 6× sodium chloride/sodium citrate (SSC) at about 45 C, followed by one or more washes in 0.2×SSC, 0.1% SDS at 50-65 C. In a particularly preferred embodiment, stringent conditions comprise hybridization in 2×SDS (0.5 M NaPO4, 1% SDS, 2 mM EDTA, 2×SSC, 4×Denhardt's solution) at 42 C for 16-24 hrs, followed by one or more washes with 2×SSC with 0.2% SDS, 2×SSC or 0.2×SSC at 42 C.

The term “altered level of expression” of a marker refers to an expression level in a test sample e.g., a sample derived from a patient suffering from a hyperproliferative disease or disorder, that is greater or less than the standard error of the assay employed to assess expression, and is preferably at least twice, and more preferably three, four, five or ten times the expression level of the marker in a control sample (e.g., sample from a healthy subjects not having the associated disease) and preferably, the average expression level of the marker in several control samples. The “normal” level of expression of a marker is the level of expression of the marker in a control sample, e.g., a sample from a subject not afflicted with a hyperproliferative disease or disorder such as cancer e.g., lung, colon, pancreatic, and ovarian cancer and autoimmune diseases.

An “overexpression” or “significantly higher level of expression” of a marker refers to an expression level in a test sample that is greater than the standard error of the assay employed to assess expression, and is preferably at least twice, and more preferably three, four, five or ten times the expression level of the marker in a control sample (e.g., sample from a healthy subject not afflicted with a hyperproliferative disease or disorder) and preferably, the average expression level of the marker in several control samples.

A “significantly lower level of expression” of a marker refers to an expression level in a test sample that is at least twice, and more preferably three, four, five or ten times lower than the expression level of the marker in a control sample (e.g., sample from a healthy subject not afflicted with a hyperproliferative disease or disorder) and preferably, the average expression level of the marker in several control samples.

As used herein, “membrane associated molecules” include transmembrane protein molecules and GPI link, ITIM, ITAM, or ITSM motif containing protein molecules. Further, for the purposes of the instant application and as set forth by the context of use, nucleic acid molecules encoding such proteins or portions thereof may be referred to as membrane associated molecules. Some membrane associated molecules are integral membrane proteins, at least a portion of which reside and operate within a cell's plasma membrane, or within the membranes of a subcellular compartment or organelle. In selected embodiments, membrane associated molecules comprise at least one transmembrane domain (TM domain). As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In one embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al. (1996) Annual Rev. Neurosci. 19: 235-263, the contents of which are incorporated herein by reference.

In another embodiment, membrane associated molecules comprise at least one GPI link, ITIM, ITAM or ITSM motif. As used herein, the term “GPI link motif” refers to proteins associated with the outer leaflet of the plasma membrane via a glycosyl linkage to the inositol head group of lipid molecules known as glycosyl-phosphatidylinositol (GPI)-anchored proteins. Preferably the GPI link motif includes a core structure composed of ethanolamine phosphate in an amide linkage to the carboxy terminus of the molecule, three mannose residues, glucosamine and phosphatidylinositol. In other embodiments variations such as the addition of extra sugars or ethanolamine phosphates to the mannose residues; acetylation of the inositol ring, changes in the fatty acids (length, saturation, hydroxylation or the linkages to the glycerol backbone) may occur with the GPI link motif. GPI link motifs are described in, for example, Mayor S. et al. (2004) Nature Rev Mol Cell Biol 5(2); 110-120, the contents of which are incorporated here. As used here, the term “ITIM motif” is characterized as an immunoreceptor tyrosine-based inhibitory motif. More preferably, ITIM motifs contain the sequence [I/V]XYXXL. As used here, the term “ITAM motif” is characterized as an immunoreceptor tyrosine-based activation motif. More preferably, ITAM motifs contain the sequence YXX[L/V]X₇₋₁₁YX[L/V]. As used here, the “ITSM motif” is characterized as an immunoreceptor tyrosine-based switch motif. More preferably, ITSM motifs contain the sequence [S/T]XTXXX[V/I]. ITIM, ITAM and ITSM motifs are described in, for example, Sidorenko S. et al. (2003) Nature Immunology 4(1):19-24, the contents of which are incorporated herein by reference.

As used herein, the term “transmembrane selection set” refers to a subset of sequences identified in a sequence database utilizing a model for membrane protein topology prediction. In one embodiment, the sequence database is the Ensemble™ database. In another embodiment, the model of membrane topology prediction is the Transmembrane Hidden Markov Model (TMHMM).

As used herein, the term “transmembrane signature set” refers to a subset of sequences identified by comparing the transmembrane selection set of molecules against a commercially available array and excluding those molecules having substantial homology to the molecules associated with the arrays.

The term “commercially available array” shall be held to mean any one of a number of arrays sold on the open market and used to screen samples for gene expression levels. Non-limiting examples of commercial arrays include Affymetrix Human Genome Focus Array, Affymetrix Human Genome U133 Plus 2.0, Affymetrix Human Genome U133 set, Affymetrix Human Genome U133A 2.0, Affymetrix Human GenomeU95 set, Affymetrix HuGeneFL Genome, Affymetrix Human X3P, Human Cancer G110, Human Genome U95 B-E, Amersham CodeLink™ Human Whole Genome 55K, Amersham CodeLink Uniset™ Human 20K, Amersham CodeLink Uniset™ Human 10K, Agilent Whole Genome, Agilent Human 1A 9 (V2), Agilent Human 1B, MWG Human 40K (A/B), MWG Human 30K (A/B/C), MWG Human Cancer and MWG Human Inflammation expression arrays. Molecules on the surface of such arrays or chips shall be termed “array associated molecules” for the purposes of the instant application. It will be appreciated that those skilled in the art can readily identify and include comparable arrays based on the foregoing exemplary list.

As used herein, the term “expression signature set” refers to a subset of sequences identified by comparing the molecules examined from the transmembrane selection set during identification of the transmembrane signature set against an expression database and excluding those molecules having a substantial intensity value in the expression database. A non-limiting example of an expression database includes the Genelogic™ expression database.

As used herein, the term “master transmembrane signature set” refers to the combination of the sequences contained within the transmembrane signature set and the expression signature set.

As used herein, the term, “motif selection set” refers to a subset of sequences identified by screening a publicly available sequence database of molecules for the presence of a sequence motif. Exemplary sequence motifs include GPI link, ITIM, ITAM and ITSM motifs.

As used herein, the term “motif signature set” refers to a subset of sequences identified by comparing the motif selection set of molecules against a commercial array and excluding those molecules having a substantial homology to sequences associated with the array. Non-limiting examples of commercial arrays are enumerated above.

As used herein, the term “motif” refers to certain sequence patterns known to code for regions of proteins having specific biological characteristics such as signal sequences, DNA binding domains, or transmembrane domains. Exemplary sequence motifs include GPI link, immunoreceptor tyrosine-based inhibitory motif (ITIM), immunoreceptor tyrosine-based activatory motif (ITAM) and immunoreceptor tyrosine-based switch motif (ITSM).

As used herein, the term “screening signature set” refers to the combination of the sequences contained within the master transmembrane signature set and the motif signature set.

As used herein, the term “substantial intensity value” refers to an intensity value for a membrane-associated molecule which is present in greater than about 5% of patient samples in 5 or more sample sets. In one embodiment, the patient sample and sample set information are derived from the Gene Logic™ cancer suite database.

As used herein, the term “sample set” refers to groups of expression data derived from patients by tissue and disease state. Examples of sample sets includes, but is not limited to: Tumor Breast, Normal Breast, Tumor Cervix, Normal Cervix, Tumor Colon, Normal Colon, Tumor Duodenum, Normal Duodenum, Tumor Endometrium, Normal Endometrium, Tumor Esophagus, Normal Esophagus, Tumor Kidney, Normal Kidney, Tumor Liver, Normal Liver, Tumor Lung, Normal Lung, Tumor Lymph Node, Normal Lymph Node, Tumor Ovary, Normal Ovary, Tumor Pancreas, Normal Pancreas, Tumor Prostate, Normal Prostate, Tumor Rectum, Normal Rectum, Tumor Spleen, Normal Spleen, Tumor Stomach, Normal Stomach, Tumor Testis, Normal Testis, Normal Bladder, Normal Bones, Normal Brain, Normal Left Ventricle, Normal Muscles, Normal Myometrium, Normal Skin, Normal Small intestine, Normal Thymus, Normal White Blood Cell. The sample set information was derived from the Gene Logic cancer suite database.

The term “nucleotide sequence” is intended to include DNA sequences (e.g., cDNA or genomic DNA sequences) and RNA sequences (e.g., mRNA sequences). The nucleotide sequence is an art-recognized means for describing a nucleic acid molecule, defined herein, for which the nucleobase sequence has been determined. A nucleotide sequence is preferably depicted as a single strand of nucleobases (e.g., including the bases A, T, C, or G for DNA nucleotide sequences and including the bases A, U, C, or G for RNA sequences) and is written in a 5′ to 3′ orientation. However, a DNA sequence may also be depicted as a double strand of complementary nucleobases. Moreover, one of skill in the art can readily determine the sequence of a complementary strand of nucleotide sequence (e.g., a 3′ to 5′ strand) given, for example, a 5′ to 3′ strand of bases.

As used herein, a “naturally-occurring” nucleic acid molecule refers to an RNA or DNA molecule having a nucleotide sequence that occurs in nature (e.g., encodes a natural protein).

As used herein, the term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue-specific manner.

A “constitutive” promoter is a nucleotide sequence which, when operably linked with a polynucleotide which encodes or specifies a gene product, causes the gene product to be produced in a living human cell under most or all physiological conditions of the cell.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a marker of the invention and normal post-transcriptional processing (e.g. splicing), if any, of the RNA transcript, and reverse transcription of the RNA transcript.

“Complementary” or “complement” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “homology” refers to sequence similarity between two polynucleotide sequences or between two polypeptide sequences. The phrases “percent homology” and “% homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Similarity can be determined by comparing a position in each sequence that may be aligned for purposes of comparison. When a position in the compared sequence is occupied by the same nucleotide base or amino acid, then the molecules are identical at that position. A degree of homology or similarity of polypeptide sequences is a function of the number of amino acids at positions shared by the polypeptide sequences. The term “substantial homology,” as used herein, refers to homology of at least 50%, more preferably, 60%, 70%, 80%, 90%, 95%, 99% or more.

A molecule is “fixed” or “affixed” to a substrate if it is covalently or non-covalently associated with the substrate such the substrate can be rinsed with a fluid (e.g. standard saline citrate, pH 7.4) without a substantial fraction of the molecule dissociating from the substrate.

A disease or disorder is “inhibited” if at least one symptom of the disease or disorder is alleviated, terminated, slowed, or prevented. As used herein, a disease or disorder such as, for example, cancer is “inhibited” if recurrence or metastasis of the cancer is reduced, slowed, delayed, or prevented.

A kit is any manufacture (e.g., a package or container) comprising at least one reagent, e.g., a probe, for specifically detecting the expression of a marker of the invention. The kit may be promoted, distributed, or sold as a unit for performing the methods of the present invention.

Binding molecules. The methods of treating hyperproliferative disorders as described herein utilize “binding molecules.” A binding molecule comprises, consists essentially of, or consists of at least one binding domain which, either alone or in combination with one or more additional binding domains, specifically binds to a target gene product (such as a protein, an antigen or other binding partner), e.g., a lung, colon, pancreatic and/or ovarian cancer tumor-associated polypeptide or fragment or variant thereof. For example, in various embodiments, a binding molecule comprises one or more immunoglobulin antigen binding domains, one or more binding domains of a receptor molecule which, either alone or together, specifically bind a ligand, or one or more binding domains of a ligand molecule which, either alone or together, specifically bind a receptor. In certain embodiments, a binding molecule comprises, consists essentially of, or consists of at least two binding domains, for example, two, three, four, five, six, or more binding domains. Each binding domain may bind to a target molecule separately, or two or more binding domains may be required to bind to a given target, for example, a combination of an immunoglobulin heavy chain and an immunoglobulin light chain.

Binding molecules, e.g., binding polypeptides, e.g., lung, colon, pancreatic and/or ovarian cancer tumor-associated polypeptide-specific antibodies used in the diagnostic and treatment methods disclosed herein may comprise, consist essentially of, or consist of two or more subunits thus forming multimers, e.g., dimers, trimers or tetramers. For example, certain binding molecules comprise a polypeptide dimer, typically, a heterodimer comprising two non-identical monomeric subunits. Other binding molecules comprise tetramers, which can include two pairs of homodimers, e.g., two identical monomeric subunits, e.g., an antibody molecule consisting of two identical heavy chains and two identical light chains.

Certain binding molecules, e.g., binding polypeptides to be utilized in the diagnostic and treatment methods disclosed herein comprise at least one amino acid sequence derived from an immunoglobulin. A polypeptide or amino acid sequence “derived from” a designated protein refers to the origin of the polypeptide. In certain cases, the polypeptide or amino acid sequence which is derived from a particular starting polypeptide or amino acid sequence has an amino acid sequence that is essentially identical to that of the starting sequence, or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the starting sequence. Alternatively, a polypeptide or amino acid sequence derived from a designated protein may be similar, e.g., have a certain percent identity to the starting sequence, e.g., it may be 60%, 70%, 75%, 80%, 85%, 90%, or 95% identical to the starting sequence, as described in more detail below.

Preferred binding polypeptides comprise, consist essentially of, or consist of an amino acid sequence derived from a human amino acid sequence. However, binding polypeptides may comprise one or more contiguous amino acids derived from another mammalian species. For example, a primate heavy chain portion, hinge portion, or binding site may be included in the subject binding polypeptides. Alternatively, one or more murine-derived amino acids may be present in a non-murine binding polypeptide, e.g., in an antigen binding site of a binding molecule. In therapeutic applications, preferred binding molecules to be used in the methods of the invention are not immunogenic in the animal to which the binding polypeptide is administered.

It will also be understood by one of ordinary skill in the art that the binding polypeptides for use in the diagnostic and treatment methods disclosed herein may be modified such that they vary in amino acid sequence from the naturally occurring binding polypeptide from which they were derived. For example, nucleotide or amino acid substitutions leading to conservative substitutions or changes at “non-essential” amino acid residues may be made.

In certain embodiments, a binding polypeptide for use in the methods of the invention comprises an amino acid sequence or one or more moieties not normally associated with that binding polypeptide. Exemplary modifications are described in more detail below. For example, a binding polypeptide of the invention may comprise a flexible linker sequence, or may be modified to add a functional moiety (e.g., PEG, a drug, a toxin, or a label).

A binding polypeptide for use in the methods of the invention may comprise, consist essentially of, or consist of a fusion protein. Fusion proteins are chimeric molecules which comprise a binding domain with at least one target binding site, and at least one heterologous portion.

A “chimeric” protein comprises a first amino acid sequence linked to a second amino acid sequence with which it is not naturally linked in nature. The amino acid sequences may normally exist in separate proteins that are brought together in the fusion polypeptide or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a genotypically distinct entity from that of the rest of the entity to which it is being compared. For instance, a heterologous antigen may be derived from a different species origin, different cell type, or the same type of cell of distinct individuals.

The term “ligand binding domain” or “ligand binding portion” as used herein refers to any native receptor (e.g., cell surface receptor) or any region or derivative thereof retaining at least a qualitative ligand binding ability, and preferably the biological activity of a corresponding native receptor.

The term “receptor binding domain” or “receptor binding portion” as used herein refers to any native ligand or any region or derivative thereof retaining at least a qualitative receptor binding ability, and preferably the biological activity of a corresponding native ligand.

Antibody or Immunoglobulin. In one embodiment, the binding molecules for use in the diagnostic and treatment methods disclosed herein are “antibody” or “immunoglobulin” molecules, or immunospecific fragments thereof, e.g., naturally occurring antibody or immunoglobulin molecules or engineered antibody molecules or fragments that bind antigen in a manner similar to antibody molecules. The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

As will be discussed in more detail below, the term “immunoglobulin” comprises five broad classes of polypeptides that can be distinguished biochemically. All five classes are clearly within the scope of the present invention, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Both the light and heavy chains are divided into regions of structural and functional homology. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (C_{L}) and the heavy chain (C_{H}1, C_{H}2 or C_{H}3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the C_{H}3 and C_{L} domains actually comprise the carboxy-terminus of the heavy and light chain, respectively.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the scope of the instant invention.

As indicated above, the variable region allows the antibody to selectively recognize and specifically bind epitopes on antigens. That is, the V_{L} domain and V_{H} domain of an antibody combine to form the variable region that defines a three dimensional antigen binding site. This quaternary antibody structure forms the antigen binding site present at the end of each arm of the Y. More specifically, the antigen binding site is defined by three complementary determining regions (CDRs) on each of the V_{H} and V_{L} chains. In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In camelid species, however, the heavy chain variable region, referred to as V_{H}H, forms the entire CDR. The main differences between camelid V_{H}H variable regions and those derived from conventional antibodies (V_{H}) include (a) more hydrophobic amino acids in the light chain contact surface of V_{H} as compared to the corresponding region in V_{H}H, (b) a longer CDR3 in V_{H}H, and (c) the frequent occurrence of a disulfide bond between CDR1 and CDR3 in V_{H}H.

In one embodiment, an antigen binding molecule of the invention comprises at least one heavy or light chain CDR of an antibody molecule. In another embodiment, an antigen binding molecule of the invention comprises at least two CDRs from one or more antibody molecules. In another embodiment, an antigen binding molecule of the invention comprises at least three CDRs from one or more antibody molecules. In another embodiment, an antigen binding molecule of the invention comprises at least four CDRs from one or more antibody molecules. In another embodiment, an antigen binding molecule of the invention comprises at least five CDRs from one or more antibody molecules. In another embodiment, an antigen binding molecule of the invention comprises at least six CDRs from one or more antibody molecules. Exemplary antibody molecules comprising at least one CDR that can be included in the subject antigen binding molecules are known in the art and exemplary molecules are described herein.

Antibodies or immunospecific fragments thereof for use in the methods of the invention include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a V_{L} or V_{H} domain, fragments produced by a Fab expression library, and anti-idiotypic (anti-Id) antibodies (including, e.g., anti-Id antibodies to binding molecules disclosed herein). ScFv molecules are known in the art and are described, e.g., in U.S. Pat. No. 5,892,019. Immunoglobulin or antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Antibody fragments, including single-chain antibodies, may comprise the variable region(s) alone or in combination with the entirety or a portion of the following: hinge region, C_{H}1, C_{H}2, and C_{H}3 domains. Also included in the invention are antigen-binding fragments also comprising any combination of variable region(s) with a hinge region, C_{H}1, C_{H}2, and C_{H}3 domains. Antibodies or immunospecific fragments thereof for use in the diagnostic and therapeutic methods disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

As used herein, the term “heavy chain portion” includes amino acid sequences derived from an immunoglobulin heavy chain. A polypeptide comprising a heavy chain portion comprises at least one of: a C_{H}1 domain, a hinge (e.g., upper, middle, and/or lower hinge region) domain, a C_{H}2 domain, a C_{H}3 domain, or a variant or fragment thereof. For example, a binding polypeptide for use in the invention may comprise a polypeptide chain comprising a C_{H}1 domain; a polypeptide chain comprising a C_{H}1 domain, at least a portion of a hinge domain, and a C_{H}2 domain; a polypeptide chain comprising a C_{H}1 domain and a C_{H}3 domain; a polypeptide chain comprising a C_{H}1 domain, at least a portion of a hinge domain, and a C_{H}3 domain, or a polypeptide chain comprising a C_{H}1 domain, at least a portion of a hinge domain, a C_{H}2 domain, and a C_{H}3 domain. In another embodiment, a polypeptide of the invention comprises a polypeptide chain comprising a C_{H}3 domain. Further, a binding polypeptide for use in the invention may lack at least a portion of a C_{H}2 domain (e.g., all or part of a C_{H}2 domain). As set forth above, it will be understood by one of ordinary skill in the art that these domains (e.g., the heavy chain portions) may be modified such that they vary in amino acid sequence from the naturally occurring immunoglobulin molecule.

In certain binding molecules, e.g., binding polypeptides, e.g., lung, colon, pancreatic and/or ovarian cancer tumor-associated polypeptide-specific antibodies or immunospecific fragments thereof for use in the diagnostic and treatment methods disclosed herein, the heavy chain portions of one polypeptide chain of a multimer are identical to those on a second polypeptide chain of the multimer. Alternatively, heavy chain portion-containing monomers for use in the methods of the invention are not identical. For example, each monomer may comprise a different target binding site, forming, for example, a bispecific antibody.

The heavy chain portions of a binding polypeptide for use in the diagnostic and treatment methods disclosed herein may be derived from different immunoglobulin molecules. For example, a heavy chain portion of a polypeptide may comprise a C_{H}1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. Preferably, the light chain portion comprises at least one of a V_{L} or C_{L} domain.

An isolated nucleic acid molecule encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Alternatively, in another embodiment, mutations may be introduced randomly along all or part of the immunoglobulin coding sequence, such as by saturation mutagenesis, and the resultant mutants can be incorporated into binding molecules for use in the diagnostic and treatment methods disclosed herein and screened for their ability to bind to the desired antigen, e.g., lung, colon, pancreatic and/or ovarian cancer tumor-associated polypeptides and variants of fragments thereof.

Antibodies or fragment thereof for use in the diagnostic and therapeutic methods disclosed herein may be described or specified in terms of the epitope(s) or portion(s) of a target polypeptide that they recognize or specifically bind. The portion of an antigen which specifically interacts with the antigen binding domain of an antibody is an “epitope,” or an “antigenic determinant.” An antigen may comprise a single epitope, but typically, an antigen comprises at least two epitopes, and can include any number of epitopes, depending on the size, conformation, and type of antigen. Antigens are typically peptides or polypeptides, but can be any molecule or compound or a combination of molecules or compounds. For example, an organic compound, e.g., dinitrophenol or DNP, a nucleic acid, a carbohydrate, or a mixture of any of these compounds either with or without a peptide or polypeptide can be a suitable antigen. Thus, for example, an “epitope” on a polypeptide may include a carbohydrate side chain.

The minimum size of a peptide or polypeptide epitope is thought to be about four to five amino acids. Peptide or polypeptide epitopes preferably contain at least seven, more preferably at least nine and most preferably between at least about 15 to about 30 amino acids. Since a CDR can recognize an antigenic peptide or polypeptide in its tertiary form, the amino acids comprising an epitope need not be contiguous, and in some cases, may not even be on the same peptide chain. In the present invention, peptide or polypeptide antigens preferably contain a sequence of at least 4, at least 5, at least 6, at least 7, more preferably at least 8, at least 9, at least 10, at least 15, at least 20, at least 25, and, most preferably, between about 15 to about 30 amino acids. Preferred peptides or polypeptides comprising, or alternatively consisting of, antigenic epitopes are at least 10, 15, 20, 25, 30, 35, 40, 45, 50, 55, 60, 65, 70, 75, 80, 85, 90, 95, or 100 amino acid residues in length.

By “specifically binds,” it is generally meant that an antibody binds to an epitope via its CDR, and that the binding entails some complementarity between the CDR and the epitope. According to this definition, an antibody is said to “specifically bind” to an epitope when it binds to that epitope, via its CDR more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

By way of non-limiting example, an antibody may be considered to bind a first epitope preferentially if it binds said first epitope with a dissociation constant (K_{D}) that is less than the antibody's K_{D} for the second epitope. In another non-limiting example, an antibody may be considered to bind a first antigen preferentially if it binds the first epitope with an affinity that is at least one order of magnitude less than the antibody's K_{D} for the second epitope. In another non-limiting example, an antibody may be considered to bind a first epitope preferentially if it binds the first epitope with an affinity that is at least two orders of magnitude less than the antibody's K_{D} for the second epitope.

In another non-limiting example, an antibody may be considered to bind a first epitope preferentially if it binds the first epitope with an off rate (k(off)) that is less than the antibody's k(off) for the second epitope. In another non-limiting example, an antibody may be considered to bind a first epitope preferentially if it binds the first epitope with an affinity that is at least one order of magnitude less than the antibody's k(off) for the second epitope. In another non-limiting example, an antibody may be considered to bind a first epitope preferentially if it binds the first epitope with an affinity that is at least two orders of magnitude less than the antibody's k(off) for the second epitope.

An antibody for use in the diagnostic and treatment methods disclosed herein may be said to bind a target polypeptide disclosed herein or a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. More preferably, an antibody of the invention may be said to bind a target polypeptide disclosed herein or a fragment or variant thereof with an off rate (k(off)) less than or equal to 5×10⁻⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹ or 10⁻⁷ sec⁻¹

An antibody or fragment thereof for use in the diagnostic and treatment methods disclosed herein may be said to bind a target polypeptide disclosed herein or a fragment or variant thereof with an on rate (k(on)) of greater than or equal to 10³ M−1 sec−1, 5×10³ M−1 sec−1, 10⁴ M−1 sec−1 or 5×10⁴ M−1 sec−1. More preferably, an antibody of the invention may be said to bind a target polypeptide disclosed herein or a fragment or variant thereof with an on rate (k(on)) greater than or equal to 10⁵ M−1 sec−1, 5×10⁵ M−1 sec−1, 10⁶ M−1 sec−1, or 5×10⁶M−1 sec−1 or 10⁷M−1 sec−1.

An antibody is said to competitively inhibit binding of a reference antibody to a given epitope if it preferentially binds to that epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

As used herein, the term “affinity” refers to a measure of the strength of the binding of an individual epitope with the CDR of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

Antibodies or immunospecific fragments thereof for use in the diagnostic and therapeutic methods disclosed herein may also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, may actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody may be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody may be deemed “highly specific” for a certain epitope, if it does not bind any other analog, ortholog, or homolog of that epitope.

Antibodies or immunospecific fragments thereof for use in the diagnostic and treatment methods disclosed herein may also be described or specified in terms of their binding affinity to a polypeptide of the invention. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Antibodies or immunospecific fragments thereof for use in the diagnostic and treatment methods disclosed herein may act as agonists or antagonists of target polypeptides described herein. For example, an antibody for use in the methods of the present invention may function as an antagonist, blocking or inhibiting the activity of the lung, colon, pancreatic and/or ovarian cancer tumor-associated polypeptide.

As used herein, the term “binding site” or “binding domain” refers to a region of a binding molecule, e.g., a binding polypeptide, e.g., an antibody or fragment thereof, which is responsible for specifically binding to a target molecule of interest (e.g., an antigen, ligand, receptor, substrate or inhibitor) Exemplary binding domains include antibody variable domains, a receptor binding domain of a ligand, or a ligand binding domain of a receptor or an enzymatic domain. A binding domain on an antibody is referred to herein as an “antigen binding domain.”

A binding molecule, binding polypeptide, or antibody for use in the diagnostic and treatment methods disclosed herein may be “multispecific,” e.g., bispecific, trispecific or of greater multispecificity, meaning that it recognizes and binds to two or more different epitopes present on one or more different antigens (e.g., proteins) at the same time. Thus, whether a binding molecule is “monospecific” or “multispecific,” e.g., “bispecific,” refers to the number of different epitopes with which a binding polypeptide reacts. Multispecific antibodies may be specific for different epitopes of a target polypeptide described herein or may be specific for a target polypeptide as well as for a heterologous epitope, such as a heterologous polypeptide or solid support material.

As used herein the term “valency” refers to the number of potential binding domains, e.g., antigen binding domains, present in a binding molecule, binding polypeptide or antibody. Each binding domain specifically binds one epitope. When a binding molecule, binding polypeptide or antibody comprises more than one binding domain, each binding domain may specifically bind the same epitope, for an antibody with two binding domains, termed “bivalent monospecific,” or to different epitopes, for an antibody with two binding domains, termed “bivalent bispecific.” An antibody may also be bispecific and bivalent for each specificity (termed “bispecific tetravalent antibodies”). In another embodiment, tetravalent minibodies or domain deleted antibodies can be made.

Bispecific bivalent antibodies, and methods of making them, are described, for instance in U.S. Pat. Nos. 5,731,168; 5,807,706; 5,821,333; and U.S. Appl. Publ. Nos. 2003/020734 and 2002/0155537, the disclosures of all of which are incorporated by reference herein. Bispecific tetravalent antibodies, and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat E A et al. op. cit. The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system). Techniques for separating or preferentially synthesizing dimers which are linked via at least one interchain disulfide linkage from dimers which are not linked via at least one interchain disulfide linkage from a mixture comprising the two types of polypeptide dimer, and the use of such molecules in the preparation of antibody molecules, domain deleted antibody molecules (e.g., lacking all or part of a CH2 domain), minibodies, diabodies, fusion proteins, etc. are described in PCT/US2004/020945 (WO2005000899), the disclosure of which is incorporated by reference herein.

As used herein, the term “chimeric antibody” will be held to mean any antibody wherein the immunoreactive region or site is obtained or derived from a first species and the constant region (which may be intact, partial or modified in accordance with the instant invention) is obtained from a second species. In preferred embodiments the target binding region or site will be from a non-human source (e.g. mouse or primate) and the constant region is human.

As used herein, the term “engineered antibody” refers to an antibody in which the variable domain in either the heavy and light chain or both is altered by at least partial replacement of one or more CDRs from an antibody of known specificity and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. An engineered antibody in which one or more “donor” CDRs from a non-human antibody of known specificity is grafted into a human heavy or light chain framework region is referred to herein as a “humanized antibody.” It may not be necessary to replace all of the CDRs with the complete CDRs from the donor variable region to transfer the antigen binding capacity of one variable domain to another. Rather, it may only be necessary to transfer those residues that are necessary to maintain the activity of the target binding site. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein, the term “antibody” (Ab) or “monoclonal antibody” (Mab) is meant to include intact molecules as well as antibody fragments (such as, for example, Fab and F(ab′)₂ fragments) which are capable of specifically binding to protein. Fab and F(ab′)₂ fragments lack the Fc fragment of intact antibody, clear more rapidly from the circulation, and may have less non-specific tissue binding than an intact antibody. (Wahl et al., J. Nucl. Med. 24:316-325 (1983).) Antibodies of the present invention also include chimeric, single chain, and humanized antibodies.

As used herein, the term “domain-deleted antibodies” refers to antibodies, or immunoreactive fragments thereof, in which at least a fraction of, or the entire region of, one or more of the constant region domains has been deleted or otherwise altered so as to provide desired biochemical characteristics such as increased tumor localization or reduced serum half-life when compared with an antibody of approximately the same immunogenicity comprising a native or unaltered constant region.

As used herein the term “properly folded polypeptide” includes polypeptides (e.g., antigen binding molecules such as antibodies) in which all of the functional domains comprising the polypeptide are distinctly active. As used herein, the term “improperly folded polypeptide” includes polypeptides in which at least one of the functional domains of the polypeptide is not active. In one embodiment, a properly folded polypeptide comprises polypeptide chains linked by at least one disulfide bond and, conversely, an improperly folded polypeptide comprises polypeptide chains not linked by at least one disulfide bond.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product and the translation of such mRNA into polypeptide(s). If the final desired product is a biochemical, expression includes the creation of that biochemical and any precursors.

As used herein, the terms “treat” or “treatment” refer to both therapeutic treatment and prophylactic or preventative measures, wherein the object is to prevent or slow down (lessen) an undesired physiological change or disorder, such as the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; fields such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject.

As used herein, phrases such as “a subject that would benefit from administration of a binding molecule” and “an animal in need of treatment” includes subjects, such as mammalian subjects, that would benefit from administration of a binding molecule used, e.g., for detection of an antigen recognized by a binding molecule (e.g., for a diagnostic procedure) and/or from treatment, i.e., palliation or prevention of a disease such as cancer, with a binding molecule which specifically binds a given target protein. As described in more detail herein, the binding molecule can be used in unconjugated form or can be conjugated, e.g., to a drug, prodrug, or an isotope.

By “hyperproliferative disease or disorder” is meant all neoplastic cell growth and proliferation, whether malignant or benign, including all transformed cells and tissues and all cancerous cells and tissues. Hyperproliferative diseases or disorders include, but are not limited to, precancerous lesions, abnormal cell growths, benign tumors, malignant tumors, and “cancer.”

Additional examples of hyperproliferative diseases, disorders, and/or conditions include, but are not limited to neoplasms, whether benign or malignant, located in the: prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital tract.

Other hyperproliferative disorders include, but are not limited to: hypergammaglobulinemia, lymphoproliferative disorders, paraproteinemias, purpura, sarcoidosis, Sezary Syndrome, Waldenstron's macroglobulinemia, Gaucher's Disease, histiocytosis, and any other hyperproliferative disease, besides neoplasia, located in an organ system listed above.

As used herein, the terms “tumor” or “tumor tissue” refer to an abnormal mass of tissue that results from excessive cell division. A tumor or tumor tissue comprises “tumor cells” which are neoplastic cells with abnormal growth properties and no useful bodily function. Tumors, tumor tissue and tumor cells may be benign or malignant. A tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “malignancy” refers to a non-benign tumor or a cancer. As used herein, the term “cancer” connotes a type of hyperproliferative disease which includes a malignancy characterized by deregulated or uncontrolled cell growth. Examples of cancer include, but are not limited to, carcinoma, lymphoma, blastoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer including adenocarcinoma of the colon, squamous cell carcinoma of the colon, sarcoma, lymphoma, melanoma, carcinoid of the colon, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Mycloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

The method of the present invention may be used to treat premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W. B. Saunders Co., Philadelphia, pp. 68-79 (1976)

Hyperplasia is a form of controlled cell proliferation, involving an increase in cell number in a tissue or organ, without significant alteration in structure or function. Hyperplastic disorders which can be treated by the method of the invention include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

Metaplasia is a form of controlled cell growth in which one type of adult or fully differentiated cell substitutes for another type of adult cell. Metaplastic disorders which can be treated by the method of the invention include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be treated by the method of the invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be treated by the method of the invention include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

In preferred embodiments, the method of the invention is used to inhibit growth, progression, and/or metastasis of cancers, in particular those listed above.

Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, menangioma, melanoma, neuroblastoma, and retinoblastoma.

The present invention is predicated, at least in part, on the discovery of novel methods for identifying and employing nucleotides that encode membrane associated molecules, i.e., nucleic acid molecules which encode transmembrane proteins or that encode molecules with membrane associated motifs. As will be set forth in more detail below, the present invention further comprises methods of using these identified nucleic acid molecules to generate custom arrays to identify markers associated with various diseases and disorders, e.g., lung, colon, pancreatic and ovarian cancer as well as autoimmune diseases or disorders. In a particularly preferred embodiment the instant invention comprises small molecules, ligands or immunoreactive species that bind to or otherwise associate with the identified membrane associated molecules. Those skilled in the art will appreciate that such moieties may advantageously be used in the prevention, diagnosis or treatment of diseases or disorders presenting the disclosed membrane associated molecules. The invention further relates to various methods, reagents and kits for diagnosing, staging, prognosing, preventing, monitoring and treating hyperproliferative diseases or disorders such as cancer or autoimmune diseases or disorders.

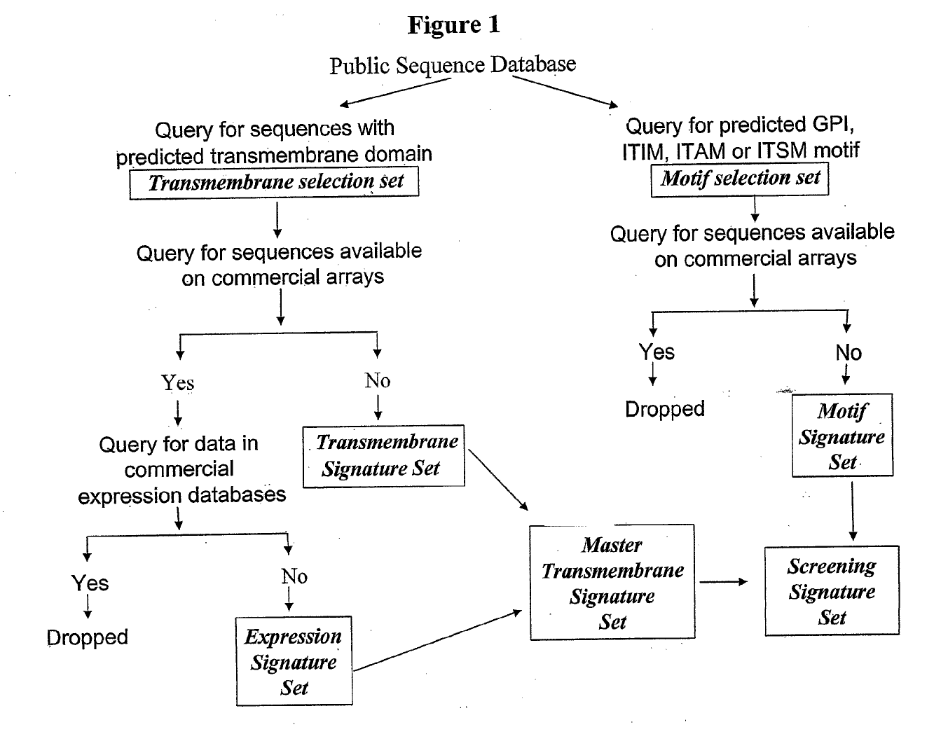

As will be explained in some detail, the present invention provides novel methods of culling information to identify and categorize membrane associated molecules (and their encoding nucleotides) that exhibit altered expression profiles associated with selected diseases or disorders. FIG. 1 provides an illustrative diagram showing preferred methods for associating a plurality of nucleotides encoding membrane associated molecules wherein such nucleotides may be employed to produce an array in accordance with the teachings herein.

Referring now to FIG. 1, a transmembrane selection set of molecules is produced using a publicly available sequence database (e.g., the Ensemble™ database), through the application of a model for membrane protein topology prediction, for example the Transmembrane hidden Markov model (TMHMM) (Krogh et al., (2001) J. Mol. Biol. 305, 567-580); incorporated herein by reference). Those skilled in the art will appreciate that the resulting transmembrane selection set comprises a plurality of membrane associated molecules. Sequences represented by the molecules in the transmembrane selection set are then screened against or compared to sequences set forth on one or more commercially available arrays e.g., an Affymetrix™ array. Those sequences from the transmembrane selection set that exhibit substantial homology to molecules set forth on the array or arrays are then excluded to provide a further subset or association of a plurality of molecules termed, for the purposes of this application, a transmembrane signature set. For the elimination of confusion, molecular sequences or homologs thereof found on commercially available arrays are referred to herein as “array associated molecules.” Those skilled in the art will appreciate that all or some of the plurality of molecules comprising the transmembrane signature set, their complement or hybridizing fragments thereof, may be associated with a substrate using well known techniques to provide an array in accordance with the teachings herein.

In another aspect of the present invention, those molecules excluded from the transmembrane selection set as set forth immediately above may be compared to or screened against sequences comprising a publicly available expression database (e.g., the Genelogic™ expression database). For such a comparison, those previously excluded sequences which do not have substantial intensity values with respect to sequences set forth in the expression database may be associated to provide a further subset of molecules termed, in this application, an expression signature set. Those skilled in the art will appreciate that the plurality of molecules comprising the expression signature set, their complement or hybridizing fragments thereof, may be associated with a substrate using well known techniques to provide an array in accordance with the teachings herein. In particularly preferred embodiments the plurality of molecules comprising the expression signature set will be combined with the plurality of molecules comprising the transmembrane signature set to provide another association of molecules termed the master transmembrane signature set. It will be appreciated that some or all of the molecules comprising this master transmembrane signature set may be used to generate an array in accordance with the present invention.

In yet another aspect, the publicly available database may be screened or interrogated for the presence of sequences exhibiting a GPI link, ITIM, ITAM, ITSM motif to yield a motif selection set. The skilled artisan will appreciate that such protein motifs are typically found in and, accordingly are representative of, membrane associated molecules. The sequences of molecules comprising the motif selection set are then compared to, or screened against, the sequences of molecules, or their homologs, set forth on one or more commercially available arrays (array associated molecules). Those molecular sequences of the motif selection set that exhibit substantial homology with one or more molecules found on the commercially available arrays are excluded, thereby yielding yet a further subset of molecules referred to herein as a motif signature set. Once again, those skilled in the art will appreciate that all or some of the plurality of molecules comprising the motif signature set, their complement or hybridizing fragments thereof, may be associated with a substrate using well known techniques to provide an array in accordance with the teachings herein. In a particularly preferred embodiment, and as set forth in the examples below, some or all of the molecules of the motif signature set may be combined with some or all of the molecules of the master transmembrane signature set to provide an association of molecules termed, for the purposes of this application, a screening signature set. It will be appreciated that some or all of the molecules comprising this screening signature set, their complement or hybridizing fragments thereof, may be used to generate an array in accordance with the present invention.

As indicated above, the methods of the present invention may use all or part of the previously identified sets of molecules, i.e., the transmembrane signature set, expression signature set, motif signature set, master transmembrane signature set and/or screening signature set, to generate one or more custom arrays. These custom arrays may be used for, among other things, the screening for potential markers or therapeutic targets associated with various hyperproliferative diseases or disorders including cancer and autoimmune diseases. The invention further relates to various methods, reagents and kits for diagnosing, staging, prognosing, monitoring and treating hyperproliferative diseases or disorders such as cancer, e.g., lung, colon, pancreatic and ovarian cancer and autoimmune diseases.

### SELECTED EMBODIMENTS

The present invention is based, in part, on membrane associated markers which have altered expression in a sample from a subject afflicted with a hyperproliferative disease or disorder, such as cancer, e.g., lung, colon, pancreatic and ovarian cancer or an autoimmune disease, as compared to their expression in a normal sample (i.e. non-diseased) cells. The enhanced expression of one or more of these markers in a sample from a subject afflicted with a hyperproliferative disease or disorder is herein correlated with a hyperproliferative disease or disorder such as cancer e.g., lung, colon, pancreatic and ovarian cancer or an autoimmune disease. The invention provides compositions, kits, and methods for assessing a hyperproliferative disease or disorder in a sample (e.g. cells obtained from a human, cultured human cells, archived or preserved human cells and in vivo cells) as well as treating patients afflicted with a hyperproliferative disease or disorder such as cancer, e.g., lung, colon, pancreatic and ovarian cancer or an autoimmune disease.

The compositions, kits, and methods of the invention have the following uses, among others:

1) assessing whether a patient is afflicted with a hyperproliferative disease or disorder;

2) assessing the stage of a hyperproliferative disease or disorder, such as cancer, in a human patient;

3) assessing the grade of a hyperproliferative disease or disorder, such as cancer, in a patient;

4) assessing the benign or malignant nature of a hyperproliferative disease or disorder, such as cancer, in a patient;

5) assessing the metastatic potential of a hyperproliferative disease or disorder, such as cancer, in a patient;

6) assessing the histological type of neoplasm associated with a hyperproliferative disease or disorder, such as cancer, in a patient;

7) making antibodies, antibody fragments or antibody derivatives that are useful for treating a hyperproliferative disease or disorder and/or assessing whether a patient is afflicted with a hyperproliferative disease or disorder;

8) assessing the presence of a hyperproliferative disease or disorder in a sample;

9) assessing the efficacy of one or more test compounds for inhibiting a hyperproliferative disease or disorder in a patient;

10) assessing the efficacy of a therapy for inhibiting a hyperproliferative disease or disorder in a patient;

11) monitoring the progression of a hyperproliferative disease or disorder in a patient;

12) selecting a composition or therapy for inhibiting a hyperproliferative disease or disorder in a patient;

13) treating a patient afflicted with a hyperproliferative disease or disorder;

14) inhibiting a hyperproliferative disease or disorder in a patient;

15) assessing the test compound for treating or preventing a hyperproliferative disease or disorder, such as cancer;

16) preventing the onset of a hyperproliferative disease or disorder in a patient at risk for developing a hyperproliferative disease or disorder;

17) assessing siRNA molecules for treating or preventing a hyperproliferative disease or disorder, such as cancer; and

18) preventing the onset of a hyperproliferative disease or disorder in a patient at risk for developing a hyperproliferative disease or disorder.

The invention thus includes a method of diagnosing whether a patient is afflicted with a hyperproliferative disease or disorder. This method comprises comparing the level of expression of a marker of the invention (for example, a marker associated with lung, colon, pancreatic and ovarian cancer as listed in Tables 7-10, respectively) in a patient sample and the normal level of expression of the marker in a control, e.g., a non-diseased sample. A significantly higher level of expression of the marker in the patient sample as compared to the normal level is an indication that the patient is afflicted with a hyperproliferative disease or disorder such as cancer e.g., lung, colon, pancreatic and ovarian cancer.

As described herein, a hyperproliferative disease or disorder in a patient is associated with an altered level of expression of one or more markers of the invention. While, as discussed above, some of these changes in expression level result from occurrence of the hyperproliferative disease or disorder, others of these changes induce, maintain, and promote cell growth from a subject associated with a hyperproliferative disease or disorder. Thus, a hyperproliferative disease or disorder characterized by an increase in the level of expression of one or more markers of the invention can be inhibited by reducing and/or interfering with the expression of the markers and/or function of the proteins encoded by those markers. Expression of a marker of the invention can be inhibited in a number of ways generally known in the art. For example, an antisense oligonucleotide can be provided to the cells from a subject associated with a hyperproliferative disease or disorder in order to inhibit transcription, translation, or both, of the marker(s). Alternately, a polynucleotide encoding an antibody, an antibody derivative, or an antibody fragment which specifically binds a marker protein, and operably linked with an appropriate promoter/regulator region, can be provided to the cell in order to generate intracellular antibodies which will inhibit the function or activity of the protein. The expression and/or function of a marker may also be inhibited by treating the cell associated with a hyperproliferative disease or disorder with an antibody, antibody derivative or antibody fragment that specifically binds a marker protein. Using the methods described herein, a variety of molecules, particularly including molecules sufficiently small that they are able to cross the cell membrane, can be screened in order to identify molecules which inhibit expression of a marker or inhibit the function of a marker protein. The compound so identified can be provided to the patient in order to inhibit a hyperproliferative disease in the patient.

Any marker or combination of markers of the invention, as well as any known markers in combination with the markers of the invention, may be used in the compositions, kits, and methods of the present invention. In general, it is preferable to use markers for which the difference between the level of expression of the marker in a sample from a subject associated with a hyperproliferative disease or disorder and the level of expression of the same marker in a normal sample, is as great as possible. Although this difference can be as small as the limit of detection of the method for assessing expression of the marker, it is preferred that the difference be at least greater than the standard error of the assessment method, and preferably a difference of at least 2-, 3-, 4-, 5-, 6-, 7-, 8-, 9-, 10-, 15-, 20-, 25-, 100-, 500-, 1000-fold or greater than the level of expression of the same marker in a normal sample.

It will be appreciated that patient samples or “target samples” may be used in the methods of the present invention. In these embodiments, the level of expression of the marker can be assessed by assessing the amount (e.g., absolute amount or concentration) of the marker in a cell sample. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample.

Expression of a marker of the invention may be assessed by any of a wide variety of well known methods for detecting expression of a transcribed nucleic acid or protein. Non-limiting examples of such methods include immunological methods for detection of cell-surface, nuclear proteins, protein purification methods, protein function or activity assays, nucleic acid hybridization methods e.g., using the arrays of the invention, nucleic acid reverse transcription methods, and nucleic acid amplification methods.

In one embodiment, expression of a marker is assessed using an antibody (e.g. a radio-labeled, chromophore-labeled, fluorophore-labeled, or enzyme-labeled antibody), an antibody derivative (e.g. an antibody conjugated with a substrate or with the protein or ligand of a protein-ligand pair {e.g. biotin-streptavidin), or an antibody fragment (e.g. a single-chain antibody, an isolated antibody hypervariable domain, etc.) which binds specifically with a marker protein or fragment thereof, including a marker protein which has undergone all or a portion of its normal post-translational modification.

In another embodiment, expression of a marker is assessed by preparing mRNA/cDNA (i.e. a transcribed polynucleotide) from cells in a patient sample, and by hybridizing the mRNA/cDNA with a reference polynucleotide which is a complement of a marker nucleic acid, or a fragment thereof. cDNA can, optionally, be amplified using any of a variety of polymerase chain reaction or in vitro transcription methods prior to hybridization with the reference polynucleotide; preferably, it is not amplified. Expression of one or more markers can likewise be detected using quantitative PCR to assess the level of expression of the marker(s). Alternatively, any of the many known methods of detecting mutations or variants (e.g. single nucleotide polymorphisms, deletions, etc.) of a marker of the invention may be used to detect occurrence of a marker in a patient.

In a related embodiment, a mixture of transcribed polynucleotides obtained from the sample is contacted with a substrate, having fixed thereto a polynucleotide complementary to or homologous with at least a portion (e.g. at least 7, 10, 15, 20, 25, 30, 40, 50, 100, 500, or more nucleotide residues) of a marker nucleic acid e.g., an array as described herein. If polynucleotides complementary to or homologous with are differentially detectable on the substrate (e.g. detectable using different chromophores or fluorophores, or fixed to different selected positions), then the levels of expression of a plurality of markers can be assessed simultaneously using a single substrate (e.g. a “gene chip” array of polynucleotides fixed at selected positions). When a method of assessing marker expression is used which involves hybridization of one nucleic acid with another, it is preferred that the hybridization be performed under stringent hybridization conditions.

Because the compositions, kits, and methods of the invention rely on detection of a difference in expression levels of one or more markers of the invention, it is preferable that the level of expression of the marker is significantly greater than the minimum detection limit of the method used to assess expression in at least one of normal cells and diseased cells.

When the compositions, kits, and methods of the invention are used for characterizing one or more of the stage, grade, histological type, and benign/malignant nature of a cancer, e.g., lung, colon, pancreatic and ovarian cancer, in a patient, it is preferred that the marker or panel of markers of the invention is selected such that a positive result is obtained in at least about 20%, and preferably at least about 40%, 60%, or 80%, and more preferably in substantially all patients afflicted with a cancer, e.g., lung, colon, pancreatic and ovarian cancer, of the corresponding stage, grade, histological type, or benign/malignant nature. Preferably, the marker or panel of markers of the invention is selected such that a positive predictive value (PPV) of greater than about 10% is obtained for the general population (more preferably coupled with an assay specificity greater than 80%).

When a plurality of markers of the invention are used in the compositions, kits, and methods of the invention, the level of expression of each marker in a patient sample can be compared with the normal level of expression of each of the plurality of markers in a non-diseased sample of the same type, either in a single reaction mixture (i.e. using reagents, such as different fluorescent probes, for each marker) or in individual reaction mixtures corresponding to one or more of the markers. In one embodiment, a significantly increased level of expression of more than one of the plurality of markers in the sample, relative to the corresponding normal levels, is an indication that the patient is afflicted with a hyperproliferative disease or disorder. When a plurality of markers is used, 2, 3, 4, 5, 8, 10, 12, 15, 20, 30, or 50 or more individual markers may be used, wherein fewer markers may also be used. In another embodiment, a plurality of markers may include 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the transmembrane signature set, screening signature set, motif signature set or the master transmembrane signature set.

In order to maximize the sensitivity of the compositions, kits, and methods of the invention it is preferable that the marker of the invention used therein be a marker which has a restricted tissue distribution.

Markers previously known to be associated with a hyperproliferative disease or disorder may be used together with one or more markers of the invention in a panel of markers, for example. It is well known that certain types of genes, such as oncogenes, tumor suppressor genes, growth factor-like genes, protease-like genes, and protein kinase-like genes are often involved with development of cancers of various types. Thus, among the markers of the invention which are associated with cancer, use of those which correspond to proteins which resemble known proteins encoded by known oncogenes and tumor suppressor genes are preferred.

It is recognized that the compositions, kits, and methods of the invention will be of particular utility to patients having an enhanced risk of developing a hyperproliferative disease or disorder and their medical advisors. Patients recognized as having an enhanced risk of developing a hyperproliferative disease or disorder include, for example, patients having a familial history of a hyperproliferative disease or disorder, such as cancer e.g., lung, colon, pancreatic and ovarian cancer.

The level of expression of a marker in normal (i.e. non-diseased) human tissue can be assessed in a variety of ways. In one embodiment, this normal level of expression is assessed by assessing the level of expression of the marker in a portion of cells which appears to be non-diseased and by comparing this normal level of expression with the level of expression in a portion of the cells which is suspected of being associated with a hyperproliferative disease or disorder. Alternately, and particularly as further information becomes available as a result of routine performance of the methods described herein, population-average values for normal expression of the markers of the invention may be used. In other embodiments, the ‘normal’ level of expression of a marker may be determined by assessing expression of the marker in a patient sample obtained from a healthy patient, from a patient sample obtained from a patient before the suspected onset of a hyperproliferative disease or disorder, from archived patient samples, and the like.

The invention includes compositions, kits, and methods for assessing the presence of cells from a subject associated with a hyperproliferative disease or disorder (e.g. an archived tissue sample or a sample obtained from a patient), using, for example, an array of the invention. These compositions, kits, and methods are substantially the same as those described above, except that, where necessary, the compositions, kits, and methods are adapted for use with samples other than patient samples. For example, when the sample to be used is a parafinized, archived human tissue sample, it can be necessary to adjust the ratio of compounds in the compositions of the invention, in the kits of the invention, or the methods used to assess levels of marker expression in the sample. Such methods are well known in the art and within the skill of the ordinary artisan.

The invention includes a kit for assessing the presence of cells from a subject associated with a hyperproliferative disease or disorder as (e.g. in a sample such as a patient sample). The kit comprises a plurality of reagents, each of which is capable of binding specifically with a marker nucleic acid or protein. Suitable reagents for binding with a marker protein include antibodies, antibody derivatives, antibody fragments, and the like. Suitable reagents for binding with a marker nucleic acid (e.g. a genomic DNA, an mRNA, a spliced mRNA, a cDNA, or the like) include complementary nucleic acid molecules. For example, the nucleic acid reagents may include oligonucleotides (labeled or non-labeled) fixed to a substrate, labeled oligonucleotides not bound with a substrate, pairs of PCR primers, molecular beacon probes, and the like.

The kit of the invention may optionally comprise additional components useful for performing the methods of the invention. By way of example, the kit may comprise fluids (e.g. SSC buffer) suitable for annealing complementary nucleic acid molecules or for binding an antibody with a protein with which it specifically binds, one or more sample compartments, an instructional material which describes performance of a method of the invention, a sample of normal cells, a sample of diseased cells, and the like.

The invention also includes a method of assessing the efficacy of a test compound for inhibiting a disease or disorder associated with a membrane molecule, using, for example, an array of the invention. As described above, differences in the level of expression of the markers of the invention correlate with cells from a subject associated with a hyperproliferative disease or disorder. Although it is recognized that changes in the levels of expression of certain of the markers of the invention likely result from the cells from a subject associated with a hyperproliferative disease or disorder, it is likewise recognized that changes in the levels of expression of other of the markers of the invention induce, maintain, and promote the diseased state of those cells. Thus, compounds which inhibit a hyperproliferative disease or disorder in a patient will cause the level of expression of one or more of the markers of the invention to change to a level nearer the normal level of expression for that marker (i.e. the level of expression for the marker in cells not associated with a hyperproliferative disease or disorder).

This method thus comprises comparing expression of a marker in a first sample and maintained in the presence of the test compound and expression of the marker in a second sample and maintained in the absence of the test compound. A significantly reduced expression of a marker of the invention in the presence of the test compound is an indication that the test compound inhibits a hyperproliferative disease or disorder. The samples may, for example, be aliquots of a single sample of normal cells obtained from a patient, pooled samples of normal cells obtained from a patient, cells of a normal cell line, aliquots of a single sample of cells from a subject associated with a hyperproliferative disease or disorder obtained from a patient, pooled samples of cells from a subject associated with a hyperproliferative disease or disorder obtained from a patient, cells of a cell line associated with a hyperproliferative disease or disorder, or the like. In one embodiment, the samples are cells obtained from a patient and a plurality of compounds known to be effective for inhibiting various hyperproliferative diseases or disorders are tested in order to identify the compound which is likely to best inhibit the hyperproliferative disease or disorder in the patient.

This method may likewise be used to assess the efficacy of a therapy for inhibiting a hyperproliferative disease or disorder in a patient. In this method, the level of expression of one or more markers of the invention in a pair of samples (one subjected to the therapy, the other not subjected to the therapy) is assessed. As with the method of assessing the efficacy of test compounds, if the therapy induces a significantly lower level of expression of a marker of the invention then the therapy is efficacious for inhibiting a hyperproliferative disease or disorder. As above, if samples from a selected patient are used in this method, then alternative therapies can be assessed in vitro in order to select a therapy most likely to be efficacious for inhibiting a hyperproliferative disease or disorder in the patient.

As described above, the disease state of human cells is correlated with changes in the levels of expression of the markers of the invention. The invention includes a method for assessing the human cell carcinogenic potential of a test compound, using, for example, an array of the invention. This method comprises maintaining separate aliquots of human cells from a subject associated with a hyperproliferative disease or disorder in the presence and absence of the test compound. Expression of a marker of the invention in each of the aliquots is compared. A significantly higher level of expression of a marker of the invention in the aliquot maintained in the presence of the test compound (relative to the aliquot maintained in the absence of the test compound) is an indication that the test compound possesses carcinogenic potential. The relative carcinogenic potentials of various test compounds can be assessed by comparing the degree of enhancement or inhibition of the level of expression of the relevant markers, by comparing the number of markers for which the level of expression is enhanced or inhibited, or by comparing both.

Various aspects of the invention are described in further detail in the following subsections.

### Nucleotide Sequences

The invention provides nucleotide sequences, e.g., nucleotide sequences as set forth in SEQ ID NO: 1-1146 (Table 6), SEQ ID NO: 3439-3445 (Table 5) and SEQ ID NO: 3453-3457 (Table 5) that encode one or more membrane proteins, a complement or fragment thereof.

One aspect of the invention pertains to isolated nucleic acid molecules or their complement, including nucleic acid molecules which encode a marker protein or a portion thereof. Isolated nucleic acid molecules of the invention also include nucleic acid molecules sufficient for use as hybridization probes to identify marker nucleic acid molecules, and fragments of marker nucleic acid molecules, e.g., those suitable for use as PCR primers for the amplification or mutation of marker nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. The terms “nucleic acid molecule,” “polynucleotide,” and “nucleotide sequence” can be used interchangeably herein.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR or in vitro transcription amplification techniques. The nucleic acid so amplified can be cloned into an appropriate vector and characterized by DNA sequence analysis. Furthermore, nucleotides corresponding to all or a portion of a nucleic acid molecule of the invention can be prepared by standard synthetic techniques, e.g., using an automated DNA synthesizer.

In another embodiment, an isolated nucleic acid molecule of the invention comprises a nucleic acid molecule which has a nucleotide sequence complementary to the nucleotide sequence of a marker nucleic acid or to the nucleotide sequence of a nucleic acid encoding a marker protein. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises a marker nucleic acid or which encodes a marker protein. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more marker proteins of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that differ, due to degeneracy of the genetic code, from the nucleotide sequence of nucleic acid molecules encoding a marker protein (e.g., a protein having one of the amino acid sequences set forth in Table 6), and thus encode the same protein.

It will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 7, 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a marker nucleic acid or to a nucleic acid encoding a marker protein.

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a variant marker protein that contain changes in amino acid residues that are not essential for activity. Such variant marker proteins differ in amino acid sequence from the naturally-occurring marker proteins, yet retain biological activity. In one embodiment, such a variant marker protein has an amino acid sequence that is at least about 40% identical, 50%, 60%, 70%, 80%, 90%, 95%, or 98% identical to the amino acid sequence of a marker protein.

An isolated nucleic acid molecule encoding a variant marker protein can be created by introducing one or more nucleotide substitutions, additions or deletions into the nucleotide sequence of marker nucleic acid molecules, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The present invention encompasses antisense nucleic acid molecules, i.e., molecules which are complementary to a sense nucleic acid of the invention, e.g., complementary to the coding strand of a double-stranded marker cDNA molecule or complementary to a marker mRNA sequence. Accordingly, an antisense nucleic acid of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a marker protein. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acid molecules, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-isopentenyladenine, 1-methylguanine, 1-methylinosine, 2,2-dimethylguanine, 2-methyladenine, 2-methylguanine, 3-methylcytosine, 5-methylcytosine, N-6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a marker protein to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al, 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a marker protein can be designed based upon the nucleotide sequence of a cDNA corresponding to the marker sequence. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The invention also encompasses nucleic acid molecules which form triple helical structures. For example, expression of a marker protein of the invention can be inhibited by targeting nucleotide sequences complementary to the regulatory region of the gene encoding the marker nucleic acid or protein (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon nucleic acid molecules having at least one region which is complementary to a nucleic acid of the invention, such that the molecular beacon is useful for quantitating the presence of the nucleic acid of the invention in a sample. A “molecular beacon” nucleic acid is a nucleic acid comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid are not annealed with one another, fluorescence of the fluorophore is quenched to a lesser degree. Molecular beacon nucleic acids are described, for example, in U.S. Pat. No. 5,876,930.

### Polynucleotides Encoding Membrane Associated Molecule-Specific Binding Molecules

The present invention also provides for nucleic acid molecules encoding membrane associated molecule-specific antibodies or other binding molecules (including molecules comprising, consisting essentially of, or consisting of, antibody fragments or variants thereof).

The polynucleotides may be produced or manufactured by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody or other binding molecule may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+RNA, isolated from, any tissue or cells expressing the antibody or other binding molecule, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody or other binding molecule. Amplified nucleic acids generated by PCR may then be cloned into replicable cloning vectors using any method well known in the art.

Once the nucleotide sequence and corresponding amino acid sequence of the antibody or other binding molecule is determined, its nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., Molecular Cloning, A Laboratory Manual, 2d Ed., Cold Spring Harbor Laboratory, Cold Spring Harbor, N.Y. (1990) and Ausubel et al., eds., Current Protocols in Molecular Biology, John Wiley & Sons, NY (1998), which are both incorporated by reference herein in their entireties), to generate antibodies having a different amino acid sequence, for example to create amino acid substitutions, deletions, and/or insertions.

A polynucleotide encoding a membrane associated molecule-specific antibody or other binding molecule can be composed of any polyribonucleotide or polydeoxyribonucleotide, which may be unmodified RNA or DNA or modified RNA or DNA. For example, a polynucleotide encoding a membrane associated molecule-specific antibody or other binding molecule can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide encoding a membrane associated molecule-specific antibody can be composed of triple-stranded regions comprising RNA or DNA or both RNA and DNA. A polynucleotide encoding a membrane associated molecule-specific antibody or other binding molecule may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

Such membrane associated molecule polynucleotides include the following polynucleotides (Table 5) and their respective nucleic acid sequences:

TABLE 5

| BI NO: | SEQ ID NO: | PROTEIN NAME |
|---|---|---|
| BI1053146 | 3439 | SDAD1 (NM_018115.1) |
| BI1053147 | 3440 | GPR101 (NM_054021) |
| BI1053148 | 3441 | |
| BI1053149 | 3442 | OFR4N4 (NM_001005241) |
| BI1053150 | 3443 | OFR2G3 (NM_001001914) |
| BI1053151 | 3444 | |
| BI1053152 | 3445 | OFR4S2 (NM_001004059) |
| BI1053153 | 3453 | MUC 4 a (NP_060876) |
| BI1053154 | 3454 | MUC 4 b (NP_612155) |
| BI1053155 | 3455 | MUC 4 c (NP_612156) |
| BI1053156 | 3456 | MUC 4 d (NP_004523) |
| BI1053157 | 3457 | MUC 4 e (NP_612154) |

SEQ ID NO: 3439

ATGTTTATGGCACAGATTAGTCACTGCTACCCAGAGTACCTAAGTA
ATTTTCCTCAAGAGGTGAAAGATCTTCTCTCCTGCAATCATACCGTA
TTGGATCCAGATCTGCGAATGACATTTTGCAAAGCTTTGATCTTGCT
GAGAAATAAGAATCTCATCAATCCATCAAGCCTGCTAGAACTCTTC
TTTGAACTTTTTCGTTGCCATGATAAACTTCTGCGAAAGACTTTATA
CACACATATTGTGACTGATATCAAGAATATAAATGCAAAACACAA
GAACAATAAAGTGAATGTAGTATTGCAAAATTTCATGTACACCATG
TTAAGAGATAGCAATGCAACCGCAGCCAAGATGTCTTTAGATGTAA
TGATTGAACTCTACAGAAGGAACATCTGGAATGATGCAAAAACTGT
CAATGTTATCACAACTGCATGTTTCTCTAAGGTCACCAAGATATTA
GTTGCCGCTTTGACATTCTTTCTTGGGAAAGATGAAGATGAAAAAC
AGGACAGTGACTCCGAATCTGAGGATGATGGACCAACAGCAAGAG
ACCTGCTAGTACAATATGCTACAGGGAAGAAAAGTTCCAAAAACA
AGAAAAAGTTGGAAAAGGCAATGAAAGTGCTCAAGAAACAAAAA
AAGAAGAAAAAACCAGAGGTGTTTAACTTTTCAGCCATTCACTTGA
TTCATGATCCCCAAGATTTTGCGGAAAAACTACTAAAGCAGCTTGA
GTGCTGTAAGGAGAGGTTTGAAGTGAAGATGATGCTCATGAACCTT
ATCTCCAGATTGGTGGGAATTCATGAGCTTTTCCTCTTCAATTTCTA
TCCCTTTTTGCAAAGGTTTCTGCAGCCCCACCAAAGAGAAGTAACC
AAGATCCTTCTGTTTGCTGCACAAGCATCTCATCACCTAGTACCCCC
AGAGATTATTCAATCATTGCTTATGACTGTGGCAAACAATTTTGTTA
CCGACAAGAACTCTGGAGAAGTCATGACAGTAGGAATCAATGCTA
TAAAGGAGATAACAGCTCGATGTCCTCTGGCCATGACTGAAGAACT
TCTCCAAGACCTGGCTCAGTATAAAACACACAAGGATAAGAATGT
AATGATGTCTGCTAGAACTTTGATTCACCTCTTCCGAACACTGAATC
CTCAGATGCTGCAGAAGAAATTCCGGGGTAAGCCTACAGAGGCCT
CCATAGAAGCAAGAGTACAAGAATATGGAGAATTAGATGCTAAAG
ATTACATTCCAGGAGCAGAAGTTCTGGAAGTTGAGAAAGAAGAGA
ATGCTGAAAATGATGAAGATGGATGGGAAAGTACCAGTCTCAGTG
AGGAGGAGGATGCTGATGGTGAATGGATTGATGTGCAACACTCTTC
CGATGAAGAACAGCAAGAAATCTCCAAGAAGCTGAACAGCATGCC
CATGGAGGAGCGGAAGGCCAAAGCTGCAGCCATCAGCACTAGCCG
AGTTTTAACTCAGGAAGACTTCCAGAAAATCCGCATGGCCCAAATG
AGAAAAGAACTTGATGCTGCCCCCGGGAAATCCCAGAAGAGGAAA
TACATTGAAATAGACAGTGATGAAGAGCCCAGGGGTGAATTACTTT
CTCTTCGGGACATTGAACGCCTTCATAAAAAGCCAAAGTCTGACAA
AGAGACAAGACTAGCAACTGCAATGGCTGGAAAGACAGACCGAAA
AGAATTTGTGAGGAAGAAAACCAAAACAAATCCATTTTCCAGTTCG
ACAAATAAAGAGAAGAAAAAACAGAAGAACTTTATGATGATGCGG
TATAGCCAGAATGTCCGGTCAAAAAATAAGCGTTCCTTCCGAGAAA
AACAGTTGGCACTACGAGATGCACTTTTGAAAAAGAGAAAAAGAA
TGAAGTAA

SEQ ID NO: 3440

ATGACGTCCA CCTGCACCAA CAGCACGCGC GAGAGTAACA
GCAGCCACAC GTGCATGCCC CTCTCCAAAA TGCCCATCAG
CCTGGCCCAC GGCATCATCCGCTCAACCGT GCTGGTTATC
TTCCTCGCCG CCTCTTTCGT CGGCAACATA GTGCTGGCGC
TAGTGTTGCA GCGCAAGCCG CAGCTGCTGC AGGTGACCAA
CCGTTTTATC TTTAACCTCC TCGTCACCGA CCTGCTGCAG
ATTTCGCTCG TGGCCCCCTG GGTGGTGGCC ACCTCTGTGC
CTCTCTTCTG GCCCCTCAAC AGCCACTTCT GCACGGCCCT
GGTTAGCCTC ACCCACCTGT TCGCCTTCGC CAGCGTCAAC
ACCATTGTCG TGGTGTCAGT GGATCGCTAC TTGTCCATCA
TCCACCCTCT CTCCTACCCG TCCAAGATGA CCCAGCGCCG
CGGTTACCTG CTCCTCTATG GCACCTGGAT TGTGGCCATC
CTGCAGAGCA CTCCTCCACT CTACGGCTGG GGCCAGGCTG
CCTTTGATGA GCGCAATGCT CTCTGCTCCA TGATCTGGGG
GGCCAGCCCC AGCTACACTA TTCTCAGCGT GGTGTCCTTC
ATCGTCATTC CACTGATTGT CATGATTGCC TGCTACTCCG
TGGTGTTCTG TGCAGCCCGG AGGCAGCATG CTCTGCTGTA
CAATGTCAAG AGACACAGCT TGGAAGTGCG AGTCAAGGAC
TGTGTGGAGA ATGAGGATGA AGAGGGAGCA GAGAAGAAGG
AGGAGTTCCA GGATGAGAGT GAGTTTCGCC GCCAGCATGA
AGGTGAGGTC AAGGCCAAGG AGGGCAGAAT GGAAGCCAAG
GACGGCAGCC TGAAGGCCAA GGAAGGAAGC ACGGGGACCA
GTGAGAGTAG TGTAGAGGCC AGGGGCAGCG AGGAGGTCAG
AGAGAGCAGC ACGGTGGCCA GCGACGGCAG CATGGAGGGT
AAGGAAGGCA GCACCAAAGT TGAGGAGAAC AGCATGAAGG
CAGACAAGGG TCGCACAGAG GTCAACCAGT GCAGCATTGA
CTTGGGTGAA GATGACATGG AGTTTGGTGA AGACGACATC
AATTTCAGTG AGGATGACGT CGAGGCAGTG AACATCCCGG
AGAGCCTCCC ACCCAGTCGT CGTAACAGCA ACAGCAACCC
TCCTCTGCCC AGGTGCTACC AGTGCAAAGC TGCTAAAGTG
ATCTTCATCA TCATTTTCTC CTATGTGCTA TCCCTGGGGC
CCTACTGCTT TTTAGCAGTC CTGGCCGTGT GGGTGGATGT
CGAAACCCAG GTACCCCAGT GGGTGATCAC CATAATCATC
TGGCTTTTCT TCCTGCAGTG CTGCATCCAC CCCTATGTCT
ATGGCTACAT GCACAAGACC ATTAAGAAGG AAATCCAGGA
CATGCTGAAG AAGTTCTTCT GCAAGGAAAA GCCCCCGAAA
GAAGATAGCC ACCCAGACCT GCCCGGAACA GAGGGTGGGA
CTGAAGGCAA GATTGTCCCT TCCTACGATT CTGCTACTTT TCCT

SEQ ID NO: 3441

ATGTTATCCCCCCACATTTACCTCCCAAAGTGCTGGAATTACAGAC
ATGAGCCACTGTGCCTGGCCGTGTGCTTTCATTTTCAGTTAAGTCTC
TGCTTTTGTTGCTTTATTCTTTCCCTGCTTTGTTTGTGTGTTTTGTTCA
ATTCTTTGTTCAAAACACCAAGAACTTGTACAGCCTCCACTGGTAA
CATATTTTGGCAAGCCAGCCAGGAGGTAATCCCAAAGTTTGGGGTT
TATTTTTCTTGTTTGTTTTTCCTGCTCCATACAGGGGAATCTCAGTCT
CTCTCTCTCTTTTCCTTTCCAACTTGGGACGGTTGGTGGGCAGCACC
TAAACACAGAGGCAACTGCAGGTTTCTGGCCGGGGCCACTCTGAA
GGACTCCTTTCTATCTTTTCCAGTTGTGGTCCCTGATCCCTACGTGT
GGCACAGCTTGGGGCGAGCTGGCATTTGTTTCAGTGACTTAAACCT
TGTTTTCTCATGC

SEQ ID NO: 3442

ATGAAGATAGCAAACAACACAGTAGTGACAGAATTTATCCTCCTTG
GTCTGACTCAGTCTCAAGATATTCAGCTCTTGGTCTTTGTGCTGATC
TTAATTTTCTACCTTATCATCCTCCCTGGAAATTTTCTCATTATTTTC
ACCATAAGGTCAGACCCTGGGCTCACAGCCCCCCTCTATTTCTTTCT
GGGCAACTTGGCCTTCCTGGATGCATCCTACTCCTTCATTGTGGCTC
CCAGGATGTTGGTGGACTTCTTCTCTGAGAAGAAGGTAATCTCCTA
CAGAGGCTGCATCACTCAGCTCTTTTTCTTGCACTTCCTTGGAGGAG
GGGAGGGATTACTCCTTGTTGTGATGGCCTTTGACCGCTACATCGC
CATCTGCCGGCCTCTGCACTGTTCAACTGTCATGAACCCTAGAGCC
TGCTATGCAATGATGTTGGCTCTGTGGCTTGGGGGTTTTGTCCACTC
CATTATCCAGGTGGTCCTCATCCTCCGCTTGCCTTTTTGTGGCCCAA
ACCAGCTGGACAACTTCTTCTGTGATGTCCGACAGGTCATCAAGCT
GGCTTGCACCGACATGTTTGTGGTGGAGCTTCTGATGGTCTTCAAC
AGTGGCCTGATGACACTCCTGTGCTTTCTGGGGCTTCTGGCTTCCTA
TGCAGTCATCCTCTGCCATGTTCGTAGGGCAGCTTCTGAAGGGAAG
AACAAGGCCATGTCCATGTGCACCACTCGTGTCATTATTATACTTCT
TATGTTTGGACCTGCTATCTTCATCTACATGTGCCCTTTCAGGGCCT
TACCAGCTGACAAGATGGTTTCTCTCTTTCACACAGTGATCTTTCCA
TTGATGAATCCTATGATTTATACCCTTCGCAACCAGGAAGTGAAAA
CTTCCATGAAGAGGTTATTGAGTCGACATGTAGTCTGTCAAGTGGA
TTTTATAATAAGAAACTGA

SEQ ID NO: 3443

ATGGGATTGGGCAATGAGAGTTCCCTAATGGATTTCATCCTTCTAG
GCTTCTCAGACCACCCTCGTCTGGAGGCTGTTCTCTTTGTATTTGTC
CTTTTCTTCTACCTCCTGACCCTTGTGGGAAACTTCACCATAATCAT
CATCTCATATCTGGATCCCCCTCTTCATACCCCAATGTACTTTTTTCT
CAGCAACCTCTCTTTACTGGACATCTGCTTCACTACTAGCCTTGCTC
CTCAGACCTTAGTTAACTTGCAAAGACCAAAGAAGACGATCACTTA
CGGTGGTTGTGTGGCGCAACTCTATATTTCTCTGGCACTGGGCTCCA
CTGAATGTATCCTCTTGGCTGACATGGCCTTGGATCGGTACATTGCT
GTCTGCAAACCCCTCCACTATGTAGTCATCATGAACCCACGGCTTT
GCCAACAGCTGGCATCTATCTCCTGGCTCAGTGGTTTGGCTAGTTCC
CTAATCCATGCAACTTTTACCTTGCAATTGCCTCTCTGTGGCAACCA
TAGGCTGGACCATTTTATTTGCGAAGTACCAGCTCTTCTCAAGTTGG
CTTGTGTGGACACCACTGTCAATGAATTGGTGCTTTTTGTTGTTAGT
GTTCTGTTTGTTGTCATTCCACCAGCACTCATCTCCATCTCCTATGG
CTTCATAACTCAAGCTGTGCTGAGGATCAAATCAGTAGAGGCAAGG
CACAAAGCCTTCAGCACCTGCTCCTCCCACCTTACAGTGGTGATTA
TATTCTATGGCACCATAATCTACGTGTACCTGCAACCTAGTGACAG
CTATGCCCAGGACCAAGGGAAGTTTATCTCCCTCTTCTACACCATG
GTGACCCCCACTTTAAATCCTATCATCTATACTTTAAGGAACAAGG
ATATGAAAGAGGCTCTGAGGAAACTTCTCTCGGGAAAATTGTGA

SEQ ID NO: 3444

ATGCTGGACCTGGAGAAGGAGAAGGACCTGTTCAGCAGGCAGAAG
GGCTACCTGGAAGAGGAGCTCGACTACCGGAAGCAAGCCCTTGAC
CAGGCTTACCTGAAAATCCAAGACCTGGAGGCCACACTGTACACA
GCGCTGCAGCAGGAGCCGGGGCGGAGGGCCGGTGAGGCGCTGAGC
GAGGGCCAGCGGGAGGACCTGCAGGCTGCTGTGGAAAAGGTGCGC
AGGCAGATCCTCAGGCAGAGCCGCGAGTTCGACAGCCAGATCCTG
CGGGAGCGCATGGAGCTGCTGCAGCAGGCCCAGCAGAGAATCCGA
GAACTGGAGGACAAACTGGAGTTTCAGAAGCGGCACCTGAAAGAA
CTGGAGGAAAAGTTTTTGTTCCTTTTTTTGTTTTTCTCACTAGCATTC
ATTCTGTGGCCTTGA

The open reading frame (ORE) for SEQ ID NO:3444 was predicted as follows. The human transcript for SEQ ID NO:3444 identified from Ensembl database (version 32) has the same 3′ end as a homologous Rat gene XM_{—}341232 but is lacking an ATG start codon at the 5′ end. Using the 5′ end of the rat sequence and its ATG start site as a model, a predicted exon structure was generated from the corresponding human genomic region. The resulting predicted human ORF has a single transmembrane domain.

SEQ ID NO: 3445

ATGGAAAAAATAAACAACGTAACTGAATTCATTTTCTGGGGTCTTT
CTCAGAGCCCAGAGATTGAGAAAGTTTGTTTTGTGGTGTTTTCTTTC
TTCTACATAATCATTCTTCTGGGAAATCTCCTCATCATGCTGACAGT
TTGCCTGAGCAACCTGTTTAAGTCACCCATGTATTTCTTTCTCAGCT
TCTTGTCTTTTGTGGACATTTGTTACTCTTCAGTCACAGCTCCCAAG
ATGATTGTTGACCTGTTAGCAAAGGACAAAACCATCTCCTATGTGG
GGTGCATGTTGCAACTGCTTGGAGTACATTTCTTTGGTTGCACTGAG
ATCTTCATCCTTACTGTAATGGCCTATGATCGTTATGTGGCTATCTG
TAAACCCCTACATTATATGACCATCATGAACCGGGAGACATGCAAT
AAAATGTTATTAGGGACGTGGGTAGGTGGGTTCTTACACTCCATTA
TCCAAGTGGCTCTGGTAGTCCAACTACCCTTTTGTGGACCCAATGA
GATAGATCACTACTTTTGTGATGTTCACCCTGTGTTGAAACTTGCCT
GCACAGAAACATACATTGTTGGTGTTGTTGTGACAGCCAACAGTGG
TACCATTGCTCTGGGGAGTTTTGTTATCTTGCTAATCTCCTACAGCA
TCATCCTAGTTTCCCTGAGAAAGCAGTCAGCAGAAGGCAGGCGCA
AAGCCCTCTCCACCTGTGGCTCCCACATTGCCATGGTCGTTATCTTT
TTCGGCCCCTGTACTTTTATGTACATGCGCCCTGATACGACCTTTTC
AGAGGATAAGATGGTGGCTGTATTTTACACCATTATCACTCCCATG
TTAAATCCTCTGATTTATACACTGAGAAATGCAGAAGTAAAGAATG
CAATGAAGAAACTGTGGGGCAGAAATGTTTTCTTGGAGGCTAAAG
GGAAATAG
SEQ ID NO: 3453
TACAGCCCCAAGGTCGCTCCCTCTGGGGCCCTTTCTTCCCCATTCTT
CCCAGCAGCCCAAAGCTCTGGTGGGACAGGGGCAGCCCCTGGGGA
GGGAGGAGAGGACCCAGGAACCCGGCTAGGAGGGTGGCCCACCCA
TTTCCAGTGTGACCTGTTCCCATTCCCCCATGTCTCCTCCCATCCCT
CCCGCCACTCAGCTCAGGCTGATGAGAAGCAGAGCAACGGGTGTA
TCGGTGTTTTCTTTCCTGGTGGGGTAGTGGGGTGGGGCTGAGGAGA
GAAAAGGGTGATTAGCGTGGGGCCCCGCCCTCTTTTGTCCTCTTCC
CAGGTTCCCTGGCCCCTTCGGAGAAACGCACTTGGTTCGGGCCAGC
CGCCTGAGGGGACGGGCTCACGTCTGCTCCTCACACTGCAGCTGCT
GGGCCGTGGAGCTTCCCCAGGGAGCCAGGGGGACTTTTGCCGCAG
CCATGAAGGGGGCACGCTGGAGGAGGGTCCCCTGGGTGTCCCTGA
GCTGCCTGTGTCTCTGCCTCCTTCCGCATGTGGTCCCAGGAACCACA
GAGGACACATTAATAACTGGAAGTAAAACTCCTGCCCCAGTCACCT
CAACAGGCTCAACAACAGCGACACTAGAGGGACAATCAACTGCAG
CTTCTTCAAGGACCTCTAATCAGGACATATCAGCTTCATCTCAGAA
CCACCAGACTAAGAGCACGGAGACCACCAGCAAAGCTCAAACCGA
CACCCTCACGCAGATGATGACATCAACTCTTTTTTCTTCCCCAAGTG
TACACAATGTGATGGAGACTGTTACGCAGGAGACAGCTCCTCCAGA
TGAAATGACCACATCATTTCCCTCCAGTGTCACCAACACACTCATG
ATGACATCAAAGACTATAACAATGACAACCTCCACAGACTCCACTC
TTGGAAACACAGAAGAGACATCAACAGCAGGAACTGAAAGTTCTA
CCCCAGTGACCTCAGCAGTCTCAATAACAGCTGGACAGGAAGGAC
AATCACGAACAACTTCCTGGAGGACCTCTATCCAAGACACATCAGC
TTCTTCTCAGAACCACTGGACTCGGAGCACGCAGACCACCAGGGAA
TCTCAAACCAGCACCCTAACACACAGAACCACTTCAACTCCTTCTT
TCTCTCCAAGTGTACACAATGTGACAGGGACTGTTTCTCAGAAGAC
ATCTCCTTCAGGTGAAACAGCTACCTCATCCCTCTGTAGTGTCACA
AACACATCCATGATGACATCAGAGAAGATAACAGTGACAACCTCC
ACAGGCTCCACTCTTGGAAACCCAGGGGAGACATCATCAGTACCTG
TTACTGGAAGTCTTATGCCAGTCACCTCAGCAGCCTTAGTAACAGT
TGATCCAGAAGGACAATCACCAGCAACTTTCTCAAGGACTTCTACT
CAGGACACAACAGCTTTTTCTAAGAACCACCAGACTCAGAGCGTGG
AGACCACCAGAGTATCTCAAATCAACACCCTCAACACCCTCACACC
GGTTACAACATCAACTGTTTTATCCTCACCAAGTGGATTCAACCCA
AGTGGAACAGTTTCTCAGGAGACATTCCCTTCTGGTGAAACAACCA
TCTCATCCCCTTCCAGTGTCAGCAATACATTCCTGGTAACATCAAA
GGTGTTCAGAATGCCAATCTCCAGAGACTCTACTCTTGGAAACACA
GAGGAGACATCACTATCTGTAAGTGGAACCATTTCTGCAATCACTT
CCAAAGTTTCAACCATATGGTGGTCAGACACTCTGTCAACAGCACT
CTCCCCCAGTTCTCTACCTCCAAAAATATCCACAGCTTTCCACACCC
AGCAGAGTGAAGGTGCAGAGACCACAGGACGGCCTCATGAGAGGA
GCTCATTCTCTCCAGGTGTGTCTCAAGAAATATTTACTCTACATGAA
ACAACAACATGGCCTTCCTCATTCTCCAGCAAAGGCCACACAACTT
GGTCACAAACAGAACTGCCCTCAACATCAACAGGTGCTGCCACTAG
GCTTGTCACAGGAAATCCATCTACAGGGGCAGCTGGCACTATTCCA
AGGGTCCCCTCTAAGGTCTCAGCAATAGGGGAACCAGGAGAGCCC
ACCACATACTCCTCCCACAGCACAACTCTCCCAAAAACAACAGGGG
CAGGCGCCCAGACACAATGGACACAAGAAACGGGGACCACTGGAG
AGGCTCTTCTCAGCAGCCCAAGCTACAGTGTGACTCAGATGATAAA
AACGGCCACATCCCCATCTTCTTCACCTATGCTGGATAGACACACA
TCACAACAAATTACAACGGCACCATCAACAAATCATTCAACAATAC
ATTCCACAAGCACCTCTCCTCAGGAATCACCAGCTGTTTCCCAAAG
GGGTCACACTCAAGCCCCGCAGACCACACAAGAATCACAAACCAC
GAGGTCCGTCTCCCCCATGACTGACACCAAGACAGTCACCACCCCA
GGTTCTTCCTTCACAGCCAGTGGGCACTCGCCCTCAGAAATTGTTCC
TCAGGACGCACCCACCATAAGTGCAGCAACAACCTTTGCCCCAGCT
CCCACCGGGGATGGTCACACAACCCAGGCCCCGACCACAGCACTG
CAGGCAGCACCCAGCAGCCATGATGCCACCCTGGGGCCCTCAGGA
GGCACGTCACTTTCCAAAACAGGTGCCCTTACTCTGGCCAACTCTG
TAGTGTCAACACCAGGGGGCCCAGAAGGACAATGGACATCAGCCT
CTGCCAGCACCTCACCTGACACAGCAGCAGCCATGACCCATACCCA
CCAGGCTGAGAGCACAGAGGCCTCTGGACAAACACAGACCAGCGA
ACCGGCCTCCTCAGGGTCACGAACCACCTCAGCGGGCACAGCTACC
CCTTCCTCATCCGGGGCGAGTGGCACAACACCTTCAGGAAGCGAAG
GAATATCCACCTCAGGAGAGACGACAAGGTTTTCATCAAACCCCTC
CAGGGACAGTCACACAACCCAGTCAACAACCGAATTGCTGTCCGCC
TCAGCCAGTCATGGTGCCATCCCAGTAAGCACAGGAATGGCGTCTT
CGATCGTCCCCGGCACCTTTCATCCCACCCTCTCTGAGGCCTCCACT
GCAGGGAGACCGACAGGACAGTCAAGCCCAACTTCTCCCAGTGCC
TCTCCTCAGGAGACAGCCGCCATTTCCCGGATGGCCCAGACTCAGA
GGACAAGAACCAGCAGAGGGTCTGACACTATCAGCCTGGCGTCCC
AGGCAACCGACACCTTCTCAACAGTCCCACCCACACCTCCATCGAT
CACATCCAGTGGGCTTACATCTCCACAAACCCAGACCCACACTCTG
TCACCTTCAGGGTCTGGTAAAACCTTCACCACGGCCCTCATCAGCA
ACGCCACCCCTCTTCCTGTCACCAGCACCTCCTCAGCCTCCACAGGT
CACGCCACCCCTCTTGCTGTCAGCAGTGCTACCTCAGCTTCCACAGT
ATCCTCGGACTCCCCTCTGAAGATGGAAACATCAGGAATGACAACA
CCGTCACTGAAGACAGACGGTGGGAGACGCACAGCCACATCACCA
CCCCCCACAACCTCCCAGACCATCATTTCCACCATTCCCAGCACTG
CCATGCACACCCGCTCCACAGCTGCCCCCATCCCCATCCTGCCTGA
GAGAGGAGTTTCCCTCTTCCCCTATGGGGCAGACGCCGGGGACCTG
GAGTTCGTCAGGAGGACCGTGGACTTCACCTCCCCACTCTTCAAGC
CGGCGACTGGCTTCCCCCTTGGCTCCTCTCTCCGTGATTCCCTCTAC
TTCACAGACAATGGCCAGATCATCTTCCCAGAGTCAGACTACCAGA
TTTTCTCCTACCCCAACCCACTCCCAACAGGCTTCACAGGCCGGGA
CCCTGTGGCCCTGGTGGCTCCGTTCTGGGACGATGCTGACTTCTCCA
CTGGTCGGGGGACCACATTTTATCAGGAATACGAGACGTTCTATGG
TGAACACAGCCTGCTAGTCCAGCAGGCCGAGTCTTGGATTAGAAAG
ATCACAAACAACGGGGGCTACAAGGCCAGGTGGGCCCTAAAGGTC
ACGTGGGTCAATGCCCACGCCTATCCTGCCCAGTGGACCCTCGGGA
GCAACACCTACCAAGCCATCCTCTCCACGGACGGGAGCAGGTCCTA
TGCCCTGTTTCTCTACCAGAGCGGTGGGATGCAGTGGGACGTGGCC
CAGCGCTCAGGCAAGCCGGTGCTCATGGGCTTCTCTAGTGGAGATG
GCTTTTTCGAAAACAGCCCACTGATGTCCCAGCCAGTGTGGGAGAG
GTATCGCCCTGATAGATTCCTGAATTCCAACTCAGGCCTCCAAGGG
CTGCAGTTCTACGGGCTACACCGGGAAGAAAGGCCCAACTACCGTC
TCGAGTGCCTGCAGTGGCTGAAGAGCCAGCCTCGGTGGCCCAGCTG
GGGCTGGAACCAGGTCTCCTGCCCTTGTTCCTGGCAGCAGGGACGA
CGGGACTTACGATTCCAACCCGTCAGCATAGGTCGCTGGGGCCTCG
GCAGTAGGCAGCTGTGCAGCTTCACCTCTTGGCGAGGAGGCGTGTG
CTGCAGCTACGGGCCCTGGGGAGAGTTTCGTGAAGGCTGGCACGTG
CAGCGTCCTTGGCAGTTGGCCCAGGAACTGGAGCCACAGAGCTGGT
GCTGCCGCTGGAATGACAAGCCCTACCTCTGTGCCCTGTACCAGCA
GAGGCGGCCCCACGTGGGCTGTGCTACATACAGGCCCCCACAGCCC
GCCTGGATGTTCGGGGACCCCCACATCACCACCTTGGATGGTGTCA
GTTACACCTTCAATGGGCTGGGGGACTTCCTGCTGGTCGGGGCCCA
AGACGGGAACTCCTCCTTCCTGCTTCAGGGCCGCACCGCCCAGACT
GGCTCAGCCCAGGCCACCAACTTCATCGCCTTTGCGGCTCAGTACC
GCTCCAGCAGCCTGGGCCCCGTCACGGTCCAATGGCTCCTTGAGCC
TCACGACGCAATCCGTGTCCTGCTGGATAACCAGACTGTGACATTT
CAGCCTGACCATGAAGACGGCGGAGGCCAGGAGACGTTCAACGCC
ACCGGAGTCCTCCTGAGCCGCAACGGCTCTGAGGCCTCCGCCAGCT
TCGACGGCTGGGCCACCGTCTCGGTGATCGCGCTCTCCAACATCCT
CCACTCCTCCGCCAGCCTCCCGCCCGAGTACCAGAACCGCACGGAG
GGGCTCCTGGGGGTCTGGAATAACAATCCAGAGGACGACTTCAGG
ATGCCCAATGGCTCCACCATTCCCCCAGGGAGCCCTGAGGAGATGC
TTTTCCACTTTGGAATGACCTGGCAGATCAACGGGACAGGCCTCCT
TGGCAAGAGGAATGACCAGCTGCCTTCCAACTTCACCCCTGTTTTC
TACTCACAACTGCAAAAAAACAGCTCCTGGGCTGAACATTTGATCT
CCAACTGTGACGGAGATAGCTCATGCATCTATGACACCCTGGCCCT
GCGCAACGCAAGCATCGGACTTCACACGAGGGAAGTCAGTAAAAA
CTACGAGCAGGCGAACGCCACCCTCAATCAGTACCCGCCCTCCATC
AATGGTGGTCGTGTGATTGAAGCCTACAAGGGGCAGACCACGCTG
ATTCAGTACACCAGCAATGCTGAGGATGCCAACTTCACGCTCAGAG
ACAGCTGCACCGACTTGGAGCTCTTTGAGAATGGGACGTTGCTGTG
GACACCCAAGTCGCTGGAGCCATTCACTCTGGAGATTCTAGCAAGA
AGTGCCAAGATTGGCTTGGCATCTGCACTCCAGCCCAGGACTGTGG
TCTGCCATTGCAATGCAGAGAGCCAGTGTTTGTACAATCAGACCAG
CAGGGTGGGCAACTCCTCCCTGGAGGTGGCTGGCTGCAAGTGTGAC
GGGGGCACCTTCGGCCGCTACTGCGAGGGCTCCGAGGATGCCTGTG
AGGAGCCGTGCTTCCCGAGTGTCCACTGCGTTCCTGGGAAGGGCTG
CGAGGCCTGCCCTCCAAACCTGACTGGGGATGGGCGGCACTGTGCG
GCTCTGGGGAGCTCTTTCCTGTGTCAGAACCAGTCCTGCCCTGTGA
ATTACTGCTACAATCAAGGCCACTGCTACATCTCCCAGACTCTGGG
CTGTCAGCCCATGTGCACCTGCCCCCCAGCCTTCACTGACAGCCGC
TGCTTCCTGGCTGGGAACAACTTCAGTCCAACTGTCAACCTAGAAC
TTCCCTTAAGAGTCATCCAGCTCTTGCTCAGTGAAGAGGAAAATGC
CTCCATGGCAGAGGTCAACGCCTCGGTGGCATACAGACTGGGGAC
CCTGGACATGCGGGCCTTTCTCCGCAACAGCCAAGTGGAACGAATC
GATTCTGCAGCACCGGCCTCGGGAAGCCCCATCCAACACTGGATGG
TCATCTCGGAGTTCCAGTACCGCCCTCGGGGCCCGGTCATTGACTT
CCTGAACAACCAGCTGCTGGCCGCGGTGGTGGAGGCGTTCTTATAC
CACGTTCCACGGAGGAGTGAGGAGCCCAGGAACGACGTGGTCTTC
CAGCCCATTTCCGGGGAAGACGTGCGCGATGTGACAGCCCTGAAC
GTGAGCACGCTGAAGGCTTACTTCAGATGCGATGGCTACAAGGGCT
ACGACCTGGTCTACAGCCCCCAGAGCGGCTTCACCTGCGTGTCCCC
GTGCAGTAGGGGCTACTGTGACCATGGAGGCCAGTGCCAGCACCT
GCCCAGTGGGCCCCGCTGCAGCTGTGTGTCCTTCTCCATCTACACG
GCCTGGGGCGAGCACTGTGAGCACCTGAGCATGAAACTCGACGCG
TTCTTCGGCATCTTCTTTGGGGCCCTGGGCGGCCTCTTGCTGCTGGG
GGTCGGGACGTTCGTGGTCCTGCGCTTCTGGGGTTGCTCCGGGGCC
AGGTTCTCCTATTTCCTGAACTCAGCTGAGGCCTTGCCTTGAAGGG
GCAGCTGTGGCCTAGGCTACCTCAAGACTCACCTCATCCTTACCGC
ACATTTAAGGCGCCATTGCTTTTGGGAGACTGGAAAAGGGAAGGT
GACTGAAGGCTGTCAGGATTCTTCAAGGAGAATGAATACTGGGAA
TCAAGACAAGACTATACCTTATCCATAGGCGCAGGTGCACAGGGG
GAGGCCATAAAGATCAAACATGCATGGATGGGTCCTCACGCAGAC
ACACCCACAGAAGGACACTAGCCTGTGCACGCGCGCGTGCACACA
CACACACACACACACGAGTTCATAATGTGGTGATGGCCCTAAGTTA
AGCAAAATGCTTCTGCACACAAAACTCTCTGGTTTACTTCAAATTA
ACTCTATTTAAATAAAGTCTCTCTGACTTTTTGTGTCTCC

SEQ ID NO: 3454

TACAGCCCCAAGGTCGCTCCCTCTGGGGCCCTTTCTTCCCCATTCTT
CCCAGCAGCCCAAAGCTCTGGTGGGACAGGGGCAGCCCCTGGGGA
GGGAGGAGAGGACCCAGGAACCCGGCTAGGAGGGTGGCCCACCCA
TTTCCAGTGTGACCTGTTCCCATTCCCCCATGTCTCCTCCCATCCCT
CCCGCCACTCAGCTCAGGCTGATGAGAAGCAGAGCAACGGGTGTA
TCGGTGTTTTCTTTCCTGGTGGGGTAGTGGGGTGGGGCTGAGGAGA
GAAAAGGGTGATTAGCGTGGGGCCCCGCCCTCTTTTGTCCTCTTCC
CAGGTTCCCTGGCCCCTTCGGAGAAACGCACTTGGTTCGGGCCAGC
CGCCTGAGGGGACGGGCTCACGTCTGCTCCTCACACTGCAGCTGCT
GGGCCGTGGAGCTTCCCCAGGGAGCCAGGGGGACTTTTGCCGCAG
CCATGAAGGGGGCACGCTGGAGGAGGGTCCCCTGGGTGTCCCTGA
GCTGCCTGTGTCTCTGCCTCCTTCCGCATGTGGTCCCAGGAACCACA
GAGGACACATTAATAACTGGAAGTAAAACTCCTGCCCCAGTCACCT
CAACAGGCTCAACAACAGCGACACTAGAGGGACAATCAACTGCAG
CTTCTTCAAGGACCTCTAATCAGGACATATCAGCTTCATCTCAGAA
CCACCAGACTAAGAGCACGGAGACCACCAGCAAAGCTCAAACCGA
CACCCTCACGCAGATGATGACATCAACTCTTTTTTCTTCCCCAAGTG
TACACAATGTGATGGAGACTGTTACGCAGGAGACAGCTCCTCCAGA
TGAAATGACCACATCATTTCCCTCCAGTGTCACCAACACACTCATG
ATGACATCAAAGACTATAACAATGACAACCTCCACAGACTCCACTC
TTGGAAACACAGAAGAGACATCAACAGCAGGAACTGAAAGTTCTA
CCCCAGTGACCTCAGCAGTCTCAATAACAGCTGGACAGGAAGGAC
AATCACGAACAACTTCCTGGAGGACCTCTATCCAAGACACATCAGC
TTCTTCTCAGAACCACTGGACTCGGAGCACGCAGACCACCAGGGAA
TCTCAAACCAGCACCCTAACACACAGAACCACTTCAACTCCTTCTT
TCTCTCCAAGTGTACACAATGTGACAGGGACTGTTTCTCAGAAGAC
ATCTCCTTCAGGTGAAACAGCTACCTCATCCCTCTGTAGTGTCACA
AACACATCCATGATGACATCAGAGAAGATAACAGTGACAACCTCC
ACAGGCTCCACTCTTGGAAACCCAGGGGAGACATCATCAGTACCTG
TTACTGGAAGTCTTATGCCAGTCACCTCAGCAGCCTTAGTAACAGT
TGATCCAGAAGGACAATCACCAGCAACTTTCTCAAGGACTTCTACT
CAGGACACAACAGCTTTTTCTAAGAACCACCAGACTCAGAGCGTGG
AGACCACCAGAGTATCTCAAATCAACACCCTCAACACCCTCACACC
GGTTACAACATCAACTGTTTTATCCTCACCAAGTGGATTCAACCCA
AGTGGAACAGTTTCTCAGGAGACATTCCCTTCTGGTGAAACAACCA
TCTCATCCCCTTCCAGTGTCAGCAATACATTCCTGGTAACATCAAA
GGTGTTCAGAATGCCAATCTCCAGAGACTCTACTCTTGGAAACACA
GAGGAGACATCACTATCTGTAAGTGGAACCATTTCTGCAATCACTT
CCAAAGTTTCAACCATATGGTGGTCAGACACTCTGTCAACAGCACT
CTCCCCCAGTTCTCTACCTCCAAAAATATCCACAGCTTTCCACACCC
AGCAGAGTGAAGGTGCAGAGACCACAGGACGGCCTCATGAGAGGA
GCTCATTCTCTCCAGGTGTGTCTCAAGAAATATTTACTCTACATGAA
ACAACAACATGGCCTTCCTCATTCTCCAGCAAAGGCCACACAACTT
GGTCACAAACAGAACTGCCCTCAACATCAACAGGTGCTGCCACTAG
GCTTGTCACAGGAAATCCATCTACAGGGGCAGCTGGCACTATTCCA
AGGGTCCCCTCTAAGGTCTCAGCAATAGGGGAACCAGGAGAGCCC
ACCACATACTCCTCCCACAGCACAACTCTCCCAAAAACAACAGGGG
CAGGCGCCCAGACACAATGGACACAAGAAACGGGGACCACTGGAG
AGGCTCTTCTCAGCAGCCCAAGCTACAGTGTGACTCAGATGATAAA
AACGGCCACATCCCCATCTTCTTCACCTATGCTGGATAGACACACA
TCACAACAAATTACAACGGCACCATCAACAAATCATTCAACAATAC
ATTCCACAAGCACCTCTCCTCAGGAATCACCAGCTGTTTCCCAAAG
GGGTCACACTCAAGCCCCGCAGACCACACAAGAATCACAAACCAC
GAGGTCCGTCTCCCCCATGACTGACACCAAGACAGTCACCACCCCA
GGTTCTTCCTTCACAGCCAGTGGGCACTCGCCCTCAGAAATTGTTCC
TCAGGACGCACCCACCATAAGTGCAGCAACAACCTTTGCCCCAGCT
CCCACCGGGGATGGTCACACAACCCAGGCCCCGACCACAGCACTG
CAGGCAGCACCCAGCAGCCATGATGCCACCCTGGGGCCCTCAGGA
GGCACGTCACTTTCCAAAACAGGTGCCCTTACTCTGGCCAACTCTG
TAGTGTCAACACCAGGGGGCCCAGAAGGACAATGGACATCAGCCT
CTGCCAGCACCTCACCTGACACAGCAGCAGCCATGACCCATACCCA
CCAGGCTGAGAGCACAGAGGCCTCTGGACAAACACAGACCAGCGA
ACCGGCCTCCTCAGGGTCACGAACCACCTCAGCGGGCACAGCTACC
CCTTCCTCATCCGGGGCGAGTGGCACAACACCTTCAGGAAGCGAAG
GAATATCCACCTCAGGAGAGACGACAAGGTTTTCATCAAACCCCTC
CAGGGACAGTCACACAACCCAGTCAACAACCGAATTGCTGTCCGCC
TCAGCCAGTCATGGTGCCATCCCAGTAAGCACAGGAATGGCGTCTT
CGATCGTCCCCGGCACCTTTCATCCCACCCTCTCTGAGGCCTCCACT
GCAGGGAGACCGACAGGACAGTCAAGCCCAACTTCTCCCAGTGCC
TCTCCTCAGGAGACAGCCGCCATTTCCCGGATGGCCCAGACTCAGA
GGACAAGAACCAGCAGAGGGTCTGACACTATCAGCCTGGCGTCCC
AGGCAACCGACACCTTCTCAACAGTCCCACCCACACCTCCATCGAT
CACATCCAGTGGGCTTACATCTCCACAAACCCAGACCCACACTCTG
TCACCTTCAGGGTCTGGTAAAACCTTCACCACGGCCCTCATCAGCA
ACGCCACCCCTCTTCCTGTCACCAGCACCTCCTCAGCCTCCACAGGT
CACGCCACCCCTCTTGCTGTCAGCAGTGCTACCTCAGCTTCCACAGT
ATCCTCGGACTCCCCTCTGAAGATGGAAACATCAGGAATGACAACA
CCGTCACTGAAGACAGACGGTGGGAGACGCACAGCCACATCACCA
CCCCCCACAACCTCCCAGACCATCATTTCCACCATTCCCAGCACTG
CCATGCACACCCGCTCCACAGCTGCCCCCATCCCCATCCTGCCTGA
GAGAGGAGTTTCCCTCTTCCCCTATGGGGCAGACGCCGGGGACCTG
GAGTTCGTCAGGAGGACCGTGGACTTCACCTCCCCACTCTTCAAGC
CGGCGACTGGCTTCCCCCTTGGCTCCTCTCTCCGTGATTCCCTCTAC
TTCACAGACAATGGCCAGATCATCTTCCCAGAGTCAGACTACCAGA
TTTTCTCCTACCCCAACCCACTCCCAACAGGCTTCACAGGCCGGGA
CCCTGTGGCCCTGGTGGCTCCGTTCTGGGACGATGCTGACTTCTCCA
CTGGTCGGGGGACCACATTTTATCAGGAATACGAGACGTTCTATGG
TGAACACAGCCTGCTAGTCCAGCAGGCCGAGTCTTGGATTAGAAAG
ATCACAAACAACGGGGGCTACAAGGCCAGGTGGGCCCTAAAGGTC
ACGTGGGTCAATGCCCACGCCTATCCTGCCCAGTGGACCCTCGGGA
GCAACACCTACCAAGCCATCCTCTCCACGGACGGGAGCAGGTCCTA
TGCCCTGTTTCTCTACCAGAGCGGTGGGATGCAGTGGGACGTGGCC
CAGCTCTAGTGGAGATGGCTTTTTCGAAAACAGCCCACTGATGTCC
CAGCCAGTGTGGGAGAGGTATCGCCCTGATAGATTCCTGAATTCCA
ACTCAGGCCTCCAAGGGCTGCAGTTCTACGGGCTACACCGGGAAG
AAAGGCCCAACTACCGTCTCGAGTGCCTGCAGTGGCTGAAGAGCC
AGCCTCGGTGGCCCAGCTGGGGCTGGAACCAGGTCTCCTGCCCTTG
TTCCTGGCAGCAGGGACGACGGGACTTACGATTCCAACCCGTCAGC
ATAGGTCGCTGGGGCCTCGGCAGTAGGCAGCTGTGCAGCTTCACCT
CTTGGCGAGGAGGCGTGTGCTGCAGCTACGGGCCCTGGGGAGAGT
TTCGTGAAGGCTGGCACGTGCAGCGTCCTTGGCAGTTGGCCCAGGA
ACTGGAGCCACAGAGCTGGTGCTGCCGCTGGAATGACAAGCCCTA
CCTCTGTGCCCTGTACCAGCAGAGGCGGCCCCACGTGGGCTGTGCT
ACATACAGGCCCCCACAGCCCGCCTGGATGTTCGGGGACCCCCACA
TCACCACCTTGGATGGTGTCAGTTACACCTTCAATGGGCTGGGGGA
CTTCCTGCTGGTCGGGGCCCAAGACGGGAACTCCTCCTTCCTGCTTC
AGGGCCGCACCGCCCAGACTGGCTCAGCCCAGGCCACCAACTTCAT
CGCCTTTGCGGCTCAGTACCGCTCCAGCAGCCTGGGCCCCGTCACG
GTCCAATGGCTCCTTGAGCCTCACGACGCAATCCGTGTCCTGCTGG
ATAACCAGACTGTGACATTTCAGCCTGACCATGAAGACGGCGGAG
GCCAGGAGACGTTCAACGCCACCGGAGTCCTCCTGAGCCGCAACG
GCTCTGAGGCCTCCGCCAGCTTCGACGGCTGGGCCACCGTCTCGGT
GATCGCGCTCTCCAACATCCTCCACTCCTCCGCCAGCCTCCCGCCCG
AGTACCAGAACCGCACGGAGGGGCTCCTGGGGGTCTGGAATAACA
ATCCAGAGGACGACTTCAGGATGCCCAATGGCTCCACCATTCCCCC
AGGGAGCCCTGAGGAGATGCTTTTCCACTTTGGAATGACCTGGCAG
ATCAACGGGACAGGCCTCCTTGGCAAGAGGAATGACCAGCTGCCTT
CCAACTTCACCCCTGTTTTCTACTCACAACTGCAAAAAAACAGCTC
CTGGGCTGAACATTTGATCTCCAACTGTGACGGAGATAGCTCATGC
ATCTATGACACCCTGGCCCTGCGCAACGCAAGCATCGGACTTCACA
CGAGGGAAGTCAGTAAAAACTACGAGCAGGCGAACGCCACCCTCA
ATCAGTACCCGCCCTCCATCAATGGTGGTCGTGTGATTGAAGCCTA
CAAGGGGCAGACCACGCTGATTCAGTACACCAGCAATGCTGAGGA
TGCCAACTTCACGCTCAGAGACAGCTGCACCGACTTGGAGCTCTTT
GAGAATGGGACGTTGCTGTGGACACCCAAGTCGCTGGAGCCATTCA
CTCTGGAGATTCTAGCAAGAAGTGCCAAGATTGGCTTGGCATCTGC
ACTCCAGCCCAGGACTGTGGTCTGCCATTGCAATGCAGAGAGCCAG
TGTTTGTACAATCAGACCAGCAGGGTGGGCAACTCCTCCCTGGAGG
TGGCTGGCTGCAAGTGTGACGGGGGCACCTTCGGCCGCTACTGCGA
GGGCTCCGAGGATGCCTGTGAGGAGCCGTGCTTCCCGAGTGTCCAC
TGCGTTCCTGGGAAGGGCTGCGAGGCCTGCCCTCCAAACCTGACTG
GGGATGGGCGGCACTGTGCGGCTCTGGGGAGCTCTTTCCTGTGTCA
GAACCAGTCCTGCCCTGTGAATTACTGCTACAATCAAGGCCACTGC
TACATCTCCCAGACTCTGGGCTGTCAGCCCATGTGCACCTGCCCCC
CAGCCTTCACTGACAGCCGCTGCTTCCTGGCTGGGAACAACTTCAG
TCCAACTGTCAACCTAGAACTTCCCTTAAGAGTCATCCAGCTCTTGC
TCAGTGAAGAGGAAAATGCCTCCATGGCAGAGGTCAACGCCTCGG
TGGCATACAGACTGGGGACCCTGGACATGCGGGCCTTTCTCCGCAA
CAGCCAAGTGGAACGAATCGATTCTGCAGCACCGGCCTCGGGAAG
CCCCATCCAACACTGGATGGTCATCTCGGAGTTCCAGTACCGCCCT
CGGGGCCCGGTCATTGACTTCCTGAACAACCAGCTGCTGGCCGCGG
TGGTGGAGGCGTTCTTATACCACGTTCCACGGAGGAGTGAGGAGCC
CAGGAACGACGTGGTCTTCCAGCCCATTTCCGGGGAAGACGTGCGC
GATGTGACAGCCCTGAACGTGAGCACGCTGAAGGCTTACTTCAGAT
GCGATGGCTACAAGGGCTACGACCTGGTCTACAGCCCCCAGAGCG
GCTTCACCTGCGTGTCCCCGTGCAGTAGGGGCTACTGTGACCATGG
AGGCCAGTGCCAGCACCTGCCCAGTGGGCCCCGCTGCAGCTGTGTG
TCCTTCTCCATCTACACGGCCTGGGGCGAGCACTGTGAGCACCTGA
GCATGAAACTCGACGCGTTCTTCGGCATCTTCTTTGGGGCCCTGGG
CGGCCTCTTGCTGCTGGGGGTCGGGACGTTCGTGGTCCTGCGCTTCT
GGGGTTGCTCCGGGGCCAGGTTCTCCTATTTCCTGAACTCAGCTGA
GGCCTTGCCTTGAAGGGGCAGCTGTGGCCTAGGCTACCTCAAGACT
CACCTCATCCTTACCGCACATTTAAGGCGCCATTGCTTTTGGGAGA
CTGGAAAAGGGAAGGTGACTGAAGGCTGTCAGGATTCTTCAAGGA
GAATGAATACTGGGAATCAAGACAAGACTATACCTTATCCATAGGC
GCAGGTGCACAGGGGGAGGCCATAAAGATCAAACATGCATGGATG
GGTCCTCACGCAGACACACCCACAGAAGGACACTAGCCTGTGCAC
GCGCGCGTGCACACACACACACACACACACGAGTTCATAATGTGGT
GATGGCCCTAAGTTAAGCAAAATGCTTCTGCACACAAAACTCTCTG
GTTTACTTCAAATTAACTCTATTTAAATAAAGTCTCTCTGACTTTTT
GTGTCTCC

SEQ ID NO: 3455

TACAGCCCCAAGGTCGCTCCCTCTGGGGCCCTTTCTTCCCCATTCTT
CCCAGCAGCCCAAAGCTCTGGTGGGACAGGGGCAGCCCCTGGGGA
GGGAGGAGAGGACCCAGGAACCCGGCTAGGAGGGTGGCCCACCCA
TTTCCAGTGTGACCTGTTCCCATTCCCCCATGTCTCCTCCCATCCCT
CCCGCCACTCAGCTCAGGCTGATGAGAAGCAGAGCAACGGGTGTA
TCGGTGTTTTCTTTCCTGGTGGGGTAGTGGGGTGGGGCTGAGGAGA
GAAAAGGGTGATTAGCGTGGGGCCCCGCCCTCTTTTGTCCTCTTCC
CAGGTTCCCTGGCCCCTTCGGAGAAACGCACTTGGTTCGGGCCAGC
CGCCTGAGGGGACGGGCTCACGTCTGCTCCTCACACTGCAGCTGCT
GGGCCGTGGAGCTTCCCCAGGGAGCCAGGGGGACTTTTGCCGCAG
CCATGAAGGGGGCACGCTGGAGGAGGGTCCCCTGGGTGTCCCTGA
GCTGCCTGTGTCTCTGCCTCCTTCCGCATGTGGTCCCAGGAACCACA
GAGGACACATTAATAACTGGAAGTAAAACTCCTGCCCCAGTCACCT
CAACAGGCTCAACAACAGCGACACTAGAGGGACAATCAACTGCAG
CTTCTTCAAGGACCTCTAATCAGGACATATCAGCTTCATCTCAGAA
CCACCAGACTAAGAGCACGGAGACCACCAGCAAAGCTCAAACCGA
CACCCTCACGCAGATGATGACATCAACTCTTTTTTCTTCCCCAAGTG
TACACAATGTGATGGAGACTGTTACGCAGGAGACAGCTCCTCCAGA
TGAAATGACCACATCATTTCCCTCCAGTGTCACCAACACACTCATG
ATGACATCAAAGACTATAACAATGACAACCTCCACAGACTCCACTC
TTGGAAACACAGAAGAGACATCAACAGCAGGAACTGAAAGTTCTA
CCCCAGTGACCTCAGCAGTCTCAATAACAGCTGGACAGGAAGGAC
AATCACGAACAACTTCCTGGAGGACCTCTATCCAAGACACATCAGC
TTCTTCTCAGAACCACTGGACTCGGAGCACGCAGACCACCAGGGAA
TCTCAAACCAGCACCCTAACACACAGAACCACTTCAACTCCTTCTT
TCTCTCCAAGTGTACACAATGTGACAGGGACTGTTTCTCAGAAGAC
ATCTCCTTCAGGTGAAACAGCTACCTCATCCCTCTGTAGTGTCACA
AACACATCCATGATGACATCAGAGAAGATAACAGTGACAACCTCC
ACAGGCTCCACTCTTGGAAACCCAGGGGAGACATCATCAGTACCTG
TTACTGGAAGTCTTATGCCAGTCACCTCAGCAGCCTTAGTAACAGT
TGATCCAGAAGGACAATCACCAGCAACTTTCTCAAGGACTTCTACT
CAGGACACAACAGCTTTTTCTAAGAACCACCAGACTCAGAGCGTGG
AGACCACCAGAGTATCTCAAATCAACACCCTCAACACCCTCACACC
GGTTACAACATCAACTGTTTTATCCTCACCAAGTGGATTCAACCCA
AGTGGAACAGTTTCTCAGGAGACATTCCCTTCTGGTGAAACAACCA
TCTCATCCCCTTCCAGTGTCAGCAATACATTCCTGGTAACATCAAA
GGTGTTCAGAATGCCAATCTCCAGAGACTCTACTCTTGGAAACACA
GAGGAGACATCACTATCTGTAAGTGGAACCATTTCTGCAATCACTT
CCAAAGTTTCAACCATATGGTGGTCAGACACTCTGTCAACAGCACT
CTCCCCCAGTTCTCTACCTCCAAAAATATCCACAGCTTTCCACACCC
AGCAGAGTGAAGGTGCAGAGACCACAGGACGGCCTCATGAGAGGA
GCTCATTCTCTCCAGGTGTGTCTCAAGAAATATTTACTCTACATGAA
ACAACAACATGGCCTTCCTCATTCTCCAGCAAAGGCCACACAACTT
GGTCACAAACAGAACTGCCCTCAACATCAACAGGTGCTGCCACTAG
GCTTGTCACAGGAAATCCATCTACAGGGGCAGCTGGCACTATTCCA
AGGGTCCCCTCTAAGGTCTCAGCAATAGGGGAACCAGGAGAGCCC
ACCACATACTCCTCCCACAGCACAACTCTCCCAAAAACAACAGGGG
CAGGCGCCCAGACACAATGGACACAAGAAACGGGGACCACTGGAG
AGGCTCTTCTCAGCAGCCCAAGCTACAGTGTGACTCAGATGATAAA
AACGGCCACATCCCCATCTTCTTCACCTATGCTGGATAGACACACA
TCACAACAAATTACAACGGCACCATCAACAAATCATTCAACAATAC
ATTCCACAAGCACCTCTCCTCAGGAATCACCAGCTGTTTCCCAAAG
GGGTCACACTCAAGCCCCGCAGACCACACAAGAATCACAAACCAC
GAGGTCCGTCTCCCCCATGACTGACACCAAGACAGTCACCACCCCA
GGTTCTTCCTTCACAGCCAGTGGGCACTCGCCCTCAGAAATTGTTCC
TCAGGACGCACCCACCATAAGTGCAGCAACAACCTTTGCCCCAGCT
CCCACCGGGGATGGTCACACAACCCAGGCCCCGACCACAGCACTG
CAGGCAGCACCCAGCAGCCATGATGCCACCCTGGGGCCCTCAGGA
GGCACGTCACTTTCCAAAACAGGTGCCCTTACTCTGGCCAACTCTG
TAGTGTCAACACCAGGGGGCCCAGAAGGACAATGGACATCAGCCT
CTGCCAGCACCTCACCTGACACAGCAGCAGCCATGACCCATACCCA
CCAGGCTGAGAGCACAGAGGCCTCTGGACAAACACAGACCAGCGA
ACCGGCCTCCTCAGGGTCACGAACCACCTCAGCGGGCACAGCTACC
CCTTCCTCATCCGGGGCGAGTGGCACAACACCTTCAGGAAGCGAAG
GAATATCCACCTCAGGAGAGACGACAAGGTTTTCATCAAACCCCTC
CAGGGACAGTCACACAACCCAGTCAACAACCGAATTGCTGTCCGCC
TCAGCCAGTCATGGTGCCATCCCAGTAAGCACAGGAATGGCGTCTT
CGATCGTCCCCGGCACCTTTCATCCCACCCTCTCTGAGGCCTCCACT
GCAGGGAGACCGACAGGACAGTCAAGCCCAACTTCTCCCAGTGCC
TCTCCTCAGGAGACAGCCGCCATTTCCCGGATGGCCCAGACTCAGA
GGACAAGAACCAGCAGAGGGTCTGACACTATCAGCCTGGCGTCCC
AGGCAACCGACACCTTCTCAACAGTCCCACCCACACCTCCATCGAT
CACATCCAGTGGGCTTACATCTCCACAAACCCAGACCCACACTCTG
TCACCTTCAGGGTCTGGTAAAACCTTCACCACGGCCCTCATCAGCA
ACGCCACCCCTCTTCCTGTCACCAGCACCTCCTCAGCCTCCACAGGT
CACGCCACCCCTCTTGCTGTCAGCAGTGCTACCTCAGCTTCCACAGT
ATCCTCGGACTCCCCTCTGAAGATGGAAACATCAGGAATGACAACA
CCGTCACTGAAGACAGACGGTGGGAGACGCACAGCCACATCACCA
CCCCCCACAACCTCCCAGACCATCATTTCCACCATTCCCAGCACTG
CCATGCACACCCGCTCCACAGCTGCCCCCATCCCCATCCTGCCTGA
GAGAGGAGTTTCCCTCTTCCCCTATGGGGCAGACGCCGGGGACCTG
GAGTTCGTCAGGAGGACCGTGGACTTCACCTCCCCACTCTTCAAGC
CGGCGACTGGCTTCCCCCTTGGCTCCTCTCTCCGTGATTCCCTCTAC
TTCACAGACAATGGCCAGATCATCTTCCCAGAGTCAGACTACCAGA
TTTTCTCCTACCCCAACCCACTCCCAACAGGCTTCACAGGCCGGGA
CCCTGTGGCCCTGGTGGCTCCGTTCTGGGACGATGCTGACTTCTCCA
CTGGTCGGGGGACCACATTTTATCAGGAATACGAGACGTTCTATGG
TGAACACAGCCTGCTAGTCCAGCAGGCCGAGTCTTGGATTAGAAAG
ATCACAAACAACGGGGGCTACAAGGCCAGGTGGGCCCTAAAGGTC
ACGTGGGTCAATGCCCACGCCTATCCTGCCCAGTGGACCCTCGGGA
GCAACACCTACCAAGCCATCCTCTCCACGGACGGGAGCAGGTCCTA
TGCCCTGTTTCTCTACCAGAGCGGTGGGATGCAGTGGGACGTGGCC
CAGCGCTCAGGCAAGCCGGTGCTCATGGGCTTCTCTAGTGGAGATG
GCTTTTTCGAAAACAGCCCACTGATGTCCCAGCCAGTGTGGGAGAG
GTATCGCCCTGATAGATTCCTGAATTCCAACTCAGGCCTCCAAGGG
CTGCAGTTCTACGGGCTACACCGGGAAGAAAGGCCCAACTACCGTC
TCGAGTGCCTGCAGTGGCTGAAGAGCCAGCCTCGGTGGCCCAGCTG
GGGCTGGAACCAGGTCTCCTGCCCTTGTTCCTGGCAGCAGGGACGA
CGGGACTTACGATTCCAACCCGTCAGCATAGGTCGCTGGGGCCTCG
GCAGTAGGCAGCTGTGCAGCTTCACCTCTTGGCGAGGAGGCGTGTG
CTGCAGCTACGGGCCCTGGGGAGAGTTTCGTGAAGGCTGGCACGTG
CAGCGTCCTTGGCAGTTGGCCCAGGAACTGGAGCCACAGAGCTGGT
GCTGCCGCTGGAATGACAAGCCCTACCTCTGTGCCCTGTACCAGCA
GAGGCGGCCCCACGTGGGCTGTGCTACATACAGGCCCCCACAGCCC
GCCTGGATGTTCGGGGACCCCCACATCACCACCTTGGATGGTGTCA
GTTACACCTTCAATGGGCTGGGGGACTTCCTGCTGGTCGGGGCCCA
AGACGGGAACTCCTCCTTCCTGCTTCAGGGCCGCACCGCCCAGACT
GGCTCAGCCCAGGCCACCAACTTCATCGCCTTTGCGGCTCAGTACC
GCTCCAGCAGCCTGGGCCCCGTCACGGTCCAATGGCTCCTTGAGCC
TCACGACGCAATCCGTGTCCTGCTGGATAACCAGACTGTGACATTT
CAGCCTGACCATGAAGACGGCGGAGGCCAGGAGACGTTCAACGCC
ACCGGAGTCCTCCTGAGCCGCAACGGCTCTGAGGCCTCCGCCAGCT
TCGACGGCTGGGCCACCGTCTCGGTGATCGCGCTCTCCAACATCCT
CCACTCCTCCGCCAGCCTCCCGCCCGAGTACCAGAACCGCACGGAG
GGGCTCCTGGGGGTCTGGAATAACAATCCAGAGGACGACTTCAGG
ATGCCCAATGGCTCCACCATTCCCCCAGGGAGCCCTGAGGAGATGC
TTTTCCACTTTGGAATGACCTGGCAGATCAACGGGACAGGCCTCCT
TGGCAAGAGGAATGACCAGCTGCCTTCCAACTTCACCCCTGTTTTC
TACTCACAACTGCAAAAAAACAGCTCCTGGGCTGAACATTTGATCT
CCAACTGTGACGGAGATAGCTCATGCATCTATGACACCCTGGCCCT
GCGCAACGCAAGCATCGGACTTCACACGAGGGAAGTCAGTAAAAA
CTACGAGCAGGCGAACGCCACCCTCAATCAGTACCCGCCCTCCATC
AATGGTGGTCGTGTGATTGAAGCCTACAAGGGGCAGACCACGCTG
ATTCAGTACACCAGCAATGCTGAGGATGCCAACTTCACGCTCAGAG
ACAGCTGCACCGACTTGGAGCTCTTTGAGAATGGGACGTTGCTGTG
GACACCCAAGTCGCTGGAGCCATTCACTCTGGAGATTCTAGCAAGA
AGTGCCAAGATTGGCTTGGCATCTGCACTCCAGCCCAGGACTGTGG
TCTGCCATTGCAATGCAGAGAGCCAGTGTTTGTACAATCAGACCAG
CAGGGTGGGCAACTCCTCCCTGGAGCTCTGGGGAGCTCTTTCCTGT
GTCAGAACCAGTCCTGCCCTGTGAATTACTGCTACAATCAAGGCCA
CTGCTACATCTCCCAGACTCTGGGCTGTCAGCCCATGTGCACCTGC
CCCCCAGCCTTCACTGACAGCCGCTGCTTCCTGGCTGGGAACAACT
TCAGTCCAACTGTCAACCTAGAACTTCCCTTAAGAGTCATCCAGCT
CTTGCTCAGTGAAGAGGAAAATGCCTCCATGGCAGAGGTCAACGC
CTCGGTGGCATACAGACTGGGGACCCTGGACATGCGGGCCTTTCTC
CGCAACAGCCAAGTGGAACGAATCGATTCTGCAGCACCGGCCTCG
GGAAGCCCCATCCAACACTGGATGGTCATCTCGGAGTTCCAGTACC
GCCCTCGGGGCCCGGTCATTGACTTCCTGAACAACCAGCTGCTGGC
CGCGGTGGTGGAGGCGTTCTTATACCACGTTCCACGGAGGAGTGAG
GAGCCCAGGAACGACGTGGTCTTCCAGCCCATTTCCGGGGAAGAC
GTGCGCGATGTGACAGCCCTGAACGTGAGCACGCTGAAGGCTTACT
TCAGATGCGATGGCTACAAGGGCTACGACCTGGTCTACAGCCCCCA
GAGCGGCTTCACCTGCGTGTCCCCGTGCAGTAGGGGCTACTGTGAC
CATGGAGGCCAGTGCCAGCACCTGCCCAGTGGGCCCCGCTGCAGCT
GTGTGTCCTTCTCCATCTACACGGCCTGGGGCGAGCACTGTGAGCA
CCTGAGCATGAAACTCGACGCGTTCTTCGGCATCTTCTTTGGGGCC
CTGGGCGGCCTCTTGCTGCTGGGGGTCGGGACGTTCGTGGTCCTGC
GCTTCTGGGGTTGCTCCGGGGCCAGGTTCTCCTATTTCCTGAACTCA
GCTGAGGCCTTGCCTTGAAGGGGCAGCTGTGGCCTAGGCTACCTCA
AGACTCACCTCATCCTTACCGCACATTTAAGGCGCCATTGCTTTTGG
GAGACTGGAAAAGGGAAGGTGACTGAAGGCTGTCAGGATTCTTCA
AGGAGAATGAATACTGGGAATCAAGACAAGACTATACCTTATCCA
TAGGCGCAGGTGCACAGGGGGAGGCCATAAAGATCAAACATGCAT
GGATGGGTCCTCACGCAGACACACCCACAGAAGGACACTAGCCTG
TGCACGCGCGCGTGCACACACACACACACACACACGAGTTCATAAT
GTGGTGATGGCCCTAAGTTAAGCAAAATGCTTCTGCACACAAAACT
CTCTGGTTTACTTCAAATTAACTCTATTTAAATAAAGTCTCTCTGAT
TTTTGTGTCTCC

SEQ ID NO: 3456

TACAGCCCCAAGGTCGCTCCCTCTGGGGCCCTTTCTTCCCCATTCTT
CCCAGCAGCCCAAAGCTCTGGTGGGACAGGGGCAGCCCCTGGGGA
GGGAGGAGAGGACCCAGGAACCCGGCTAGGAGGGTGGCCCACCCA
TTTCCAGTGTGACCTGTTCCCATTCCCCCATGTCTCCTCCCATCCCT
CCCGCCACTCAGCTCAGGCTGATGAGAAGCAGAGCAACGGGTGTA
TCGGTGTTTTCTTTCCTGGTGGGGTAGTGGGGTGGGGCTGAGGAGA
GAAAAGGGTGATTAGCGTGGGGCCCCGCCCTCTTTTGTCCTCTTCC
CAGGTTCCCTGGCCCCTTCGGAGAAACGCACTTGGTTCGGGCCAGC
CGCCTGAGGGGACGGGCTCACGTCTGCTCCTCACACTGCAGCTGCT
GGGCCGTGGAGCTTCCCCAAGGGAGCCAGGGGGACTTTTGCCGCA
GCCATGAAGGGGGCACGCTGGAGGAGGGTCCCCTGGGTGTCCCTG
AGCTGCCTGTGTCTCTGCCTCCTTCCGCATGTGGTCCCAGGAATGAC
AACACCGTCACTGAAGACAGACGGTGGGAGACGCACAGCCACATC
ACCACCCCCCACAACCTCCCAGACCATCATTTCCACCATTCCCAGC
ACTGCCATGCACACCCGCTCCACAGCTGCCCCCATCCCCATCCTGC
CTGAGAGAGGAGTTTCCCTCTTCCCCTATGGGGCAGACGCCGGGGA
CCTGGAGTTCGTCAGGAGGACCGTGGACTTCACCTCCCCACTCTTC
AAGCCGGCGACTGGCTTCCCCCTTGGCTCCTCTCTCCGTGATTCCCT
CTACTTCACAGACAATGGCCAGATCATCTTCCCAGAGTCAGACTAC
CAGATTTTCTCCTACCCCAACCCACTCCCAACAGGCTTCACAGGCC
GGGACCCTGTGGCCCTGGTGGCTCCGTTCTGGGACGATGCTGACTT
CTCCACTGGTCGGGGGACCACATTTTATCAGGAATACGAGACGTTC
TATGGTGAACACAGCCTGCTAGTCCAGCAGGCCGAGTCTTGGATTA
GAAAGATCACAAACAACGGGGGCTACAAGGCCAGGTGGGCCCTAA
AGGTCACGTGGGTCAATGCCCACGCCTATCCTGCCCAGTGGACCCT
CGGGAGCAACACCTACCAAGCCATCCTCTCCACGGACGGGAGCAG
GTCCTATGCCCTGTTTCTCTACCAGAGCGGTGGGATGCAGTGGGAC
GTGGCCCAGCGCTCAGGCAAGCCGGTGCTCATGGGCTTCTCTAGTG
GAGATGGCTATTTCGAAAACAGCCCACTGATGTCCCAGCCAGTGTG
GGAGAGGTATCGCCCTGATAGATTCCTGAATTCCAACTCAGGCCTC
CAAGGGCTGCAGTTCTACAGGCTACACCGGGAAGAAAGGCCCAAC
TACCGTCTCGAGTGCCTGCAGTGGCTGAAGAGCCAGCCTCGGTGGC
CCAGCTGGGGCTGGAACCAGGTCTCCTGCCCTTGTTCCTGGCAGCA
GGGACGACGGGACTTACGATTCCAACCCGTCAGCATAGGTCGCTGG
GGCCTCGGCAGTAGGCAGCTGTGCAGCTTCACCTCTTGGCGAGGAG
GCGTGTGCTGCAGCTACGGGCCCTGGGGAGAGTTTCGTGAAGGCTG
GCACGTGCAGCGTCCTTGGCAGTTGGCCCAGGAACTGGAGCCACA
GAGCTGGTGCTGCCGCTGGAATGACAAGCCCTACCTCTGTGCCCTG
TACCAGCAGAGGCGGCCCCACGTGGGCTGTGCTACATACAGGCCCC
CACAGCCCGCCTGGATGTTCGGGGACCCCCACATCACCACCTTGGA
TGGTGTCAGTTACACCTTCAATGGGCTGGGGGACTTCCTGCTGGTC
GGGGCCCAAGACGGGAACTCCTCCTTCCTGCTTCAGGGCCGCACCG
CCCAGACTGGCTCAGCCCAGGCCACCAACTTCATCGCCTTTGCGGC
TCAGTACCGCTCCAGCAGCCTGGGCCCCGTCACGGTCCAATGGCTC
CTTGAGCCTCACGACGCAATCCGTGTCCTGCTGGATAACCAGACTG
TGACATTTCAGCCTGACCATGAAGACGGCGGAGGCCAGGAGACGT
TCAACGCCACCGGAGTCCTCCTGAGCCGCAACGGCTCTGAGGTCTC
GGCCAGCTTCGACGGCTGGGCCACCGTCTCGGTGATCGCGCTCTCC
AACATCCTCCACGCCTCCGCCAGCCTCCCGCCCGAGTACCAGAACC
GCACGGAGGGGCTCCTGGGGGTCTGGAATAACAATCCAGAGGACG
ACTTCAGGATGCCCAATGGCTCCACCATTCCCCCAGGGAGCCCTGA
GGAGATGCTTTTCCACTTTGGAATGACCTGGCAGATCAACGGGACA
GGCCTCCTTGGCAAGAGGAATGACCAGCTGCCTTCCAACTTCACCC
CTGTTTTCTACTCACAACTGCAAAAAAACAGCTCCTGGGCTGAACA
TTTGATCTCCAACTGTGACGGAGATAGCTCATGCATCTATGACACC
CTGGCCCTGCGCAACGCAAGCATCGGACTTCACACGAGGGAAGTC
AGTAAAAACTACGAGCAGGCGAACGCCACCCTCAATCAGTACCCG
CCCTCCATCAATGGTGGTCGTGTGATTGAAGCCTACAAGGGGCAGA
CCACGCTGATTCAGTACACCAGCAATGCTGAGGATGCCAACTTCAC
GCTCAGAGACAGCTGCACCGACTTGGAGCTCTTTGAGAATGGGACG
TTGCTGTGGACACCCAAGTCGCTGGAGCCATTCACTCTGGAGATTC
TAGCAAGAAGTGCCAAGATTGGCTTGGCATCTGCACTCCAGCCCAG
GACTGTGGTCTGCCATTGCAATGCAGAGAGCCAGTGTTTGTACAAT
CAGACCAGCAGGGTGGGCAACTCCTCCCTGGAGGTGGCTGGCTGC
AAGTGTGACGGGGGCACCTTCGGCCGCTACTGCGAGGGCTCCGAG
GATGCCTGTGAGGAGCCGTGCTTCCCGAGTGTCCACTGCGTTCCTG
GGAAGGGCTGCGAGGCCTGCCCTCCAAACCTGACTGGGGATGGGC
GGCACTGTGCGGCTCTGGGGAGCTCTTTCCTGTGTCAGAACCAGTC
CTGCCCTGTGAATTACTGCTACAATCAAGGCCACTGCTACATCTCC
CAGACTCTGGGCTGTCAGCCCATGTGCACCTGCCCCCCAGCCTTCA
CTGACAGCCGCTGCTTCCTGGCTGGGAACAACTTCAGTCCAACTGT
CAACCTAGAACTTCCCTTAAGAGTCATCCAGCTCTTGCTCAGTGAA
GAGGAAAATGCCTCCATGGCAGAGGTCAACGCCTCGGTGGCATAC
AGACTGGGGACCCTGGACATGCGGGCCTTTCTCCGCAACAGCCAAG
TGGAACGAATCGATTCTGCAGCACCGGCCTCGGGAAGCCCCATCCA
ACACTGGATGGTCATCTCGGAGTTCCAGTACCGCCCTCGGGGCCCG
GTCATTGACTTCCTGAACAACCAGCTGCTGGCCGCGGTGGTGGAGG
CGTTCTTATACCACGTTCCACGGAGGAGTGAGGAGCCCAGGAACG
ACGTGGTCTTCCAGCCCATCTCCGGGGAAGACGTGCGCGATGTGAC
AGCCCTGAACGTGAGCACGCTGAAGGCTTACTTCAGATGCGATGGC
TACAAGGGCTACGACCTGGTCTACAGCCCCCAGAGCGGCTTCACCT
GCGTGTCCCCGTGCAGTAGGGGCTACTGTGACCATGGAGGCCAGTG
CCAGCACCTGCCCAGTGGGCCCCGCTGCAGCTGTGTGTCCTTCTCC
ATCTACACGGCCTGGGGCGAGCACTGTGAGCACCTGAGCATGAAA
CTCGACGCGTTCTTCGGCATCTTCTTTGGGGCCCTGGGCGGCCTCTT
GCTGCTGGGGGTCGGGACGTTCGTGGTCCTGCGCTTCTGGGGTTGC
TCCGGGGCCAGGTTCTCCTATTTCCTGAACTCAGCTGAGGCCTTGCC
TTGAAGGGGCAGCTGTGGCCTAGGCTACCTCAAGACTCACCTCATC
CTTACCGCACATTTAAGGCGCCATTGCTTTTGGGAGACTGGAAAAG
GGAAGGTGACTGAAGGCTGTCAGGATTCTTCAAGGAGAATGAATA
CTGGGAATCAAGACAGGACTATACCTTATCCATAGGCGCAGGTGCA
CAGGGGGAGGCCATAAAGATCAAACATGCATGGATGGGTCCTCAC
GCAGACACACCCACAGAAGGACACTAGCCTGGCGCGCGTGCACAC
ACACACACACACACACGAGTTCATAATGTGGTGATGGCCCTAAGTT
AAGCAAAATGCTTCTGCACACAAAACTCTCTGGTTTACTTCAAATT
AACTCTATTTAAATAAAGTCTCTCTGACTTTTTGTGTCTCCAAAAAA
AAAAAAAAAAA

SEQ ID NO: 3457

TACAGCCCCAAGGTCGCTCCCTCTGGGGCCCTTTCTTCCCCATTCTT
CCCAGCAGCCCAAAGCTCTGGTGGGACAGGGGCAGCCCCTGGGGA
GGGAGGAGAGGACCCAGGAACCCGGCTAGGAGGGTGGCCCACCCA
TTTCCAGTGTGACCTGTTCCCATTCCCCCATGTCTCCTCCCATCCCT
CCCGCCACTCAGCTCAGGCTGATGAGAAGCAGAGCAACGGGTGTA
TCGGTGTTTTCTTTCCTGGTGGGGTAGTGGGGTGGGGCTGAGGAGA
GAAAAGGGTGATTAGCGTGGGGCCCCGCCCTCTTTTGTCCTCTTCC
CAGGTTCCCTGGCCCCTTCGGAGAAACGCACTTGGTTCGGGCCAGC
CGCCTGAGGGGACGGGCTCACGTCTGCTCCTCACACTGCAGCTGCT
GGGCCGTGGAGCTTCCCCAAGGGAGCCAGGGGGACTTTTGCCGCA
GCCATGAAGGGGGCACGCTGGAGGAGGGTCCCCTGGGTGTCCCTG
AGCTGCCTGTGTCTCTGCCTCCTTCCGCATGTGGTCCCAGGAGTTTC
CCTCTTCCCCTATGGGGCAGACGCCGGGGACCTGGAGTTCGTCAGG
AGGACCGTGGACTTCACCTCCCCACTCTTCAAGCCGGCGACTGGCT
TCCCCCTTGGCTCCTCTCTCCGTGATTCCCTCTACTTCACAGACAAT
GGCCAGATCATCTTCCCAGAGTCAGACTACCAGATTTTCTCCTACC
CCAACCCACTCCCAACAGGCTTCACAGGCCGGGACCCTGTGGCCCT
GGTGGCTCCGTTCTGGGACGATGCTGACTTCTCCACTGGTCGGGGG
ACCACATTTTATCAGGAATACGAGACGTTCTATGGTGAACACAGCC
TGCTAGTCCAGCAGGCCGAGTCTTGGATTAGAAAGATCACAAACA
ACGGGGGCTACAAGGCCAGGTGGGCCCTAAAGGTCACGTGGGTCA
ATGCCCACGCCTATCCTGCCCAGTGGACCCTCGGGAGCAACACCTA
CCAAGCCATCCTCTCCACGGACGGGAGCAGGTCCTATGCCCTGTTT
CTCTACCAGAGCGGTGGGATGCAGTGGGACGTGGCCCAGCGCTCA
GGCAAGCCGGTGCTCATGGGCTTCTCTAGTGGAGATGGCTATTTCG
AAAACAGCCCACTGATGTCCCAGCCAGTGTGGGAGAGGTATCGCC
CTGATAGATTCCTGAATTCCAACTCAGGCCTCCAAGGGCTGCAGTT
CTACAGGCTACACCGGGAAGAAAGGCCCAACTACCGTCTCGAGTG
CCTGCAGTGGCTGAAGAGCCAGCCTCGGTGGCCCAGCTGGGGCTG
GAACCAGGTCTCCTGCCCTTGTTCCTGGCAGCAGGGACGACGGGAC
TTACGATTCCAACCCGTCAGCATAGGTCGCTGGGGCCTCGGCAGTA
GGCAGCTGTGCAGCTTCACCTCTTGGCGAGGAGGCGTGTGCTGCAG
CTACGGGCCCTGGGGAGAGTTTCGTGAAGGCTGGCACGTGCAGCGT
CCTTGGCAGTTGGCCCAGGAACTGGAGCCACAGAGCTGGTGCTGCC
GCTGGAATGACAAGCCCTACCTCTGTGCCCTGTACCAGCAGAGGCG
GCCCCACGTGGGCTGTGCTACATACAGGCCCCCACAGCCCGCCTGG
ATGTTCGGGGACCCCCACATCACCACCTTGGATGGTGTCAGTTACA
CCTTCAATGGGCTGGGGGACTTCCTGCTGGTCGGGGCCCAAGACGG
GAACTCCTCCTTCCTGCTTCAGGGCCGCACCGCCCAGACTGGCTCA
GCCCAGGCCACCAACTTCATCGCCTTTGCGGCTCAGTACCGCTCCA
GCAGCCTGGGCCCCGTCACGGTCCAATGGCTCCTTGAGCCTCACGA
CGCAATCCGTGTCCTGCTGGATAACCAGACTGTGACATTTCAGCCT
GACCATGAAGACGGCGGAGGCCAGGAGACGTTCAACGCCACCGGA
GTCCTCCTGAGCCGCAACGGCTCTGAGGTCTCGGCCAGCTTCGACG
GCTGGGCCACCGTCTCGGTGATCGCGCTCTCCAACATCCTCCACGC
CTCCGCCAGCCTCCCGCCCGAGTACCAGAACCGCACGGAGGGGCTC
CTGGGGGTCTGGAATAACAATCCAGAGGACGACTTCAGGATGCCC
AATGGCTCCACCATTCCCCCAGGGAGCCCTGAGGAGATGCTTTTCC
ACTTTGGAATGACCTGGCAGATCAACGGGACAGGCCTCCTTGGCAA
GAGGAATGACCAGCTGCCTTCCAACTTCACCCCTGTTTTCTACTCAC
AACTGCAAAAAAACAGCTCCTGGGCTGAACATTTGATCTCCAACTG
TGACGGAGATAGCTCATGCATCTATGACACCCTGGCCCTGCGCAAC
GCAAGCATCGGACTTCACACGAGGGAAGTCAGTAAAAACTACGAG
CAGGCGAACGCCACCCTCAATCAGTACCCGCCCTCCATCAATGGTG
GTCGTGTGATTGAAGCCTACAAGGGGCAGACCACGCTGATTCAGTA
CACCAGCAATGCTGAGGATGCCAACTTCACGCTCAGAGACAGCTGC
ACCGACTTGGAGCTCTTTGAGAATGGGACGTTGCTGTGGACACCCA
AGTCGCTGGAGCCATTCACTCTGGAGATTCTAGCAAGAAGTGCCAA
GATTGGCTTGGCATCTGCACTCCAGCCCAGGACTGTGGTCTGCCAT
TGCAATGCAGAGAGCCAGTGTTTGTACAATCAGACCAGCAGGGTG
GGCAACTCCTCCCTGGAGGTGGCTGGCTGCAAGTGTGACGGGGGC
ACCTTCGGCCGCTACTGCGAGGGCTCCGAGGATGCCTGTGAGGAGC
CGTGCTTCCCGAGTGTCCACTGCGTTCCTGGGAAGGGCTGCGAGGC
CTGCCCTCCAAACCTGACTGGGGATGGGCGGCACTGTGCGGCTCTG
GGGAGCTCTTTCCTGTGTCAGAACCAGTCCTGCCCTGTGAATTACT
GCTACAATCAAGGCCACTGCTACATCTCCCAGACTCTGGGCTGTCA
GCCCATGTGCACCTGCCCCCCAGCCTTCACTGACAGCCGCTGCTTC
CTGGCTGGGAACAACTTCAGTCCAACTGTCAACCTAGAACTTCCCT
TAAGAGTCATCCAGCTCTTGCTCAGTGAAGAGGAAAATGCCTCCAT
GGCAGAGGTCAACGCCTCGGTGGCATACAGACTGGGGACCCTGGA
CATGCGGGCCTTTCTCCGCAACAGCCAAGTGGAACGAATCGATTCT
GCAGCACCGGCCTCGGGAAGCCCCATCCAACACTGGATGGTCATCT
CGGAGTTCCAGTACCGCCCTCGGGGCCCGGTCATTGACTTCCTGAA
CAACCAGCTGCTGGCCGCGGTGGTGGAGGCGTTCTTATACCACGTT
CCACGGAGGAGTGAGGAGCCCAGGAACGACGTGGTCTTCCAGCCC
ATCTCCGGGGAAGACGTGCGCGATGTGACAGCCCTGAACGTGAGC
ACGCTGAAGGCTTACTTCAGATGCGATGGCTACAAGGGCTACGACC
TGGTCTACAGCCCCCAGAGCGGCTTCACCTGCGTGTCCCCGTGCAG
TAGGGGCTACTGTGACCATGGAGGCCAGTGCCAGCACCTGCCCAGT
GGGCCCCGCTGCAGCTGTGTGTCCTTCTCCATCTACACGGCCTGGG
GCGAGCACTGTGAGCACCTGAGCATGAAACTCGACGCGTTCTTCGG
CATCTTCTTTGGGGCCCTGGGCGGCCTCTTGCTGCTGGGGGTCGGG
ACGTTCGTGGTCCTGCGCTTCTGGGGTTGCTCCGGGGCCAGGTTCTC
CTATTTCCTGAACTCAGCTGAGGCCTTGCCTTGAAGGGGCAGCTGT
GGCCTAGGCTACCTCAAGACTCACCTCATCCTTACCGCACATTTAA
GGCGCCATTGCTTTTGGGAGACTGGAAAAGGGAAGGTGACTGAAG
GCTGTCAGGATTCTTCAAGGAGAATGAATACTGGGAATCAAGACA
GGACTATACCTTATCCATAGGCGCAGGTGCACAGGGGGAGGCCAT
AAAGATCAAACATGCATGGATGGGTCCTCACGCAGACACACCCAC
AGAAGGACACTAGCCTGGCGCGCGTGCACACACACACACACACAC
ACGAGTTCATAATGTGGTGATGGCCCTAAGTTAAGCAAAATGCTTC
TGCACACAAAACTCTCTGGTTTACTTCAAATTAACTCTATTTAAATA
AAGTCTCTCTGACTTTTTGTGTCTCCAAAAAAAAAAAAAAAAA

Variant polynucleotides of the present invention include polypeptides which are at least 70%, 75%, 80%, 85%, 90%, 95% or 100% identical to membrane associated molecules selected from the group consisting of SEQ ID NO:142, SEQ ID NOs: 3439-3445 and SEQ ID NOs: 3453-3457.

### Membrane Associated Molecules

In certain embodiments, the present invention is directed to methods of treating or diagnosing hyperproliferative diseases such as cancer, comprising the use of binding molecules which specifically bind to colon, lung, pancreatic or ovarian tumor-associated proteins. These polypeptides were identified from the malignant tumor samples of patients with colon cancer, as described in the Examples herein, and are overexpressed relative to normal nonmalignant colon tissue. All polypeptides described herein were isolated from the membranes of cells of tumor-associated cells. Thus, all membrane associated molecules described herein are membrane proteins and contain at least one or all of the following domains: extracellular domain, transmembrane domain or intracellular domain.

Table 1 lists membrane associated molecules of the present invention which were isolated from the cellular membranes of tumors from human patients with colon cancer and were identified via quantitative PCR (QPCR) analysis as described in Example 4.

TABLE 1

| BI NO: | SEQ ID NO: | PROTEIN NAME |
|---|---|---|
| BI6000000 | 3446 | SDAD1 (NM_018115.1) |
| BI6000001 | 3447 | GPR101 (NM_054021) |
| BI6000002 | 3448 | |
| BI6000003 | 3449 | OFR4N4 (NM_001005241) |
| BI6000004 | 3450 | OFR2G3 (NM_001001914) |
| BI6000005 | 3451 | |
| BI6000006 | 3452 | OFR4S2 (NM_001004059) |
| BI6000007 | 3458 | MUC 4 a (NP_060876) |
| BI6000008 | 3459 | MUC 4 b (NP_612155) |
| BI6000009 | 3460 | MUC 4 c (NP_612156) |
| BI6000010 | 3461 | MUC 4 d (NP_004523) |
| BI6000011 | 3462 | MUC 4 e (NP_612154) |

In certain embodiments of the present invention antibodies are employed which recognize polypeptides, variants or fragments thereof of the membrane associated molecules described herein. In certain embodiments polypeptides, variants or fragments thereof, of the membrane associated molecules include a predicted domain or region of the membrane associated molecules described herein. In certain embodiments, binding molecules such as antibodies which bind polypeptides, variants or fragments thereof, of the extracellular domains of the membrane associated molecules are employed.

Domains of the membrane associated molecules have been predicted based on homology to known polypeptide domains using the pfam program (see Bateman, A., et al., Nucl. Acids Res., 2004, Vol. 32, Database Issue, D138-D141). Table 2 below describes exemplary fragments based on homologies to known domains and the amino acid sequence positions which define the approximate beginning and end of the domains.

TABLE 2

| BI | SEQ ID | From AA | To AA | |
|---|---|---|---|---|
| NO. | NO: | Position | Position | Domain Description |
| BI6000000 | 3446 | 193 | 218 | coiled coil |
| | | 349 | 626 | SDA1 |
| BI6000001 | 3447 | 1 | 129 | IL6 |
| | | 1 | 23 | EGF_CA |
| | | 5 | 223 | Pfam:DUF975 |
| | | 5 | 131 | Pfam:FtsX |
| | | 5 | 510 | Pfam:Sre |
| | | 12 | 220 | PSN |
| | | 20 | 277 | Pfam:DUF914 |
| | | 25 | 145 | Pfam:PIG-U |
| | | 28 | 232 | Pfam:UPF0259 |
| | | 29 | 391 | Pfam:Dicty_CAR |
| | | 30 | 457 | Pfam:7tm_5 |
| | | 30 | 427 | Pfam:NADHdh |
| | | 34 | 216 | TLC |
| | | 35 | 457 | Pfam:7tm_2 |
| | | 37 | 59 | transmembrane |
| | | 38 | 140 | VKc |
| | | 38 | 212 | Pfam:Rnf-Nqr |
| | | 62 | 458 | Pfam:Presenilin |
| | | 66 | 303 | Pfam:PBP_sp32 |
| | | 72 | 94 | transmembrane |
| | | 97 | 250 | Pfam:DUF280 |
| | | 105 | 207 | Pfam:DUF895 |
| | | 106 | 259 | Pfam:DUF21 |
| | | 106 | 458 | Pfam:YjgP_YjgQ |
| | | 109 | 131 | transmembrane |
| | | 147 | 429 | Pfam:BTV_NS2 |
| | | 152 | 174 | transmembrane |
| | | 176 | 289 | Pfam:DUF809 |
| | | 197 | 417 | Pfam:MSP4 |
| | | 197 | 422 | Pfam:Neur_chan_memb |
| | | 198 | 220 | transmembrane |
| | | 202 | 239 | Pfam:DUF1443 |
| | | 207 | 279 | HMG |
| | | 212 | 407 | Pfam:S-antigen |
| | | 217 | 271 | GLA |
| | | 220 | 256 | LDLa |
| | | 221 | 297 | PTN |
| | | 243 | 401 | Pfam:Neuromodulin |
| | | 247 | 321 | HMG17 |
| | | 247 | 263 | low complexity |
| | | 271 | 364 | internal repeat 1 |
| | | 271 | 338 | internal repeat 2 |
| | | 278 | 371 | internal repeat 1 |
| | | 292 | 301 | low complexity |
| | | 312 | 373 | internal repeat 2 |
| | | 321 | 481 | Pfam:Sec62 |
| | | 369 | 470 | Pfam:Colicin_im |
| | | 379 | 395 | low complexity |
| | | 387 | 402 | ZnF_RBZ |
| | | 406 | 428 | transmembrane |
| | | 418 | 464 | LITAF |
| BI6000002 | 3448 | 1 | 43 | signal peptide |
| | | 29 | 51 | transmembrane |
| BI6000003 | 3449 | 1 | 21 | signal peptide |
| | | 41 | 287 | Pfam:7tm_1 |
| | | 25 | 47 | transmembrane |
| | | 59 | 78 | transmembrane |
| | | 98 | 120 | transmembrane |
| | | 141 | 163 | transmembrane |
| | | 200 | 222 | transmembrane |
| | | 243 | 265 | transmembrane |
| | | 270 | 289 | transmembrane |
| BI6000004 | 3450 | 1 | 39 | signal peptide |
| | | 41 | 290 | Pfam:7tm_1 |
| | | 26 | 48 | transmembrane |
| | | 61 | 83 | transmembrane |
| | | 98 | 120 | transmembrane |
| | | 144 | 166 | transmembrane |
| | | 199 | 221 | transmembrane |
| | | 241 | 263 | transmembrane |
| | | 273 | 292 | transmembrane |
| BI6000005 | 3451 | 125 | 139 | transmembrane |
| | | 1 | 123 | Pfam:DivIVA |
| | | 1 | 83 | Pfam:MbeD_MobD |
| | | 3 | 72 | Pfam:SecA_PP_bind |
| | | 4 | 77 | LER |
| | | 7 | 109 | SynN |
| | | 8 | 119 | RasGEFN |
| | | 14 | 108 | HPT |
| | | 17 | 107 | GED |
| | | 24 | 99 | Pfam:DUF709 |
| | | 25 | 137 | BBC |
| | | 25 | 124 | Pfam:Vicilin_N |
| | | 27 | 87 | Pfam:Exonuc_VII_S |
| | | 30 | 119 | SPEC |
| | | 30 | 50 | ITAM |
| | | 32 | 58 | GIT |
| | | 41 | 112 | DnaJ |
| | | 44 | 119 | Pfam:DUF526 |
| | | 44 | 123 | Pfam:Occludin_ELL |
| | | 53 | 108 | Pfam:HLH |
| | | 60 | 128 | coiled coil |
| | | 63 | 115 | L27 |
| | | 63 | 84 | NMU |
| | | 63 | 102 | Pfam:HTH_psq |
| | | 65 | 94 | RIIa |
| | | 67 | 140 | CARD |
| | | 69 | 113 | HLH |
| | | 69 | 128 | Hr1 |
| | | 95 | 117 | Pfam:Pox_A_type_inc |
| | | 110 | 124 | Pfam:Integrin_alpha |
| BI6000006 | 3452 | 39 | 285 | Pfam:7tm_1 |
| | | 1 | 49 | signal peptide |
| | | 31 | 53 | transmembrane |
| | | 58 | 80 | transmembrane |
| | | 100 | 122 | transmembrane |
| | | 143 | 165 | transmembrane |
| | | 201 | 223 | transmembrane |
| | | 236 | 258 | transmembrane |
| | | 268 | 287 | transmembrane |
| BI6000007 | 3458 | 1154 | 1311 | NIDO |
| | | 1310 | 1425 | AMOP |
| | | 1428 | 1610 | VWD |
| | | 1834 | 1866 | EGF |
| | | 1873 | 1914 | EGF |
| | | 2080 | 2117 | EGF |
| | | 2129 | 2151 | transmembrane |
| | | 40 | 344 | OSTEO |
| | | 66 | 122 | IGc1 |
| | | 69 | 273 | MA |
| | | 145 | 311 | MACPF |
| | | 205 | 328 | CBD_IV |
| | | 223 | 304 | BID_2 |
| | | 358 | 385 | PBD |
| | | 372 | 470 | B_lectin |
| | | 715 | 763 | ChtBD3 |
| | | 895 | 967 | H15 |
| | | 909 | 940 | CTNS |
| | | 957 | 1155 | AAA |
| | | 1118 | 1264 | CLECT |
| | | 1350 | 1577 | PRP |
| | | 1365 | 1422 | ZnF_UBR1 |
| | | 1469 | 1661 | LamG |
| | | 1471 | 1509 | RIIa |
| | | 1641 | 1665 | BTK |
| | | 1652 | 1750 | calpain_III |
| | | 1661 | 1689 | PTI |
| | | 1710 | 1845 | EPEND |
| | | 1716 | 1794 | C4 |
| | | 1793 | 1833 | FN1 |
| | | 1799 | 1854 | VWC |
| | | 1814 | 1885 | DISIN |
| | | 1817 | 1865 | EGF_Lam |
| | | 1825 | 1874 | C1 |
| | | 1825 | 1885 | FU |
| | | 1829 | 1953 | DM6 |
| | | 1829 | 1891 | PSA |
| | | 1833 | 1877 | LIM |
| | | 1840 | 1896 | FYVE |
| | | 1844 | 1865 | FES |
| | | 1846 | 1890 | AWS |
| | | 1848 | 1886 | ZnF_C4 |
| | | 1850 | 1885 | ShKT |
| | | 1851 | 1884 | BowB |
| | | 1852 | 1900 | ChtBD2 |
| | | 1854 | 1902 | VWC_out |
| | | 1860 | 1894 | BBOX |
| | | 1865 | 1898 | ANATO |
| | | 1873 | 1898 | DEFSN |
| | | 1873 | 1902 | ChtBD1 |
| | | 1874 | 1913 | GRAN |
| | | 1879 | 1902 | RING |
| | | 1890 | 1940 | Ubox |
| | | 1897 | 1934 | LRRNT |
| | | 1968 | 2082 | RPR |
| | | 2058 | 2117 | PSI |
| | | 2072 | 2096 | ZnF_C3H1 |
| | | 2080 | 2102 | FOLN |
| | | 2081 | 2117 | EGF_CA |
| | | 2082 | 2113 | SAPA |
| BI6000008 | 3459 | 1153 | 1255 | NIDO |
| | | 23 | 163 | VWD |
| | | 40 | 344 | OSTEO |
| | | 66 | 122 | IGc1 |
| | | 69 | 273 | MA |
| | | 145 | 311 | MACPF |
| | | 205 | 328 | CBD_IV |
| | | 223 | 304 | BID_2 |
| | | 358 | 385 | PBD |
| | | 372 | 470 | B_lectin |
| | | 594 | 702 | THN |
| | | 715 | 763 | ChtBD3 |
| | | 743 | 857 | SR |
| | | 831 | 968 | DoH |
| | | 894 | 966 | H15 |
| | | 908 | 939 | CTNS |
| | | 956 | 1154 | AAA |
| BI6000009 | 3460 | 1154 | 1311 | NIDO |
| | | 1310 | 1425 | AMOP |
| | | 1428 | 1610 | VWD |
| | | 40 | 344 | OSTEO |
| | | 66 | 122 | IGc1 |
| | | 69 | 273 | MA |
| | | 145 | 311 | MACPF |
| | | 205 | 328 | CBD_IV |
| | | 223 | 304 | BID_2 |
| | | 358 | 385 | PBD |
| | | 372 | 470 | B_lectin |
| | | 594 | 702 | THN |
| | | 715 | 763 | ChtBD3 |
| | | 895 | 967 | H15 |
| | | 909 | 940 | CTNS |
| | | 957 | 1155 | AAA |
| | | 1118 | 1264 | CLECT |
| | | 1350 | 1577 | PRP |
| | | 1363 | 1378 | DEFSN |
| | | 1365 | 1422 | ZnF_UBR1 |
| | | 1377 | 1409 | ANATO |
| | | 1469 | 1661 | LamG |
| | | 1471 | 1509 | RIIa |
| | | 1641 | 1665 | BTK |
| | | 1652 | 1750 | calpain_III |
| | | 1661 | 1689 | PTI |
| | | 1716 | 1794 | C4 |
| | | 1785 | 1826 | ZnF_TAZ |
| | | 1790 | 1820 | KAZAL |
| BI6000010 | 3461 | 161 | 318 | NIDO |
| | | 317 | 432 | AMOP |
| | | 435 | 617 | VWD |
| | | 841 | 873 | EGF |
| | | 880 | 921 | EGF |
| | | 1087 | 1124 | EGF |
| | | 1136 | 1158 | transmembrane |
| | | 125 | 271 | CLECT |
| | | 214 | 252 | ChtBD3 |
| | | 357 | 584 | PRP |
| | | 372 | 429 | ZnF_UBR1 |
| | | 476 | 668 | LamG |
| | | 478 | 516 | RIIa |
| | | 480 | 510 | CTNS |
| | | 648 | 672 | BTK |
| | | 659 | 757 | calpain_III |
| | | 668 | 696 | PTI |
| | | 717 | 852 | EPEND |
| | | 723 | 801 | C4 |
| | | 800 | 840 | FN1 |
| | | 806 | 861 | VWC |
| | | 821 | 892 | DISIN |
| | | 824 | 872 | EGF_Lam |
| | | 832 | 881 | C1 |
| | | 832 | 892 | FU |
| | | 836 | 960 | DM6 |
| | | 836 | 898 | PSA |
| | | 840 | 884 | LIM |
| | | 847 | 903 | FYVE |
| | | 851 | 872 | FES |
| | | 853 | 897 | AWS |
| | | 855 | 893 | ZnF_C4 |
| | | 857 | 892 | ShKT |
| | | 858 | 891 | BowB |
| | | 859 | 907 | ChtBD2 |
| | | 861 | 909 | VWC_out |
| | | 867 | 901 | BBOX |
| | | 872 | 905 | ANATO |
| | | 880 | 909 | ChtBD1 |
| | | 880 | 905 | DEFSN |
| | | 881 | 920 | GRAN |
| | | 886 | 909 | RING |
| | | 897 | 947 | Ubox |
| | | 904 | 941 | LRRNT |
| | | 975 | 1089 | RPR |
| | | 1021 | 1089 | CT |
| | | 1065 | 1124 | PSI |
| | | 1079 | 1103 | ZnF_C3H1 |
| | | 1087 | 1109 | FOLN |
| | | 1088 | 1124 | EGF_CA |
| | | 1089 | 1120 | SAPA |
| BI6000011 | 3462 | 12 | 34 | transmembrane |
| | | 110 | 267 | NIDO |
| | | 266 | 381 | AMOP |
| | | 384 | 566 | VWD |
| | | 790 | 822 | EGF |
| | | 829 | 870 | EGF |
| | | 1036 | 1073 | EGF |
| | | 1085 | 1107 | transmembrane |
| | | 74 | 220 | CLECT |
| | | 163 | 201 | ChtBD3 |
| | | 306 | 533 | PRP |
| | | 321 | 378 | ZnF_UBR1 |
| | | 425 | 617 | LamG |
| | | 427 | 465 | RIIa |
| | | 429 | 459 | CTNS |
| | | 597 | 621 | BTK |
| | | 608 | 706 | calpain_III |
| | | 617 | 645 | PTI |
| | | 666 | 801 | EPEND |
| | | 672 | 750 | C4 |
| | | 749 | 789 | FN1 |
| | | 755 | 810 | VWC |
| | | 770 | 841 | DISIN |
| | | 773 | 821 | EGF_Lam |
| | | 781 | 830 | C1 |
| | | 781 | 841 | FU |
| | | 785 | 909 | DM6 |
| | | 785 | 847 | PSA |
| | | 789 | 833 | LIM |
| | | 796 | 852 | FYVE |
| | | 800 | 821 | FES |
| | | 802 | 846 | AWS |
| | | 804 | 842 | ZnF_C4 |
| | | 806 | 841 | ShKT |
| | | 807 | 840 | BowB |
| | | 808 | 856 | ChtBD2 |
| | | 810 | 858 | VWC_out |
| | | 816 | 850 | BBOX |
| | | 821 | 854 | ANATO |
| | | 829 | 858 | ChtBD1 |
| | | 829 | 854 | DEFSN |
| | | 830 | 869 | GRAN |
| | | 835 | 858 | RING |
| | | 846 | 896 | Ubox |
| | | 853 | 890 | LRRNT |
| | | 924 | 1038 | RPR |
| | | 970 | 1038 | CT |
| | | 1014 | 1073 | PSI |
| | | 1028 | 1052 | ZnF_C3H1 |
| | | 1036 | 1058 | FOLN |
| | | 1037 | 1073 | EGF_CA |
| | | 1038 | 1069 | SAPA |

Intracellular, extracellular and nontransmembrane domains of the membrane associated molecules were predicted by the Kyte and Doolittle hydropatliy algorithm (Kyte, J. and Doolittle, R., J. Mol. Biol. 157: 105-132 (1982)), Chou and Fasman method to predict secondary structure (Chou and Fasman, Adv. Enz.:47, 45-147 (1978), and Goldman, Engelman and Steitz Transbilayer Helices Prediction algorithm (Engelman, D. M. et al. Annu. Rev. Biophys. Biophys. Chem. 15:321-353 (1986)).

Table 3 below provides portions of each membrane associated molecules which are predicted to be part of the intracellular, extracellular and nontransmembrane portions of the polypeptide.

TABLE 3

| | | Predicted | Predicted | Predicted Non- |
|---|---|---|---|---|
| SEQ ID | | Intracellular | Extracellular | Transmembrane |
| NO | BI Number | Regions | Regions | Regions |
| 3446 | BI6000000 | | 1-627 | 1-627 |
| 3447 | BI6000001 | 60-71; 132-151; | 1-36; 95-108; 175-197; | 1-36; 60-71; 95-108; |
| | | 221-405; 461-512 | 429-437 | 132-151; 175-197; 221-405; |
| | | | | 429-437; 461-512 |
| 3448 | BI6000002 | 52-160 | 1-28; | 1-28; 52-160 |
| 3449 | BI6000003 | 48-58; 121-140; | 1-24; 79-97; 164-199; | 1-24; 48-58; 79-97; 121-149; |
| | | 223-242; 290-316 | 166-169 | 164-199; 223-242; |
| | | | | 266-269; 290-316 |
| 3450 | BI6000004 | 49-60; 121-143; | 1-25; 84-97; 167-198; | 1-25; 49-60; 84-97; 121-143; |
| | | 222-240; 293-309 | 264-272 | 167-198; 222-240; |
| | | | | 266-272; 293-309 |
| 3451 | BI6000005 | 1-124 | 140 | 1-125; 140 |
| 3452 | BI6000006 | 54-57; 123-142; | 1-30; 81-99; 166-200; | 1-40; 54-57; 81-99; 123-142; |
| | | 224-235; 288-311 | 259-267 | 166-200; 224-235; |
| | | | | 259-267; 288-311 |
| 3458 | BI6000007 | 2152-2169 | 1-2128 | 1-2128; 2152-2169 |
| 3459 | BI6000008 | | 1-1255 | 1-1255 |
| 3460 | BI6000009 | | 1-1827 | 1-1827 |
| 3461 | BI6000010 | 1159-1176 | 1-1135 | 1-1135; 1159-1176 |
| 3462 | BI6000011 | 1-11; 1108-1125 | 35-1084 | 1-11; 35-1084; 1108-1125 |

In the context of the amino acids comprising the various structural and functional domains of a membrane associated molecule, the term “about” includes the particularly recited value and values larger or smaller by several (e.g., 10, 9, 8, 7, 6, 5, 4, 3, 2, or 1) amino acids. One of ordinary skill would appreciate that the amino acid residues constituting these domains may vary slightly (e.g., by about 1 to 15 residues) depending on the criteria used to define the domain. Thus in various embodiments, the extracellular domain of a colon associated polypeptide comprises, consists essentially of, or consists of, for example, the amino acid residues listed in Table 3 as comprising the extracellular domain.

### Treatment Methods Using Therapeutic Binding Molecules, in Particular, Membrane Associated Molecule-Specific Antibodies, or Immunospecific Fragments Thereof

One embodiment of the present invention provides methods for treating a hyperproliferative disease or disorder, e.g., cancer, a malignancy, a tumor, or a metastasis thereof, in an animal suffering from such disease or predisposed to contract such disease, the method comprising, consisting essentially of, or consisting of administering to the animal an effective amount of a binding molecule, more specifically a binding polypeptide, and even more specifically an antibody or immunospecific fragment thereof, that binds to a membrane associated molecule described herein. A specific embodiment of the present invention is a method of treatment as above, where the binding molecule binds specifically to at least one epitope of a polypeptide selected from the group consisting of SEQ ID NOs: 1288, 3446-3452 or SEQ ID NOs: 3458-3462.

A therapeutic binding molecule, e.g., a binding polypeptide, e.g., an antibody that binds specifically to a membrane associated molecule described herein, to be used in treatment methods disclosed herein can be prepared and used as a therapeutic agent that stops, reduces, prevents, or inhibits cellular activities involved in cellular hyperproliferation, e.g., cellular activities that induce the altered or abnormal pattern of vascularization that is often associated with hyperproliferative diseases or disorders. Characteristics of membrane associated molecules that are suitable targets for such binding molecules include membrane associated molecules located on the cell surface and disease- or disorder-specific expression; e.g., by cells of tumor-induced or inflammatory vascular tissue. Therapeutic binding molecules that bind specifically to such disease- or disorder-associated proteins are referred to herein as binding molecules or binding polypeptides. In certain embodiments, the binding molecule has at least one binding domain which specifically binds to a target molecule such as a polypeptide, e.g., a tumor-expressed or tumor-associated cell surface antigen.

Binding polypeptides include antibodies or immunospecific fragments thereof such as monoclonal, chimeric or humanized antibodies, domain-deleted antibodies, and fragments of antibodies that bind specifically to membrane associated molecules. The antibodies may be monovalent, bivalent, polyvalent, or bifunctional antibodies, and the antibody fragments include Fab F(ab′)₂, and Fv. Therapeutic binding molecules produced according to the invention also include fusion proteins that target a ligand or receptor of a membrane associated molecule described herein which is expressed on the surface of a disease-associated cell. Another type of binding polypeptide, also used herein as an immunogen, comprises a non-antigen-specific fragment of an immunoglobulin joined to the extracellular domain of a transmembrane membrane associated molecule, e.g., amino acid residues 1-627 of SEQ ID NO:3446, to generate a receptor:Ig fusion protein that antagonizes and neutralizes the cellular function of the target protein.

Therapeutic binding molecules according to the invention can be used in unlabeled or unconjugated form, or can be coupled or linked to cytotoxic moieties such as radiolabels and biochemical cytotoxins to produce agents that exert therapeutic effects.

In certain embodiments, a binding domain on a binding molecule or binding polypeptide is an antigen binding domain, and the binding polypeptide is an antibody, or immunospecific fragment thereof. An antigen binding domain is formed by antibody variable regions that vary from one antibody to another. Naturally occurring antibodies comprise at least two antigen binding domains, i.e., they are at least bivalent. As used herein, the term “antigen binding domain” includes a site that specifically binds an epitope on an antigen (e.g., a cell surface or soluble antigen). The antigen binding domain of an antibody typically includes at least a portion of an immunoglobulin heavy chain variable region and at least a portion of an immunoglobulin light chain variable region. The binding site formed by these variable regions determines the specificity of the antibody.

The present invention provides methods for treating various hyperproliferative disorders, e.g., by inhibiting tumor growth, in a mammal, comprising, consisting essentially of, or consisting of administering to the mammal an effective amount of a binding agent that binds specifically to a transmembrane protein identified by the invention as being specifically or predominantly present in tumor cells or tumor-associate tissue, preferably colon tumor cells or colon tumor-associated tissue.

In addition to antibodies and immunospecific fragments thereof, binding molecules of the present invention include a fusion protein, an agent which elicits a T-cell response specific for the membrane associated molecules, variants or fragments described herein, and a small molecule. Similar binding molecules may be used in the in vitro and in vivo diagnostic methods described in more detail below.

The present invention is more specifically directed to a method of treating a hyperproliferative disease, e.g., inhibiting or preventing tumor formation, tumor growth, tumor invasiveness, and/or metastasis formation, in an animal, e.g., a mammal, e.g., a human, comprising, consisting essentially of, or consisting of administering to an animal in need thereof an effective amount of a binding agent, e.g., a binding molecule, more specifically a binding polypeptide, and even more specifically an antibody or immunospecific fragment thereof, which specifically binds to one or more epitopes of a membrane associated molecule, variant polypeptide or fragment thereof described herein.

In particular, the present invention includes a method for treating a hyperproliferative disease, e.g., inhibiting tumor formation, tumor growth, tumor invasiveness, and/or metastasis formation in an animal, e.g., a mammal, e.g., a human patient, or prolonging survival of the animal, where the method comprises, consists essentially of, or consists of administering to an animal in need of such treatment an effective amount of a composition comprising, consisting essentially of, or consisting of, in addition to a pharmaceutically acceptable carrier, a binding molecule which specifically binds to a membrane associated molecule, variant or fragment thereof.

Such membrane associated molecules include the following polypeptides and their respective amino acid sequences:

SEQ ID NO: 3446

MFMAQISHCYPEYLSNFPQEVKDLLSCNHTVLDPDLRMTFCKALILLR
NKNLINPSSLLELFFELFRCHDKLLRKTLYTHIVTDIKINAKHKNNKV
NVVLQNFMYTMLRDSNATAAKMSLDVMIELYRRNIWNDAKTVNVIT
TACFSKVTKILVAALTFFLGKDEDEKQDSDSESEDDGPTARDLLVQYA
TGKKSSKNKKKLEKAMKVLKKQKKKKKPEVFNFSAIHLIHDPQDFAE
KLLKQLECCKERFEVKMMLMNLISRLVGIHELFLFNEYPFLQRFLQPH
QREVTKILLFAAQASHHLVPPEIIQSLLMTVANNFVTDKNSGEVMTVGI
NAIKEITARCPLAMTEELLQDLAQYKTHKDKNVMMSARTLIHLFRTLN
PQMLQKKFRGKPTEASIEARVQEYGELDAKDYIPGAEVLEVEKEENAE
NDEDGWESTSLSEEEDADGEWIDVQHSSDEEQQEISKKLNSMPMEER
KAKAAAISTSRVLTQEDFQKIRMAQMRKELDAAPGKSQKRKYIEDSD
EEPRGELLSLRDIERLHKKPKSDKETRLATAMAGKTDRKEFVRKKTKT
NPFSSSTNKEKKKQKNFMMMRYSQNVRSKNKRSFREKQLALRDALL
KKRKRMK

SEQ ID NO: 3447

MTSTCTNSTRESNSSHTCMPLSKMPISLAHGIIRSTVLVIFLAASFVGNI
VLALVLQRKPQLLQVTNRFIFNLLVTDLLQISLVAPWVVATSVPLFWP
LNSHFCTALVSLTHLFAFASVNTIVVVSVDRYLSIIHPLSYPSKMTQRR
GYLLLYGTWIVAILQSTPPLYGWGQAAFDERNALCSMIWGASPSYTIL
SVVSFIVIPLIVMIACYSVVFCAARRQHALLYNVKRHSLEVRVKDCVE
NEDEEGAEKKEEFQDESEFRRQHEGEVKAKEGRMEAKDGSLKAKEGS
TGTSESSVEARGSEEVRESSTVASDGSMEGKEGSTKVEENSMKADKG
RTEVNQCSIDLGEDDMEFGEDDINFSEDDVEAVNIPESLPPSRRNSNSN
PPLPRCYQCKAAKVIFIIIFSYVLSLGPYCFLAVLAVWVDVETQVPQWV
ITIIIWLFFLQCCIHPYVYGYMHKTIKKEIQDMLKKFFCKEKPPKEDSHP
DLPGTEGGTEGKIVPSYDSATFP

SEQ ID NO: 3448

MLSPHIYLPKCWNYRHEPLCLAVCFHFQLSLCFCCFILSLLCLCVLFNS
LFKTPRTCTASTGNIFWQASQEVIPKFGVYFSCLFFLLHTGESQSLSLFS
FPTWDGWWAAPKHRGNCRFLAGATLKDSFLSFPVVVPDPYVWHSLG
RAGICFSDLNLVFSC

SEQ ID NO: 3449

MKIANNTVVTEFILLGLTQSQDIQLLVFVLILIFYLIILPGNFLIIFTIR
SDPGLTAPLYFFLGNLAFLDASYSFIVAPRMLVDFFSEKKVISYRGCITQ
LFFLHFLGGGEGLLLVVMAFDRYIAICRPLHCSTVMNPRACYAMMLALWL
GGFVHSIIQVVLILRLPFCGPNQLDNFFCDVRQVIKLACTDMFVVELLMV
FNSGLMTLLCFLGLLASYAVILCHVRRAASEGKNKAMSMCTTRVIIILLM
FGPAIFIYMCPFRALPADKMVSLFHTVIFPLMNPMIYTLRNQEVKTSMKR
LLSRHVVCQVDFIIRN

SEQ ID NO: 3450

MGLGNESSLMDFILLGFSDHPRLEAVLFVFVLFFYLLTLVGNFTIIIISY
LDPPLHTPMYFFLSNLSLLDICFTTSLAPQTLVNLQRPRKTITYGGCVAQ
LYISLALGSTECILLADMALDRYIAVCKPLHYVVIMNPRLCQQLASISWL
SGLASSLIHATFTLQLPLCGNHRLDHFICEVPALLKLACVDTTVNELVLF
VVSVLFVVIPPALISISYGFITQAVLRIKSVEARHKAFSTCSSHLTVVII
FYGTIIYVYLQPSDSYAQDQGKFISLFYTMVTPTLNPIIYTLRNKDMKEA
LRKLLSGKL

SEQ ID NO: 3451

MLDLEKEKDLFSRQKGYLEEELDYRKQALDQAYLKIQDLEATLYTAL
QQEPGRRAGEALSEGQREDLQAAVEKVRRQILRQSREFDSQILRERME
LLQQAQQRIRELEDKLEFQKRHLKELEEKFLFLFLFFSLAFILWP

SEQ ID NO: 3452

MEKINNVTEFIFWGLSQSPEIEKVCFVVFSFFYIIILLGNLLIMLTVCLS
NLFKSPMYFFLSFLSFVDICYSSVTAPKMIVDLLAKDKTISYVGCMLQLL
GVHFFGCTEIFILTVMAYDRYVAICKPLHYMTIMNRETCNKMLLGTWVGG
FLHSIIQVALVVQLPFCGPNEIDHYFCDVHPVLKLACTETYIVGVVVTAN
SGTIALGSFVILLISYSIILVSLRKQSAEGRRKALSTCGSHIAMVVIFFG
PCTFMYMRPDTTFSEDKMVAVFYTIITPMLNPLIYTLRNAEVKNAMKKLW
GRNVFLEAKGK

SEQ ID NO: 3458

MKGARWRRVPWYSLSCLCLCLLPHVVPGTTEDTLITGSKTPAPVTSTGST
TATLEGQSTAASSRTSNQDISASSQNHQTKSTETTSKAQTDTLTQMMTST
LFSSPSVHNVMETVTQETAPPDEMTTSFPSSVTNTLMMTSKTITMTTSTD
STLGNTEETSTAGTESSTPVTSAVSITAGQEGQSRTTSWRTSIQDTSASS
QNHWTRSTQTTRESQTSTLTHRTTSTPSFSPSVHNVTGTVSQKTSPSGET
ATSSLCSVTNTSMMTSEKITVTTSTGSTLGNPGETSSVPVTGSLMPVTSA
ALVTVDPEGQSPATFSRTSTQDTTAFSKNHQTQSVETTRVSQINTLNTLT
PVTTSTVLSSPSGFNPSGTVSQETFPSGETTISSPSSVSNTFLVTSKVFR
MPISRDSTLGNTEETSLSVSGTISAITSKVSTIWWSDTLSTALSPSSLPP
KISTAFHTQQSEGAETTGRPHERSSFSPGVSQEIFTLHETTTWPSSFSSK
GHTTWSQTELPSTSTGAATRLVTGKPSTGAAGTIPRVPSKVSAIGEPGEP
TTYSSHSTTLPKTTGAGAQTQWTQETGTTGEALLSSPSYSVTQMIKTATS
PSSSPMLDRHTSQQITTAPSTNHSTIHSTSTSPQESPAVSQRGHTQAPQT
TQESQTTRSVSPMTDTKTVTTPGSSFTASGHSPSEIVPQDAPTISAATTF
APAPTGDGHTTQAPTTALQAAPSSHDATLGPSGGTSLSKTGALTLANSVV
STPGGPEGQWTSASASTSPDTAAAMTHTHQAESTEASGQTQTSEPASSGS
RTTSAGTATPSSSGASGTTPSGSEGISTSGETTRFSSNPSRDSHTTQSTT
ELLSASASHGAIPVSTGMASSIVPGTFHPTLSEASTAGRPTGQSSPTSPS
ASPQETAAISRMAQTQRTRTSRGSDTISLASQATDTFSTVPPTPPSITSS
GLTSPQTQTHTLSPSGSGKTFTTALISNATPLPVTSTSSASTGHATPLAV
SSATSASTVSSDSPLKMETSGMTTPSLKTDGGRRTATSPPPTTSQTIIST
IPSTAMHTRSTAAPIPILPERGVSLFPYGADAGDLEFVRRTVDFTSPLFK
PATGFPLGSSLRDSLYFTDNGQIIFPESDYQIFSYPNPLPTGFTGRDPVA
LVAPFWDDADFSTGRGTTFYQEYETFYGEHSLLVQQAESWIRKITNNGGY
KARWALKVTWVNAHAYPAQWTLGSNTYQAILSTDGSRSYALFLYQSGGMQ
WDVAQRSGKPVLMGFSSGDGFFENSPLMSQPVWERYRPDRFLNSNSGLQG
LQFYGLHRFERPNYRLECLQWLKSQPRWPSWGWNQVSCPCSWQQGRRDLR
FQPVSIGRWGLGSRQLCSFTSWRGGVCCSYGPWGEFREGWHVQRPWQLAQ
ELEPQSWCCRWNDKPYLCALYQQPRPHVGCATYRPPQPAWMFGDPHITTL
DGVSYTFNGLGDFLLVGAQDGNSSFLLQGRTAQTGSAQATNFIAFAAQYR
SSSLGPVTVQWLLEPHDAIRVLLDNQTVTFQPDHEDGGGQETFNATGVLL
SRNGSEASASFDGWATVSVIALSNILHSSASLPPEYQNRTEGLLGVWNNN
PEDDFRMPNGSTIPPGSPEEMLFHFGMTWQINGTGLLGKRNDQLPSNFTP
VFYSQLQKNSSWAEHLISNCDGDSSCJYDTLALRNASIGLHTREVSKNYE
QANATLNQYPPSINGGRVIEAYKGQTTLIQYTSNAEDANFTLRDSCTDLE
LFENGTLLWTPKSLEPFTLEILARSAKIGLASALQPRTVVCHCNAESQCL
YNQTSRVGNSSLEVAGCKCDGGTFGRYCEGSEDACEEPCFPSVHCVPGKG
CEACPPNLTGDGRHCAALGSSFLCQNQSCPVNYCYNQGHCYISQTLGCQP
MCTCPPAFTDSRCFLAGNNFSPTVNLELPLRVIQLLLSEEENASMAEVNA
SVAYRLGTLDMRAFLRNSQVERIDSAAPASGSPIQHWMVISEFQYRPRGP
VIDFLNNQLLAAVVEAFLYHVPRRSEEPRNDVVFQPISGEDVPDVTALNV
STLKAYFRCDGYKGYDLVYSPQSGFTCVSPCSRGYCDHGGQCQHLPSGPR
CSCVSFSIYTAWGEHCEHLSMKLDAFFGIFFGALGGLLLLGVGTFVVLRF
WGCSGARFSYFLNSAFALP

SEQ ID NO: 3459

MKGARWRRVPWVSLSCLCLCLLPHVVPGTTEDTLITGSKTPAPVTSTGST
TATLEGQSTAASSRTSNQDISASSQNHQTKSTETTSKAQTDTLTQMMTST
LFSSPSVHNVMETVTQETAPPDEMTTSFPSSVTNTLMMTSKTITMTTSTD
STLGNTEETSTAGTESSTPVTSAVSITAGQEGQSRTTSWRTSIQDTSASS
QNHWTRSTQTTRESQTSTLTHRTTSTPSFSPSVHNVTGTVSQKTSPSGET
ATSSLCSVTNTSMMTSEKITVTTSTGSTLGNPGETSSVPVTGSLMPVTSA
ALVTVDPEGQSPATFSRTSTQDTTAFSKNHQTQSVETTRVSQINTLNTLT
PVTTSTVLSSPSGFKPSGTVSQETFPSGETTISSPSSVSNTFLVTSKVFR
MPISRDSTLGNTEETSLSVSGTISAITSKVSTIWWSDTLSTALSPSSLPP
KISTAFHTQQSEGAETTGRPHERSSFSPGVSQEIFTLHETTTWPSSFSSK
GHTTWSQTELPSTSTGAATRLVTGKPSTGAAGTIPRVPSKVSAIGEPGEP
TTYSSHSTTLPKTTGAGAQTQWTQETGTTGEALLSSPSYSVTQMIKTATS
PSSSPMLDRHTSQQITTAPSTNHSTIHSTSTSPQESPAVSQRGHTQAPQT
TQESQTTRSVSPMTDTKTVTTPGSSFTASGHSPSEIVPQDAPTISAATTF
APAPTGDGHTTQAPTTALQAAPSSHDATLGPSGGTSLSKTGALTLANSVV
STPGGPEGQWTSASASTSPDTAAAMTHTHQAESTEASGQTQTSEPASSGS
RTTSAGTATPSSSGASGTTPSGSEGISTSGETTFSSNPSRDSHTTQSTTE
LLSASASHGAIPVSTGMASSIVPGTFHPTLSEASTAGRPTGQSSPTSPSA
SPQETAAISRMAQTQRTRTSRGSDTISLASQATDTFSTVPPTPPSITSSG
LTSPQTQTHTLSPSGSGKTFTTALISNATPLPVTSTSSASTGHATPLAVS
SATSASTVSSDSPLKMETSGMTTPSLKTDGGRRTATSPPPTTSQTIISTI
PSTAMHTRSTAAPIPILPERGVSLFPYGADAGDLEFVRRTVDFTSPLFKP
ATGFPLGSSLRDSLYFTDNGQIIFPESDYQIFSYPNPLPTGFTGRDPVAL
VAPFWDDADFSTGRGTTFYQEYETFYGEHSLLVQQAESWIRKITNNGGYK
ARWALKVTWVNAHAYPAQWTLGSNTYQAILSTDGSRSYALFLYQSGGMQW
DVAQL

SEQ ID NO: 3460

MKGARWRRVPWVSLSCLCLCLLPHVVPGTTEDTLITGSKTPAPVTSTGST
TATLEGQSTAASSRTSNQDISASSQNHQTKSTETTSKAQTDTLTQMMTST
LFSSPSVHNVMETVTQETAPPDEMTTSFPSSVTNTLMMTSKTITMTTSTD
STLGNTEETSTAGTESSTPVTSAVSITAGQEGQSRTTSWRTSIQDTSASS
QNHWTRSTQTTRESQTSTLTHRTTSTPSFSPSVHNVTGTVSQKTSPSGET
ATSSLCSVTNTSMMTSEKITVTTSTGSTLGNPGETSSVPVTGSLMPVTSA
ALVTVDPEGQSPATFSRTSTQDTTAFSKNHQTQSVETTRVSQINTLNTLT
PVTTSTVLSSPSGFNPSGTVSQETFPSGETTISSPSSVSNTFLVTSKVFR
MPISRDSTLGNTEETSLSVSGTISAITSKVSTIWWSDTLSTALSPSSLPP
KISTALFHTQQSEGAETTGRPHERSSFSPGVSQEIFTLHETTTWPSSFSS
KGHTTWSQTELPSTSTGAATRLVTGNPSTGAAGTIPRVPSKVSAIGEPGE
PTTYSSHSTTLPKTTGAGAQTQWTQETGTTGEALLSSPSYSVTQMIKTAT
SPSSSPMLDRHTSQQITTAPSTNHSTIHSTSTSPQESPAVSQRGHTQAPQ
TTQESQTTRSVSPMTDTKTVTTPGSSFTASGHSPSEIVPQDAPTISAATT
FAPAPTGDGHTTQAPTTALQAAPSSHDATLGPSGGTSLSKTGALTLANSV
VSTPGGPEGQWTSASASTSPDTAAAMTHTHQAESTEASGQTQTSEPASSG
SRTTSAGTATPSSSGASGTTPSGSEGISTSGETTRFSSNPSRDSHTTQST
TELLSASASHGAIPVSTGMASSIVPGTFHPTLSEASTAGRPTGQSSPTSP
SASPQETAAISRMAQTQRTRTSRGSDTISLASQATDTFSTVPPTPPSITS
SGLTSPQTQTHTLSPSGSGKTFTTALISNATPLPVTSTSSASTGHATPLA
VSSATSASTVSSDSPLKMETSGMTTPSLKTDGGRRTATSPPPTTSQTIIS
TIPSTAMHTRSTAAPIPILPERGVSLFPYGADAGDLEFVRRTVDPTSPLF
KPATGFPLGSSLRDSLYFTDNGQIIFPESDYQIFSYPNPLPTGFTGRDPV
ALVAPFWDDADFSTGRGTTFYQEYETFYGEHSLLVQQAESWIRKITNNGG
YKARWALKVTWVNAHAYPAQWTLGSNTYQAILSTDGSRSYALFLYQSGGM
QWDVAQRSGKPVLMGFSSGDGFFENSPLMSQPVWERYRPDRFLNSNSGLQ
GLQFYGLHREERPNYRLECLQWLKSQPRWPSWGWNQVSCPCSWQQGRRDL
RFQPVSIGRWGLGSRQLCSFTSWRGGVCCSYGPWGEFREGWHVQRPWQLA
QELEPQSWCCRWNDKPYLCALYQQRRPHVGCATYRPPQPAWMFGDPHITT
LDGVSYTFNGLGDFLLVGAQDGNSSFLLQGRTAQTGSAQATNFIAFAAQY
RSSSLGPVTVQWLLEPHDAIRVLLDNQTVTFQPDHEDGGGQETFNATGVL
LSRNGSEASASFDGWATVSVIALSNILHSSASLPPEYQNRTEGLLGVWNN
NPEDDFRMPNGSTIPPGSPEEMLFHFGMTWQINGTGLLGKRNDQLPSNFT
PVFYSQLQKNSSWAEHLISNCDGDSSCIYDTLALRNASIGLHTREVSKNY
EQANATLNQYPPSINGGRVIEAYKGQTTLIQYTSNAEDANFTLRDSCTDL
ELFENGTLLWTPKSLEPFTLEILARSAKIGLASALQPRTVVCHCNAESQC
LYNQTSRVGNSSLELWGALSCVRTSPAL

SEQ ID NO: 3461

MKGARWRRVPWVSLSCLCLCLLPHVVPGMTTPSLKTDGGRRTATSPPPTT
SQTIISTIPSTAMHTRSTAAPIPILPERGVSLFPYGADAGDLEFVRRTVD
FTSPLFKPATGFPLGSSLRDSLYFTDNGQIIFPESDYQIFSYPNPLPTGF
TGRDPVALVAPFWDDADFSTGRGTTFYQEYETFYGEHSLLVQQAESWIRK
ITNNGGYKARWALKVTWVNAHAYPAQWTLGSNTYQAILSTDGSRSYALFL
YQSGGMQWDVAQRSGKPVLMGFSSGDGYFENSPLMSQPVWERYRPDRPLN
SNSGLQGLQFYRLHREERPNYRLECLQWLKSQPRWPSWGWNQVSCPCSWQ
QGRRDLRFQPVSIGRWGLGSRQLCSFTSWRGGVCCSYGPWGEFRLGWHVQ
RPWQLAQELEPQSWCCRWNDKPYLCALYQQRRPHVGCATYRPPQPAWMFG
DPHITTLDGVSYTFNGLGDFLLVGAQDGNSSFLLQGRTAQTGSAQATNFI
AFAAQYRSSSLGPVTVQWLLEPHDAIRVLLDNQTVTFQPDHEDGGGQETF
NATGVLLSRNGSEVSASFDGWATVSVIALSNILHASASLPPEYQNRTEGL
LGVWNNNPEDDFRMPNGSTIPPGSPEEMLFHFGMTWQINGTGLLGKRNDQ
LPSNFTPVFYSQLQKNSSWAEHLISNCDGDSSCIYDTLALRNASIGLHTR
EVSKNYEQANATLNQYPPSINGGRVIEAYKGQTTLIQYTSNAEDANFTLR
DSCTDLELFENGTLLWTPKSLEPETLEILARSAKIGLASALQPRTVVCHC
NAESQCLYNQTSRVGNSSLEVAGCKCDGGTFGRYCEGSEDACEEPCFPSV
HCVPGKGCEACPPNLTGDGRHCAALGSSFLCQNQSCPVNYCYNQGHCYIS
QTLGCQPMCTCPPAFTDSRCFLAGNNFSPTVNLELPLRVIQLLLSEEENA
SMAEVNASVAYRLGTLDMRAFLRNSQVERIDSAAPASGSPIQHWMVISEF
QYRPRGPVIDFLNNQLLAAVVEAFLYHVPRRSEEPRNDVVFQPISGEDVR
DVTALNVSTLKAYFRCDGYKGYDLVYSPQSGFTCVSPCSRGYCDHGGQCQ
HLPSGPRCSCVSFSIYTAWGEHCEHLSMKLDAFFGIFFGALGGLLLLGVG
TFVVLRFWGCSGARFSYFLNSAEALP

SEQ ID NO: 3462

MKGARWRRVPWVSLSCLCLCLLPHVVPGVSLFPYGADAGDLEFVRRTVDF
TSPLFKPATGFPLGSSLRDSLYFTDNGQIIFPESDYQIFSYPNPLPTGFT
GRDPVALVAPFWDDADFSTGRGTTFYQEYETFYGEHSLLVQQAESWIRKI
TNNGGYKARWALKVTWVNAHAYPAQWTLGSNTYQAILSTDGSRSYALFLY
QSGGMQWDVAQRSGKPVLMGFSSGDGYFENSPLMSQPVWERYRPDRFLNS
NSGLQGLQFYRLHREERPNYRLECLQWLKSQPRWPSWGWNQVSCPCSWQQ
GRRDLRFQPVSIGRWGLGSRQLCSFTSWRGGVCCSYGPWGEFREGWHVQR
PWQLAQELEPQSWCCRWNPKPYLCALYQQRRPHVGCATYRPPQPAWMFGD
PHITTLDGVSYTFNGLGDFLLVGAQDGNSSFLLQGRTAQTGSAQATNFIA
IFAAQYRSSSLGPVTVQWLLEPHDAIRVLLDNQTVTFQPDHEDGGGQETF
NATGVLLSRNGSEVSASFDGWATVSVIALSNILHASASLPPEYQNRTEGL
LGVWNNNPEDDFRMPNGSTIPPGSPEEMLFHFGMTWQINGTGLLGKRNDQ
LPSNFTPVFYSQLQKNSSWAEHLISNCDGDSSCIYDTLALRNASIGLHTR
EVSKNYEQANATLNQYPPSINGGRVIEAYKGQTTLIQYTSNAEDANFTLR
DSCTDLELFENGTLLWTPKSLEPFTLEILARSAKIGLASALQPRTVVCHC
NAESQCLYNQTSRVGNSSLEVAGCKCDGGTFGRYCEGSEDACEEPCFPSV
HCVPGKGCEACPPNLTGDGRHCAALGSSFLCQNQSCPVNYCYNQGHCYIS
QTLGCQPMCTCPPAFTDSRCFLAGNNFSPTVNLELPLRVIQLLLSEEENA
SMAEVNASVAYRLGTLDMRAFLRNSQVERIDSAAPASGSPIQHWMVISEF
QYRPRGPVIDFLNNQLLAAVVEAFLYHVPRRSEEPRNDVVFQPISGEDVR
DVTALNVSTLKAYFRCDGYKGYDLVYSPQSGFTCVSPCSRGYCDHGGQCQ
HLPSGPRCSCVSFSIYTAWGEHCEHLSMKLDAFFGIFFGALGGLLLLGVG
TFVVLRFWGCSGARFSYFLNSAEALP

Polypeptides, variants, or fragments thereof of the present invention include polypeptides which are at least 70%, 75%, 80%, 85%, 90%, 95% or 100% identical to membrane associated molecules selected from the group consisting of SEQ ID NOs: 1288, 3446-3452 and SEQ ID NOs:3458-3462.

In the above embodiments, exemplary “fragments” of a colon tumor associate-polypeptide or variant polypeptide include but are not limited to: a fragment comprising, consisting essentially of, or consisting of a fragment selected from the group consisting of: amino acids 193-218 of SEQ ID NO:3446; amino acids 349-626 of SEQ ID NO:3446; amino acids 1-129 of SEQ ID NO:3447; amino acids 1-23 of SEQ ID NO:3447; amino acids 5-223 of SEQ ID NO:3447; amino acids 5-131 of SEQ ID NO:3447; amino acids 5-510 of SEQ ID NO:3447; amino acids 12-220 of SEQ ID NO:3447; amino acids 20-227 of SEQ ID NO:3447; amino acids 25-145 of SEQ ID NO:3447; amino acids 28-232 of SEQ ID NO:3447; amino acids 29-391 of SEQ ID NO:3447; amino acids 30-457 of SEQ ID NO:3447; amino acids 30-427 of SEQ ID NO:3447; amino acids 34-216 of SEQ ID NO:3447; amino acids 35-457 of SEQ ID NO:3447; amino acids 37-59 of SEQ ID NO:3447; amino acids 38-140 of SEQ ID NO:3447; amino acids 38-212 of SEQ ID NO:3447; amino acids 62-458 of SEQ ID NO:3447; amino acids 66-303 of SEQ ID NO:3447; amino acids 72-94 of SEQ ID NO:3447; amino acids 97-250 of SEQ ID NO:3447; amino acids 105-207 of SEQ ID NO:3447; amino acids 106-259 of SEQ ID NO:3447; amino acids 106-458 of SEQ ID NO:3447; amino acids 109-131 of SEQ ID NO:3447; amino acids 147-429 of SEQ ID NO:3447; amino acids 152-174 of SEQ ID NO:3447; amino acids 176-289 of SEQ ID NO:3447; amino acids 197-417 of SEQ ID NO:3447; amino acids 197-422 of SEQ ID NO:3447; amino acids 198-220 of SEQ ID NO:3447; amino acids 202-239 of SEQ ID NO:3447; amino acids 207-279 of SEQ ID NO:3447; amino acids 212-407 of SEQ ID NO:3447; amino acids 217-271 of SEQ ID NO:3447; amino acids 220-256 of SEQ ID NO:3447; amino acids 221-297 of SEQ ID NO:3447; amino acids 243-401 of SEQ ID NO:3447; amino acids 247-321 of SEQ ID NO:3447; amino acids 247-263 of SEQ ID NO:3447; amino acids 271-364 of SEQ ID NO:3447; amino acids 271-338 of SEQ ID NO:3447; amino acids 278-371 of SEQ ID NO:3447; amino acids 292-301 of SEQ ID NO:3447; amino acids 312-373 of SEQ ID NO:3447; amino acids 321-481 of SEQ ID NO:3447; amino acids 369-470 of SEQ ID NO:3447; amino acids 379-395 of SEQ ID NO:3447; amino acids 387-402 of SEQ ID NO:3447; amino acids 406-428 of SEQ ID NO:3447; amino acids 418-464 of SEQ ID NO:3447; amino acids 1-43 of SEQ ID NO:3448; amino acids 29-51 of SEQ ID NO:3448; amino acids 1-21 of SEQ ID NO:3449; amino acids 41-287 of SEQ ID NO:3449; amino acids 25-47 of SEQ ID NO:3449; amino acids 59-78 of SEQ ID NO:3449; amino acids 98-120 of SEQ ID NO:3449; amino acids 141-163 of SEQ ID NO:3449; amino acids 200-222 of SEQ ID NO:3449; amino acids 243-265 of SEQ ID NO:3449; amino acids 270-289 of SEQ ID NO:3449; amino acids 1-39 of SEQ ID NO:3450; amino acids 41-290 of SEQ ID NO:3450; amino acids 26-48 of SEQ ID NO:3450; amino acids 61-83 of SEQ ID NO:3450; amino acids 98-120 of SEQ ID NO:3450; amino acids 144-166 of SEQ ID NO:3450; amino acids 199-221 of SEQ ID NO:3450; amino acids 241-263 of SEQ ID NO:3450; amino acids 273-292 of SEQ ID NO:3450; amino acids 125-139 of SEQ ID NO:3451; amino acids 1-123 of SEQ ID NO:3451; amino acids 1-83 of SEQ ID NO:3451; amino acids 3-72 of SEQ ID NO:3451; amino acids 4-77 of SEQ ID NO:3451; amino acids 7-109 of SEQ ID NO:3451; amino acids 8-119 of SEQ ID NO:3451; amino acids 14-108 of SEQ ID NO:3451; amino acids 17-107 of SEQ ID NO:3451; amino acids 24-99 of SEQ ID NO:3451; amino acids 25-137 of SEQ ID NO:3451; amino acids 25-124 of SEQ ID NO:3451; amino acids 27-87 of SEQ ID NO:3451; amino acids 30-119 of SEQ ID NO:3451; amino acids 30-50 of SEQ ID NO:3451; amino acids 32-58 of SEQ ID NO:3451; amino acids 41-112 of SEQ ID NO:3451; amino acids 44-119 of SEQ ID NO:3451; amino acids 44-123 of SEQ ID NO:3451; amino acids 53-108 of SEQ ID NO:3451; amino acids 60-128 of SEQ ID NO:3451; amino acids 63-115 of SEQ ID NO:3451; amino acids 63-84 of SEQ ID NO:3451; amino acids 63-102 of SEQ ID NO:3451; amino acids 65-94 of SEQ ID NO:3451; amino acids 67-140 of SEQ ID NO:3451; amino acids 69-113 of SEQ ID NO:3451; amino acids 69-128 of SEQ ID NO:3451; amino acids 95-117 of SEQ ID NO:3451; amino acids 110-124 of SEQ ID NO:3451; amino acids 39-285 of SEQ ID NO:3452; amino acids 1-49 of SEQ ID NO:3452; amino acids 31-53 of SEQ ID NO:3452; amino acids 58-80 of SEQ ID NO:3452; amino acids 100-122 of SEQ ID NO:3452; amino acids 143-165 of SEQ ID NO:3452; amino acids 201-223 of SEQ ID NO:3452; amino acids 236-258 of SEQ ID NO:3452; amino acids 268-287 of SEQ ID NO:3452; amino acids 1154-1311 of SEQ ID NO:3458; amino acids 1310-1425 of SEQ ID NO:3458; amino acids 1428-1610 of SEQ ID NO:3458; amino acids 1834-1866 of SEQ ID NO:3458; amino acids 1873-1914 of SEQ ID NO:3458; amino acids 2080-2117 of SEQ ID NO:3458; amino acids 2129-2151 of SEQ ID NO:3458; amino acids 40-344 of SEQ ID NO:3458; amino acids 66-122 of SEQ ID NO:3458; amino acids 69-273 of SEQ ID NO:3458; amino acids 145-311 of SEQ ID NO:3458; amino acids 205-328 of SEQ ID NO:3458; amino acids 223-304 of SEQ ID NO:3458; amino acids 358-385 of SEQ ID NO:3458; amino acids 372-470 of SEQ ID NO:3458; amino acids 715-763 of SEQ ID NO:3458; amino acids 895-967 of SEQ ID NO:3458; amino acids 909-940 of SEQ ID NO:3458; amino acids 957-1155 of SEQ ID NO:3458; amino acids 1118-1264 of SEQ ID NO:3458; amino acids 1350-1577 of SEQ ID NO:3458; amino acids 1365-1422 of SEQ ID NO:3458; amino acids 1469-1661 of SEQ ID NO:3458; amino acids 1471-1509 of SEQ ID NO:3458; amino acids 1641-1665 of SEQ ID NO:3458; amino acids 1652-1750 of SEQ ID NO:3458; amino acids 1661-1689 of SEQ ID NO:3458; amino acids 1710-1845 of SEQ ID NO:3458; amino acids 1716-1794 of SEQ ID NO:3458; amino acids 1793-1833 of SEQ ID NO:3458; amino acids 1799-1854 of SEQ ID NO:3458; amino acids 1814-1885 of SEQ ID NO:3458; amino acids 1817-1865 of SEQ ID NO:3458; amino acids 1825-1874 of SEQ ID NO:3458; amino acids 1825-1885 of SEQ ID NO:3458; amino acids 1829-1953 of SEQ ID NO:3458; amino acids 1829-1891 of SEQ ID NO:3458; amino acids 1833-1877 of SEQ ID NO:3458; amino acids 1840-1896 of SEQ ID NO:3458; amino acids 1844-1865 of SEQ ID NO:3458; amino acids 1846-1890 of SEQ ID NO:3458; amino acids 1848-1886 of SEQ ID NO:3458; amino acids 1850-1885 of SEQ ID NO:3458; amino acids 1851-1884 of SEQ ID NO:3458; amino acids 1852-1900 of SEQ ID NO:3458; amino acids 1854-1902 of SEQ ID NO:3458; amino acids 1860-1894 of SEQ ID NO:3458; amino acids 1865-1898 of SEQ ID NO:3458; amino acids 1873-1898 of SEQ ID NO:3458; amino acids 1873-1902 of SEQ ID NO:3458; amino acids 1874-1913 of SEQ ID NO:3458; amino acids 1879-1902 of SEQ ID NO:3458; amino acids 1890-1940 of SEQ ID NO:3458; amino acids 1897-1934 of SEQ ID NO:3458; amino acids 1968-2082 of SEQ ID NO:3458; amino acids 2058-2117 of SEQ ID NO:3458; amino acids 2072-2096 of SEQ ID NO:3458; amino acids 2080-2102 of SEQ ID NO:3458; amino acids 2081-2117 of SEQ ID NO:3458; amino acids 2082-2113 of SEQ ID NO:3458; amino acids 1153-1255 of SEQ ID NO:3459; amino acids 23-163 of SEQ ID NO:3459; amino acids 40-344 of SEQ ID NO:3459; amino acids 66-122 of SEQ ID NO:3459; amino acids 69-273 of SEQ ID NO:3459; amino acids 145-311 of SEQ ID NO:3459; amino acids 205-328 of SEQ ID NO:3459; amino acids 223-304 of SEQ ID NO:3459; amino acids 358-385 of SEQ ID NO:3459; amino acids 372-470 of SEQ ID NO:3459; amino acids 594-702 of SEQ ID NO:3459; amino acids 715-763 of SEQ ID NO:3459; amino acids 743-857 of SEQ ID NO:3459; amino acids 831-968 of SEQ ID NO:3459; amino acids 894-966 of SEQ ID NO:3459; amino acids 908-939 of SEQ ID NO:3459; amino acids 956-1154 of SEQ ID NO:3459; amino acids 1154-1311 of SEQ ID NO:3460; amino acids 1310-1425 of SEQ ID NO:3460; amino acids 1428-1610 of SEQ ID NO:3460; amino acids 40-344 of SEQ ID NO:3460; amino acids 66-122 of SEQ ID NO:3460; amino acids 69-273 of SEQ ID NO:3460; amino acids 145-311 of SEQ ID NO:3460; amino acids 205-328 of SEQ ID NO:3460; amino acids 223-304 of SEQ ID NO:3460; amino acids 358-385 of SEQ ID NO:3460; amino acids 372-470 of SEQ ID NO:3460; amino acids 594-702 of SEQ ID NO:3460; amino acids 715-763 of SEQ ID NO:3460; amino acids 895-967 of SEQ ID NO:3460; amino acids 909-940 of SEQ ID NO:3460; amino acids 957-1155 of SEQ ID NO:3460; amino acids 1118-1264 of SEQ ID NO:3460; amino acids 1350-1577 of SEQ ID NO:3460; amino acids 1363-1378 of SEQ ID NO:3460; amino acids 1365-1422 of SEQ ID NO:3460; amino acids 1377-1409 of SEQ ID NO:3460; amino acids 1469-1661 of SEQ ID NO:3460; amino acids 1471-1509 of SEQ ID NO:3460; amino acids 1641-1665 of SEQ ID NO:3460; amino acids 1652-1750 of SEQ ID NO:3460; amino acids 1661-1689 of SEQ ID NO:3460; amino acids 1716-1794 of SEQ ID NO:3460; amino acids 1785-1826 of SEQ ID NO:3460; amino acids 1790-1820 of SEQ ID NO:3460; amino acids 161-318 of SEQ ID NO:3461; amino acids 317-432 of SEQ ID NO:3461; amino acids 435-617 of SEQ ID NO:3461; amino acids 841-873 of SEQ ID NO:3461; amino acids 880-921 of SEQ ID NO:3461; amino acids 1087-1124 of SEQ ID NO:3461; amino acids 1136-1158 of SEQ ID NO:3461; amino acids 125-271 of SEQ ID NO:3461; amino acids 214-252 of SEQ ID NO:3461; amino acids 357-584 of SEQ ID NO:3461; amino acids 372-429 of SEQ ID NO:3461; amino acids 476-668 of SEQ ID NO:3461; amino acids 478-516 of SEQ ID NO:3461; amino acids 480-510 of SEQ ID NO:3461; amino acids 648-672 of SEQ ID NO:3461; amino acids 659-757 of SEQ ID NO:3461; amino acids 668-696 of SEQ ID NO:3461; amino acids 717-852 of SEQ ID NO:3461; amino acids 723-801 of SEQ ID NO:3461; amino acids 800-840 of SEQ ID NO:3461; amino acids 806-861 of SEQ ID NO:3461; amino acids 821-892 of SEQ ID NO:3461; amino acids 824-872 of SEQ ID NO:3461; amino acids 832-881 of SEQ ID NO:3461; amino acids 832-892 of SEQ ID NO:3461; amino acids 836-960 of SEQ ID NO:3461; amino acids 836-898 of SEQ ID NO:3461; amino acids 840-884 of SEQ ID NO:3461; amino acids 847-903 of SEQ ID NO:3461; amino acids 851-872 of SEQ ID NO:3461; amino acids 853-897 of SEQ ID NO:3461; amino acids 855-893 of SEQ ID NO:3461; amino acids 857-892 of SEQ ID NO:3461; amino acids 858-891 of SEQ ID NO:3461; amino acids 859-907 of SEQ ID NO:3461; amino acids 861-909 of SEQ ID NO:3461; amino acids 867-901 of SEQ ID NO:3461; amino acids 872-905 of SEQ ID NO:3461; amino acids 880-909 of SEQ ID NO:3461; amino acids 880-905 of SEQ ID NO:3461; amino acids 881-920 of SEQ ID NO:3461; amino acids 886-909 of SEQ ID NO:3461; amino acids 897-947 of SEQ ID NO:3461; amino acids 904-941 of SEQ ID NO:3461; amino acids 975-1089 of SEQ ID NO:3461; amino acids 1021-1089 of SEQ ID NO:3461; amino acids 1065-1124 of SEQ ID NO:3461; amino acids 1079-1103 of SEQ ID NO:3461; amino acids 1087-1109 of SEQ ID NO:3461; amino acids 1088-1124 of SEQ ID NO:3461; amino acids 1089-1120 of SEQ ID NO:3461; amino acids 12-34 of SEQ ID NO:3462; amino acids 110-267 of SEQ ID NO:3462; amino acids 266-381 of SEQ ID NO:3462; amino acids 384-566 of SEQ ID NO:3462; amino acids 790-822 of SEQ ID NO:3462; amino acids 829-870 of SEQ ID NO:3462; amino acids 1036-1073 of SEQ BD NO:3462; amino acids 1085-1107 of SEQ ID NO:3462; amino acids 74-220 of SEQ ID NO:3462; amino acids 163-201 of SEQ ID NO:3462; amino acids 306-533 of SEQ ID NO:3462; amino acids 321-378 of SEQ ID NO:3462; amino acids 425-617 of SEQ ID NO:3462; amino acids 427-465 of SEQ ID NO:3462; amino acids 429-459 of SEQ ID NO:3462; amino acids 597-621 of SEQ ID NO:3462; amino acids 608-706 of SEQ ID NO:3462; amino acids 617-645 of SEQ ID NO:3462; amino acids 666-801 of SEQ ID NO:3462; amino acids 672-750 of SEQ ID NO:3462; amino acids 749-789 of SEQ ID NO:3462; amino acids 755-810 of SEQ ID NO:3462; amino acids 770-841 of SEQ ID NO:3462; amino acids 773-821 of SEQ ID NO:3462; amino acids 781-830 of SEQ ID NO:3462; amino acids 781-841 of SEQ ID NO:3462; amino acids 785-909 of SEQ ID NO:3462; amino acids 785-847 of SEQ ID NO:3462; amino acids 789-833 of SEQ ID NO:3462; amino acids 796-852 of SEQ ID NO:3462; amino acids 800-821 of SEQ ID NO:3462; amino acids 802-846 of SEQ ID NO:3462; amino acids 804-842 of SEQ ID NO:3462; amino acids 806-841 of SEQ ID NO:3462; amino acids 807-840 of SEQ ID NO:3462; amino acids 808-856 of SEQ ID NO:3462; amino acids 810-858 of SEQ ID NO:3462; amino acids 816-850 of SEQ ID NO:3462; amino acids 821-854 of SEQ ID NO:3462; amino acids 829-858 of SEQ ID NO:3462; amino acids 829-854 of SEQ ID NO:3462; amino acids 830-869 of SEQ ID NO:3462; amino acids 835-858 of SEQ ID NO:3462; amino acids 846-896 of SEQ ID NO:3462; amino acids 853-890 of SEQ ID NO:3462; amino acids 924-1038 of SEQ ID NO:3462; amino acids 970-1038 of SEQ ID NO:3462; amino acids 1014-1073 of SEQ ID NO:3462; amino acids 1028-1052 of SEQ ID NO:3462; amino acids 1036-1058 of SEQ ID NO:3462; amino acids 1037-1073 of SEQ ID NO:3462; amino acids 1038-1069 of SEQ ID NO:3462.

Corresponding polypeptides, variants or fragments thereof comprising, consisting essentially of, or consisting of polypeptides which are at least 70%, 75%, 80%, 85%, 90%, 95%, or 100% identical to the membrane associated molecule fragments disclosed above are also contemplated by the invention.

As known in the art, “sequence identity” between two polypeptides is determined by comparing the amino acid sequence of one polypeptide to the sequence of a second polypeptide. When discussed herein, whether any particular polypeptide is at least about 40%, 45%, 50%, 55%, 60%, 65%, 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

In other embodiments, the present invention includes a method for treating a hyperproliferative disease, e.g., inhibiting tumor formation, tumor growth, tumor invasiveness, and/or metastasis formation in an animal, e.g., a human patient, where the method comprises administering to an animal in need of such treatment an effective amount of a composition comprising, consisting essentially of, or consisting of, in addition to a pharmaceutically acceptable carrier, a binding molecule which specifically binds to at least one epitope of a colon tumor-associate peptide described herein, where the epitope comprises, consists essentially of, or consists of at least about four to five amino acids amino acids of a polypeptide selected from the group consisting of SEQ ID NO:3446; SEQ ID NO:3447; SEQ ID NO:3448; SEQ ID NO:3449; SEQ ID NO:3450; SEQ ID NO:3451; SEQ ID NO:3452; SEQ ID NO:3458; SEQ ID NO:3459; SEQ ID NO:3460; SEQ ID NO:3461; SEQ ID NO:3462; and SEQ ID NO:1288, at least seven, at least nine, or between at least about 15 to about 30 amino acids of a polypeptide selected from the group consisting of SEQ ID NO:3446; SEQ ID NO:3447; SEQ ID NO:3448; SEQ ID NO:3449; SEQ ID NO:3450; SEQ ID NO:3451; SEQ ID NO:3452; SEQ ID NO:3458; SEQ ID NO:3459; SEQ ID NO:3460; SEQ ID NO:3461; SEQ ID NO:3462; and SEQ ID NO:1288. The amino acids of a given epitope of a polypeptide selected from the group consisting of SEQ ID NO:3446; SEQ ID NO:3447; SEQ ID NO:3448; SEQ ID NO:3449; SEQ ID NO:3450; SEQ ID NO:3451; SEQ ID NO:3452; SEQ ID NO:3458; SEQ ID NO:3459; SEQ ID NO:3460; SEQ ID NO:3461; SEQ ID NO:3462; and SEQ ID NO:1288 as described may be, but need not be contiguous. In certain embodiments, the at least one epitope of a membrane associated molecule comprises, consists essentially of, or consists of a non-linear epitope formed by the extracellular domain of a colon associated polypeptide as expressed on the surface of a cell. Thus, in certain embodiments the at least one epitope of a membrane associated molecule comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of a polypeptide selected from the group consisting of SEQ ID NO:3446; SEQ ID NO:3447; SEQ ID NO:3448; SEQ ID NO:3449; SEQ ID NO:3450; SEQ ID NO:3451; SEQ ID NO:3452; SEQ ID NO:3458; SEQ ID NO:3459; SEQ ID NO:3460; SEQ ID NO:3461; SEQ ID NO:3462; and SEQ ID NO:1288, where non-contiguous amino acids form an epitope through protein folding.

In other embodiments, the present invention includes a method for treating a hyperproliferative disease, e.g., inhibiting tumor formation, tumor growth, tumor invasiveness, and/or metastasis formation in an animal, e.g., a human patient, where the method comprises administering to an animal in need of such treatment an effective amount of a composition comprising, consisting essentially of, or consisting of, in addition to a pharmaceutically acceptable carrier, a binding molecule which specifically binds to at least one epitope of a membrane associated molecule, where the epitope comprises, consists essentially of, or consists of, in addition to one, two, three, four, five, six or more contiguous or non-contiguous amino acids of a polypeptide selected from the group consisting of SEQ ID NO:3446; SEQ ID NO:3447; SEQ ID NO:3448; SEQ ID NO:3449; SEQ ID NO:3450; SEQ ID NO:3451; SEQ ID NO:3452; SEQ ID NO:3458; SEQ ID NO:3459; SEQ ID NO:3460; SEQ ID NO:3461; SEQ ID NO:3462; and SEQ ID NO:1288 as described above, an additional moiety which modifies the protein, e.g., a carbohydrate moiety may be included such that the binding molecule binds with higher affinity to modified target protein than it does to an unmodified version of the protein. Alternatively, the binding molecule does not bind the unmodified version of the target protein at all.

More specifically, the present invention provides a method of treating cancer in a human, comprising administering to a human in need of treatment a composition comprising an effective amount of a membrane associated molecule-specific antibody or immunospecific fragment thereof, and a pharmaceutically acceptable carrier. Types of cancer to be treated include, but are not limited to, colon cancer, lung cancer, breast cancer, pancreatic cancer, and prostate cancer.

A binding molecule for use in the present invention is typically a binding polypeptide, in particular an antibody or immunospecific fragment thereof. In certain embodiments, an antibody or fragment thereof binds specifically to at least one epitope of a membrane associated molecule or fragment or variant described above, i.e., binds to such an epitope more readily than it would bind to an unrelated, or random epitope; binds preferentially to at least one epitope of a membrane associated molecule or fragment or variant described above, i.e., binds to such an epitope more readily than it would bind to a related, similar, homologous, or analogous epitope; competitively inhibits binding of a reference antibody which itself binds specifically or preferentially to a certain epitope of a membrane associated molecule or fragment or variant described above; or binds to at least one epitope of a membrane associated molecule or fragment or variant described above with an affinity characterized by a dissociation constant K_{D} of less than about 5×10⁻² M, about 10⁻² M, about 5×10⁻³ M, about 10⁻³ M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷ M, about 10⁻⁷ M, about 5×10⁻⁸ M, about 10⁻⁸ M, about 5×10⁻⁹ M, about 10⁻⁹ M, about 5×10⁻¹⁰ M, about 10⁻¹⁰ M, about 5×10⁻¹¹ M, about 10⁻¹¹ M, about 5×10⁻¹² M, about 10⁻¹² M, about 5×10⁻¹³ M, about 10⁻¹³ M, about 5×10⁻¹⁴ M, about 10⁻¹⁴ M, about 5×10⁻¹⁵ M, or about 10⁻¹⁵ M. As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻² M” might include, for example, from 0.05 M to 0.005 M.

In specific embodiments, binding molecules, e.g., antibodies or immunospecific fragments thereof for use in the diagnostic and treatment methods disclosed herein bind membrane associated molecules or fragments or variants thereof with an off rate (k(off)) of less than or equal to 5×10⁻² sec⁻¹, 10⁻² sec⁻¹, 5×10⁻³ sec⁻¹ or 10⁻³ sec⁻¹. More preferably, binding molecules, e.g., antibodies or immunospecific fragments thereof for use in the diagnostic and treatment methods disclosed herein bind membrane associated molecules or fragments or variants thereof with an off rate (k(off)) of less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

In other embodiments, binding molecules, e.g., antibodies or immunospecific fragments thereof for use in the diagnostic and treatment methods disclosed herein bind membrane associated molecules or fragments or variants thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. More preferably, binding molecules, e.g., antibodies or immunospecific fragments thereof for use in the diagnostic and treatment methods disclosed herein bind membrane associated molecules or fragments or variants thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

### Diagnostic or Prognostic Methods Using Membrane Associated Molecule-Specific Binding Molecules

Membrane associated molecule-specific binding molecules, e.g., antibodies, or fragments, derivatives, or analogs thereof, can be used for diagnostic purposes to detect, diagnose, or monitor diseases, disorders, and/or conditions associated with the aberrant expression and/or activity of the tumor-associated polypeptides described herein.

Membrane associated molecule-specific binding molecules disclosed herein, e.g., antibodies or fragments thereof, are useful for diagnosis, treatment, prevention and/or prognosis of hyperproliferative disorders in mammals, preferably humans. Such disorders include, but are not limited to, cancer, neoplasms, tumors and/or as described under elsewhere herein, especially membrane associated molecule-associated cancers such as lung cancers, bronchogenic cancers, small cell lung cancers, non-small cell lung cancers, oat cell carcinomas, small cell undifferentiated carcinomas, squamous cell carcinomas, adenocarcinomas, large-cell undifferentiated carcinomas, pancreatic cancers, cervical cancers, ovarian cancers, liver cancers, bladder cancers, breast cancers, colon cancers, renal cancers, prostate cancers, testicular cancers, thyroid cancers, head and neck cancers, carcinoid tumors, adenoid cystic carcinomas, and hamartomas. In a preferred embodiment, such colon cancers includes, but are not limited to adenocarcinomas. In a preferred embodiment, such membrane associated molecule-associated cancers include, but are not limited to, lung cancers, non-small cell lung cancers, squamous cell carcinomas, adenocarcinomas, pancreatic cancers, cervical cancers, renal cancers, prostate cancers, and testicular cancers.

In particular, it is believed that certain tumor-associated tissues express significantly enhanced levels of the polypeptides, disclosed herein, when compared to corresponding “standard” levels. Indeed, the proteins described herein were identified based on their increased expression in malignant colon cells relative to nonmalignant colon cells.

For example, binding molecules, e.g., antibodies (and antibody fragments) directed against membrane associated molecules, variants and fragments thereof may be used to detect particular tissues expressing these proteins. These diagnostic assays may be performed in vivo or in vitro, such as, for example, on biopsy tissue or autopsy tissue.

Thus, the invention provides a diagnostic method useful during diagnosis of a cancers and other hyperproliferative disorders, which involves measuring the expression level of membrane associated molecules described herein in tissue and blood or other bodily fluids, which may contain secreted forms of membrane associated molecules, variant polypeptides, or fragments thereof, from an individual and comparing the measured expression level with a standard membrane associated molecule expression levels in normal tissue, whereby an increase in the expression level compared to the standard is indicative of a disorder.

With respect to cancer, the presence of a relatively high amount of membrane associated molecules in biopsied tissue from an individual may indicate the presence of a tumor or other malignant growth, may indicate a predisposition for the development of such malignancies or tumors, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Membrane associated molecule-specific binding molecules can be used to assay protein levels in a biological sample using classical immunohistological methods known to those of skill in the art (e.g., see Jalkanen, et al., J. Cell. Biol. 101:976-985 (1985); Jalkanen, et al., J. Cell Biol. 105:3087-3096 (1987)). Other antibody-based methods useful for detecting protein expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹¹²In), and technetium (⁹⁹Tc); luminescent labels, such as luminol; and fluorescent labels, such as fluorescein and rhodamine, and biotin. Suitable assays are described in more detail elsewhere herein.

One aspect of the invention is a method for the in vivo detection or diagnosis of a hyperproliferative disease or disorder associated with aberrant expression of a membrane associated molecule, variant or fragment thereof in an animal, preferably a mammal and most preferably a human. In one embodiment, diagnosis comprises: a) administering (for example, parenterally, subcutaneously, or intraperitoneally) to a subject an effective amount of a labeled binding molecule, e.g., an antibody or fragment thereof, which specifically binds to a membrane associated molecule described herein; b) waiting for a time interval following the administering for permitting the labeled binding molecule to preferentially concentrate at sites in the subject where the membrane associated molecule is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of a membrane associated molecule. Background level can be determined by various methods including comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of, e.g., ⁹⁹Tc. The labeled binding molecule, e.g., antibody or antibody fragment, will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 7 to 10 days.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the binding molecule is labeled with a radioisotope and is detected in the patient using a radiation responsive surgical instrument (Thurston et al., U.S. Pat. No. 5,441,050). In another embodiment, the binding molecule is labeled with a fluorescent compound and is detected in the patient using a fluorescence responsive scanning instrument. In another embodiment, the binding molecule is labeled with a positron emitting metal and is detected in the patent using positron emission-tomography. In yet another embodiment, the binding molecule is labeled with a paramagnetic label and is detected in a patient using magnetic resonance imaging (MRI).

Antibody labels or markers for in vivo imaging of membrane associated molecule expression include those detectable by X-radiography, nuclear magnetic resonance imaging (NMR), MRI, CAT-scans or electron spin resonance imaging (ESR). For X-radiography, suitable labels include radioisotopes such as barium or cesium, which emit detectable radiation but are not overtly harmful to the subject. Suitable markers for NMR and ESR. include those with a detectable characteristic spin, such as deuterium, which may be incorporated into the antibody by labeling of nutrients for the relevant hybridoma. Where in vivo imaging is used to detect enhanced levels of membrane associated molecule expression for diagnosis in humans, it may be preferable to use human antibodies or “humanized” chimeric monoclonal antibodies. Such antibodies can be produced using techniques described herein or otherwise known in the art. For example methods for producing chimeric antibodies are known in the art. See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

In a related embodiment to those described above, monitoring of an already diagnosed disease or disorder is carried out by repeating any one of the methods for diagnosing the disease or disorder, for example, one month after initial diagnosis, six months after initial diagnosis, one year after initial diagnosis, etc.

Where a diagnosis of a disorder, including diagnosis of a tumor, has already been made according to conventional methods, detection methods as disclosed herein are useful as a prognostic indicator, whereby patients continuing to exhibit enhanced membrane associated molecule expression will experience a worse clinical outcome relative to patients whose expression level decreases nearer the standard level.

By “assaying the expression level of the membrane associated molecule” is intended qualitatively or quantitatively measuring or estimating the level of the membrane associated molecule in a first biological sample either directly (e.g., by determining or estimating absolute protein level) or relatively (e.g., by comparing to the membrane associated molecule level in a second biological sample). Preferably, membrane associated molecule expression level in the first biological sample is measured or estimated and compared to a standard membrane associated molecule level, the standard being taken from a second biological sample obtained from an individual not having the disorder or being determined by averaging levels from a population of individuals not having the disorder. As will be appreciated in the art, once the “standard” membrane associated molecule level is known, it can be used repeatedly as a standard for comparison.

By “biological sample” is intended any biological sample obtained from an individual, cell line, tissue culture, or other source of cells potentially expressing the membrane associated molecule. As indicated, biological samples include tissue sources which contain cells potentially expressing the membrane associated molecule. Methods for obtaining tissue biopsies and body fluids from mammals are well known in the art.

In an additional embodiment, antibodies, or immunospecific fragments of antibodies directed to a conformational epitope of a membrane associated molecule may be used to quantitatively or qualitatively detect the presence of membrane associated molecules or conserved variants or peptide fragments thereof. This can be accomplished, for example, by immunofluorescence techniques employing a fluorescently labeled antibody coupled with light microscopic, flow cytometric, or fluorimetric detection.

Binding molecules for use in the diagnostic methods described above include any binding molecule which specifically binds to a membrane associated molecule. Such polypeptides include, but are not limited to, a membrane associated molecule comprising, consisting essentially of or consisting of a polypeptide selected from the group consisting of: SEQ ID NO:3446; SEQ ID NO:3447; SEQ ID NO:3448; SEQ ID NO:3449; SEQ ID NO:3450; SEQ ID NO:3451; SEQ ID NO:3452; SEQ ID NO:3458; SEQ ID NO:3459; SEQ ID NO:3460; SEQ ID NO:3461; SEQ ID NO:3462; and SEQ ID NO:1288. Corresponding polypeptides, variants or fragments thereof comprising, consisting essentially of, or consisting of polypeptides which are at least 70%, 75%, 80%, 85%, 90%, 95%, or 100% identical to the membrane associated molecules selected from the group consisting of SEQ ID NOs: 1288, 3446-3452 and 3458-3462 are also contemplated by the invention.

In the above embodiments, exemplary “fragments” of a colon tumor associate-polypeptide or variant polypeptides include but are not limited to: a fragment comprising, consisting essentially of, or consisting of a fragment selected from the group consisting of: amino acids 193-218 of SEQ ID NO:3446; amino acids 349-626 of SEQ ID NO:3446; amino acids 1-129 of SEQ ID NO:3447; amino acids 1-23 of SEQ ID NO:3447; amino acids 5-223 of SEQ ID NO:3447; amino acids 5-131 of SEQ ID NO:3447; amino acids 5-510 of SEQ ID NO:3447; amino acids 12-220 of SEQ ID NO:3447; amino acids 20-227 of SEQ ID NO:3447; amino acids 25-145 of SEQ ID NO:3447; amino acids 28-232 of SEQ ID NO:3447; amino acids 29-391 of SEQ ID NO:3447; amino acids 30-457 of SEQ ID NO:3447; amino acids 30-427 of SEQ ID NO:3447; amino acids 34-216 of SEQ ID NO:3447; amino acids 35-457 of SEQ ID NO:3447; amino acids 37-59 of SEQ ID NO:3447; amino acids 38-140 of SEQ ID NO:3447; amino acids 38-212 of SEQ ID NO:3447; amino acids 62-458 of SEQ ID NO:3447; amino acids 66-303 of SEQ ID NO:3447; amino acids 72-94 of SEQ ID NO:3447; amino acids 97-250 of SEQ ID NO:3447; amino acids 105-207 of SEQ ID NO:3447; amino acids 106-259 of SEQ ID NO:3447; amino acids 106-458 of SEQ ID NO:3447; amino acids 109-131 of SEQ ID NO:3447; amino acids 147-429 of SEQ ID NO:3447; amino acids 152-174 of SEQ ID NO:3447; amino acids 176-289 of SEQ ID NO:3447; amino acids 197-417 of SEQ ID NO:3447; amino acids 197-422 of SEQ ID NO:3447; amino acids 198-220 of SEQ ID NO:3447; amino acids 202-239 of SEQ ID NO:3447; amino acids 207-279 of SEQ ID NO:3447; amino acids 212-407 of SEQ ID NO:3447; amino acids 217-271 of SEQ ID NO:3447; amino acids 220-256 of SEQ ID NO:3447; amino acids 221-297 of SEQ ID NO:3447; amino acids 243-401 of SEQ ID NO:3447; amino acids 247-321 of SEQ ID NO:3447; amino acids 247-263 of SEQ ID NO:3447; amino acids 271-364 of SEQ ID NO:3447; amino acids 271-338 of SEQ ID NO:3447; amino acids 278-371 of SEQ ID NO:3447; amino acids 292-301 of SEQ ID NO:3447; amino acids 312-373 of SEQ ID NO:3447; amino acids 321-481 of SEQ ID NO:3447; amino acids 369-470 of SEQ ID NO:3447; amino acids 379-395 of SEQ ID NO:3447; amino acids 387-402 of SEQ ID NO:3447; amino acids 406-428 of SEQ ID NO:3447; amino acids 418-464 of SEQ ID NO:3447; amino acids 1-43 of SEQ ID NO:3448; amino acids 29-51 of SEQ ID NO:3448; amino acids 1-21 of SEQ ID NO:3449; amino acids 41-287 of SEQ ID NO:3449; amino acids 25-47 of SEQ ID NO:3449; amino acids 59-78 of SEQ ID NO:3449; amino acids 98-120 of SEQ ID NO:3449; amino acids 141-163 of SEQ ID NO:3449; amino acids 200-222 of SEQ ID NO:3449; amino acids 243-265 of SEQ ID NO:3449; amino acids 270-289 of SEQ ID NO:3449; amino acids 1-39 of SEQ ID NO:3450; amino acids 41-290 of SEQ ID NO:3450; amino acids 26-48 of SEQ ID NO:3450; amino acids 61-83 of SEQ ID NO:3450; amino acids 98-120 of SEQ ID NO:3450; amino acids 144-166 of SEQ ID NO:3450; amino acids 199-221 of SEQ ID NO:3450; amino acids 241-263 of SEQ ID NO:3450; amino acids 273-292 of SEQ ID NO:3450; amino acids 125-139 of SEQ ID NO:3451; amino acids 1-123 of SEQ ID NO:3451; amino acids 1-83 of SEQ ID NO:3451; amino acids 3-72 of SEQ ID NO:3451; amino acids 4-77 of SEQ ID NO:3451; amino acids 7-109 of SEQ ID NO:3451; amino acids 8-119 of SEQ ID NO:3451; amino acids 14-108 of SEQ ID NO:3451; amino acids 17-107 of SEQ ID NO:3451; amino acids 24-99 of SEQ ID NO:3451; amino acids 25-137 of SEQ ID NO:3451; amino acids 25-124 of SEQ ID NO:3451; amino acids 27-87 of SEQ ID NO:3451; amino acids 30-119 of SEQ ID NO:3451; amino acids 30-50 of SEQ ID NO:3451; amino acids 32-58 of SEQ ID NO:3451; amino acids 41-112 of SEQ ID NO:3451; amino acids 44-119 of SEQ ID NO:3451; amino acids 44-123 of SEQ ID NO:3451; amino acids 53-108 of SEQ ID NO:3451; amino acids 60-128 of SEQ ID NO:3451; amino acids 63-115 of SEQ ID NO:3451; amino acids 63-84 of SEQ ID NO:3451; amino acids 63-102 of SEQ ID NO:3451; amino acids 65-94 of SEQ ID NO:3451; amino acids 67-140 of SEQ ID NO:3451; amino acids 69-113 of SEQ ID NO:3451; amino acids 69-128 of SEQ ID NO:3451; amino acids 95-117 of SEQ ID NO:3451; amino acids 110-124 of SEQ ID NO:3451; amino acids 39-285 of SEQ ID NO:3452; amino acids 1-49 of SEQ ID NO:3452; amino acids 31-53 of SEQ BD NO:3452; amino acids 58-80 of SEQ ID NO:3452; amino acids 100-122 of SEQ ID NO:3452; amino acids 143-165 of SEQ ID NO:3452; amino acids 201-223 of SEQ ID NO:3452; amino acids 236-258 of SEQ ID NO:3452; amino acids 268-287 of SEQ ID NO:3452; amino acids 1154-1311 of SEQ ID NO:3458; amino acids 1310-1425 of SEQ ID NO:3458; amino acids 1428-1610 of SEQ ID NO:3458; amino acids 1834-1866 of SEQ ID NO:3458; amino acids 1873-1914 of SEQ ID NO:3458; amino acids 2080-2117 of SEQ ID NO:3458; amino acids 2129-2151 of SEQ ID NO:3458; amino acids 40-344 of SEQ ID NO:3458; amino acids 66-122 of SEQ ID NO:3458; amino acids 69-273 of SEQ ID NO:3458; amino acids 145-311 of SEQ ID NO:3458; amino acids 205-328 of SEQ ID NO:3458; amino acids 223-304 of SEQ ID NO:3458; amino acids 358-385 of SEQ ID NO:3458; amino acids 372-470 of SEQ ID NO:3458; amino acids 715-763 of SEQ ID NO:3458; amino acids 895-967 of SEQ ID NO:3458; amino acids 909-940 of SEQ ID NO:3458; amino acids 957-1155 of SEQ ID NO:3458; amino acids 1118-1264 of SEQ ID NO:3458; amino acids 1350-1577 of SEQ ID NO:3458; amino acids 1365-1422 of SEQ ID NO:3458; amino acids 1469-1661 of SEQ ID NO:3458; amino acids 1471-1509 of SEQ ID NO:3458; amino acids 1641-1665 of SEQ ID NO:3458; amino acids 1652-1750 of SEQ ID NO:3458; amino acids 1661-1689 of SEQ ID NO:3458; amino acids 1710-1845 of SEQ ID NO:3458; amino acids 1716-1794 of SEQ ID NO:3458; amino acids 1793-1833 of SEQ ID NO:3458; amino acids 1799-1854 of SEQ ID NO:3458; amino acids 1814-1885 of SEQ ID NO:3458; amino acids 1817-1865 of SEQ ID NO:3458; amino acids 1825-1874 of SEQ ID NO:3458; amino acids 1825-1885 of SEQ ID NO:3458; amino acids 1829-1953 of SEQ ID NO:3458; amino acids 1829-1891 of SEQ ID NO:3458; amino acids 1833-1877 of SEQ ID NO:3458; amino acids 1840-1896 of SEQ ID NO:3458; amino acids 1844-1865 of SEQ ID NO:3458; amino acids 1846-1890 of SEQ ID NO:3458; amino acids 1848-1886 of SEQ ID NO:3458; amino acids 1850-1885 of SEQ ID NO:3458; amino acids 1851-1884 of SEQ ID NO:3458; amino acids 1852-1900 of SEQ ID NO:3458; amino acids 1854-1902 of SEQ ID NO:3458; amino acids 1860-1894 of SEQ ID NO:3458; amino acids 1865-1898 of SEQ ID NO:3458; amino acids 1873-1898 of SEQ ID NO:3458; amino acids 1873-1902 of SEQ ID NO:3458; amino acids 1874-1913 of SEQ ID NO:3458; amino acids 1879-1902 of SEQ ID NO:3458; amino acids 1890-1940 of SEQ ID NO:3458; amino acids 1897-1934 of SEQ ID NO:3458; amino acids 1968-2082 of SEQ ID NO:3458; amino acids 2058-2117 of SEQ ID NO:3458; amino acids 2072-2096 of SEQ ID NO:3458; amino acids 2080-2102 of SEQ ID NO:3458; amino acids 2081-2117 of SEQ ID NO:3458; amino acids 2082-2113 of SEQ ID NO:3458; amino acids 1153-1255 of SEQ ID NO:3459; amino acids 23-163 of SEQ ID NO:3459; amino acids 40-344 of SEQ ID NO:3459; amino acids 66-122 of SEQ ID NO:3459; amino acids 69-273 of SEQ ID NO:3459; amino acids 145-311 of SEQ ID NO:3459; amino acids 205-328 of SEQ ID NO:3459; amino acids 223-304 of SEQ ID NO:3459; amino acids 358-385 of SEQ ID NO:3459; amino acids 372-470 of SEQ ID NO:3459; amino acids 594-702 of SEQ ID NO:3459; amino acids 715-763 of SEQ ID NO:3459; amino acids 743-857 of SEQ ID NO:3459; amino acids 831-968 of SEQ ID NO:3459; amino acids 894-966 of SEQ ID NO:3459; amino acids 908-939 of SEQ ID NO:3459; amino acids 956-1154 of SEQ ID NO:3459; amino acids 1154-1311 of SEQ ID NO:3460; amino acids 1310-1425 of SEQ ID NO:3460; amino acids 1428-1610 of SEQ ID NO:3460; amino acids 40-344 of SEQ ID NO:3460; amino acids 66-122 of SEQ ID NO:3460; amino acids 69-273 of SEQ ID NO:3460; amino acids 145-311 of SEQ ID NO:3460; amino acids 205-328 of SEQ ID NO:3460; amino acids 223-304 of SEQ ID NO:3460; amino acids 358-385 of SEQ ID NO:3460; amino acids 372-470 of SEQ ID NO:3460; amino acids 594-702 of SEQ ID NO:3460; amino acids 715-763 of SEQ ID NO:3460; amino acids 895-967 of SEQ ID NO:3460; amino acids 909-940 of SEQ ID NO:3460; amino acids 957-1155 of SEQ ID NO:3460; amino acids 1118-1264 of SEQ ID NO:3460; amino acids 1350-1577 of SEQ ID NO:3460; amino acids 1363-1378 of SEQ ID NO:3460; amino acids 1365-1422 of SEQ ID NO:3460; amino acids 1377-1409 of SEQ ID NO:3460; amino acids 1469-1661 of SEQ ID NO:3460; amino acids 1471-1509 of SEQ ID NO:3460; amino acids 1641-1665 of SEQ ID NO:3460; amino acids 1652-1750 of SEQ ID NO:3460; amino acids 1661-1689 of SEQ ID NO:3460; amino acids 1716-1794 of SEQ ID NO:3460; amino acids 1785-1826 of SEQ ID NO:3460; amino acids 1790-1820 of SEQ ID NO:3460; amino acids 161-318 of SEQ ID NO:3461; amino acids 317-432 of SEQ ID NO:3461; amino acids 435-617 of SEQ ID NO:3461; amino acids 841-873 of SEQ ID NO:3461; amino acids 880-921 of SEQ ID NO:3461; amino acids 1087-1124 of SEQ ID NO:3461; amino acids 1136-1158 of SEQ ID NO:3461; amino acids 125-271 of SEQ ID NO:3461; amino acids 214-252 of SEQ ID NO:3461; amino acids 357-584 of SEQ ID NO:3461; amino acids 372-429 of SEQ ID NO:3461; amino acids 476-668 of SEQ ID NO:3461; amino acids 478-516 of SEQ ID NO:3461; amino acids 480-510 of SEQ ID NO:3461; amino acids 648-672 of SEQ ID NO:3461; amino acids 659-757 of SEQ ID NO:3461; amino acids 668-696 of SEQ ID NO:3461; amino acids 717-852 of SEQ ID NO:3461; amino acids 723-801 of SEQ ID NO:3461; amino acids 800-840 of SEQ ID NO:3461; amino acids 806-861 of SEQ ID NO:3461; amino acids 821-892 of SEQ ID NO:3461; amino acids 824-872 of SEQ ID NO:3461; amino acids 832-881 of SEQ ID NO:3461; amino acids 832-892 of SEQ ID NO:3461; amino acids 836-960 of SEQ ID NO:3461; amino acids 836-898 of SEQ ID NO:3461; amino acids 840-884 of SEQ ID NO:3461; amino acids 847-903 of SEQ ID NO:3461; amino acids 851-872 of SEQ ID NO:3461; amino acids 853-897 of SEQ ID NO:3461; amino acids 855-893 of SEQ ID NO:3461; amino acids 857-892 of SEQ ID NO:3461; amino acids 858-891 of SEQ ID NO:3461; amino acids 859-907 of SEQ ID NO:3461; amino acids 861-909 of SEQ ID NO:3461; amino acids 867-901 of SEQ ID NO:3461; amino acids 872-905 of SEQ ID NO:3461; amino acids 880-909 of SEQ ID NO:3461; amino acids 880-905 of SEQ ID NO:3461; amino acids 881-920 of SEQ ID NO:3461; amino acids 886-909 of SEQ ID NO:3461; amino acids 897-947 of SEQ ID NO:3461; amino acids 904-941 of SEQ ID NO:3461; amino acids 975-1089 of SEQ ID NO:3461; amino acids 1021-1089 of SEQ ID NO:3461; amino acids 1065-1124 of SEQ ID NO:3461; amino acids 1079-1103 of SEQ ID NO:3461; amino acids 1087-1109 of SEQ ID NO:3461; amino acids 1088-1124 of SEQ ID NO:3461; amino acids 1089-1120 of SEQ ID NO:3461; amino acids 12-34 of SEQ ID NO:3462; amino acids 110-267 of SEQ ID NO:3462; amino acids 266-381 of SEQ ID NO:3462; amino acids 384-566 of SEQ ID NO:3462; amino acids 790-822 of SEQ ID NO:3462; amino acids 829-870 of SEQ ID NO:3462; amino acids 1036-1073 of SEQ ID NO:3462; amino acids 1085-1107 of SEQ ID NO:3462; amino acids 74-220 of SEQ ID NO:3462; amino acids 163-201 of SEQ ID NO:3462; amino acids 306-533 of SEQ ID NO:3462; amino acids 321-378 of SEQ ID NO:3462; amino acids 425-617 of SEQ ID NO:3462; amino acids 427-465 of SEQ ID NO:3462; amino acids 429-459 of SEQ ID NO:3462; amino acids 597-621 of SEQ ID NO:3462; amino acids 608-706 of SEQ ID NO:3462; amino acids 617-645 of SEQ ID NO:3462; amino acids 666-801 of SEQ ID NO:3462; amino acids 672-750 of SEQ ID NO:3462; amino acids 749-789 of SEQ ID NO:3462; amino acids 755-810 of SEQ ID NO:3462; amino acids 770-841 of SEQ ID NO:3462; amino acids 773-821 of SEQ ID NO:3462; amino acids 781-830 of SEQ ID NO:3462; amino acids 781-841 of SEQ ID NO:3462; amino acids 785-909 of SEQ ID NO:3462; amino acids 785-847 of SEQ ID NO:3462; amino acids 789-833 of SEQ ID NO:3462; amino acids 796-852 of SEQ ID NO:3462; amino acids 800-821 of SEQ ID NO:3462; amino acids 802-846 of SEQ ID NO:3462; amino acids 804-842 of SEQ ID NO:3462; amino acids 806-841 of SEQ ID NO:3462; amino acids 807-840 of SEQ ID NO:3462; amino acids 808-856 of SEQ ID NO:3462; amino acids 810-858 of SEQ ID NO:3462; amino acids 816-850 of SEQ ID NO:3462; amino acids 821-854 of SEQ ID NO:3462; amino acids 829-858 of SEQ ID NO:3462; amino acids 829-854 of SEQ ID NO:3462; amino acids 830-869 of SEQ ID NO:3462; amino acids 835-858 of SEQ ID NO:3462; amino acids 846-896 of SEQ ID NO:3462; amino acids 853-890 of SEQ ID NO:3462; amino acids 924-1038 of SEQ ID NO:3462; amino acids 970-1038 of SEQ ID NO:3462; amino acids 1014-1073 of SEQ ID NO:3462; amino acids 1028-1052 of SEQ ID NO:3462; amino acids 1036-1058 of SEQ ID NO:3462; amino acids 1037-1073 of SEQ ID NO:3462; amino acids 1038-1069 of SEQ ID NO:3462.

Additionally, exemplary fragments of the membrane associated molecules and variant polypeptides include but are limited to fragments of the extracellular domains of the membrane associated molecules described herein. For example, fragments selected from the group consisting of: amino acids 1-627 of SEQ ID NO:3446; amino acids 1-36 of SEQ ID NO:3447; amino acids 95-108 of SEQ ID NO:3447; amino acids 175-197 of SEQ ID NO:3447; amino acids 429-437 of SEQ ID NO:3447; amino acids 1-28 of SEQ ID NO:3448; amino acids 1-24 of SEQ ID NO:3449; amino acids 79-97 of SEQ ID NO:3449; amino acids 164-199 of SEQ ID NO:3449; amino acids 166-169 of SEQ ID NO:3449; amino acids 1-25 of SEQ ID NO:3450; amino acids 84-87 of SEQ ID NO:3450; amino acids 167-198 of SEQ ID NO:3450; amino acids 264-172 of SEQ ID NO:3450; amino acids 1-30 of SEQ ID NO:3452; amino acids 81-99 of SEQ ID NO:3452; amino acids 166-200 of SEQ ID NO:3452; amino acids 259-267 of SEQ ID NO:3452; amino acids 1-2128 of SEQ ID NO:3458; amino acids 1-1255 of SEQ ID NO:3459; amino acids 1-1827 of SEQ ID NO:3460; amino acids 1-1135 of SEQ ID NO:3461; and amino acids 35-1084 of SEQ ID NO:3462.

Additionally, exemplary fragments of the membrane associated molecules and variant polypeptides include but are limited to fragments of the intracellular domains of the membrane associated molecules described herein. For example, fragments selected from the group consisting of: amino acids 60-71 of SEQ ID NO:3447; amino acids 132-151 of SEQ ID NO:3447; amino acids 221-405 of SEQ ID NO:3447; amino acids 461-512 of SEQ ID NO:3447; amino acids 52-160 of SEQ ID NO:3448; amino acids 48-58 of SEQ ID NO:3449; amino acids 121-140 of SEQ ID NO:3449; amino acids 223-242 of SEQ ID NO:3449; amino acids 290-316 of SEQ ID NO:3449; amino acids 49-60 of SEQ ID NO:3450; amino acids 121-143 of SEQ ID NO:3450; amino acids 222-240 of SEQ ID NO:3450; amino acids 293-309 of SEQ ID NO:3450; amino acids 1-124 of SEQ ID NO:3451; amino acids 54-57 of SEQ ID NO:3452; amino acids 123-142 of SEQ ID NO:3452; amino acids 224-235 of SEQ ID NO:3452; amino acids 288-311 of SEQ ID NO:3452; amino acids 2152-2169 of SEQ ID NO:3458; amino acids 1159-1176 of SEQ ID NO:3461; amino acids 1-11 of SEQ ID NO:3462; and amino acids 1108-1125 of SEQ ID NO:3462.

Corresponding polypeptides, variants or fragments thereof comprising, consisting essentially of, or consisting of polypeptides which are at least 70%, 75%, 80%, 85%, 90%, 95%, or 100% identical to the membrane associated molecules selected from the group consisting of SEQ ID NO:3446; SEQ ID NO:3447; SEQ ID NO:3448; SEQ ID NO:3449; SEQ ID NO:3450; SEQ ID NO:3451; SEQ ID NO:3452; SEQ ID NO:3458; SEQ ID NO:3459; SEQ ID NO:3460; SEQ ID NO:3461; SEQ ID NO:3462; and SEQ ID NO:1288 are also contemplated by the invention.

Other binding molecules for use in the diagnostic methods described herein include binding molecules which specifically bind to at least one epitope of a membrane associated molecule where the epitope comprises, consists essentially of, or consists of at least about four to five amino acids amino acids of a membrane associated molecule, at least seven, at least nine, or between at least about 15 to about 30 amino acids of a membrane associated molecule. The amino acids of a given epitope of a membrane associated molecule as described may be, but need not be contiguous. In certain embodiments, the at least one epitope of a membrane associated molecule comprises, consists essentially of, or consists of a non-linear epitope formed by the extracellular domain of a membrane associated molecule as expressed on the surface of a cell. Thus, in certain embodiments the at least one epitope of a membrane associated molecule comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of a membrane associated molecule, where non-contiguous amino acids form an epitope through protein folding.

Additional binding molecules include those which specifically bind to at least one epitope of a membrane associated molecule, where the epitope comprises, consists essentially of, or consists of, in addition to one, two, three, four, five, six or more contiguous or non-contiguous amino acids of a membrane associated molecule as described above, an additional moiety which modifies the protein, e.g., a carbohydrate moiety may be included such that the binding molecule binds with higher affinity to modified target protein than it does to an unmodified version of the protein. Alternatively, the binding molecule does not bind the unmodified version of the target protein at all.

Cancers that may be diagnosed, and/or prognosed using the methods described above include but are not limited to, colorectal cancer, breast cancer, ovarian cancer, prostate cancer, pancreatic cancer, lung cancer, liver cancer, uterine cancer, and/or skin cancer.

### Antibodies or Immunospecific Fragments Thereof

In one embodiment, a binding molecule for use in the methods of the invention is an antibody molecule, or immunospecific fragment thereof. Unless it is specifically noted, as used herein a “fragment thereof” in reference to an antibody refers to an immunospecific fragment, i.e., an antigen-specific fragment. In one embodiment, a binding molecule, e.g., an antibody of the invention is a bispecific binding molecule, binding polypeptide, or antibody, e.g., a bispecific antibody, minibody, domain deleted antibody, or fusion protein having binding specificity for more than one epitope, e.g., more than one antigen or more than one epitope on the same antigen. In one embodiment, a bispecific binding molecule, binding polypeptide, or antibody has at least one binding domain specific for at least one epitope on a target polypeptide disclosed herein, e.g., a membrane associated molecule. In another embodiment, a bispecific binding molecule, binding polypeptide, or antibody has at least one binding domain specific for an epitope on a target polypeptide and at least one target binding domain specific for a drug or toxin. In yet another embodiment, a bispecific binding molecule, binding polypeptide, or antibody has at least one binding domain specific for an epitope on a membrane associated molecule disclosed herein, and at least one binding domain specific for a prodrug. A bispecific binding molecule, binding polypeptide, or antibody may be a tetravalent antibody that has two target binding domains specific for an epitope of a target polypeptide disclosed herein and two target binding domains specific for a second target. Thus, a tetravalent bispecific binding molecule, binding polypeptide, or antibody may be bivalent for each specificity.

Antibody binding molecules for use in the treatment methods of the present invention, as known by those of ordinary skill in the art, can comprise a constant region which mediates one or more effector functions. For example, binding of the C1 component of complement to an antibody constant region may activate the complement system. Activation of complement is important in the opsonisation and lysis of cell pathogens. The activation of complement also stimulates the inflammatory response and may also be involved in autoimmune hypersensitivity. Further, antibodies bind to receptors on various cells via the Fc region, with a Fc receptor binding site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

In certain embodiments, methods of treating hyperproliferative diseases according to the present invention comprise administration of an antibody, or immunospecific fragment thereof, in which at least a fraction of one or more of the constant region domains has been deleted or otherwise altered so as to provide desired biochemical characteristics such as reduced effector functions, the ability to non-covalently dimerize, increased ability to localize at the site of a tumor, reduced serum half-life, or increased serum half-life when compared with a whole, unaltered antibody of approximately the same immunogenicity. For example, certain antibodies for use in the diagnostic and treatment methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the C_{H}2 domain will be deleted.

In certain antibodies or immunospecific fragments thereof for use in the diagnostic and therapeutic methods described herein, the Fc portion may be mutated to decrease effector function using techniques known in the art. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, may easily be measured and quantified using well know immunological techniques without undue experimentation.

Modified forms of antibodies or immunospecific fragments thereof for use in the diagnostic and therapeutic methods disclosed herein can be made from whole precursor or parent antibodies using techniques known in the art. Exemplary techniques are discussed in more detail herein.

In certain embodiments both the variable and constant regions of membrane associated molecule-specific antibodies or immunospecific fragments thereof for use in the diagnostic and treatment methods disclosed herein are fully human. Fully human antibodies can be made using techniques that are known in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140. Other techniques are known in the art. Fully human antibodies can likewise be produced by various display technologies, e.g., phage display or other viral display systems, as described in more detail elsewhere herein.

Binding molecules, e.g., binding polypeptides, e.g., membrane associated molecule-specific antibodies or immunospecific fragments thereof for use in the diagnostic and treatment methods disclosed herein can be made or manufactured using techniques that are known in the art. In certain embodiments, antibody molecules or fragments thereof are “recombinantly produced,” i.e., are produced using recombinant DNA technology. Exemplary techniques for making antibody molecules or fragments thereof are discussed in more detail elsewhere herein.

Binding molecules, e.g., binding polypeptides, e.g., membrane associated molecule-specific antibodies or immunospecific fragments thereof for use in the diagnostic and treatment methods disclosed herein include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

In preferred embodiments, a binding molecule, e.g., a binding polypeptide, e.g., a membrane associated molecule-specific antibody or immunospecific fragment thereof for use in the diagnostic and treatment methods disclosed herein will not elicit a deleterious immune response in the animal to be treated, e.g., in a human. In one embodiment, binding molecules, e.g., binding polypeptides, e.g., membrane associated molecule-specific antibodies or immunospecific fragments thereof for use in the diagnostic and treatment methods disclosed herein be modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

De-immunization can also be used to decrease the immunogenicity of an antibody. As used herein, the term “de-immunization” includes alteration of an antibody to modify T cell epitopes (see, e.g., WO9852976A1, WO0034317A2). For example, V_{H} and V_{L} sequences from the starting antibody are analyzed and a human T cell epitope “map” from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative V_{H} and V_{L} sequences are designed comprising combinations of amino acid substitutions and these sequences are subsequently incorporated into a range of binding polypeptides, e.g., membrane associated molecule-specific antibodies or immunospecific fragments thereof for use in the diagnostic and treatment methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

In the therapeutic methods described herein, administration is to an animal, e.g., a human, in need of treatment for cancer or other hyperproliferative disorder. For example, a binding molecule, e.g., a binding polypeptide, e.g., a membrane associated molecule-specific antibody or immunospecific fragment thereof may be administered to a human patient diagnosed with a tumor, other cancerous lesion, or other hyperproliferative disorder, a human patient who has been treated for cancer and is in remission, but is in need of further chronic treatment to prevent recurrence or spread of cancer, a human who exhibits early warning signs for a certain cancer or hyperproliferative disorder and is a candidate for preventative treatment, or preventatively to a human who is genetically predisposed to contract a certain cancer.

The methods of treatment of hyperproliferative disorders as described herein are typically tested in vitro, and then in vivo in an acceptable animal model, for the desired therapeutic or prophylactic activity, prior to use in humans. Suitable animal models, including transgenic animals, are well known to those of ordinary skill in the art. For example, in vitro assays to demonstrate the therapeutic utility of binding molecule described herein include the effect of a binding molecule on a cell line or a patient tissue sample. The effect of the binding molecule on the cell line and/or tissue sample can be determined utilizing techniques known to those of skill in the art including, but not limited to, apoptosis assays and cell lysis assays. In accordance with the invention, in vitro assays which can be used to determine whether administration of a specific binding molecule is indicated, include in vitro cell culture assays in which a patient tissue sample is grown in culture, and exposed to or otherwise administered a compound, and the effect of such compound upon the tissue sample is observed.

Antibodies or fragments thereof for use as therapeutic binding molecules may be generated by any suitable method known in the art. Polyclonal antibodies to an antigen of interest can be produced by various procedures well known in the art. For example, a binding molecule, e.g., a binding polypeptide, e.g., a membrane associated molecule-specific antibody or immunospecific fragment thereof can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. Monoclonal antibodies can be prepared using a wide variety of techniques known in the art including the use of hybridoma and recombinant and phage display technology.

Using art recognized protocols, in one example, antibodies are raised in mammals by multiple subcutaneous or intraperitoneal injections of the relevant antigen (e.g., purified tumor associated antigens such as a membrane associated molecules, varient polypeptides, fragments thereof, or cells or cellular extracts comprising such antigens) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Preferably, the lymphocytes are obtained from the spleen.

In this well known process (Kohler et al., Nature 256:495 (1975)) the relatively short-lived, or mortal, lymphocytes from a mammal which has been injected with antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by in vitro assays such as immunoprecipitation, radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, Academic Press, pp 59-103 (1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

The polypeptide sequence of the membrane associated molecules of the present invention was determined via mass spectroscopy. Accordingly, the source of antigen (e.g. a membrane associated molecule, variant polypeptide, or fragment thereof may be prepared according to methods well known in the art. For example, the protein may be isolated from large amounts of the disease-associated tissue, smaller fragments of the membrane associated molecule or variant polypeptide (about 10 to 125 amino acids) can be produced synthetically, the corresponding polynucleotide which encodes the membrane associated molecule can also be isolated and cloned according to methods known in the art.

Finally, small polynucleotide fragments can be produced based on deducing the polynucleotide sequence from the amino acid sequence. For example a polynucleotide sequence which encoded a 12 amino acid fragment of a membrane associated molecule could be synthesized based on the genetic code. Table 4 below indicates all of the bases which code for an amino acid. Thus, one of skill in the art could deduce a polynucleotide coding sequence based on the amino acid sequences described herein. The coding sequence could then be cloned into an expression vector described elsewhere herein and the resulting polypeptide could be purified, using methods known to one of skill in the art, see for example, the techniques described in “Methods In Enzymology”, 1990, Academic Press, Inc., San Diego, “Protein Purification: Principles and Practice”, 1982, Springer-Verlag, New York, which are incorporated by reference herein in their entirety. The purified polypeptide then could be used to immunize animals in the antibody production method described above. Additionally, the deduced polynucleotide sequences can be use to clone the polynucleotide which encodes the membrane associated molecules described herein.

TABLE 4
The Standard Genetic Code

| | T | C | A | G |
|---|---|---|---|---|
| T | TTT Phe (F) | TCT Ser (S) | TAT Tyr (Y) | TGT Cys (C) |
| | TTC Phe (F) | TCC Ser (S) | TAC Tyr (Y) | TGC |
| | TTA Leu (L) | TCA Ser (S) | TAA Ter | TGA Ter |
| | TTG Leu (L) | TCG Ser (S) | TAG Ter | TGG Trp (W) |
| C | CTT Leu (L) | CCT Pro (P) | CAT His (H) | CGT Arg (R) |
| | CTC Leu (L) | CCC Pro (P) | CAC His (H) | CGC Arg (R) |
| | CTA Leu (L) | CCA Pro (P) | CAA Gln (Q) | CGA Arg (R) |
| | CTG Leu (L) | CCG Pro (P) | CAG Gln (Q) | CGG Arg (R) |
| A | ATT Ile (I) | ACT Thr (T) | AAT Asn (N) | AGT Ser (S) |
| | ATC Ile (I) | ACC Thr (T) | AAC Asn (N) | AGC Ser (S) |
| | ATA Ile (I) | ACA Thr (T) | AAA Lys (K) | AGA Arg (R) |
| | ATG Met (M) | ACG Thr (T) | AAG Lys (K) | AGG Arg (R) |
| G | GTT Val (V) | GCT Ala (A) | GAT Asp (D) | GGT Gly (G) |
| | GTC Val (V) | GCC Ala (A) | GAC Asp (D) | GGC Gly (G) |
| | GTA Val (V) | GCA Ala (A) | GAA Glu (E) | GGA Gly (G) |
| | GTG Val (V) | GCG Ala (A) | GAG Glu (E) | GGG Gly (G) |

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a desired target polypeptide, e.g., a membrane associated molecule.

Antibody fragments that recognize specific epitopes may be generated by known techniques. For example, Fab and F(ab′)₂ fragments may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the C_{H}1 domain of the heavy chain.

Those skilled in the art will also appreciate that DNA encoding antibodies or antibody fragments (e.g., antigen binding sites) may also be derived from antibody phage libraries. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Exemplary methods are set forth, for example, in EP 368 684 B1; U.S. Pat. No. 5,969,108, Hoogenboom, H. R. and Chames, Immunol. Today 21:371 (2000); Nagy et al. Nat. Med. 8:801 (2002); Huie et al., Proc. Natl. Acad. Sci. USA 98:2682 (2001); Lui et al., J. Mol. Biol. 315:1063 (2002), each of which is incorporated herein by reference. Several publications (e.g., Marks et al., Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al., Nat. Biotechnol. 18:1287 (2000); Wilson et al., Proc. Natl. Acad. Sci. USA 98:3750 (2001); or Irving et al., J. Immunol. Methods 248:31 (2001)). In yet another embodiment, cell surface libraries can be screened for antibodies (Boder et al., Proc. Natl. Acad. Sci. USA 97:10701 (2000); Daugherty et al., J. Immunol. Methods 243:211 (2000)). Such procedures provide alternatives to traditional hybridoma techniques for the isolation and subsequent cloning of monoclonal antibodies.

In phage display methods, functional antibody domains are displayed on the surface of phage particles which carry the polynucleotide sequences encoding them. In particular, DNA sequences encoding V_{H} and V_{L} regions are amplified from animal cDNA libraries (e.g., human or murine cDNA libraries of lymphoid tissues) or synthetic cDNA libraries. In certain embodiments, the DNA encoding the V_{H} and V_{L} regions are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the V_{H} or V_{L} regions are usually recombinantly fused to either the phage gene III or gene VIII. Phage expressing an antigen binding domain that binds to an antigen of interest (i.e., a membrane associated molecule or a fragment thereof) can be selected or identified with antigen, e.g., using labeled antigen or antigen bound or captured to a solid surface or bead.

Additional examples of phage display methods that can be used to make the antibodies include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, which are incorporated herein by reference in their entireties. Humanized antibodies are antibody molecules that bind the desired antigen having one or more complementarity determining regions (CDRs) from a non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar Int. Rev. Immunol. 13:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Abgenix, Inc. (Freemont, Calif.) and GenPharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 12:899-903 (1988)). See also, U.S. Pat. No. 5,565,332.

Further, antibodies to target polypeptides of the invention can, in turn, be utilized to generate anti-idiotype antibodies that “mimic” target polypeptides using techniques well known to those skilled in the art. (See, e.g., Greenspan & Bona, FASEB J. 7(5):437-444 (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a desired target polypeptide and/or to bind its ligands/receptors, and thereby block its biological activity.

In another embodiment, DNA encoding desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be synthetic as described herein) may be used to clone constant and variable region sequences for the manufacture of antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody. The framework regions may be naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds to at least one epitope of a desired polypeptide, e.g., a membrane associated molecule. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As used herein, a chimeric antibody is a molecule in which different portions are derived from different animal species, such as those having a variable region derived from a murine monoclonal antibody and a human immunoglobulin constant region, e.g., humanized antibodies.

Alternatively, techniques described for the production of single chain antibodies (U.S. Pat. No. 4,694,778; Bird, Science 242:423-442 (1988); Huston et al., Proc. Natl. Acad. Sci. USA 85:5879-5883 (1988); and Ward et al., Nature 334:544-554 (1989)) can be adapted to produce single chain antibodies. Single chain antibodies are formed by linking the heavy and light chain fragments of the Fv region via an amino acid bridge, resulting in a single chain antibody. Techniques for the assembly of functional Fv fragments in E. coli may also be used (Skerra et al., Science 242:1038-1041 (1988)).

Yet other embodiments of the present invention comprise the generation of human or substantially human antibodies in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the V_{H} and V_{L} genes can be amplified using, e.g., RT-PCR. The V_{H} and V_{L} genes can be cloned into an antibody expression vector and transfected into cells (e.g., eukaryotic or prokaryotic cells) for expression.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Antibodies for use in the diagnostic and therapeutic methods disclosed herein can be produced by any method known in the art for the synthesis of antibodies, in particular, by chemical synthesis or preferably, by recombinant expression techniques as described herein.

It will further be appreciated that the scope of this invention further encompasses all alleles, variants and mutations of antigen binding DNA sequences.

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo dT cellulose. Suitable techniques are familiar in the art.

In one embodiment, cDNAs that encode the light and the heavy chains of the antibody may be made, either simultaneously or separately, using reverse transcriptase and DNA polymerase in accordance with well known methods. PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis.

Recombinant expression of an antibody, or fragment, derivative or analog thereof, e.g., a heavy or light chain of an antibody which binds to a target molecule described herein, e.g., a membrane associated molecule, requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody for use in the methods described herein. Thus, the invention includes host cells containing a polynucleotide encoding an antibody of the invention, or a heavy or light chain thereof, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express antibody molecules for use in the methods described herein. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lacZ coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is typically used as a vector to express foreign genes. The virus grows in Spodoptera frugiperda cells. The antibody coding sequence may be cloned individually into non-essential regions (for example, the polyhedrin gene) of the virus and placed under control of an AcNPV promoter (for example, the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 1980) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); TIB TECH 11(5):155-215 (May, 1993); and hygro, which confers resistance to hygromycin (Santerre et al., Gene 30:147 (1984). Methods commonly known in the art of recombinant DNA technology which can be used are described in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Academic Press, New York, Vol. 3. (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain is advantageously placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the invention has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, a preferred method for increasing the affinity of antibodies of the invention is disclosed in US 2002 0123057 A1.

Alterations to the variable region notwithstanding, those skilled in the art will appreciate that, in preferred embodiments, antibodies to membrane associated molecules of the instant invention may comprise antibodies, or immunoreactive fragments thereof, in which at least a fraction of, or the entire region of, one or more of the constant region domains has been deleted (“domain-deleted antibodies”) or otherwise altered so as to provide desired biochemical characteristics such as increased tumor localization or reduced serum half-life when compared with an antibody of approximately the same immunogenicity comprising a native or unaltered constant region.

In selected embodiments, the constant region of these antibodies to membrane associated molecules will comprise a human constant region. Modifications to the constant region compatible with the instant invention comprise additions, deletions or substitutions of one or more amino acids in one or more domains. That is, the antibodies to membrane associated molecules disclosed herein may comprise alterations or modifications to one or more of the three heavy chain constant domains (C_{H}1, C_{H}2 or C_{H}3) and/or to the light chain constant domain (C_{L}) (as described in PCT/US02/02373 which is incorporated herein by reference).

In especially preferred embodiments the modified antibodies will comprise domain deleted constructs or variants wherein the entire C_{H}2 domain has been removed (ΔC_{H}2 constructs). Additionally, alterations to the hinge region of the antibody can also be used in the present invention. Modified hinge region between the heavy chains of antibodies can improve purification, stability and production of the antibodies (described in PCT/US2004/020944 which is incorporated herein by reference). For other embodiments a short connecting peptide may be substituted for the deleted domain to provide flexibility and freedom of movement for the variable region. Those skilled in the art will appreciate that such constructs are particularly preferred due to the regulatory properties of the C_{H}2 domain on the catabolic rate of the antibody.

In certain embodiments, modified antibodies for use in the methods disclosed herein are minibodies. Minibodies can be made using methods described in the art (see, e.g., U.S. Pat. No. 5,837,821 or WO 94/09817A1).

In another embodiment, modified antibodies for use in the methods disclosed herein are C_{H}2 domain deleted antibodies which are known in the art. Domain deleted constructs can be derived using a vector (e.g., from Biogen Idec Incorporated) encoding an IgG₁ human constant domain (see, e.g., WO 02/060955A2 and WO02/096948A2). This exemplary vector was engineered to delete the C_{H}2 domain and provide a synthetic vector expressing a domain deleted IgG₁ constant region.

In one embodiment, a binding molecule, e.g., a binding polypeptide, e.g., a membrane associated molecule-specific antibody or immunospecific fragment thereof for use in the diagnostic and treatment methods disclosed herein comprises an immunoglobulin heavy chain having deletion or substitution of a few or even a single amino acid as long as it permits association between the monomeric subunits. For example, the mutation of a single amino acid in selected areas of the C_{H}2 domain may be enough to substantially reduce Fc binding and thereby increase tumor localization. Similarly, it may be desirable to simply delete that part of one or more constant region domains that control the effector function (e.g. complement binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be synthetic through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified antibody. Yet other embodiments comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

The present invention also provides the use of antibodies that comprise, consist essentially of, or consist of, variants (including derivatives) of antibody molecules (e.g., the V_{H} regions and/or V_{L} regions) described herein, which antibodies or fragments thereof immunospecifically bind to a membrane associated molecule, variant polypeptide or fragment thereof. Standard techniques known to those of skill in the art can be used to introduce mutations in the nucleotide sequence encoding a binding molecule, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference V_{H} region, V_{H}CDR1, V_{H}CDR2, V_{H}CDR3, V_{L} region, V_{L}CDR1, V_{L}CDR2, or V_{L}CDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind a membrane associated molecule).

For example, it is possible to introduce mutations only in framework regions or only in CDR regions of an antibody molecule. Introduced mutations may be silent or neutral missense mutations, i.e., have no, or little, effect on an antibody's ability to bind antigen. These types of mutations may be useful to optimize codon usage, or improve a hybridoma's antibody production. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein, (e.g., ability to immunospecifically bind at least one epitope of a membrane associated molecule) can be determined using techniques described herein or by routinely modifying techniques known in the art.

### Fusion Proteins and Antibody Conjugates

As discussed in more detail elsewhere herein, binding molecules, e.g., binding polypeptides, e.g., membrane associated molecule-specific antibodies or immunospecific fragments thereof for use in the diagnostic and treatment methods disclosed herein may further be recombinantly fused to a heterologous polypeptide at the N- or C-terminus or chemically conjugated (including covalent and non-covalent conjugations) to polypeptides or other compositions. For example, membrane associated molecule-specific binding molecules may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

Binding molecules, e.g., binding polypeptides, e.g., membrane associated molecule-specific antibodies or immunospecific fragments thereof for use in the diagnostic and treatment methods disclosed herein include derivatives that are modified, i.e., by the covalent attachment of any type of molecule to the antibody such that covalent attachment does not prevent the antibody binding a membrane associated molecule. For example, but not by way of limitation, the antibody derivatives include antibodies that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Binding molecules, e.g., binding polypeptides, e.g., membrane associated molecule-specific antibodies or immunospecific fragments thereof for use in the diagnostic and treatment methods disclosed herein can be composed of amino acids joined to each other by peptide bonds or modified peptide bonds, i.e., peptide isosteres, and may contain amino acids other than the 20 gene-encoded amino acids. Membrane associated molecule-specific antibodies may be modified by natural processes, such as posttranslational processing, or by chemical modification techniques which are well known in the art. Such modifications are well described in basic texts and in more detailed monographs, as well as in a voluminous research literature. Modifications can occur anywhere in the membrane associated molecule-specific antibody, including the peptide backbone, the amino acid side-chains and the amino or carboxyl termini, or on moieties such as carbohydrates. It will be appreciated that the same type of modification may be present in the same or varying degrees at several sites in a given membrane associated molecule-specific antibody. Also, a given membrane associated molecule-specific antibody may contain many types of modifications. Membrane associated molecule-specific antibodies may be branched, for example, as a result of ubiquitination, and they may be cyclic, with or without branching. Cyclic, branched, and branched cyclic membrane associated molecule-specific antibodies may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, T. E. Creighton, W. H. Freeman and Company, New York 2nd Ed., (1993); Posttranslational Covalent Modification Of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

The present invention also provides for fusion proteins comprising, consisting essentially of, or consisting of, an antibody (including molecules comprising, consisting essentially of, or consisting of, antibody fragments or variants thereof), that immunospecifically binds to a membrane associated molecule, and a heterologous polypeptide. Preferably, the heterologous polypeptide to which the antibody is fused is useful for function or is useful to target the membrane associated molecule expressing cells, including but not limited to a breast, ovarian, bladder, colon, lung, prostate, and pancreatic cancer cell. In an alternative preferred embodiment, the heterologous polypeptide to which the antibody is fused is useful for T cell, macrophage, and/or monocyte cell function or is useful to target the antibody to a T cell, macrophage, or monocyte. In one embodiment, a fusion protein of the invention comprises, consists essentially of, or consists of, a polypeptide having the amino acid sequence of any one or more of the V_{H} regions of an antibody of the invention or the amino acid sequence of any one or more of the V_{L} regions of an antibody of the invention or fragments or variants thereof, and a heterologous polypeptide sequence. In another embodiment, a fusion protein for use in the diagnostic and treatment methods disclosed herein comprises, consists essentially of, or consists of a polypeptide having the amino acid sequence of any one, two, three, or more of the V_{H} CDRs of an membrane associated molecule-specific antibody, or the amino acid sequence of any one, two, three, or more of the V_{L} CDRs of a membrane associated molecule-specific antibody, or fragments or variants thereof, and a heterologous polypeptide sequence. In one embodiment, the fusion protein comprises, consists essentially of, or consists of a polypeptide having the amino acid sequence of a V_{H} CDR3 of an membrane associated molecule-specific antibody, or fragment or variant thereof, and a heterologous polypeptide sequence, which fusion protein specifically binds to at least one epitope of membrane associated molecule. In another embodiment, a fusion protein comprises, consists essentially of, or consists of a polypeptide having the amino acid sequence of at least one V_{H} region of a membrane associated molecule-specific antibody and the amino acid sequence of at least one V_{L} region of a membrane associated molecule-specific antibody or immunospecific fragments thereof, and a heterologous polypeptide sequence. Preferably, the V_{H} and V_{L} regions of the fusion protein correspond to a single source antibody (or scFv or Fab fragment) which specifically binds at least one epitope of a membrane associated molecule. In yet another embodiment, a fusion protein for use in the diagnostic and treatment methods disclosed herein comprises, consists essentially of, or consists of a polypeptide having the amino acid sequence of any one, two, three or more of the V_{H} CDRs of a membrane associated molecule-specific antibody and the amino acid sequence of any one, two, three or more of the V_{L} CDRs of a membrane associated molecule-specific antibody, or fragments or variants thereof, and a heterologous polypeptide sequence. Preferably, two, three, four, five, six, or more of the V_{H}CDR(s) or V_{L}CDR(s) correspond to single source antibody (or scFv or Fab fragment) of the invention. Nucleic acid molecules encoding these fusion proteins are also encompassed by the invention.

The invention also pertains to the use of binding molecules which comprise one or more immunoglobulin domains. Fusion proteins for use in the diagnostic and therapeutic methods disclosed herein comprise a binding domain (which comprises at least one binding site) and a dimerization domain (which comprises at least one heavy chain portion). The subject fusion proteins may be bispecific (with one binding site for a first target and a second binding site for a second target) or may be multivalent (with two binding sites for the same target).

Exemplary fusion proteins reported in the literature include fusions of the T cell receptor (Gascoigne et al., Proc. Natl. Acad. Sci. USA 84:2936-2940 (1987)); CD4 (Capon et al., Nature 337:525-531 (1989); Traunecker et al., Nature 339:68-70 (1989); Zettmeissl et al., DNA Cell Biol. USA 9:347-353 (1990); and Byrn et al., Nature 344:667-670 (1990)); L-selectin (homing receptor) (Watson et al., J. Cell. Biol. 110:2221-2229 (1990); and Watson et al., Nature 349:164-167 (1991)); CD44 (Aruffo et al., Cell 61:1303-1313 (1990)); CD28 and B7 (Linsley et al., J. Exp. Med. 173:721-730 (1991)); CTLA-4 (Lisley et al., J. Exp. Med. 174:561-569 (1991)); CD22 (Stamenkovic et al., Cell 66:1133-1144 (1991)); TNF receptor (Ashkenazi et al., Proc. Natl. Acad. Sci. USA 88:10535-10539 (1991); Lesslauer et al., Eur. J. Immunol. 27:2883-2886 (1991); and Peppel et al., J. Exp. Med. 174:1483-1489 (1991)); and IgE receptor a (Ridgway and Gorman, J. Cell. Biol. Vol. 115, Abstract No. 1448 (1991)).

In one embodiment a fusion protein combines the binding domain(s) of the ligand or receptor (e.g. the extracellular domain (ECD) of a receptor) with at least one heavy chain domain and a synthetic connecting peptide. In one embodiment, when preparing the fusion proteins of the present invention, nucleic acid encoding the binding domain of the ligand or receptor domain will be fused C-terminally to nucleic acid encoding the N-terminus of an immunoglobulin constant domain sequence. N-terminal fusions are also possible. In one embodiment, a fusion protein includes a C_{H}2 and a C_{H}3 domain. Fusions may also be made to the C-terminus of the Fc portion of a constant domain, or immediately N-terminal to the CHI of the heavy chain or the corresponding region of the light chain.

In one embodiment, the sequence of the ligand or receptor binding domain is fused to the N-terminus of the Fc domain of an immunoglobulin molecule. It is also possible to fuse the entire heavy chain constant region to the ligand or receptor binding domain sequence. In one embodiment, a sequence beginning in the hinge region just upstream of the papain cleavage site which defines IgG Fc chemically (i.e. residue 216, taking the first residue of heavy chain constant region to be 114 according to the Kabat system), or analogous sites of other immunoglobulins is used in the fusion. The precise site at which the fusion is made is not critical; particular sites are well known and may be selected in order to optimize the biological activity, secretion, or binding characteristics of the molecule. Methods for making fusion proteins are known in the art.

For bispecific fusion proteins, the fusion proteins can be assembled as multimers, and particularly as heterodimers or heterotetramers. Generally, these assembled immunoglobulin-like proteins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

As discussed elsewhere herein, binding molecules, e.g., binding polypeptides, e.g., membrane associated molecule-specific antibodies or immunospecific fragments thereof for use in the diagnostic and treatment methods disclosed herein may be fused to heterologous polypeptides to increase the in vivo half life of the polypeptides or for use in immunoassays using methods known in the art. In many cases, the Fc part in a fusion protein is beneficial in therapy and diagnosis, and thus can result in, for example, improved pharmacokinetic properties. (EP A 232,262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5 receptor, have been fused with Fc portions for the purpose of high-throughput screening assays to identify antagonists of hIL-5. (See, D. Bennett et al., J. Molecular Recognition 8:52-58 (1995); K. Johanson et al. J. Biol. Chem. 270:9459-9471 (1995).

Moreover, binding molecules, e.g., binding polypeptides, e.g., membrane associated molecule-specific antibodies or immunospecific fragments thereof for use in the diagnostic and treatment methods disclosed herein can be fused to marker sequences, such as a peptide to facilitate their purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

Fusion proteins can be prepared using methods that are well known in the art (see, e.g., U.S. Pat. Nos. 5,116,964 and 5,225,538). Ordinarily, the ligand or ligand binding partner is fused C-terminally to the N-terminus of the constant region of the heavy chain (or heavy chain portion) and in place of the variable region. Any transmembrane regions or lipid or phospholipid anchor recognition sequences of ligand binding receptor are preferably inactivated or deleted prior to fusion. DNA encoding the ligand or ligand binding partner is cleaved by a restriction enzyme at or proximal to the 5′ and 3′ ends of the DNA encoding the desired ORF segment. The resultant DNA fragment is then readily inserted into DNA encoding a heavy chain constant region. The precise site at which the fusion is made may be selected empirically to optimize the secretion or binding characteristics of the soluble fusion protein. DNA encoding the fusion protein is then transfected into a host cell for expression.

Binding molecules for use in the methods of the present invention may be used in non-conjugated form or may be conjugated to at least one of a variety of molecules, e.g., to improve the therapeutic properties of the molecule, to facilitate target detection, or for imaging or therapy of the patient. Binding molecules, e.g., binding polypeptides, e.g., membrane associated molecule-specific antibodies or immunospecific fragments thereof for use in the diagnostic and treatment methods disclosed herein can be labeled or conjugated either before or after purification, when purification is performed.

In particular, binding molecules, e.g., binding polypeptides, e.g., membrane associated molecule-specific antibodies or immunospecific fragments thereof for use in the diagnostic and treatment methods disclosed herein may be conjugated to cytotoxins (such as radioisotopes, cytotoxic drugs, or toxins) therapeutic agents, cytostatic agents, biological toxins, prodrugs, peptides, proteins, enzymes, viruses, lipids, biological response modifiers, pharmaceutical agents, immunologically active ligands (e.g., lymphokines or other antibodies wherein the resulting molecule binds to both the neoplastic cell and an effector cell such as a T cell), or PEG. In another embodiment, a binding molecule, e.g., a binding polypeptide, e.g., a membrane associated molecule-specific antibody or immunospecific fragment thereof for use in the diagnostic and treatment methods disclosed herein can be conjugated to a molecule that decreases vascularization of tumors. In other embodiments, the disclosed compositions may comprise binding molecules, e.g., binding polypeptides, e.g., membrane associated molecule-specific antibodies or immunospecific fragments thereof coupled to drugs or prodrugs. Still other embodiments of the present invention comprise the use of binding molecules, e.g., binding polypeptides, e.g., membrane associated molecule-specific antibodies or immunospecific fragments thereof conjugated to specific biotoxins or their cytotoxic fragments such as ricin, gelonin, pseudomonas exotoxin or diphtheria toxin. The selection of which conjugated or unconjugated binding molecule to use will depend on the type and stage of cancer, use of adjunct treatment (e.g., chemotherapy or external radiation) and patient condition. It will be appreciated that one skilled in the art could readily make such a selection in view of the teachings herein.

It will be appreciated that, in previous studies, anti-tumor antibodies labeled with isotopes have been used successfully to destroy cells in solid tumors as well as lymphomas/leukemias in animal models, and in some cases in humans. Exemplary radioisotopes include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. The radionuclides act by producing ionizing radiation which causes multiple strand breaks in nuclear DNA, leading to cell death. The isotopes used to produce therapeutic conjugates typically produce high energy α- or β-particles which have a short path length. Such radionuclides kill cells to which they are in close proximity, for example neoplastic cells to which the conjugate has attached or has entered. They have little or no effect on non-localized cells. Radionuclides are essentially non-immunogenic.

With respect to the use of radiolabeled conjugates in conjunction with the present invention, binding molecules, e.g., binding polypeptides, e.g., membrane associated molecule-specific antibodies or immunospecific fragments thereof may be directly labeled (such as through iodination) or may be labeled indirectly through the use of a chelating agent. As used herein, the phrases “indirect labeling” and “indirect labeling approach” both mean that a chelating agent is covalently attached to a binding molecule and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

As used herein, the phrases “direct labeling” and “direct labeling approach” both mean that a radionuclide is covalently attached directly to a polypeptide (typically via an amino acid residue). More specifically, these linking technologies include random labeling and site-directed labeling. In the latter case, the labeling is directed at specific sites on the polypeptide, such as the N-linked sugar residues present only on the Fc portion of the conjugates. Further, various direct labeling techniques and protocols are compatible with the instant invention. For example, Technetium-99 labeled polypeptides may be prepared by ligand exchange processes, by reducing pertechnate (TcO₄⁻) with stannous ion solution, chelating the reduced technetium onto a Sephadex column and applying the binding polypeptides to this column, or by batch labeling techniques, e.g. by incubating pertechnate, a reducing agent such as SnCl₂, a buffer solution such as a sodium-potassium phthalate-solution, and the antibodies. In any event, preferred radionuclides for directly labeling antibodies are well known in the art and a particularly preferred radionuclide for direct labeling is ¹³¹I covalently attached via tyrosine residues. Binding molecules, e.g., binding polypeptides, e.g., membrane associated molecule-specific antibodies or immunospecific fragments thereof for use in the diagnostic and treatment methods disclosed herein may be derived, for example, with radioactive sodium or potassium iodide and a chemical oxidizing agent, such as sodium hypochlorite, chloramine T or the like, or an enzymatic oxidizing agent, such as lactoperoxidase, glucose oxidase and glucose.

Patents relating to chelators and chelator conjugates are known in the art. For instance, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, 5,434,287 and 5,124,471 of Gansow also relate to polysubstituted DTPA chelates. These patents are incorporated herein by reference in their entireties. Other examples of compatible metal chelators are ethylenediaminetetraacetic acid (EDTA), diethylenetriaminepentaacetic acid (DPTA), 1,4,8,11-tetraazatetradecane, 1,4,8,11-tetraazatetradecane-1,4,8,11-tetraacetic acid, 1-oxa-4,7,12,15-tetraazaheptadecane-4,7,12,15-tetraacetic acid, or the like. Cyclohexyl-DTPA or CHX-DTPA is particularly preferred and is exemplified extensively below. Still other compatible chelators, including those yet to be discovered, may easily be discerned by a skilled artisan and are clearly within the scope of the present invention.

Compatible chelators, including the specific bifunctional chelator used to facilitate chelation U.S. Pat. Nos. 6,682,134, 6,399,061, and 5,843,439, incorporated herein by reference in their entireties, are preferably selected to provide high affinity for trivalent metals, exhibit increased tumor-to-non-tumor ratios and decreased bone uptake as well as greater in vivo retention of radionuclide at target sites, i.e., B-cell lymphoma tumor sites. However, other bifunctional chelators that may or may not possess all of these characteristics are known in the art and may also be beneficial in tumor therapy.

It will also be appreciated that, in accordance with the teachings herein, binding molecules may be conjugated to different radiolabels for diagnostic and therapeutic purposes. To this end the aforementioned U.S. Pat. Nos. 6,682,134, 6,399,061, and 5,843,439 disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ¹¹¹In via a bifunctional chelator, i.e., Mx-DTPA (diethylene-triaminepentaacetic acid), which comprises a 1:1 mixture of 1-isothiocyanato-benzyl-3-methyl-DTPA and 1-methyl-3-isothiocyanatobenzyl-DTPA. ¹¹¹In is particularly preferred as a diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985).

As indicated above, a variety of radionuclides are applicable to the present invention and those skilled in the can readily determine which radionuclide is most appropriate under various circumstances. For example, ¹³¹I is a well known radionuclide used for targeted immunotherapy. However, the clinical usefulness of ¹³¹I can be limited by several factors including: eight-day physical half-life; dehalogenation of iodinated antibody both in the blood and at tumor sites; and emission characteristics (e.g., large gamma component) which can be suboptimal for localized dose deposition in tumor. With the advent of superior chelating agents, the opportunity for attaching metal chelating groups to proteins has increased the opportunities to utilize other radionuclides such as ¹¹¹In and ⁹⁰Y. ⁹⁰Y provides several benefits for utilization in radioimmunotherapeutic applications: the 64 hour half-life of ⁹⁰Y is long enough to allow antibody accumulation by tumor and, unlike e.g., ¹³¹I, ⁹⁰Y is a pure beta emitter of high energy with no accompanying gamma irradiation in its decay, with a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the local emission of ionizing radiation should be lethal for adjacent tumor cells lacking the target molecule.

Those skilled in the art will appreciate that non-radioactive conjugates may also be assembled using a variety of techniques depending on the selected agent to be conjugated. For example, conjugates with biotin are prepared e.g. by reacting a binding polypeptide with an activated ester of biotin such as the biotin N-hydroxysuccinimide ester. Similarly, conjugates with a fluorescent marker may be prepared in the presence of a coupling agent, e.g. those listed herein, or by reaction with an isothiocyanate, preferably fluorescein-isothiocyanate. Conjugates of the binding molecules, e.g., binding polypeptides, e.g., membrane associated molecule-specific antibodies or immunospecific fragments thereof with cytostatic/cytotoxic substances and metal chelates are prepared in an analogous manner.

Additional preferred agents for conjugation to binding molecules, e.g., binding polypeptides, e.g., membrane associated molecule-specific antibodies or immunospecific fragments thereof are cytotoxic drugs, particularly those which are used for cancer therapy. As used herein, “a cytotoxin or cytotoxic agent” means any agent that is detrimental to the growth and proliferation of cells and may act to reduce, inhibit or destroy a cell or malignancy. Exemplary cytotoxins include, but are not limited to, radionuclides, biotoxins, enzymatically active toxins, cytostatic or cytotoxic therapeutic agents, prodrugs, immunologically active ligands and biological response modifiers such as cytokines. Any cytotoxin that acts to retard or slow the growth of immunoreactive cells or malignant cells is within the scope of the present invention.

Exemplary cytotoxins include, in general, cytostatic agents, alkylating agents, antimetabolites, anti-proliferative agents, tubulin binding agents, hormones and hormone antagonists, and the like. Exemplary cytostatics that are compatible with the present invention include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine. Other preferred classes of cytotoxic agents include, for example, the maytansinoid family of drugs. Other preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, caminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. One skilled in the art may make chemical modifications to the desired compound in order to make reactions of that compound more convenient for purposes of preparing conjugates of the invention.

One example of particularly preferred cytotoxins comprise members or derivatives of the enediyne family of anti-tumor antibiotics, including calicheamicin, esperamicins or dynemicins. These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins and other enediynes are small molecules which are essentially non-immunogenic. These non-peptide toxins are chemically-linked to the dimers or tetramers by techniques which have been previously used to label monoclonal antibodies and other molecules. These linking technologies include site-specific linkage via the N-linked sugar residues present only on the Fc portion of the constructs. Such site-directed linking methods have the advantage of reducing the possible effects of linkage on the binding properties of the constructs.

As previously alluded to, compatible cytotoxins for preparation of conjugates may comprise a prodrug. As used herein, the term “prodrug” refers to a precursor or derivative form of a pharmaceutically active substance that is less cytotoxic to tumor cells compared to the parent drug and is capable of being enzymatically activated or converted into the more active parent form. Prodrugs compatible with the invention include, but are not limited to, phosphate-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. Further examples of cytotoxic drugs that can be derivatized into a prodrug form for use in the present invention comprise those chemotherapeutic agents described above.

Among other cytotoxins, it will be appreciated that binding molecules, e.g., binding polypeptides, e.g., membrane associated molecule-specific antibodies or immunospecific fragments thereof for use in the diagnostic and treatment methods disclosed herein can also be associated with or conjugated to a biotoxin such as ricin subunit A, abrin, diptheria toxin, botulinum, cyanginosins, saxitoxin, shigatoxin, tetanus, tetrodotoxin, trichothecene, verrucologen or a toxic enzyme. Preferably, such constructs will be made using genetic engineering techniques that allow for direct expression of the antibody-toxin construct. Other biological response modifiers that may be associated with the binding molecules, e.g., binding polypeptides, e.g., membrane associated molecule-specific antibodies or immunospecific fragments thereof disclosed herein comprise cytokines such as lymphokines and interferons. In view of the instant disclosure it is submitted that one skilled in the art could readily form such constructs using conventional techniques.

Another class of compatible cytotoxins that may be used in association with or conjugated to the disclosed binding molecules, e.g., binding polypeptides, e.g., membrane associated molecule-specific antibodies or immunospecific fragments thereof, are radiosensitizing drugs that may be effectively directed to tumor or immunoreactive cells. Such drugs enhance the sensitivity to ionizing radiation, thereby increasing the efficacy of radiotherapy. An antibody conjugate internalized by the tumor cell would deliver the radiosensitizer nearer the nucleus where radiosensitization would be maximal. The unbound radiosensitizer linked binding molecules of the invention would be cleared quickly from the blood, localizing the remaining radiosensitization agent in the target tumor and providing minimal uptake in normal tissues. After rapid clearance from the blood, adjunct radiotherapy would be administered in one of three ways: 1.) external beam radiation directed specifically to the tumor, 2.) radioactivity directly implanted in the tumor or 3.) systemic radioimmunotherapy with the same targeting antibody. A potentially attractive variation of this approach would be the attachment of a therapeutic radioisotope to the radiosensitized immunoconjugate, thereby providing the convenience of administering to the patient a single drug.

In certain embodiments, a moiety that enhances the stability or efficacy of a binding molecule, e.g., a binding polypeptide, e.g., a membrane associated molecule-specific antibody or immunospecific fragment thereof can be conjugated. For example, in one embodiment, PEG can be conjugated to the binding molecules of the invention to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

The present invention further encompasses the use of binding molecules, e.g., binding polypeptides, e.g., membrane associated molecule-specific antibodies or immunospecific fragments conjugated to a diagnostic or therapeutic agent. The binding molecules can be used diagnostically to, for example, monitor the development or progression of a tumor as part of a clinical testing procedure to, e.g., determine the efficacy of a given treatment and/or prevention regimen. Detection can be facilitated by coupling the binding molecule, e.g., binding polypeptide, e.g., membrane associated molecule-specific antibody or immunospecific fragment thereof to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

A binding molecule, e.g., a binding polypeptide, e.g., a membrane associated molecule-specific antibody or immunospecific fragment thereof also can be detectably labeled by coupling it to a chemiluminescent compound. The presence of the chemiluminescent-tagged binding molecule is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which a binding molecule, e.g., a binding polypeptide, e.g., a membrane associated molecule-specific antibody or immunospecific fragment thereof can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md., Diagnostic Horizons 2:1-7 (1978)); Voller et al., J. Clin. Pathol. 31:507-520 (1978); Butler, J. E., Meth. Enrymol. 73:482-523 (1981); Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981). The enzyme, which is bound to the binding molecule will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the binding molecule, e.g., binding polypeptide, e.g., membrane associated molecule-specific antibody or immunospecific fragment thereof, it is possible to detect cancer antigens through the use of a radioimmunoassay (RIA) (see, for example, Weintraub, B., Principles of Radioimmunoassays, Seventh Training Course on Radioligand Assay Techniques, The Endocrine Society, (March, 1986)), which is incorporated by reference herein). The radioactive isotope can be detected by means including, but not limited to, a gamma counter, a scintillation counter, or autoradiography.

A binding molecule, e.g., a binding polypeptide, e.g., a membrane associated molecule-specific antibody or immunospecific fragment thereof can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

Techniques for conjugating various moieties to a binding molecule, e.g., a binding polypeptide, e.g., a membrane associated molecule-specific antibody or immunospecific fragment thereof are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982).

### Antibody Expression

Following manipulation of the isolated genetic material to provide binding molecules, e.g., binding polypeptides, e.g., membrane associated molecule-specific antibodies or immunospecific fragments thereof for use in the diagnostic and treatment methods disclosed herein, the polynucleotides encoding the binding molecules are typically inserted in an expression vector for introduction into host cells that may be used to produce the desired quantity of binding molecule.

The term “vector” or “expression vector” is used herein to mean vectors used in accordance with the present invention as a vehicle for introducing into and expressing a desired gene in a host cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals.

In particularly preferred embodiments the cloned variable region genes are inserted into an expression vector along with the heavy and light chain constant region genes (preferably human) synthetic as discussed above. In one embodiment, this is effected using a proprietary expression vector of Biogen Idec Inc., referred to as NEOSPLA (U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in CHO cells, followed by selection in G418 containing medium and methotrexate amplification. Of course, any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). In general, screening large numbers of transformed cells for those which express suitably high levels if immunoglobulin heavy and light chains is routine experimentation which can be carried out, for example, by robotic systems. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other preferred embodiments the binding molecules, e.g., binding polypeptides, e.g., membrane associated molecule-specific antibodies or immunospecific fragments thereof for use in the diagnostic and treatment methods disclosed herein may be expressed using polycistronic constructs such as those disclosed in United States Patent Application Publication No. 2003-0157641 A1, filed Nov. 18, 2002 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of binding molecules, e.g., binding polypeptides, e.g., membrane associated molecule-specific antibodies or immunospecific fragments thereof in eukaryotic host cells. Compatible IRES sequences are disclosed in U.S. Pat. No. 6,193,980 which is also incorporated herein. Those skilled in the art will appreciate that such expression systems may be used to effectively produce the full range of binding molecules disclosed in the instant application.

More generally, once the vector or DNA sequence encoding a monomeric subunit of the binding polypeptide (e.g. a modified antibody) has been prepared, the expression vector may be introduced into an appropriate host cell. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Vectors, Rodriguez and Denhardt, Eds., Butterworths, Boston, Mass., Chapter 24.2, pp. 470-472 (1988). Typically, plasmid introduction into the host is via electroporation. The host cells harboring the expression construct are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

Along those same lines, “host cells” refers to cells which harbor vectors constructed using recombinant DNA techniques and encoding at least one heterologous gene. In descriptions of processes for isolation of antibodies from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

The host cell line used for protein expression is most preferably of mammalian origin; those skilled in the art are credited with ability to preferentially determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CV1 (monkey kidney line), COS (a derivative of CV1 with SV40 T antigen), R1610 (Chinese hamster fibroblast) BALBC/3T3 (mouse fibroblast), HAK (hamster kidney line), SP2/O (mouse myeloma), P3x63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature.

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein.

Genes encoding binding molecules, e.g., binding polypeptides, e.g., membrane associated molecule-specific antibodies or immunospecific fragments thereof for use in the diagnostic and treatment methods disclosed herein can also be expressed non-mammalian cells such as bacteria or yeast or plant cells. Bacteria which readily take up nucleic acids include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the heterologous polypeptides typically become part of inclusion bodies. The heterologous polypeptides must be isolated, purified and then assembled into functional molecules. Where tetravalent forms of antibodies are desired, the subunits will then self-assemble into tetravalent antibodies (WO02/096948A2).

In addition to prokaryotes, eukaryotic microbes may also be used. Saccharomyces cerevisiae, or common baker's yeast, is the most commonly used among eukaryotic microorganisms although a number of other strains are commonly available, e.g., Pichia pastoris.

For expression in Saccharomyces, the plasmid YRp7, for example, (Stinchcomb et al., Nature 282:39 (1979); Kingsman et al., Gene 7:141 (1979); Tschemper et al., Gene 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics 85:12 (1977)). The presence of the trp1 lesion as a characteristic of the yeast host cell genome then provides an effective environment for detecting transformation by growth in the absence of tryptophan.

### Immunoassays

Binding molecules, e.g., binding polypeptides, e.g., membrane associated molecule-specific antibodies or immunospecific fragments thereof for use in the diagnostic and treatment methods disclosed herein may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, Current Protocols in Molecular Biology, John Wiley & Sons, Inc., New York, Vol. 1 (1994), which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4.degree. C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, Current Protocols in Molecular Biology, John Wiley & Sons, Inc., New York, Vol. 1 (1994) at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32p or 1251) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, Current Protocols in Molecular Biology, John Wiley & Sons, Inc., New York Vol. 1 (1994) at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, Current Protocols in Molecular Biology, John Wiley & Sons, Inc., New York, Vol. 1 (1994) at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest is conjugated to a labeled compound (e.g., ³H or ¹²⁵I) in the presence of increasing amounts of an unlabeled second antibody.

Membrane associated molecule-specific binding molecules may, additionally, be employed histologically, as in immunofluorescence, immunoelectron microscopy or non-immunological assays, for in situ detection of cancer antigen gene products or conserved variants or peptide fragments thereof. In situ detection may be accomplished by removing a histological specimen from a patient, and applying thereto a labeled membrane associated molecule-specific antibody or fragment thereof, preferably applied by overlaying the labeled antibody (or fragment) onto a biological sample. Through the use of such a procedure, it is possible to determine not only the presence of colon tumor associated protein, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays for membrane associated molecule or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of binding to membrane associated molecules or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled membrane associated molecule-specific antibody. The solid phase support may then be washed with the buffer a second time to remove unbound antibody. Optionally the antibody is subsequently labeled. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of membrane associated molecule-specific antibody may be determined according to well known methods. Those skilled in the art will be able to determine operative and optimal assay conditions for each determination by employing routine experimentation.

There are a variety of methods available for measuring the affinity of an antibody-antigen interaction, but relatively few for determining rate constants. Most of the methods rely on either labeling antibody or antigen, which inevitably complicates routine measurements and introduces uncertainties in the measured quantities.

Surface plasmon reasonance (SPR) as performed on BIAcore offers a number of advantages over conventional methods of measuring the affinity of antibody-antigen interactions: (i) no requirement to label either antibody or antigen; (ii) antibodies do not need to be purified in advance, cell culture supernatant can be used directly; (iii) real-time measurements, allowing rapid semi-quantitative comparison of different monoclonal antibody interactions, are enabled and are sufficient for many evaluation purposes; (iv) biospecific surface can be regenerated so that a series of different monoclonal antibodies can easily be compared under identical conditions; (v) analytical procedures are fully automated, and extensive series of measurements can be performed without user intervention. BIAapplications Handbook, version AB (reprinted 1998), BIACORE code No. BR-1001-86; BIAtechnology Handbook, version AB (reprinted 1998), BIACORE code No. BR-1001-84.

SPR based binding studies require that one member of a binding pair be immobilized on a sensor surface. The binding partner immobilized is referred to as the ligand. The binding partner in solution is referred to as the analyte. In some cases, the ligand is attached indirectly to the surface through binding to another immobilized molecule, which is referred as the capturing molecule. SPR response reflects a change in mass concentration at the detector surface as analytes bind or dissociate.

Based on SPR, real-time BIAcore measurements monitor interactions directly as they happen. The technique is well suited to determination of kinetic parameters. Comparative affinity ranking is extremely simple to perform, and both kinetic and affinity constants can be derived from the sensorgram data.

When analyte is injected in a discrete pulse across a ligand surface, the resulting sensorgram can be divided into three essential phases: (i) Association of analyte with ligand during sample injection; (ii) Equilibrium or steady state during sample injection, where the rate of analyte binding is balanced by dissociation from the complex; (iii) Dissociation of analyte from the surface during buffer flow.

The association and dissociation phases provide information on the kinetics of analyte-ligand interaction (kₐ and k_{d}, the rates of complex formation and dissociation, k_{d}/kₐ=K_{D}). The equilibrium phase provides information on the affinity of the analyte-ligand interaction (K_{D}).

BIAevaluation software provides comprehensive facilities for curve fitting using both numerical integration and global fitting algorithms. With suitable analysis of the data, separate rate and affinity constants for interaction can be obtained from simple BIAcore investigations. The range of affinities measurable by this technique is very broad ranging from mM to pM.

Epitope specificity is an important characteristic of a monoclonal antibody. Epitope mapping with BIAcore, in contrast to conventional techniques using radioimmunoassay, ELISA or other surface adsorption methods, does not require labeling or purified antibodies, and allows multi-site specificity tests using a sequence of several monoclonal antibodies. Additionally, large numbers of analyses can be processed automatically.

Pair-wise binding experiments test the ability of two MAbs to bind simultaneously to the same antigen. MAbs directed against separate epitopes will bind independently, whereas MAbs directed against identical or closely related epitopes will interfere with each other's binding. These binding experiments with BIAcore are straightforward to carry out.

For example, one can use a capture molecule to bind the first Mab, followed by addition of antigen and second MAb sequentially. The sensorgrams will reveal: 1. how much of the antigen binds to first Mab, 2. to what extent the second MAb binds to the surface-attached antigen, 3. if the second MAb does not bind, whether reversing the order of the pair-wise test alters the results.

Peptide inhibition is another technique used for epitope mapping. This method can complement pair-wise antibody binding studies, and can relate functional epitopes to structural features when the primary sequence of the antigen is known. Peptides or antigen fragments are tested for inhibition of binding of different MAbs to immobilized antigen. Peptides which interfere with binding of a given MAb are assumed to be structurally related to the epitope defined by that MAb.

### Pharmaceutical Compositions and Administration Methods

Methods of preparing and administering binding molecules, e.g., binding polypeptides, e.g., membrane associated molecule-specific antibodies or immunospecific fragments thereof to a subject in need thereof are well known to or are readily determined by those skilled in the art. The route of administration of the binding molecule, e.g., binding polypeptide, e.g., membrane associated molecule-specific antibody or immunospecific fragment thereof may be, for example, oral, parenteral, by inhalation or topical. The term parenteral as used herein includes, e.g., intravenous, intraarterial, intraperitoneal, intramuscular, subcutaneous, rectal or vaginal administration. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, binding molecules, e.g., binding polypeptides, e.g., membrane associated molecule-specific antibodies or immunospecific fragments thereof can be delivered directly to the site of the adverse cellular population thereby increasing the exposure of the diseased tissue to the therapeutic agent.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a binding molecule, e.g., a binding polypeptide, e.g., a membrane associated molecule-specific antibody or immunospecific fragment thereof, by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampules, bags, bottles, syringes or vials, and sealed under aseptic conditions according to methods known in the art. Further, the preparations may be packaged and sold in the form of a kit such as those described in co-pending U.S. Ser. No. 09/259,337 (US-2002-0102208 A1), which is incorporated herein by reference in its entirety. Such articles of manufacture will preferably have labels or package inserts indicating that the associated compositions are useful for treating a subject suffering from, or predisposed to hyperproliferative disorders.

Effective doses of the compositions of the present invention, for treatment of hyperproliferative disorders as described herein vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

For treatment of hyperproliferative disorders with an antibody or other binding molecule, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

Binding molecules, e.g., binding polypeptides, e.g., membrane associated molecule-specific antibodies or immunospecific fragments thereof for use in the diagnostic and treatment methods disclosed herein can be administered on multiple occasions. Intervals between single dosages can be weekly, monthly or yearly. Intervals can also be irregular as indicated by measuring blood levels of target polypeptide or target molecule in the patient. In some methods, dosage is adjusted to achieve a plasma polypeptide concentration of 1-1000 μg/ml and in some methods 25-300 μg/ml. Alternatively, binding molecules can be administered as a sustained release formulation, in which case less frequent administration is required. Dosage and frequency vary depending on the half-life of the antibody in the patient. The half-life of a binding molecule can also be prolonged via fusion to a stable polypeptide or moeity, e.g., albumin or PEG. In general, humanized antibodies show the longest half-life, followed by chimeric antibodies and nonhuman antibodies. In one embodiment, the binding molecules of the invention can be administered in unconjugated form, In another embodiment, the binding molecules, e.g., binding polypeptides, e.g., membrane associated molecule-specific antibodies or immunospecific fragments thereof for use in the methods disclosed herein can be administered multiple times in conjugated form. In still another embodiment, the binding molecules of the invention can be administered in unconjugated form, then in conjugated form, or vise versa.

The dosage and frequency of administration can vary depending on whether the treatment is prophylactic or therapeutic. In prophylactic applications, compositions comprising antibodies or a cocktail thereof are administered to a patient not already in the disease state or in a pre-disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of binding molecule, e.g., antibody per dose, with dosages of from 5 to 25 mg being more commonly used for radioimmunoconjugates and higher doses for cytotoxin-drug conjugated molecules) at relatively short intervals is sometimes required until progression of the disease is reduced or terminated, and preferably until the patient shows partial or complete amelioration of symptoms of disease. Thereafter, the patent can be administered a prophylactic regime.

In one embodiment, a subject can be treated with a nucleic acid molecule encoding a binding molecule, e.g., a binding polypeptide, e.g., a membrane associated molecule-specific antibody or immunospecific fragment thereof (e.g., in a vector). Doses for nucleic acids encoding polypeptides range from about 10 ng to 1 g, 100 ng to 100 mg, 1 μg to 10 mg, or 30-300 μg DNA per patient. Doses for infectious viral vectors vary from 10-100, or more, virions per dose.

Polynucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al., Nucl. Acids Res. 16:3209 (1988), methylphosphonate oligonucleotides can be prepared by use of controlled pore glass polymer supports (Sarin et al., Proc. Natl. Acad. Sci. U.S.A. 85:7448-7451 (1988)), etc.

Therapeutic agents can be administered by parenteral, topical, intravenous, oral, subcutaneous, intraarterial, intracranial, intraperitoneal, intranasal or intramuscular means for prophylactic and/or therapeutic treatment. In some methods, agents are injected directly into a particular tissue where membrane associated molecule-expressing cells have accumulated, for example intracranial injection. Intramuscular injection or intravenous infusion are preferred for administration of antibody. In some methods, particular therapeutic antibodies are injected directly into the cranium. In some methods, antibodies are administered as a sustained release composition or device, such as a Medipad™ device.

Agents of the invention can optionally be administered in combination with other agents that are effective in treating the disorder or condition in need of treatment (e.g., prophylactic or therapeutic).

Effective single treatment dosages (i.e., therapeutically effective amounts) of ⁹⁰Y-labeled antibodies range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-à-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While a great deal of clinical experience has been gained with ¹³¹I and ⁹⁰Y, other radiolabels are known in the art and have been used for similar purposes. Still other radioisotopes are used for imaging. For example, additional radioisotopes which are compatible with the scope of the instant invention include, but are not limited to, ¹²³I, ¹²⁵I, ³²P, ⁵⁷Co, ⁶⁴Cu, ⁶⁷Cu, ⁷⁷Br, ⁸¹Rb, ⁸¹Kr, ⁸⁷Sr, ¹¹³In, ¹²⁷Cs, ¹²⁹Cs, ¹³²I, ¹⁹⁷Hg, ²⁰³Pb, ²⁰⁶Bi, ¹⁷⁷Lu, ¹⁸⁶Re, ²¹²Pb, ²¹²Bi, 47Sc, ¹⁰⁵Rd, ¹⁰⁹Pd, ¹⁵³Sm, ¹⁸⁸Re, ¹⁹⁹Au, ²²⁵Ac, ²¹¹At, and ²¹³Bi. In this respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include ¹²⁵I, ¹²³I, ⁹⁹Tc, ⁴³K, ⁵²Fe, ⁶⁷Ga, ⁶⁸Ga, as well as ¹¹¹In. Antibodies have also been labeled with a variety of radionuclides for potential use in targeted immunotherapy (Peirersz et al. Immunol. Cell Biol. 65: 111-125 (1987)). These radionuclides include ¹⁸⁸Re and ¹⁸⁶Re as well as ¹⁹⁹Au and ⁶⁷Cu to a lesser extent. U.S. Pat. No. 5,460,785 provides additional data regarding such radioisotopes and is incorporated herein by reference.

Whether or not binding molecules, e.g., binding polypeptides, e.g., membrane associated molecule-specific antibodies or immunospecific fragments thereof for use in the diagnostic and treatment methods disclosed herein are used in a conjugated or unconjugated form, it will be appreciated that a major advantage of the present invention is the ability to use these molecules in myelosuppressed patients, especially those who are undergoing, or have undergone, adjunct therapies such as radiotherapy or chemotherapy. That is, the beneficial delivery profile (i.e. relatively short serum dwell time, high binding affinity and enhanced localization) of the molecules makes them particularly useful for treating patients that have reduced red marrow reserves and are sensitive to myelotoxicity. In this regard, the unique delivery profile of the molecules make them very effective for the administration of radiolabeled conjugates to myelosuppressed cancer patients. As such, binding molecules, e.g., binding polypeptides, e.g., membrane associated molecule-specific antibodies or immunospecific fragments thereof for use in the treatment methods disclosed herein are useful in a conjugated or unconjugated form in patients that have previously undergone adjunct therapies such as external beam radiation or chemotherapy. In other preferred embodiments, binding molecules, e.g., binding polypeptides, e.g., membrane associated molecule-specific antibodies or immunospecific fragments thereof (again in a conjugated or unconjugated form) may be used in a combined therapeutic regimen with chemotherapeutic agents. Those skilled in the art will appreciate that such therapeutic regimens may comprise the sequential, simultaneous, concurrent or coextensive administration of the disclosed antibodies or other binding molecules and one or more chemotherapeutic agents. Particularly preferred embodiments of this aspect of the invention will comprise the administration of a radiolabeled binding polypeptide.

While binding molecules, e.g., binding polypeptides, e.g., membrane associated molecule-specific antibodies or immunospecific fragments thereof may be administered as described immediately above, it must be emphasized that in other embodiments conjugated and unconjugated binding molecules may be administered to otherwise healthy patients as a first line therapeutic agent. In such embodiments binding molecules may be administered to patients having normal or average red marrow reserves and/or to patients that have not, and are not, undergoing adjunct therapies such as external beam radiation or chemotherapy.

However, as discussed above, selected embodiments of the invention comprise the administration of binding molecules, e.g., binding polypeptides, e.g., membrane associated molecule-specific antibodies or immunospecific fragments thereof to myelosuppressed patients or in combination or conjunction with one or more adjunct therapies such as radiotherapy or chemotherapy (i.e. a combined therapeutic regimen). As used herein, the administration of binding molecules, e.g., binding polypeptides, e.g., membrane associated molecule-specific antibodies or immunospecific fragments thereof in conjunction or combination with an adjunct therapy means the sequential, simultaneous, coextensive, concurrent, concomitant or contemporaneous administration or application of the therapy and the disclosed binding molecules. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates described herein. Conversely, cytotoxin-conjugated binding molecules could be administered intravenously followed by tumor localized external beam radiation. In yet other embodiments, binding molecules may be administered concurrently with one or more selected chemotherapeutic agents in a single office visit. A skilled artisan (e.g. an experienced oncologist) would be readily be able to discern effective combined therapeutic regimens without undue experimentation based on the selected adjunct therapy and the teachings of the instant specification.

In this regard it will be appreciated that the combination of a binding molecule (with or without cytotoxin) and the chemotherapeutic agent may be administered in any order and within any time frame that provides a therapeutic benefit to the patient. That is, the chemotherapeutic agent and binding molecule, e.g., binding polypeptide, e.g., membrane associated molecule-specific antibody or immunospecific fragment thereof, may be administered in any order or concurrently. In selected embodiments binding molecules, e.g., binding polypeptides, e.g., membrane associated molecule-specific antibodies or immunospecific fragments thereof for use in treatment methods disclosed herein will be administered to patients that have previously undergone chemotherapy. In yet other embodiments, binding molecules, e.g., binding polypeptides, e.g., membrane associated molecule-specific antibodies or immunospecific fragments thereof for use in treatment methods disclosed herein will be administered substantially simultaneously or concurrently with the chemotherapeutic treatment. For example, the patient may be given the binding molecule while undergoing a course of chemotherapy. In preferred embodiments the binding molecule will be administered within 1 year of any chemotherapeutic agent or treatment. In other preferred embodiments the polypeptide will be administered within 10, 8, 6, 4, or 2 months of any chemotherapeutic agent or treatment. In still other preferred embodiments the binding molecule will be administered within 4, 3, 2 or 1 week of any chemotherapeutic agent or treatment. In yet other embodiments the binding molecule will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

Moreover, in accordance with the present invention a myelosuppressed patient shall be held to mean any patient exhibiting lowered blood counts. Those skilled in the art will appreciate that there are several blood count parameters conventionally used as clinical indicators of myelosuppresion and one can easily measure the extent to which myelosuppresion is occurring in a patient. Examples of art accepted myelosuppression measurements are the Absolute Neutrophil Count (ANC) or platelet count. Such myelosuppression or partial myeloablation may be a result of various biochemical disorders or diseases or, more likely, as the result of prior chemotherapy or radiotherapy. In this respect, those skilled in the art will appreciate that patients who have undergone traditional chemotherapy typically exhibit reduced red marrow reserves. As discussed above, such subjects often cannot be treated using optimal levels of cytotoxin (i.e. radionuclides) due to unacceptable side effects such as anemia or immunosuppression that result in increased mortality or morbidity.

More specifically conjugated or unconjugated binding molecules, e.g., binding polypeptides, e.g., membrane associated molecule-specific antibodies or immunospecific fragments thereof for use in treatment methods disclosed herein may be used to effectively treat patients having ANCs lower than about 2000/mm³ or platelet counts lower than about 150,000/mm³. More preferably binding molecules, e.g., binding polypeptides, e.g., membrane associated molecule-specific antibodies or immunospecific fragments thereof for use in treatment methods disclosed herein may be used to treat patients having ANCs of less than about 1500/mm³, less than about 1000/mm³ or even more preferably less than about 500/mm³. Similarly, binding molecules, e.g., binding polypeptides, e.g., membrane associated molecule-specific antibodies or immunospecific fragments thereof for use in treatment methods disclosed herein may be used to treat patients having a platelet count of less than about 75,000/mm³, less than about 50,000/mm³ or even less than about 10,000/mm³. In a more general sense, those skilled in the art will easily be able to determine when a patient is myelosuppressed using government implemented guidelines and procedures.

As indicated above, many myelosuppressed patients have undergone courses of treatment including chemotherapy, implant radiotherapy or external beam radiotherapy. In the case of the latter, an external radiation source is for local irradiation of a malignancy. For radiotherapy implantation methods, radioactive reagents are surgically located within the malignancy, thereby selectively irradiating the site of the disease. In any event, the disclosed binding molecules, e.g., binding polypeptides, e.g., membrane associated molecule-specific antibodies or immunospecific fragments thereof for use in treatment methods disclosed herein may be used to treat disorders in patients exhibiting myelosuppression regardless of the cause.

In this regard it will further be appreciated that binding molecules, e.g., binding polypeptides, e.g., membrane associated molecule-specific antibodies or immunospecific fragments thereof for use in treatment methods disclosed herein may be used in conjunction or combination with any chemotherapeutic agent or agents (e.g. to provide a combined therapeutic regimen) that eliminates, reduces, inhibits or controls the growth of neoplastic cells in vivo. As discussed, such agents often result in the reduction of red marrow reserves. This reduction may be offset, in whole or in part, by the diminished myelotoxicity of the compounds of the present invention that advantageously allow for the aggressive treatment of neoplasias in such patients. In other embodiments, radiolabeled immunoconjugates disclosed herein may be effectively used with radiosensitizers that increase the susceptibility of the neoplastic cells to radionuclides. For example, radiosensitizing compounds may be administered after the radiolabeled binding molecule has been largely cleared from the bloodstream but still remains at therapeutically effective levels at the site of the tumor or tumors.

With respect to these aspects of the invention, exemplary chemotherapeutic agents that are compatible with the instant invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in Harrison's Principles of Internal Medicine 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with one or more binding molecules, e.g., binding polypeptides, e.g., membrane associated molecule-specific antibodies or immunospecific fragments thereof as described herein.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade malignancies, who fail to achieve remission or relapse, salvage therapy is used. Salvage therapies employ drugs such as cytosine arabinoside, cisplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

The amount of chemotherapeutic agent to be used in combination with the binding molecules disclosed herein may vary by subject or may be administered according to what is known in the art. See for example, Bruce A Chabner et al., Antineoplastic Agents, in Goodman & Gilman's The Pharmacological Basis of Therapeutics 1233-1287 ((Joel G. Hardman et al., eds., 9^{th} ed. (1996).

As previously discussed, binding molecules, e.g., binding polypeptides, e.g., membrane associated molecule-specific antibodies or immunospecific fragments thereof, or recombinants thereof may be administered in a pharmaceutically effective amount for the in vivo treatment of mammalian hyperproliferative disorders. In this regard, it will be appreciated that the disclosed antibodies will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of binding molecule, e.g., binding polypeptide, e.g., membrane associated molecule-specific antibody or immunospecific fragment thereof, or recombinant thereof, conjugated or unconjugated to a therapeutic agent, shall be held to mean an amount sufficient to achieve effective binding to a target and to achieve a benefit, e.g., to ameliorate symptoms of a disease or disorder or to detect a substance or a cell. In the case of tumor cells, the binding molecule will be preferably be capable of interacting with selected immunoreactive antigens on neoplastic or immunoreactive cells, or on non neoplastic cells, e.g., vascular cells associated with neoplastic cells. and provide for an increase in the death of those cells. Of course, the pharmaceutical compositions of the present invention may be administered in single or multiple doses to provide for a pharmaceutically effective amount of the binding molecule.

In keeping with the scope of the present disclosure, binding molecules, e.g., binding polypeptides, e.g., membrane associated molecule-specific antibodies or immunospecific fragments thereof for use in treatment methods disclosed herein may be administered to a human or other animal in accordance with the aforementioned methods of treatment in an amount sufficient to produce a therapeutic or prophylactic effect. The binding molecules, e.g., binding polypeptides, e.g., membrane associated molecule-specific antibodies or immunospecific fragments thereof for use in treatment methods disclosed herein can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding molecules according to the present invention may prove to be particularly effective.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical-Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984).

To create the arrays of the invention, single-stranded nucleic acid molecules, e.g., polynucleotide probes, can be spotted onto a substrate in a two-dimensional matrix or array. Each single-stranded polynucleotide probe can comprise at least 5, 10, 15, 20, 25, 30, 35, 40, or 50 or more contiguous nucleotides. In one embodiment, the polynucleotide probes can be selected from the nucleotide sequences shown in Table 6 (for example, SEQ ID NOs:2,385-3,438).

The invention also includes an array comprising a marker of the present invention e.g., some or all of the sets of molecules set forth in Tables 7-10, complements or fragments thereof. The array can be used to assay expression of one or more genes in the array. In one embodiment, the array can be used to assay gene expression in a tissue to ascertain tissue specificity of genes in the array. This allows a profile to be developed showing a battery of genes specifically expressed in one or more tissues, e.g., cancerous tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a hyperproliferative disease or disorder, progression of a hyperproliferative disease or disorder, and processes, such a cellular transformation, e.g., cellular proliferation, associated with a hyperproliferative disease or disorder. The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and abnormal cells, e.g., cancerous cells. This provides a battery of genes that could serve as a molecular target for diagnosis or therapeutic intervention.

### Preparation of Arrays

Arrays are known in the art and consist of a surface to which probes that correspond in sequence to gene products (e.g., cDNAs, mRNAs, cRNAs, polypeptides, and fragments thereof), can be specifically hybridized or bound at a known position. In one embodiment, the array is a matrix in which each position represents a discrete binding site for a product encoded by a gene (e.g., a protein or RNA), and in which binding sites are present for products of most or almost all of the genes in the organism's genome. In one embodiment, the “binding site” (hereinafter, “site”) is a nucleic acid or nucleic acid analogue to which a particular cognate cDNA can specifically hybridize. The nucleic acid or analogue of the binding site can be, e.g., a synthetic oligomer, a full-length cDNA, a less-than full length cDNA, or a gene fragment.

### Preparing Nucleic Acid Molecules for Arrays

As noted above, the “binding site” to which a particular cognate cDNA specifically hybridizes is usually a nucleic acid or nucleic acid analogue attached at that binding site. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (i.e., fragments that do not share more than 10 bases of contiguous identical sequence with any other fragment on the array). Computer programs are useful in the design of primers with the required specificity and optimal amplification properties. See, e.g., Oligo version 5.0 (National Biosciences™). In the case of binding sites corresponding to very long genes, it will sometimes be desirable to amplify segments near the 3′ end of the gene so that when oligo-dT primed cDNA probes are hybridized to the array, less-than-full length probes will bind efficiently. Typically each gene fragment on the array will be between about 50 bp and about 2000 bp, more typically between about 100 bp and about 1000 bp, and usually between about 300 bp and about 800 bp in length. PCR methods are well known and are described, for example, in Innis et al. eds., 1990, PCR Protocols: A Guide to Methods and Applications, Academic Press Inc. San Diego, Calif., which is incorporated by reference in its entirety. It will be apparent that computer controlled robotic systems are useful for isolating and amplifying nucleic acids.

An alternative means for generating the nucleic acid molecules for the array is by synthesis of synthetic polynucleotides or oligonucleotides, e.g., using N-phosphonate or phosphoramidite chemistries (Froehler et al. (1986) Nucleic Acid Res 14:5399-5407; McBride et al. (1983) Tetrahedron Lett. 24:245-248). Synthetic sequences are between about 15 and about 500 bases in length, more typically between about 20 and about 50 bases. In some embodiments, synthetic nucleic acids include non-natural bases, e.g., inosine. As noted above, nucleic acid molecule analogues may be used as binding sites for hybridization. An example of a suitable nucleic acid analogue is peptide nucleic acid (see, e.g., Eghohn et al. (1993) Nature 365:566-568; see also U.S. Pat. No. 5,539,083).

In an alternative embodiment, the binding (hybridization) sites are made from plasmid or phage clones of genes, cDNAs (e.g., expressed sequence tags), or inserts therefrom (Nguyen et al. (1995) Genomics 29:207-209). In yet another embodiment, the polynucleotide of the binding sites is RNA.

### Attaching Nucleic Acid Molecules to the Solid Surface

The nucleic acid molecule or analogue are attached to a solid support, which may be made from glass, plastic (e.g., polypropylene, nylon), polyacrylamide, nitrocellulose, or other materials. An example of a method for attaching the nucleic acid molecules to a surface is by printing on glass plates, as is described generally by Schena et al. (1995) Science 270:467-470, the contents of which are expressly incorporated herein by reference. This method is especially useful for preparing arrays of cDNA. See also DeRisi et al (1996) Nature Genetics 14:457-460; Shalon et al. (1996) Genome Res. 6:639-645; and Schena et al. (1995) Proc. Natl. Acad. Sci. USA 93:10539-11286. Each of the aforementioned articles is incorporated by reference in its entirety.

A second example of a method for making arrays is by making high-density oligonucleotide arrays. Techniques are known for producing arrays containing thousands of oligonucleotides complementary to defined sequences, at defined locations on a surface using photolithographic techniques for synthesis in situ (see, Fodor et al, (1991) Science 251:767-773; Pease et al., (1994) Proc. Natl. Acad. Sci. USA 91:5022-5026; Lockhart et al. (1996) Nature Biotech 14:1675; U.S. Pat. Nos. 5,578,832; 5,556,752; and 5,510,270, each of which is incorporated by reference in its entirety for all purposes) or other methods for rapid synthesis and deposition of defined oligonucleotides (Blanchard et al. (1996) Biosensors & Bioelectronics 11: 687-90). When these methods are used, oligonucleotides (e.g., 20-mers) of known sequence are synthesized directly on a surface such as a derivatized glass slide. In one embodiment, the array produced is redundant, with several oligonucleotide molecules per RNA. Oligonucleotide probes can be chosen to detect alternatively spliced mRNAs.

Other methods for making arrays, e.g., by masking (Maskos and Southern, 1992, Nuc. Acids Res. 20:1679-1684), may also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989, which is hereby incorporated in its entirety), could be used, although, as will be recognized by those of skill in the art, very small arrays will be preferred because hybridization volumes will be smaller.

Another method for making arrays is to directly deposit the probe on to the array surface. In such an embodiment probes will bind non-covalently or covalently to the array depending on the surface of the array and characteristics of the probe. In preferred embodiments the array has an epoxy coating on top of a glass microscope slide and the probe is modified at the amino terminal by an amine group. This combination of array surface and probe modification results in the covalent binding of the probe. Other methods of coating the array surface include using acrylamide, sialinization and nitrocellulose. There are several methods for direct deposit of the probes on to the array surface. In one embodiment, the probes are deposited using a pin dispense technique. In this technique, pins deposit probes onto the surface either using contact or non-contact printing. One preferred embodiment is non-contact printing using quill tip pins. Another embodiment uses piezo electric dispensing to deposit the probes.

Control composition may be present on the array including compositions comprising oligonucleotides or polynucleotides corresponding to genomic DNA, housekeeping genes, negative and positive control genes, and the like. These latter types of compositions are not “unique”, i.e., they are “common.” In other words, they are calibrating or control genes whose function is not to tell whether a particular “key” gene of interest is expressed, but rather to provide other useful information, such as background or basal level of expression. The percentage of samples which are made of unique oligonucleotides or polynucleotide that correspond to the same type of gene is generally at least about 30%, and usually at least about 60% and more usually at least about 80%. Preferably, the arrays of the present invention will be of a specific type, e.g., cancer arrays e.g., lung, colon, pancreatic and ovarian cancer.

### Generating Labeled Probes

Methods for preparing total and poly(A)+ RNA are well known and are described generally in Sambrook et al., supra. In one embodiment, RNA is extracted from cells of the various types of interest in this invention using guanidinium thiocyanate lysis followed by CsCl centrifugation (Chirgwin et al. (1979) Biochemistry 18:5294-5299). Poly(A)+ RNA is selected by selection with oligo-dT cellulose (see Sambrook et al., supra).

Labeled cDNA is prepared from mRNA by oligo dT-primed or random-primed reverse transcription, both of which are well known in the art (see e.g., Klug and Berger, (1987) Methods Enzymol. 152:316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al. (1996) Nature Biotech. 14:1675, the contents of which are expressly incorporated herein by reference). In alternative embodiments, the cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

When fluorescently-labeled probes are used, many suitable fluorophores are known, including fluorescein, lissamine, phycoerythrin, rhodamine (Perkin Elmer Cetus™), Cy2, Cy3, Cy3.5, Cy5, Cy5.5, Cy7, Fluor X (Amersham™) and others (see, e.g., Kricka (1992) Nonisotopic DNA Probe Techniques, Academic Press San Diego, Calif.). It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used (see Zhao et al. (1995) Gene 156:207; Pietu et al. (1996) Genome Res. 6:492). However, because of scattering of radioactive particles, and the consequent requirement for widely spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham™)) with reverse transcriptase (e.g., SuperScript™. II, LTI Inc.) at 42° C. for 60 min.

### Generation of Targets

In one detection method, the array of immobilized nucleic acid molecules, or probes, is contacted with a target sample containing target nucleic acid molecules, to which a fluorescent label is attached. Target nucleic acid molecules hybridize to the probes on the array and any non-hybridized nucleic acid molecules are removed. The array containing the hybridized target nucleic acid molecules are exposed to light which excites the flourescent label. The resulting fluorescent intensity, or brightness, is detected.

Tissue samples which are suspected of being metastatic or the metastatic potential of which is unknown can be treated to form single-stranded polynucleotides, for example by heating or by chemical denaturation, as is known in the art. The single-stranded polynucleotides in the tissue sample can then be labeled and hybridized to the polynucleotide probes on the array. Detectable labels which can be used include but are not limited to radiolabels, biotinylated labels, fluorophors, and chemiluminescent labels. Double stranded polynucleotides, comprising the labeled sample polynucleotides bound to polynucleotide probes, can be detected once the unbound portion of the sample is washed away. Detection can be visual or with computer assistance.

In another method, the target cDNA is generated from RNA derived from selected tissue samples (target samples). In one embodiment, the cDNA is labeled with a molecule which specifically binds with a second molecule which is labeled with one of the detection labels mentioned above for the detection of hybridization. In one embodiment, the cDNA is synthesized using a biotinylated dNTP. The biotinylated target cDNA is then hybridized to the array. There is then a second hybridization using streptavidin labeled with an appropriate fluorphore. The streptavidin will bind specifically to the biotinylated cDNA resulting in the detection of cDNA hybridization to the probe. In another embodiment, the cDNA is synthesized using specific primer sequences which add a capture sequence as the cDNA is being synthesized. The cDNA with the capture sequence is hybridized to the probes on the array. A second hybridization is performed using a fluorescently labeled molecule which binds specifically to the capture sequence. resulting in the detection of cDNA hybridization to the probe. Detection can be visual or with computer assistance.

### Hybridization to Arrays

Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al., supra, and Chee et al., supra).

Optimal hybridization conditions will depend on the length (e.g., oligomer versus polynucleotide greater than 200 bases) and type (e.g., RNA, DNA, PNA) of labeled probe and immobilized polynucleotide or oligonucleotide. General parameters for specific (i.e., stringent) hybridization conditions for nucleic acid molecules are described in Sambrook et al., supra, and in Ausubel et al., 1987, Current Protocols in Molecular Biology, Greene Publishing and Wiley-Interscience, New York, which is incorporated in its entirety for all purposes. Such stringent conditions are known to those skilled in the art and can be found in sections 6.3.1-6.3.6 of Current Protocols in Molecular Biology, John Wiley & Sons, N.Y. (1989). A non-limiting example of stringent hybridization conditions are hybridization in 6× sodium chloride/sodium citrate (SSC) at about 45° C., followed by one or more washes in 0.2×SSC, 0.1% SDS at 50-65° C. Useful hybridization conditions are also provided in, e.g., Tijessen, 1993, Hybridization With Nucleic Acid Probes, Elsevier Science Publishers B.V. and Kricka, 1992, Nonisotopic DNA Probe Techniques, Academic Press San Diego, Calif.

### Signal Detection and Data Analysis

When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, A DNA array system for analyzing complex DNA samples using two-color fluorescent probe hybridization, Genome Research 6:639-645, which is incorporated by reference in its entirety for all purposes). The arrays may be scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices 35 are described in Schena et al., 1996, Genome Res. 6:639-645 and in other references cited herein. Alternatively, the fiber-optic bundle described by Ferguson et al., 1996, Nature Biotech. 14:1681-1684, may be used to monitor mRNA abundance levels at a large number of sites simultaneously.

Signals are recorded and, in one embodiment, analyzed by computer, e.g., using a 12 bit analog to digital board. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated by drug administration, gene deletion, or any other tested event. According to the method of the invention, the relative abundance of an mRNA in two cells or cell lines is scored as a perturbation (i.e., the abundance is different in the two sources of mRNA tested), or as not perturbed (i.e., the relative abundance is the same). As used herein, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Present detection methods allow reliable detection of difference of an order of about 3-fold to about 5-fold, but more sensitive methods are expected to be developed.

In some cases, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

In another embodiment, a single fluorophore is used and all of the hybridizations from the samples are detected at a single wave length. In this method, the samples are all compared with each other to determine expression levels. The expression levels for the membrane associated molecules are determined by comparing fluorescence intensity values from all of the samples from the same wavelength. There are several different methods used for data analysis using a single fluorophore for hybridization. One method is using global normalization. Briefly, the intensity values from all of the sequences are averaged for each sample. All of the sample intensity averages are then averaged to determine the experimental intensity average. A correction factor is calculated for each sample by dividing the experimental intensity average by the sample averages. All of the sequence intensity values are multiplied by the correction factor. Following normalization, the malignant sample values are divided by the nonmalignant sample values to determine the fold expression change.

Another method to analyze the intensity values uses a nonparametric analysis. Nonparametric statistical analysis of microarray data is performed by Spearman Rank Analysis. In the first method, each gene is ranked in order of measured fluorescence intensity within each sample and ranks are compared between tumor samples and grouped normal samples. The statistical significance of each comparison is recorded. In the second method, each gene is ranked in order of measured fluorescence intensity across samples and ranks are compared between tumor samples and grouped normal samples. The statistical significance of each comparison is recorded. For each method, each gene is counted for the number of tumor samples that had statistically higher rank than the normal samples for each tumor indication.

### Diagnostic Assays

An exemplary method for detecting the presence or absence of a marker protein or nucleic acid in a biological sample involves obtaining a biological sample from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a marker protein include enzyme linked immunosorbent assays (ELISAs), Western blots, immunoprecipitations and immunofluorescence. In vitro techniques for detection of genomic DNA include Southern hybridizations. Furthermore, in vivo techniques for detection of a marker protein include introducing into a subject a labeled antibody directed against the protein or fragment thereof. For example, the antibody can be labeled with a radioactive marker whose presence and location in a subject can be detected by standard imaging techniques.

A general principle of such diagnostic and prognostic assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the markers or probes onto a solid phase support to create an array, as described herein, where more than one marker or probe is used, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In one embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S, and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of marker mRNA can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an array as described herein. A skilled artisan can readily adapt known mRNA detection methods for use in detecting the level of mRNA encoded by the markers of the present invention.

An alternative method for determining the level of mRNA marker in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., sample not associated with a hyperproliferative disease or disorder, or between samples from different sources.

Alternatively, the expression level can be provided as a relative expression level. To determine a relative expression level of a marker, the level of expression of the marker is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from cells from a subject associated with a hyperproliferative disease or disorder or from cells not associated with a hyperproliferative disease or disorder. The choice of the cell source is dependent on the use of the relative expression level. Using expression found in normal tissues as a mean expression score aids in validating whether the marker assayed is specific to cells associated with a hyperproliferative disease or disorder (versus normal cells). In addition, as more data is accumulated, the mean expression value can be revised, providing improved relative expression values based on accumulated data. Expression data from cells provides a means for grading the severity of the hyperproliferative disease or disorder

In another embodiment of the present invention, a marker protein is detected. An example of an agent for detecting marker protein of the invention is an antibody capable of binding to such a protein or a fragment thereof, preferably an antibody with a detectable label. Antibodies can be polyclonal, or more preferably, monoclonal. An intact antibody, or a fragment or derivative thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether cells express a marker of the present invention.

In one format, antibodies, or antibody fragments or derivatives, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

The invention also encompasses kits for detecting the presence of a marker protein or nucleic acid in a biological sample. Such kits can be used to determine if a subject is suffering from or is at increased risk of developing a hyperproliferative disease or disorder. For example, the kit can comprise a labeled compound or agent capable of detecting a marker protein or nucleic acid in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for interpreting the results obtained using the kit.

For antibody-based kits, the kit can comprise, for example: (1) a first antibody (e.g., attached to a solid support) which binds to a marker protein; and, optionally, (2) a second, different antibody which binds to either the protein or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a marker protein or (2) a pair of primers useful for amplifying a marker nucleic acid molecule. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

### Surrogate Markers

The markers of the invention may serve as surrogate markers for one or more disorders or disease states or for conditions leading up to disease states, and in particular, a hyperproliferative disease or disorder. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The markers of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker transcription or expression, the amplified marker may be in a quantity which is more readily detectable than the drug itself. Also, the marker may be more easily detected due to the nature of the marker itself; for example, using the methods described herein, antibodies may be employed in an immune-based detection system for a protein marker, or marker-specific radiolabeled probes may be used to detect a mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Pharm. 56 Suppl. 3: S16-S20.

All of the references cited above, as well as all references cited herein, are incorporated herein by reference in their entireties.

### EXEMPLIFICATION

This invention is further illustrated by the following examples, which should not be construed as limiting. The contents of all references, Tables, Figures, Sequence Listings, patents and published patent applications cited throughout this application are hereby incorporated by reference. Additionally, all nucleotide and amino acid sequences deposited in public databases referred to herein are also hereby incorporated by reference.

### Example 1

This example illustrates the process used to assemble the screening signature set outlined in FIG. 1.

The Ensemble™ predicted protein database was queried using a model of membrane topology prediction, the Transmembrane Hidden Markov Model (TMHMM), algorithm to select sequences predicted to contain a transmembrane domain. This group of sequences was termed the transmembrane selection set and contains 5661 sequences. The sequences in the transmembrane selection set were then compared to the sequences on the Affymetrix U133 set and Affymetrix U95 set expression arrays. Those sequences without substantial homology (<96% identity to sequences on the Affymetrix arrays) were then associated to form the transmembrane signature set. This transmembrane signature set was composed of 768 sequences.

Those sequences with substantial homology to the array associated molecules (>96% identity to sequences on the Affymetrix arrays), that were excluded from the transmembrane signature set were then used to query an expression database. The GeneLogic cancer suite was queried and any sequence which was associated with substantial intensity values was omitted. Here, sequences which were present in >5% of the patient samples in at most 5 sample sets were selected for the expression signature set. Upon completion of the screening the expression signature set contained 1175 sequences.

The expression signature set and the transmembrane signature set were then combined and manually validated. Manual validation consisted of determining cellular localization. Any sequences which were known to be cell surface molecules or which localization was unknown were grouped together to provide a master transmembrane signature set. The master transmembrane signature set contained 984 sequences.

To further improve the identification power of the array, the Ensemble™ predicted protein database was queried for sequences predicted to contain ITIM, ITAM, ITSM or GPI link motifs. The database was queried for [I/V]XYXXL sequence to identify ITIM motifs, YXX[L/V]X7-11YXX[L/V] to identify ITAM motifs and [S/T]XTXXX[V/I] to identify ITSM motifs. GPI link motifs were identified using several methods. Each sequence was analyzed by a signal sequence prediction program, Signal P, for the presence of an amino terminal signal sequence. Each sequence was analyzed by a hydropathy determination program for the presence of hydrophobic residues at the carboxy terminal. Each sequence was additionally analyzed for the presence of cleavage sites at both ends of the molecule typically found in GPI link motif molecules. The pattern at the amino cleavage site is [GAS]X[GASCT], pattern at the carboxy cleavage site is [GASCDN]X[GAS]. Together the sequences identified as having ITIM, ITAM, ITSM or GPI motifs were selected for the group termed motif selection set which contained 636 sequences.

The motif selection set was then compared to the sequences on the Affymetrix U133 set and Affymetrix U95 set expression arrays. Those sequences without substantial homology to the array associated molecules (<96% identity to sequences on the Affymetrix arrays) were selected for the group termed the motif signature set which contained 149 sequences.

The master transmembrane signature set (984 sequences) and the motif signature set (149 sequences) were combined to form the screening signature set (1133 sequences), which was used to generate an array for use in further experiments to identify membrane associated molecules exhibiting altered expression profiles in tumor samples.

In addition, 13 control sequences were selected for inclusion with the screening signature set. One skilled in the art will appreciate the use of these sequences as positive and negative controls for the experiment. The final screening signature set contained 1146 sequences, of which SEQ ID NOs: 1134-1146 are the control sequences (see Table 6).

### Example 2

This example illustrates the methods used to design and manufacture an expression array using the screening set of sequences.

The probe design, oligo synthesis and array manufacture were completed by MWG Biotech AG (Ebersberg, Germany). MWG Biotech used the CodeSeq Database and oligonucleotides4Array Design software to design unique probes for each sequence. oligonucleotides4Array software is used to design oligonucleotides with the correct physical requirements (e.g., melting temperature, GC content, secondary structure probability, dimerization), and performs Blast and Smith-Waterman alignments of oligonucleotides to the Codeseq Database. Smith-Waterman alignments algorithm is used to ensure that homology between the probe and any other gene sequence is >75%. The CodeSeq Database is a proprietary database of gene sequences used to ensure that the oligonucleotides bind uniquely to the appropriate sequence. MWG Biotech designed oligonucleotides which are 50 nucleotides in length.

MWG Biotech then synthesized the selected oligonucleotides. All of the oligonucleotides were quality controlled using analysis on MALDI TOF which ensures the sequence identity by molecular weight and verifies complete and successful purification of the oligonucleotides. The oligonucleotides are synthesized with the amino terminal modified with an MMT group.

The oligonucleotides were then arrayed on MWG Biotech epoxy coated slides to provide arrays in accordance with the present invention. The slides used in this embodiment are glass microscope slides with an epoxy coating. The epoxy surface allows for covalent binding with the amino group of the modified oligonucleotides. The oligonucleotides were arrayed using pin printing technology with quill tips. The resulting arrays were then used in further experiments to identify membrane associated molecules which are indicative of proliferative disorders.

### Example 3

This example illustrates the identification of SEQ ID NO:142 (a polynucleotide corresponding to a membrane associated molecule) as a marker or therapeutic target for colon cancer. The hybridization of target with the expression arrays was outsourced to Genisphere Inc (Hatfield, Pa.). Genisphere uses a proprietary dendramer technique for detection of target hybridization.

Target samples were generated from tumor tissue obtained from patients along with appropriate controls. RNA was isolated from malignant and nonmalignant tissue samples using Qiagen RNeasy maxi kit according to the manufacturer's instructions. The resulting total RNA was analyzed by spectrophotometer and agarose gel to ensure the quality and integrity of the RNA. Good quality RNA was DNase treated using Invitrogen's DNase I and then cleaned up and concentrated using Qiagen's MinElute kit. The DNase treated RNA was then used in Invitrogen's First Strand Synthesis Kit to synthesize cDNA. During cDNA synthesis primers from Genisphere were used. These primers tag the cDNA as it is being synthesized with a capture sequence that is used in the detection phase of the experiment.

The cDNA was hybridized to the array generated from the screening signature set of sequences by Genisphere Inc. Briefly, the cDNA was incubated with the arrays in 2×SDS buffer (0.5 M NaPO4), 1% SDS, 2 mM EDTA, 2×SSC, 4×Denhardt's solution) for 16-24 hrs at 55-60° C., and washed using 2×SSC with 0.2% SDS, 2×SSC and 0.2×SSC at 42° C. Following the washes the arrays were incubate with the fluorescently labeled (CY3) dendramer from Genisphere for 2.5-3 hours. The arrays were then washed using 2×SSC with 0.2% SDS at 42° C. and 2×SSC and 0.2×SSC at room temperature. The arrays were then scanned on an Axon scanner at 546 wavelength to measure intensity and detect hybridization.

The data from the arrays was analyzed using global analysis methods. Briefly, the intensity values from all of the sequences were averaged for each sample. All of the sample intensity averages were then averaged to determine the experimental intensity average. A correction factor was calculated for each sample by dividing the experimental intensity average by the sample averages. All of the sequence intensity values were multiplied by the correction factor. Following normalization, the malignant sample values were divided by the nonmalignant sample values to determine the fold expression change.

The fold expression values for SEQ ID NO:142 in malignant colon samples are shown in Table 11. On average, SEQ ID NO:142 is overexpressed 4.9 fold in malignant colon samples compared to nonmalignant colon and is considered a potential marker or therapeutic target for colon cancer. The membrane associated molecule encoded by SEQ ID NO:142 is known in the literature as MUC-4 (mucin 4).

Those skilled in the art will recognize, or be able to ascertain using no more than routine experimentation, many equivalents to the specific embodiments of the invention described herein. Such equivalents are intended to be encompassed by the following claims.

### Example 4

### Quantitative PCR (QPCR)

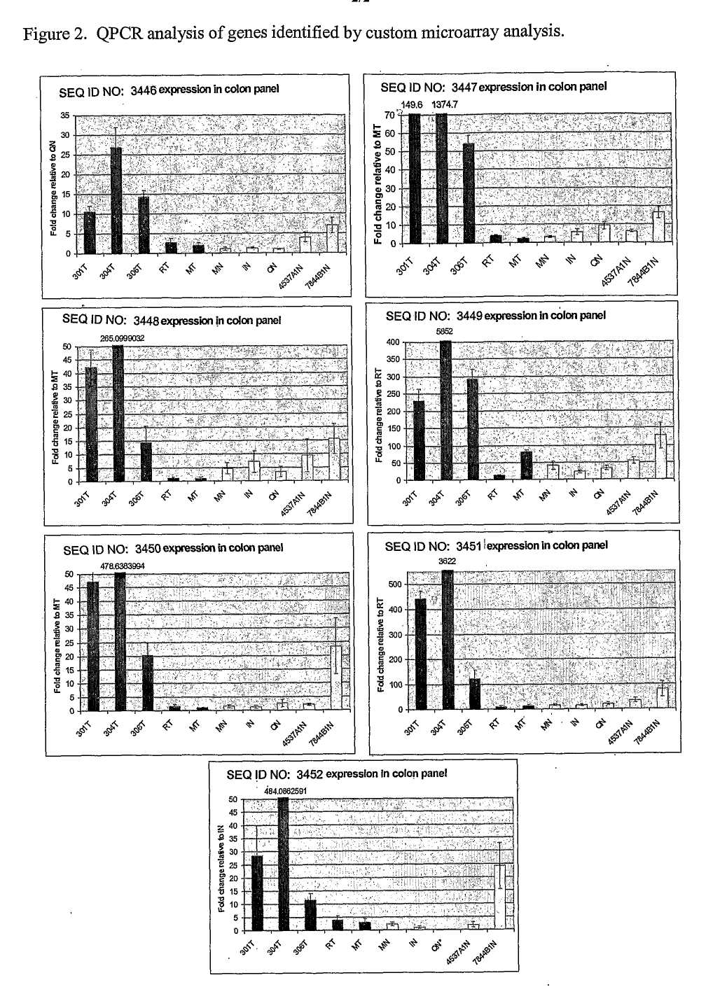

A subset of genes identified from the custom chip as upregulated in colon cancer was evaluated by quantitative PCR. FIG. 2 depicts the results for seven genes; SEQ ID NO:3446, SEQ ID NO:3447, SEQ ID NO:3448, SEQ ID NO:3449, SEQ ID NO:3450, SEQ ID NO:3451 and SEQ ID NO:3452. These genes showed higher expression in colon tumors compared to normal colon, consistent with results obtained from the microarray. The human tissue samples were obtained through Sharp Grossmont Hospital (La Mesa, Calif.) and from commercial sources (Asterand Inc., Detroit Mich. and the Cooperative Human Tissue Network, Nashville Tenn.) RNA and cDNA were prepared from samples using Qiagen RNEasy kits (Qiagen Inc, Valencia, Calif.) and Superscript First Strand Synthesis System (Invitrogen, Carlsbad, Calif.) essentially according to manufacturer specifications. cDNA integrity was measured using the RNA 6000 Nano Assay on the Agilent 2100 Bioanalyzer (Agilent, Palo Alto, Calif.). Concentrations were normalized and used for TaqMan™ gene expression analysis. Assays were performed in triplicate on the ABI Prism 7900 instrument (Applied Biosystems) using 25 μl reactions containing 1×TaqMan Universal PCR Master Mix (Applied Biosystems), 900 nM each primer, 250 nM probe and approximately 1-20 ng cDNA. For every gene target analyzed, cycle threshold values were determined for both GAPDH and the target of interest. Comparative CT analysis was used to establish an expression value for each target of interest normalized to GAPDH. Finally, the data were presented as fold change in expression, relative to the tissue having the lowest normalized value for that experiment.

The present invention is not to be limited in scope by the specific embodiments described which are intended as single illustrations of individual aspects of the invention, and any compositions or methods which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference.

TABLE 6

| Nucleotide ID | Protein ID | Probe ID | Colon | Lung | Pancreas | Ovarian |
|---|---|---|---|---|---|---|
| Seq ID 001 | Seq ID 1147 | Seq ID 2293 | X | | | |
| Seq ID 002 | Seq ID 1148 | Seq ID 2294 | | | | |
| Seq ID 003 | Seq ID 1149 | Seq ID 2295 | | | | |
| Seq ID 004 | Seq ID 1150 | Seq ID 2296 | | | | |
| Seq ID 005 | Seq ID 1151 | Seq ID 2297 | | | | |
| Seq ID 006 | Seq ID 1152 | Seq ID 2298 | | | | |
| Seq ID 007 | Seq ID 1153 | Seq ID 2299 | | | | |
| Seq ID 008 | Seq ID 1154 | Seq ID 2300 | X | | | |
| Seq ID 009 | Seq ID 1155 | Seq ID 2301 | X | | | |
| Seq ID 010 | Seq ID 1156 | Seq ID 2302 | | | | |
| Seq ID 011 | Seq ID 1157 | Seq ID 2303 | | | | |
| Seq ID 012 | Seq ID 1158 | Seq ID 2304 | | | | |
| Seq ID 013 | Seq ID 1159 | Seq ID 2305 | X | | | |
| Seq ID 014 | Seq ID 1160 | Seq ID 2306 | X | | | X |
| Seq ID 015 | Seq ID 1161 | Seq ID 2307 | | | | |
| Seq ID 016 | Seq ID 1162 | Seq ID 2308 | | | | |
| Seq ID 017 | Seq ID 1163 | Seq ID 2309 | | | | |
| Seq ID 018 | Seq ID 1164 | Seq ID 2310 | | | | |
| Seq ID 019 | Seq ID 1165 | Seq ID 2311 | X | | | |
| Seq ID 020 | Seq ID 1166 | Seq ID 2312 | | | | |
| Seq ID 021 | Seq ID 1167 | Seq ID 2313 | | | | |
| Seq ID 022 | Seq ID 1168 | Seq ID 2314 | | | | |
| Seq ID 023 | Seq ID 1169 | Seq ID 2315 | X | | | |
| Seq ID 024 | Seq ID 1170 | Seq ID 2316 | | | | |
| Seq ID 025 | Seq ID 1171 | Seq ID 2317 | X | | | |
| Seq ID 026 | Seq ID 1172 | Seq ID 2318 | | | | |
| Seq ID 027 | Seq ID 1173 | Seq ID 2319 | | | | |
| Seq ID 028 | Seq ID 1174 | Seq ID 2320 | X | | | |
| Seq ID 029 | Seq ID 1175 | Seq ID 2321 | | | | |
| Seq ID 030 | Seq ID 1176 | Seq ID 2322 | | | | |
| Seq ID 031 | Seq ID 1177 | Seq ID 2323 | | | | |
| Seq ID 032 | Seq ID 1178 | Seq ID 2324 | | | | |
| Seq ID 033 | Seq ID 1179 | Seq ID 2325 | | | | |
| Seq ID 034 | Seq ID 1180 | Seq ID 2326 | | X | | |
| Seq ID 035 | Seq ID 1181 | Seq ID 2327 | | | | |
| Seq ID 036 | Seq ID 1182 | Seq ID 2328 | | | | |
| Seq ID 037 | Seq ID 1183 | Seq ID 2329 | | | | |
| Seq ID 038 | Seq ID 1184 | Seq ID 2330 | | | | |
| Seq ID 039 | Seq ID 1185 | Seq ID 2331 | | | | |
| Seq ID 040 | Seq ID 1186 | Seq ID 2332 | | | | |
| Seq ID 041 | Seq ID 1187 | Seq ID 2333 | | | | |
| Seq ID 042 | Seq ID 1188 | Seq ID 2334 | | | | |
| Seq ID 043 | Seq ID 1189 | Seq ID 2335 | | | | |
| Seq ID 044 | Seq ID 1190 | Seq ID 2336 | | | | |
| Seq ID 045 | Seq ID 1191 | Seq ID 2337 | | | | |
| Seq ID 046 | Seq ID 1192 | Seq ID 2338 | X | | | X |
| Seq ID 047 | Seq ID 1193 | Seq ID 2339 | | | | |
| Seq ID 048 | Seq ID 1194 | Seq ID 2340 | | | | |
| Seq ID 049 | Seq ID 1195 | Seq ID 2341 | | | | |
| Seq ID 050 | Seq ID 1196 | Seq ID 2342 | | | | |
| Seq ID 051 | Seq ID 1197 | Seq ID 2343 | | | | |
| Seq ID 052 | Seq ID 1198 | Seq ID 2344 | X | | | |
| Seq ID 053 | Seq ID 1199 | Seq ID 2345 | | | | |
| Seq ID 054 | Seq ID 1200 | Seq ID 2346 | | | | |
| Seq ID 055 | Seq ID 1201 | Seq ID 2347 | | | | |
| Seq ID 056 | Seq ID 1202 | Seq ID 2348 | | | | |
| Seq ID 057 | Seq ID 1203 | Seq ID 2349 | | | | |
| Seq ID 058 | Seq ID 1204 | Seq ID 2350 | | | | |
| Seq ID 059 | Seq ID 1205 | Seq ID 2351 | | | | |
| Seq ID 060 | Seq ID 1206 | Seq ID 2352 | | | | |
| Seq ID 061 | Seq ID 1207 | Seq ID 2353 | | | | |
| Seq ID 062 | Seq ID 1208 | Seq ID 2354 | | | | X |
| Seq ID 063 | Seq ID 1209 | Seq ID 2355 | | | | |
| Seq ID 064 | Seq ID 1210 | Seq ID 2356 | | | | |
| Seq ID 065 | Seq ID 1211 | Seq ID 2357 | | | | |
| Seq ID 066 | Seq ID 1212 | Seq ID 2358 | | | | |
| Seq ID 067 | Seq ID 1213 | Seq ID 2359 | | | | |
| Seq ID 068 | Seq ID 1214 | Seq ID 2360 | | | | |
| Seq ID 069 | Seq ID 1215 | Seq ID 2361 | | | | |
| Seq ID 070 | Seq ID 1216 | Seq ID 2362 | | | | |
| Seq ID 071 | Seq ID 1217 | Seq ID 2363 | | | | |
| Seq ID 072 | Seq ID 1218 | Seq ID 2364 | X | | | |
| Seq ID 073 | Seq ID 1219 | Seq ID 2365 | | | | |
| Seq ID 074 | Seq ID 1220 | Seq ID 2366 | | | | |
| Seq ID 075 | Seq ID 1221 | Seq ID 2367 | | | | |
| Seq ID 076 | Seq ID 1222 | Seq ID 2368 | | | | |
| Seq ID 077 | Seq ID 1223 | Seq ID 2369 | | | | |
| Seq ID 078 | Seq ID 1224 | Seq ID 2370 | X | | X | |
| Seq ID 079 | Seq ID 1225 | Seq ID 2371 | X | | | |
| Seq ID 080 | Seq ID 1226 | Seq ID 2372 | | | | |
| Seq ID 081 | Seq ID 1227 | Seq ID 2373 | | | | |
| Seq ID 082 | Seq ID 1228 | Seq ID 2374 | | | | |
| Seq ID 083 | Seq ID 1229 | Seq ID 2375 | | | | |
| Seq ID 084 | Seq ID 1230 | Seq ID 2376 | | | | |
| Seq ID 085 | Seq ID 1231 | Seq ID 2377 | | | | |
| Seq ID 086 | Seq ID 1232 | Seq ID 2378 | X | | | |
| Seq ID 087 | Seq ID 1233 | Seq ID 2379 | | | | |
| Seq ID 088 | Seq ID 1234 | Seq ID 2380 | | | | |
| Seq ID 089 | Seq ID 1235 | Seq ID 2381 | | | X | |
| Seq ID 090 | Seq ID 1236 | Seq ID 2382 | | | | |
| Seq ID 091 | Seq ID 1237 | Seq ID 2383 | | | | |
| Seq ID 092 | Seq ID 1238 | Seq ID 2384 | | | | |
| Seq ID 093 | Seq ID 1239 | Seq ID 2385 | | | | |
| Seq ID 094 | Seq ID 1240 | Seq ID 2386 | | | | |
| Seq ID 095 | Seq ID 1241 | Seq ID 2387 | | | | |
| Seq ID 096 | Seq ID 1242 | Seq ID 2388 | | | | |
| Seq ID 097 | Seq ID 1243 | Seq ID 2389 | | | | |
| Seq ID 098 | Seq ID 1244 | Seq ID 2390 | | | | |
| Seq ID 099 | Seq ID 1245 | Seq ID 2391 | | | | |
| Seq ID 100 | Seq ID 1246 | Seq ID 2392 | | | | |
| Seq ID 101 | Seq ID 1247 | Seq ID 2393 | | | | |
| Seq ID 102 | Seq ID 1248 | Seq ID 2394 | | | | |
| Seq ID 103 | Seq ID 1249 | Seq ID 2395 | | | | |
| Seq ID 104 | Seq ID 1250 | Seq ID 2396 | | | | |
| Seq ID 105 | Seq ID 1251 | Seq ID 2397 | | | | |
| Seq ID 106 | Seq ID 1252 | Seq ID 2398 | | | | |
| Seq ID 107 | Seq ID 1253 | Seq ID 2399 | | | | |
| Seq ID 108 | Seq ID 1254 | Seq ID 2400 | | | | |
| Seq ID 109 | Seq ID 1255 | Seq ID 2401 | | | | |
| Seq ID 110 | Seq ID 1256 | Seq ID 2402 | | | | |
| Seq ID 111 | Seq ID 1257 | Seq ID 2403 | | | | |
| Seq ID 112 | Seq ID 1258 | Seq ID 2404 | | | | |
| Seq ID 113 | Seq ID 1259 | Seq ID 2405 | | | | |
| Seq ID 114 | Seq ID 1260 | Seq ID 2406 | | | | |
| Seq ID 115 | Seq ID 1261 | Seq ID 2407 | | | | |
| Seq ID 116 | Seq ID 1262 | Seq ID 2408 | X | | | |
| Seq ID 117 | Seq ID 1263 | Seq ID 2409 | | | | |
| Seq ID 118 | Seq ID 1264 | Seq ID 2410 | | | | |
| Seq ID 119 | Seq ID 1265 | Seq ID 2411 | | | | |
| Seq ID 120 | Seq ID 1266 | Seq ID 2412 | | | | |
| Seq ID 121 | Seq ID 1267 | Seq ID 2413 | | | | |
| Seq ID 122 | Seq ID 1268 | Seq ID 2414 | | | | |
| Seq ID 123 | Seq ID 1269 | Seq ID 2415 | | | | |
| Seq ID 124 | Seq ID 1270 | Seq ID 2416 | | | | |
| Seq ID 125 | Seq ID 1271 | Seq ID 2417 | | | | |
| Seq ID 126 | Seq ID 1272 | Seq ID 2418 | | | | |
| Seq ID 127 | Seq ID 1273 | Seq ID 2419 | | | | |
| Seq ID 128 | Seq ID 1274 | Seq ID 2420 | | | | |
| Seq ID 129 | Seq ID 1275 | Seq ID 2421 | X | | X | |
| Seq ID 130 | Seq ID 1276 | Seq ID 2422 | | | | |
| Seq ID 131 | Seq ID 1277 | Seq ID 2423 | | | | |
| Seq ID 132 | Seq ID 1278 | Seq ID 2424 | | | | |
| Seq ID 133 | Seq ID 1279 | Seq ID 2425 | | | | |
| Seq ID 134 | Seq ID 1280 | Seq ID 2426 | | | | |
| Seq ID 135 | Seq ID 1281 | Seq ID 2427 | | | | |
| Seq ID 136 | Seq ID 1282 | Seq ID 2428 | | | | |
| Seq ID 137 | Seq ID 1283 | Seq ID 2429 | | | | |
| Seq ID 138 | Seq ID 1284 | Seq ID 2430 | | | | |
| Seq ID 139 | Seq ID 1285 | Seq ID 2431 | | | | |
| Seq ID 140 | Seq ID 1286 | Seq ID 2432 | | | | |
| Seq ID 141 | Seq ID 1287 | Seq ID 2433 | | | | |
| Seq ID 142 | Seq ID 1288 | Seq ID 2434 | X | | | |
| Seq ID 143 | Seq ID 1289 | Seq ID 2435 | | | | |
| Seq ID 144 | Seq ID 1290 | Seq ID 2436 | | | | |
| Seq ID 145 | Seq ID 1291 | Seq ID 2437 | | | | |
| Seq ID 146 | Seq ID 1292 | Seq ID 2438 | | | | |
| Seq ID 147 | Seq ID 1293 | Seq ID 2439 | | | | |
| Seq ID 148 | Seq ID 1294 | Seq ID 2440 | | | | |
| Seq ID 149 | Seq ID 1295 | Seq ID 2441 | | | | |
| Seq ID 150 | Seq ID 1296 | Seq ID 2442 | | | | |
| Seq ID 151 | Seq ID 1297 | Seq ID 2443 | | | | |
| Seq ID 152 | Seq ID 1298 | Seq ID 2444 | | | | |
| Seq ID 153 | Seq ID 1299 | Seq ID 2445 | | | | |
| Seq ID 154 | Seq ID 1300 | Seq ID 2446 | | | | |
| Seq ID 155 | Seq ID 1301 | Seq ID 2447 | | | | |
| Seq ID 156 | Seq ID 1302 | Seq ID 2448 | | | | |
| Seq ID 157 | Seq ID 1303 | Seq ID 2449 | | | | |
| Seq ID 158 | Seq ID 1304 | Seq ID 2450 | | | | |
| Seq ID 159 | Seq ID 1305 | Seq ID 2451 | | | | |
| Seq ID 160 | Seq ID 1306 | Seq ID 2452 | X | | | |
| Seq ID 161 | Seq ID 1307 | Seq ID 2453 | | | | |
| Seq ID 162 | Seq ID 1308 | Seq ID 2454 | | | | |
| Seq ID 163 | Seq ID 1309 | Seq ID 2455 | | | | |
| Seq ID 164 | Seq ID 1310 | Seq ID 2456 | | | | |
| Seq ID 165 | Seq ID 1311 | Seq ID 2457 | | | | |
| Seq ID 166 | Seq ID 1312 | Seq ID 2458 | | | | |
| Seq ID 167 | Seq ID 1313 | Seq ID 2459 | | | | |
| Seq ID 168 | Seq ID 1314 | Seq ID 2460 | | | | |
| Seq ID 169 | Seq ID 1315 | Seq ID 2461 | | | | |
| Seq ID 170 | Seq ID 1316 | Seq ID 2462 | | | | |
| Seq ID 171 | Seq ID 1317 | Seq ID 2463 | | | | |
| Seq ID 172 | Seq ID 1318 | Seq ID 2464 | | | | |
| Seq ID 173 | Seq ID 1319 | Seq ID 2465 | | | | |
| Seq ID 174 | Seq ID 1320 | Seq ID 2466 | X | | | |
| Seq ID 175 | Seq ID 1321 | Seq ID 2467 | | | | |
| Seq ID 176 | Seq ID 1322 | Seq ID 2468 | | | | |
| Seq ID 177 | Seq ID 1323 | Seq ID 2469 | | | | |
| Seq ID 178 | Seq ID 1324 | Seq ID 2470 | X | | | |
| Seq ID 179 | Seq ID 1325 | Seq ID 2471 | | | | |
| Seq ID 180 | Seq ID 1326 | Seq ID 2472 | | | | |
| Seq ID 181 | Seq ID 1327 | Seq ID 2473 | | | | |
| Seq ID 182 | Seq ID 1328 | Seq ID 2474 | | | | |
| Seq ID 183 | Seq ID 1329 | Seq ID 2475 | | | | |
| Seq ID 184 | Seq ID 1330 | Seq ID 2476 | | | | |
| Seq ID 185 | Seq ID 1331 | Seq ID 2477 | | | | |
| Seq ID 186 | Seq ID 1332 | Seq ID 2478 | | | | |
| Seq ID 187 | Seq ID 1333 | Seq ID 2479 | | | | |
| Seq ID 188 | Seq ID 1334 | Seq ID 2480 | | | | |
| Seq ID 189 | Seq ID 1335 | Seq ID 2481 | | | | |
| Seq ID 190 | Seq ID 1336 | Seq ID 2482 | | | | |
| Seq ID 191 | Seq ID 1337 | Seq ID 2483 | | | | |
| Seq ID 192 | Seq ID 1338 | Seq ID 2484 | | | | |
| Seq ID 193 | Seq ID 1339 | Seq ID 2485 | | | | |
| Seq ID 194 | Seq ID 1340 | Seq ID 2486 | | | | |
| Seq ID 195 | Seq ID 1341 | Seq ID 2487 | | | | |
| Seq ID 196 | Seq ID 1342 | Seq ID 2488 | | | | |
| Seq ID 197 | Seq ID 1343 | Seq ID 2489 | | | | |
| Seq ID 198 | Seq ID 1344 | Seq ID 2490 | X | | | X |
| Seq ID 199 | Seq ID 1345 | Seq ID 2491 | | | | |
| Seq ID 200 | Seq ID 1346 | Seq ID 2492 | | | | |
| Seq ID 201 | Seq ID 1347 | Seq ID 2493 | | | | |
| Seq ID 202 | Seq ID 1348 | Seq ID 2494 | | | | |
| Seq ID 203 | Seq ID 1349 | Seq ID 2495 | X | | | |
| Seq ID 204 | Seq ID 1350 | Seq ID 2496 | | | | |
| Seq ID 205 | Seq ID 1351 | Seq ID 2497 | | | | |
| Seq ID 206 | Seq ID 1352 | Seq ID 2498 | | | | |
| Seq ID 207 | Seq ID 1353 | Seq ID 2499 | | | | |
| Seq ID 208 | Seq ID 1354 | Seq ID 2500 | | | | |
| Seq ID 209 | Seq ID 1355 | Seq ID 2501 | | | | |
| Seq ID 210 | Seq ID 1356 | Seq ID 2502 | | | | |
| Seq ID 211 | Seq ID 1357 | Seq ID 2503 | | | | |
| Seq ID 212 | Seq ID 1358 | Seq ID 2504 | X | | | |
| Seq ID 213 | Seq ID 1359 | Seq ID 2505 | | | | |
| Seq ID 214 | Seq ID 1360 | Seq ID 2506 | | | | |
| Seq ID 215 | Seq ID 1361 | Seq ID 2507 | | | | |
| Seq ID 216 | Seq ID 1362 | Seq ID 2508 | | | | |
| Seq ID 217 | Seq ID 1363 | Seq ID 2509 | | | | |
| Seq ID 218 | Seq ID 1364 | Seq ID 2510 | | | | |
| Seq ID 219 | Seq ID 1365 | Seq ID 2511 | | | | |
| Seq ID 220 | Seq ID 1366 | Seq ID 2512 | | | | |
| Seq ID 221 | Seq ID 1367 | Seq ID 2513 | | | | |
| Seq ID 222 | Seq ID 1368 | Seq ID 2514 | | | | |
| Seq ID 223 | Seq ID 1369 | Seq ID 2515 | | | | |
| Seq ID 224 | Seq ID 1370 | Seq ID 2516 | | | | |
| Seq ID 225 | Seq ID 1371 | Seq ID 2517 | | | | |
| Seq ID 226 | Seq ID 1372 | Seq ID 2518 | | | | |
| Seq ID 227 | Seq ID 1373 | Seq ID 2519 | | | | |
| Seq ID 228 | Seq ID 1374 | Seq ID 2520 | | | | |
| Seq ID 229 | Seq ID 1375 | Seq ID 2521 | | | | |
| Seq ID 230 | Seq ID 1376 | Seq ID 2522 | | | | |
| Seq ID 231 | Seq ID 1377 | Seq ID 2523 | | | | |
| Seq ID 232 | Seq ID 1378 | Seq ID 2524 | | | | |
| Seq ID 233 | Seq ID 1379 | Seq ID 2525 | | | | |
| Seq ID 234 | Seq ID 1380 | Seq ID 2526 | | | | |
| Seq ID 235 | Seq ID 1381 | Seq ID 2527 | X | | | |
| Seq ID 236 | Seq ID 1382 | Seq ID 2528 | | | | |
| Seq ID 237 | Seq ID 1383 | Seq ID 2529 | | | | |
| Seq ID 238 | Seq ID 1384 | Seq ID 2530 | | | | |
| Seq ID 239 | Seq ID 1385 | Seq ID 2531 | | | | |
| Seq ID 240 | Seq ID 1386 | Seq ID 2532 | | | | |
| Seq ID 241 | Seq ID 1387 | Seq ID 2533 | | | | |
| Seq ID 242 | Seq ID 1388 | Seq ID 2534 | | | | |
| Seq ID 243 | Seq ID 1389 | Seq ID 2535 | | | | |
| Seq ID 244 | Seq ID 1390 | Seq ID 2536 | | | | |
| Seq ID 245 | Seq ID 1391 | Seq ID 2537 | | | | |
| Seq ID 246 | Seq ID 1392 | Seq ID 2538 | | | | |
| Seq ID 247 | Seq ID 1393 | Seq ID 2539 | | | | |
| Seq ID 248 | Seq ID 1394 | Seq ID 2540 | | | | |
| Seq ID 249 | Seq ID 1395 | Seq ID 2541 | | | | |
| Seq ID 250 | Seq ID 1396 | Seq ID 2542 | | | | |
| Seq ID 251 | Seq ID 1397 | Seq ID 2543 | | | | |
| Seq ID 252 | Seq ID 1398 | Seq ID 2544 | | | | |
| Seq ID 253 | Seq ID 1399 | Seq ID 2545 | | | | |
| Seq ID 254 | Seq ID 1400 | Seq ID 2546 | | | | |
| Seq ID 255 | Seq ID 1401 | Seq ID 2547 | | | | |
| Seq ID 256 | Seq ID 1402 | Seq ID 2548 | | | | |
| Seq ID 257 | Seq ID 1403 | Seq ID 2549 | | | | |
| Seq ID 258 | Seq ID 1404 | Seq ID 2550 | | | | |
| Seq ID 259 | Seq ID 1405 | Seq ID 2551 | | | | |
| Seq ID 260 | Seq ID 1406 | Seq ID 2552 | | | | |
| Seq ID 261 | Seq ID 1407 | Seq ID 2553 | | | | |
| Seq ID 262 | Seq ID 1408 | Seq ID 2554 | | | | |
| Seq ID 263 | Seq ID 1409 | Seq ID 2555 | | | | |
| Seq ID 264 | Seq ID 1410 | Seq ID 2556 | | | | |
| Seq ID 265 | Seq ID 1411 | Seq ID 2557 | | | | |
| Seq ID 266 | Seq ID 1412 | Seq ID 2558 | | | | |
| Seq ID 267 | Seq ID 1413 | Seq ID 2559 | | | | |
| Seq ID 268 | Seq ID 1414 | Seq ID 2560 | | | | |
| Seq ID 269 | Seq ID 1415 | Seq ID 2561 | X | | | |
| Seq ID 270 | Seq ID 1416 | Seq ID 2562 | X | | | X |
| Seq ID 271 | Seq ID 1417 | Seq ID 2563 | | | | |
| Seq ID 272 | Seq ID 1418 | Seq ID 2564 | | | | |
| Seq ID 273 | Seq ID 1419 | Seq ID 2565 | | | | |
| Seq ID 274 | Seq ID 1420 | Seq ID 2566 | X | | | |
| Seq ID 275 | Seq ID 1421 | Seq ID 2567 | | | | |
| Seq ID 276 | Seq ID 1422 | Seq ID 2568 | | | | |
| Seq ID 277 | Seq ID 1423 | Seq ID 2569 | | | | |
| Seq ID 278 | Seq ID 1424 | Seq ID 2570 | | | | |
| Seq ID 279 | Seq ID 1425 | Seq ID 2571 | | | | |
| Seq ID 280 | Seq ID 1426 | Seq ID 2572 | | | | |
| Seq ID 281 | Seq ID 1427 | Seq ID 2573 | | | | |
| Seq ID 282 | Seq ID 1428 | Seq ID 2574 | X | | | X |
| Seq ID 283 | Seq ID 1429 | Seq ID 2575 | | | | |
| Seq ID 284 | Seq ID 1430 | Seq ID 2576 | | | | |
| Seq ID 285 | Seq ID 1431 | Seq ID 2577 | | | | |
| Seq ID 286 | Seq ID 1432 | Seq ID 2578 | X | | | |
| Seq ID 287 | Seq ID 1433 | Seq ID 2579 | | | | |
| Seq ID 288 | Seq ID 1434 | Seq ID 2580 | X | | | |
| Seq ID 289 | Seq ID 1435 | Seq ID 2581 | | | | |
| Seq ID 290 | Seq ID 1436 | Seq ID 2582 | | | | |
| Seq ID 291 | Seq ID 1437 | Seq ID 2583 | | | | |
| Seq ID 292 | Seq ID 1438 | Seq ID 2584 | X | | X | |
| Seq ID 293 | Seq ID 1439 | Seq ID 2585 | | | | |
| Seq ID 294 | Seq ID 1440 | Seq ID 2586 | | | | |
| Seq ID 295 | Seq ID 1441 | Seq ID 2587 | | | | |
| Seq ID 296 | Seq ID 1442 | Seq ID 2588 | | | | |
| Seq ID 297 | Seq ID 1443 | Seq ID 2589 | | | | |
| Seq ID 298 | Seq ID 1444 | Seq ID 2590 | | | | |
| Seq ID 299 | Seq ID 1445 | Seq ID 2591 | | | | |
| Seq ID 300 | Seq ID 1446 | Seq ID 2592 | | | | |
| Seq ID 301 | Seq ID 1447 | Seq ID 2593 | | | | |
| Seq ID 302 | Seq ID 1448 | Seq ID 2594 | | | | |
| Seq ID 303 | Seq ID 1449 | Seq ID 2595 | | | | |
| Seq ID 304 | Seq ID 1450 | Seq ID 2596 | | | | |
| Seq ID 305 | Seq ID 1451 | Seq ID 2597 | X | | | |
| Seq ID 306 | Seq ID 1452 | Seq ID 2598 | | | | |
| Seq ID 307 | Seq ID 1453 | Seq ID 2599 | | | | |
| Seq ID 308 | Seq ID 1454 | Seq ID 2600 | | | | |
| Seq ID 309 | Seq ID 1455 | Seq ID 2601 | | | | |
| Seq ID 310 | Seq ID 1456 | Seq ID 2602 | | X | | |
| Seq ID 311 | Seq ID 1457 | Seq ID 2603 | | | | X |
| Seq ID 312 | Seq ID 1458 | Seq ID 2604 | X | | | |
| Seq ID 313 | Seq ID 1459 | Seq ID 2605 | | | | |
| Seq ID 314 | Seq ID 1460 | Seq ID 2606 | | | | |
| Seq ID 315 | Seq ID 1461 | Seq ID 2607 | | | | |
| Seq ID 316 | Seq ID 1462 | Seq ID 2608 | X | | | |
| Seq ID 317 | Seq ID 1463 | Seq ID 2609 | | | | |
| Seq ID 318 | Seq ID 1464 | Seq ID 2610 | | | | |
| Seq ID 319 | Seq ID 1465 | Seq ID 2611 | | | | |
| Seq ID 320 | Seq ID 1466 | Seq ID 2612 | | | | |
| Seq ID 321 | Seq ID 1467 | Seq ID 2613 | | | | |
| Seq ID 322 | Seq ID 1468 | Seq ID 2614 | | | | |
| Seq ID 323 | Seq ID 1469 | Seq ID 2615 | | | | |
| Seq ID 324 | Seq ID 1470 | Seq ID 2616 | | | | |
| Seq ID 325 | Seq ID 1471 | Seq ID 2617 | | | | |
| Seq ID 326 | Seq ID 1472 | Seq ID 2618 | | | | |
| Seq ID 327 | Seq ID 1473 | Seq ID 2619 | | | | |
| Seq ID 328 | Seq ID 1474 | Seq ID 2620 | | | | |
| Seq ID 329 | Seq ID 1475 | Seq ID 2621 | | | | |
| Seq ID 330 | Seq ID 1476 | Seq ID 2622 | | | | |
| Seq ID 331 | Seq ID 1477 | Seq ID 2623 | | | | |
| Seq ID 332 | Seq ID 1478 | Seq ID 2624 | | | | |
| Seq ID 333 | Seq ID 1479 | Seq ID 2625 | | | | |
| Seq ID 334 | Seq ID 1480 | Seq ID 2626 | | | | |
| Seq ID 335 | Seq ID 1481 | Seq ID 2627 | | | | |
| Seq ID 336 | Seq ID 1482 | Seq ID 2628 | | | | |
| Seq ID 337 | Seq ID 1483 | Seq ID 2629 | | | | |
| Seq ID 338 | Seq ID 1484 | Seq ID 2630 | | | | |
| Seq ID 339 | Seq ID 1485 | Seq ID 2631 | | | | |
| Seq ID 340 | Seq ID 1486 | Seq ID 2632 | | | | |
| Seq ID 341 | Seq ID 1487 | Seq ID 2633 | | | | |
| Seq ID 342 | Seq ID 1488 | Seq ID 2634 | | | | |
| Seq ID 343 | Seq ID 1489 | Seq ID 2635 | | | | |
| Seq ID 344 | Seq ID 1490 | Seq ID 2636 | | | | |
| Seq ID 345 | Seq ID 1491 | Seq ID 2637 | | | | |
| Seq ID 346 | Seq ID 1492 | Seq ID 2638 | | | | |
| Seq ID 347 | Seq ID 1493 | Seq ID 2639 | X | | | |
| Seq ID 348 | Seq ID 1494 | Seq ID 2640 | X | | | |
| Seq ID 349 | Seq ID 1495 | Seq ID 2641 | | | | |
| Seq ID 350 | Seq ID 1496 | Seq ID 2642 | | | | |
| Seq ID 351 | Seq ID 1497 | Seq ID 2643 | | | | |
| Seq ID 352 | Seq ID 1498 | Seq ID 2644 | | | | |
| Seq ID 353 | Seq ID 1499 | Seq ID 2645 | | | | |
| Seq ID 354 | Seq ID 1500 | Seq ID 2646 | X | | | |
| Seq ID 355 | Seq ID 1501 | Seq ID 2647 | X | | | X |
| Seq ID 356 | Seq ID 1502 | Seq ID 2648 | | | | |
| Seq ID 357 | Seq ID 1503 | Seq ID 2649 | X | | | |
| Seq ID 358 | Seq ID 1504 | Seq ID 2650 | | | | |
| Seq ID 359 | Seq ID 1505 | Seq ID 2651 | | | | |
| Seq ID 360 | Seq ID 1506 | Seq ID 2652 | X | | | X |
| Seq ID 361 | Seq ID 1507 | Seq ID 2653 | | | | |
| Seq ID 362 | Seq ID 1508 | Seq ID 2654 | | | | |
| Seq ID 363 | Seq ID 1509 | Seq ID 2655 | | | | |
| Seq ID 364 | Seq ID 1510 | Seq ID 2656 | | | | |
| Seq ID 365 | Seq ID 1511 | Seq ID 2657 | | | | |
| Seq ID 366 | Seq ID 1512 | Seq ID 2658 | | | | |
| Seq ID 367 | Seq ID 1513 | Seq ID 2659 | | | | |
| Seq ID 368 | Seq ID 1514 | Seq ID 2660 | | X | | |
| Seq ID 369 | Seq ID 1515 | Seq ID 2661 | | | | |
| Seq ID 370 | Seq ID 1516 | Seq ID 2662 | | | | |
| Seq ID 371 | Seq ID 1517 | Seq ID 2663 | X | X | | |
| Seq ID 372 | Seq ID 1518 | Seq ID 2664 | X | | | |
| Seq ID 373 | Seq ID 1519 | Seq ID 2665 | | | | |
| Seq ID 374 | Seq ID 1520 | Seq ID 2666 | | | | |
| Seq ID 375 | Seq ID 1521 | Seq ID 2667 | | | | |
| Seq ID 376 | Seq ID 1522 | Seq ID 2668 | | | | |
| Seq ID 377 | Seq ID 1523 | Seq ID 2669 | | | | |
| Seq ID 378 | Seq ID 1524 | Seq ID 2670 | | | | |
| Seq ID 379 | Seq ID 1525 | Seq ID 2671 | | | | |
| Seq ID 380 | Seq ID 1526 | Seq ID 2672 | | | | |
| Seq ID 381 | Seq ID 1527 | Seq ID 2673 | | | | |
| Seq ID 382 | Seq ID 1528 | Seq ID 2674 | | | | |
| Seq ID 383 | Seq ID 1529 | Seq ID 2675 | | | | |
| Seq ID 384 | Seq ID 1530 | Seq ID 2676 | | | | |
| Seq ID 385 | Seq ID 1531 | Seq ID 2677 | | | | |
| Seq ID 386 | Seq ID 1532 | Seq ID 2678 | | | | |
| Seq ID 387 | Seq ID 1533 | Seq ID 2679 | | | | |
| Seq ID 388 | Seq ID 1534 | Seq ID 2680 | | | | |
| Seq ID 389 | Seq ID 1535 | Seq ID 2681 | | | | |
| Seq ID 390 | Seq ID 1536 | Seq ID 2682 | | | | |
| Seq ID 391 | Seq ID 1537 | Seq ID 2683 | | | | |
| Seq ID 392 | Seq ID 1538 | Seq ID 2684 | | | | |
| Seq ID 393 | Seq ID 1539 | Seq ID 2685 | | | | |
| Seq ID 394 | Seq ID 1540 | Seq ID 2686 | | | | |
| Seq ID 395 | Seq ID 1541 | Seq ID 2687 | | | | |
| Seq ID 396 | Seq ID 1542 | Seq ID 2688 | | | | |
| Seq ID 397 | Seq ID 1543 | Seq ID 2689 | | | | |
| Seq ID 398 | Seq ID 1544 | Seq ID 2690 | | | | |
| Seq ID 399 | Seq ID 1545 | Seq ID 2691 | | | | |
| Seq ID 400 | Seq ID 1546 | Seq ID 2692 | | | | |
| Seq ID 401 | Seq ID 1547 | Seq ID 2693 | | | | |
| Seq ID 402 | Seq ID 1548 | Seq ID 2694 | | | | |
| Seq ID 403 | Seq ID 1549 | Seq ID 2695 | | | | |
| Seq ID 404 | Seq ID 1550 | Seq ID 2696 | X | | | |
| Seq ID 405 | Seq ID 1551 | Seq ID 2697 | | | | |
| Seq ID 406 | Seq ID 1552 | Seq ID 2698 | | | | |
| Seq ID 407 | Seq ID 1553 | Seq ID 2699 | | | | |
| Seq ID 408 | Seq ID 1554 | Seq ID 2700 | | | | |
| Seq ID 409 | Seq ID 1555 | Seq ID 2701 | | | | |
| Seq ID 410 | Seq ID 1556 | Seq ID 2702 | | | | |
| Seq ID 411 | Seq ID 1557 | Seq ID 2703 | | | | |
| Seq ID 412 | Seq ID 1558 | Seq ID 2704 | | | | |
| Seq ID 413 | Seq ID 1559 | Seq ID 2705 | | | | |
| Seq ID 414 | Seq ID 1560 | Seq ID 2706 | | | | |
| Seq ID 415 | Seq ID 1561 | Seq ID 2707 | | | | |
| Seq ID 416 | Seq ID 1562 | Seq ID 2708 | | | | |
| Seq ID 417 | Seq ID 1563 | Seq ID 2709 | | | | |
| Seq ID 418 | Seq ID 1564 | Seq ID 2710 | | | | |
| Seq ID 419 | Seq ID 1565 | Seq ID 2711 | | | | |
| Seq ID 420 | Seq ID 1566 | Seq ID 2712 | | | | |
| Seq ID 421 | Seq ID 1567 | Seq ID 2713 | | | | |
| Seq ID 422 | Seq ID 1568 | Seq ID 2714 | | | | |
| Seq ID 423 | Seq ID 1569 | Seq ID 2715 | | | | |
| Seq ID 424 | Seq ID 1570 | Seq ID 2716 | | | | |
| Seq ID 425 | Seq ID 1571 | Seq ID 2717 | | | | |
| Seq ID 426 | Seq ID 1572 | Seq ID 2718 | | | | |
| Seq ID 427 | Seq ID 1573 | Seq ID 2719 | | | | |
| Seq ID 428 | Seq ID 1574 | Seq ID 2720 | | | | |
| Seq ID 429 | Seq ID 1575 | Seq ID 2721 | | | | |
| Seq ID 430 | Seq ID 1576 | Seq ID 2722 | | | | |
| Seq ID 431 | Seq ID 1577 | Seq ID 2723 | | | | |
| Seq ID 432 | Seq ID 1578 | Seq ID 2724 | | | | |
| Seq ID 433 | Seq ID 1579 | Seq ID 2725 | X | | | |
| Seq ID 434 | Seq ID 1580 | Seq ID 2726 | | | | |
| Seq ID 435 | Seq ID 1581 | Seq ID 2727 | X | | | |
| Seq ID 436 | Seq ID 1582 | Seq ID 2728 | | | | |
| Seq ID 437 | Seq ID 1583 | Seq ID 2729 | X | | | |
| Seq ID 438 | Seq ID 1584 | Seq ID 2730 | | | | |
| Seq ID 439 | Seq ID 1585 | Seq ID 2731 | | | | |
| Seq ID 440 | Seq ID 1586 | Seq ID 2732 | | | | |
| Seq ID 441 | Seq ID 1587 | Seq ID 2733 | | | | |
| Seq ID 442 | Seq ID 1588 | Seq ID 2734 | | | | |
| Seq ID 443 | Seq ID 1589 | Seq ID 2735 | | | | |
| Seq ID 444 | Seq ID 1590 | Seq ID 2736 | | X | | |
| Seq ID 445 | Seq ID 1591 | Seq ID 2737 | | | | |
| Seq ID 446 | Seq ID 1592 | Seq ID 2738 | | | | |
| Seq ID 447 | Seq ID 1593 | Seq ID 2739 | | | | |
| Seq ID 448 | Seq ID 1594 | Seq ID 2740 | | | | |
| Seq ID 449 | Seq ID 1595 | Seq ID 2741 | | | | |
| Seq ID 450 | Seq ID 1596 | Seq ID 2742 | | | | |
| Seq ID 451 | Seq ID 1597 | Seq ID 2743 | | | | |
| Seq ID 452 | Seq ID 1598 | Seq ID 2744 | | | | |
| Seq ID 453 | Seq ID 1599 | Seq ID 2745 | | | | |
| Seq ID 454 | Seq ID 1600 | Seq ID 2746 | | | | |
| Seq ID 455 | Seq ID 1601 | Seq ID 2747 | | | | |
| Seq ID 456 | Seq ID 1602 | Seq ID 2748 | | | | |
| Seq ID 457 | Seq ID 1603 | Seq ID 2749 | | | | |
| Seq ID 458 | Seq ID 1604 | Seq ID 2750 | | | | |
| Seq ID 459 | Seq ID 1605 | Seq ID 2751 | | | | |
| Seq ID 460 | Seq ID 1606 | Seq ID 2752 | | | | |
| Seq ID 461 | Seq ID 1607 | Seq ID 2753 | | | | |
| Seq ID 462 | Seq ID 1608 | Seq ID 2754 | | | | |
| Seq ID 463 | Seq ID 1609 | Seq ID 2755 | X | | | |
| Seq ID 464 | Seq ID 1610 | Seq ID 2756 | | | | |
| Seq ID 465 | Seq ID 1611 | Seq ID 2757 | | | | |
| Seq ID 466 | Seq ID 1612 | Seq ID 2758 | | | | |
| Seq ID 467 | Seq ID 1613 | Seq ID 2759 | | | | |
| Seq ID 468 | Seq ID 1614 | Seq ID 2760 | | | | |
| Seq ID 469 | Seq ID 1615 | Seq ID 2761 | X | | | |
| Seq ID 470 | Seq ID 1616 | Seq ID 2762 | | | | |
| Seq ID 471 | Seq ID 1617 | Seq ID 2763 | X | | | |
| Seq ID 472 | Seq ID 1618 | Seq ID 2764 | | | | |
| Seq ID 473 | Seq ID 1619 | Seq ID 2765 | | | | |
| Seq ID 474 | Seq ID 1620 | Seq ID 2766 | | | | |
| Seq ID 475 | Seq ID 1621 | Seq ID 2767 | | | | |
| Seq ID 476 | Seq ID 1622 | Seq ID 2768 | | | | |
| Seq ID 477 | Seq ID 1623 | Seq ID 2769 | X | | | |
| Seq ID 478 | Seq ID 1624 | Seq ID 2770 | | | | |
| Seq ID 479 | Seq ID 1625 | Seq ID 2771 | X | | | |
| Seq ID 480 | Seq ID 1626 | Seq ID 2772 | | | | |
| Seq ID 481 | Seq ID 1627 | Seq ID 2773 | | | | |
| Seq ID 482 | Seq ID 1628 | Seq ID 2774 | | | | |
| Seq ID 483 | Seq ID 1629 | Seq ID 2775 | | | | |
| Seq ID 484 | Seq ID 1630 | Seq ID 2776 | | | | |
| Seq ID 485 | Seq ID 1631 | Seq ID 2777 | | | | |
| Seq ID 486 | Seq ID 1632 | Seq ID 2778 | X | | | X |
| Seq ID 487 | Seq ID 1633 | Seq ID 2779 | | | | |
| Seq ID 488 | Seq ID 1634 | Seq ID 2780 | | | | |
| Seq ID 489 | Seq ID 1635 | Seq ID 2781 | | | | |
| Seq ID 490 | Seq ID 1636 | Seq ID 2782 | | | | |
| Seq ID 491 | Seq ID 1637 | Seq ID 2783 | | | | |
| Seq ID 492 | Seq ID 1638 | Seq ID 2784 | | | | |
| Seq ID 493 | Seq ID 1639 | Seq ID 2785 | | | | |
| Seq ID 494 | Seq ID 1640 | Seq ID 2786 | | | | |
| Seq ID 495 | Seq ID 1641 | Seq ID 2787 | | | | |
| Seq ID 496 | Seq ID 1642 | Seq ID 2788 | | | | |
| Seq ID 497 | Seq ID 1643 | Seq ID 2789 | | | | |
| Seq ID 498 | Seq ID 1644 | Seq ID 2790 | | | | |
| Seq ID 499 | Seq ID 1645 | Seq ID 2791 | | | | |
| Seq ID 500 | Seq ID 1646 | Seq ID 2792 | X | | | X |
| Seq ID 501 | Seq ID 1647 | Seq ID 2793 | | | | |
| Seq ID 502 | Seq ID 1648 | Seq ID 2794 | | | | |
| Seq ID 503 | Seq ID 1649 | Seq ID 2795 | | | | |
| Seq ID 504 | Seq ID 1650 | Seq ID 2796 | | | | |
| Seq ID 505 | Seq ID 1651 | Seq ID 2797 | | | | |
| Seq ID 506 | Seq ID 1652 | Seq ID 2798 | | | | |
| Seq ID 507 | Seq ID 1653 | Seq ID 2799 | X | | | X |
| Seq ID 508 | Seq ID 1654 | Seq ID 2800 | | | | |
| Seq ID 509 | Seq ID 1655 | Seq ID 2801 | X | | | X |
| Seq ID 510 | Seq ID 1656 | Seq ID 2802 | | | | |
| Seq ID 511 | Seq ID 1657 | Seq ID 2803 | | | | |
| Seq ID 512 | Seq ID 1658 | Seq ID 2804 | | | | |
| Seq ID 513 | Seq ID 1659 | Seq ID 2805 | | | | |
| Seq ID 514 | Seq ID 1660 | Seq ID 2806 | | | | |
| Seq ID 515 | Seq ID 1661 | Seq ID 2807 | | | | |
| Seq ID 516 | Seq ID 1662 | Seq ID 2808 | | | | |
| Seq ID 517 | Seq ID 1663 | Seq ID 2809 | | | | |
| Seq ID 518 | Seq ID 1664 | Seq ID 2810 | | | | |
| Seq ID 519 | Seq ID 1665 | Seq ID 2811 | | | | |
| Seq ID 520 | Seq ID 1666 | Seq ID 2812 | | | | |
| Seq ID 521 | Seq ID 1667 | Seq ID 2813 | | | | |
| Seq ID 522 | Seq ID 1668 | Seq ID 2814 | | | | |
| Seq ID 523 | Seq ID 1669 | Seq ID 2815 | | | | |
| Seq ID 524 | Seq ID 1670 | Seq ID 2816 | | | | |
| Seq ID 525 | Seq ID 1671 | Seq ID 2817 | X | | | |
| Seq ID 526 | Seq ID 1672 | Seq ID 2818 | | | | |
| Seq ID 527 | Seq ID 1673 | Seq ID 2819 | | | | |
| Seq ID 528 | Seq ID 1674 | Seq ID 2820 | | | | |
| Seq ID 529 | Seq ID 1675 | Seq ID 2821 | | | | |
| Seq ID 530 | Seq ID 1676 | Seq ID 2822 | | | | |
| Seq ID 531 | Seq ID 1677 | Seq ID 2823 | | | | |
| Seq ID 532 | Seq ID 1678 | Seq ID 2824 | | | | |
| Seq ID 533 | Seq ID 1679 | Seq ID 2825 | | | | |
| Seq ID 534 | Seq ID 1680 | Seq ID 2826 | X | | | |
| Seq ID 535 | Seq ID 1681 | Seq ID 2827 | | | | |
| Seq ID 536 | Seq ID 1682 | Seq ID 2828 | | | | |
| Seq ID 537 | Seq ID 1683 | Seq ID 2829 | | | | |
| Seq ID 538 | Seq ID 1684 | Seq ID 2830 | | | | |
| Seq ID 539 | Seq ID 1685 | Seq ID 2831 | | | | |
| Seq ID 540 | Seq ID 1686 | Seq ID 2832 | | | | |
| Seq ID 541 | Seq ID 1687 | Seq ID 2833 | | | | |
| Seq ID 542 | Seq ID 1688 | Seq ID 2834 | | | | |
| Seq ID 543 | Seq ID 1689 | Seq ID 2835 | X | | | |
| Seq ID 544 | Seq ID 1690 | Seq ID 2836 | | | | |
| Seq ID 545 | Seq ID 1691 | Seq ID 2837 | | | | |
| Seq ID 546 | Seq ID 1692 | Seq ID 2838 | | | | |
| Seq ID 547 | Seq ID 1693 | Seq ID 2839 | | | | |
| Seq ID 548 | Seq ID 1694 | Seq ID 2840 | | | | |
| Seq ID 549 | Seq ID 1695 | Seq ID 2841 | | | | |
| Seq ID 550 | Seq ID 1696 | Seq ID 2842 | | | | |
| Seq ID 551 | Seq ID 1697 | Seq ID 2843 | | | | |
| Seq ID 552 | Seq ID 1698 | Seq ID 2844 | | | | |
| Seq ID 553 | Seq ID 1699 | Seq ID 2845 | X | | | |
| Seq ID 554 | Seq ID 1700 | Seq ID 2846 | | | | |
| Seq ID 555 | Seq ID 1701 | Seq ID 2847 | | | | |
| Seq ID 556 | Seq ID 1702 | Seq ID 2848 | | | | |
| Seq ID 557 | Seq ID 1703 | Seq ID 2849 | | | | |
| Seq ID 558 | Seq ID 1704 | Seq ID 2850 | | | | |
| Seq ID 559 | Seq ID 1705 | Seq ID 2851 | | | X | X |
| Seq ID 560 | Seq ID 1706 | Seq ID 2852 | | | | |
| Seq ID 561 | Seq ID 1707 | Seq ID 2853 | | | | |
| Seq ID 562 | Seq ID 1708 | Seq ID 2854 | X | | | |
| Seq ID 563 | Seq ID 1709 | Seq ID 2855 | | | | |
| Seq ID 564 | Seq ID 1710 | Seq ID 2856 | | | | |
| Seq ID 565 | Seq ID 1711 | Seq ID 2857 | X | | | |
| Seq ID 566 | Seq ID 1712 | Seq ID 2858 | | | | |
| Seq ID 567 | Seq ID 1713 | Seq ID 2859 | | | | |
| Seq ID 568 | Seq ID 1714 | Seq ID 2860 | | | | |
| Seq ID 569 | Seq ID 1715 | Seq ID 2861 | | | | |
| Seq ID 570 | Seq ID 1716 | Seq ID 2862 | | | | |
| Seq ID 571 | Seq ID 1717 | Seq ID 2863 | | | | |
| Seq ID 572 | Seq ID 1718 | Seq ID 2864 | | | | |
| Seq ID 573 | Seq ID 1719 | Seq ID 2865 | | | | |
| Seq ID 574 | Seq ID 1720 | Seq ID 2866 | | | | |
| Seq ID 575 | Seq ID 1721 | Seq ID 2867 | | | | |
| Seq ID 576 | Seq ID 1722 | Seq ID 2868 | | | | |
| Seq ID 577 | Seq ID 1723 | Seq ID 2869 | | | | |
| Seq ID 578 | Seq ID 1724 | Seq ID 2870 | | | | |
| Seq ID 579 | Seq ID 1725 | Seq ID 2871 | | | | |
| Seq ID 580 | Seq ID 1726 | Seq ID 2872 | | | | |
| Seq ID 581 | Seq ID 1727 | Seq ID 2873 | | | | X |
| Seq ID 582 | Seq ID 1728 | Seq ID 2874 | | | | |
| Seq ID 583 | Seq ID 1729 | Seq ID 2875 | | | | |
| Seq ID 584 | Seq ID 1730 | Seq ID 2876 | | | | |
| Seq ID 585 | Seq ID 1731 | Seq ID 2877 | | | | |
| Seq ID 586 | Seq ID 1732 | Seq ID 2878 | X | | | |
| Seq ID 587 | Seq ID 1733 | Seq ID 2879 | | | | |
| Seq ID 588 | Seq ID 1734 | Seq ID 2880 | | | | |
| Seq ID 589 | Seq ID 1735 | Seq ID 2881 | | | | |
| Seq ID 590 | Seq ID 1736 | Seq ID 2882 | | | | |
| Seq ID 591 | Seq ID 1737 | Seq ID 2883 | | | | |
| Seq ID 592 | Seq ID 1738 | Seq ID 2884 | X | | | X |
| Seq ID 593 | Seq ID 1739 | Seq ID 2885 | | | | |
| Seq ID 594 | Seq ID 1740 | Seq ID 2886 | | | | |
| Seq ID 595 | Seq ID 1741 | Seq ID 2887 | | | | |
| Seq ID 596 | Seq ID 1742 | Seq ID 2888 | | | | |
| Seq ID 597 | Seq ID 1743 | Seq ID 2889 | | | | |
| Seq ID 598 | Seq ID 1744 | Seq ID 2890 | | | | |
| Seq ID 599 | Seq ID 1745 | Seq ID 2891 | | | | |
| Seq ID 600 | Seq ID 1746 | Seq ID 2892 | | | | |
| Seq ID 601 | Seq ID 1747 | Seq ID 2893 | | | | |
| Seq ID 602 | Seq ID 1748 | Seq ID 2894 | | | | |
| Seq ID 603 | Seq ID 1749 | Seq ID 2895 | | | | |
| Seq ID 604 | Seq ID 1750 | Seq ID 2896 | | | | |
| Seq ID 605 | Seq ID 1751 | Seq ID 2897 | | | | |
| Seq ID 606 | Seq ID 1752 | Seq ID 2898 | | | | |
| Seq ID 607 | Seq ID 1753 | Seq ID 2899 | | | | |
| Seq ID 608 | Seq ID 1754 | Seq ID 2900 | | | | |
| Seq ID 609 | Seq ID 1755 | Seq ID 2901 | | | | |
| Seq ID 610 | Seq ID 1756 | Seq ID 2902 | | | | |
| Seq ID 611 | Seq ID 1757 | Seq ID 2903 | | | | |
| Seq ID 612 | Seq ID 1758 | Seq ID 2904 | | | | |
| Seq ID 613 | Seq ID 1759 | Seq ID 2905 | X | | | |
| Seq ID 614 | Seq ID 1760 | Seq ID 2906 | | | X | |
| Seq ID 615 | Seq ID 1761 | Seq ID 2907 | | | | |
| Seq ID 616 | Seq ID 1762 | Seq ID 2908 | | | | |
| Seq ID 617 | Seq ID 1763 | Seq ID 2909 | | | | |
| Seq ID 618 | Seq ID 1764 | Seq ID 2910 | | | | |
| Seq ID 619 | Seq ID 1765 | Seq ID 2911 | | | | |
| Seq ID 620 | Seq ID 1766 | Seq ID 2912 | | | | |
| Seq ID 621 | Seq ID 1767 | Seq ID 2913 | | | | |
| Seq ID 622 | Seq ID 1768 | Seq ID 2914 | | | | |
| Seq ID 623 | Seq ID 1769 | Seq ID 2915 | | | | |
| Seq ID 624 | Seq ID 1770 | Seq ID 2916 | | | | |
| Seq ID 625 | Seq ID 1771 | Seq ID 2917 | | | | |
| Seq ID 626 | Seq ID 1772 | Seq ID 2918 | | | | |
| Seq ID 627 | Seq ID 1773 | Seq ID 2919 | | | | |
| Seq ID 628 | Seq ID 1774 | Seq ID 2920 | | | | |
| Seq ID 629 | Seq ID 1775 | Seq ID 2921 | | | | |
| Seq ID 630 | Seq ID 1776 | Seq ID 2922 | | | | |
| Seq ID 631 | Seq ID 1777 | Seq ID 2923 | | | | |
| Seq ID 632 | Seq ID 1778 | Seq ID 2924 | | | | |
| Seq ID 633 | Seq ID 1779 | Seq ID 2925 | | | | |
| Seq ID 634 | Seq ID 1780 | Seq ID 2926 | | | | |
| Seq ID 635 | Seq ID 1781 | Seq ID 2927 | | | | |
| Seq ID 636 | Seq ID 1782 | Seq ID 2928 | | | | |
| Seq ID 637 | Seq ID 1783 | Seq ID 2929 | | | | |
| Seq ID 638 | Seq ID 1784 | Seq ID 2930 | | | | |
| Seq ID 639 | Seq ID 1785 | Seq ID 2931 | | | | |
| Seq ID 640 | Seq ID 1786 | Seq ID 2932 | | | | |
| Seq ID 641 | Seq ID 1787 | Seq ID 2933 | | | | |
| Seq ID 642 | Seq ID 1788 | Seq ID 2934 | | | | |
| Seq ID 643 | Seq ID 1789 | Seq ID 2935 | | | | |
| Seq ID 644 | Seq ID 1790 | Seq ID 2936 | | | | |
| Seq ID 645 | Seq ID 1791 | Seq ID 2937 | | | | |
| Seq ID 646 | Seq ID 1792 | Seq ID 2938 | | | | |
| Seq ID 647 | Seq ID 1793 | Seq ID 2939 | | | | |
| Seq ID 648 | Seq ID 1794 | Seq ID 2940 | | | | |
| Seq ID 649 | Seq ID 1795 | Seq ID 2941 | | X | | |
| Seq ID 650 | Seq ID 1796 | Seq ID 2942 | | | | |
| Seq ID 651 | Seq ID 1797 | Seq ID 2943 | X | | | X |
| Seq ID 652 | Seq ID 1798 | Seq ID 2944 | | | | |
| Seq ID 653 | Seq ID 1799 | Seq ID 2945 | | | | |
| Seq ID 654 | Seq ID 1800 | Seq ID 2946 | | | | |
| Seq ID 655 | Seq ID 1801 | Seq ID 2947 | | | | |
| Seq ID 656 | Seq ID 1802 | Seq ID 2948 | | | | |
| Seq ID 657 | Seq ID 1803 | Seq ID 2949 | X | | | X |
| Seq ID 658 | Seq ID 1804 | Seq ID 2950 | X | | | |
| Seq ID 659 | Seq ID 1805 | Seq ID 2951 | | | | |
| Seq ID 660 | Seq ID 1806 | Seq ID 2952 | | | | |
| Seq ID 661 | Seq ID 1807 | Seq ID 2953 | | | | |
| Seq ID 662 | Seq ID 1808 | Seq ID 2954 | | | | |
| Seq ID 663 | Seq ID 1809 | Seq ID 2955 | | | | |
| Seq ID 664 | Seq ID 1810 | Seq ID 2956 | | | | |
| Seq ID 665 | Seq ID 1811 | Seq ID 2957 | | | | |
| Seq ID 666 | Seq ID 1812 | Seq ID 2958 | | | | |
| Seq ID 667 | Seq ID 1813 | Seq ID 2959 | | | | |
| Seq ID 668 | Seq ID 1814 | Seq ID 2960 | X | | | X |
| Seq ID 669 | Seq ID 1815 | Seq ID 2961 | | | | |
| Seq ID 670 | Seq ID 1816 | Seq ID 2962 | | | | |
| Seq ID 671 | Seq ID 1817 | Seq ID 2963 | | | | |
| Seq ID 672 | Seq ID 1818 | Seq ID 2964 | | | | |
| Seq ID 673 | Seq ID 1819 | Seq ID 2965 | | | | |
| Seq ID 674 | Seq ID 1820 | Seq ID 2966 | | | | |
| Seq ID 675 | Seq ID 1821 | Seq ID 2967 | | | | X |
| Seq ID 676 | Seq ID 1822 | Seq ID 2968 | | | | |
| Seq ID 677 | Seq ID 1823 | Seq ID 2969 | | | | |
| Seq ID 678 | Seq ID 1824 | Seq ID 2970 | | | | |
| Seq ID 679 | Seq ID 1825 | Seq ID 2971 | | | | |
| Seq ID 680 | Seq ID 1826 | Seq ID 2972 | | | | |
| Seq ID 681 | Seq ID 1827 | Seq ID 2973 | | | | |
| Seq ID 682 | Seq ID 1828 | Seq ID 2974 | | | | |
| Seq ID 683 | Seq ID 1829 | Seq ID 2975 | | | | |
| Seq ID 684 | Seq ID 1830 | Seq ID 2976 | | | | |
| Seq ID 685 | Seq ID 1831 | Seq ID 2977 | | | | |
| Seq ID 686 | Seq ID 1832 | Seq ID 2978 | X | | | |
| Seq ID 687 | Seq ID 1833 | Seq ID 2979 | | | | |
| Seq ID 688 | Seq ID 1834 | Seq ID 2980 | X | | | |
| Seq ID 689 | Seq ID 1835 | Seq ID 2981 | | | | |
| Seq ID 690 | Seq ID 1836 | Seq ID 2982 | | | | |
| Seq ID 691 | Seq ID 1837 | Seq ID 2983 | | | | |
| Seq ID 692 | Seq ID 1838 | Seq ID 2984 | | | | |
| Seq ID 693 | Seq ID 1839 | Seq ID 2985 | | | | |
| Seq ID 694 | Seq ID 1840 | Seq ID 2986 | | | | |
| Seq ID 695 | Seq ID 1841 | Seq ID 2987 | | | | |
| Seq ID 696 | Seq ID 1842 | Seq ID 2988 | | | | |
| Seq ID 697 | Seq ID 1843 | Seq ID 2989 | | | | |
| Seq ID 698 | Seq ID 1844 | Seq ID 2990 | | | | |
| Seq ID 699 | Seq ID 1845 | Seq ID 2991 | | | | |
| Seq ID 700 | Seq ID 1846 | Seq ID 2992 | | | | |
| Seq ID 701 | Seq ID 1847 | Seq ID 2993 | | | | |
| Seq ID 702 | Seq ID 1848 | Seq ID 2994 | | | | |
| Seq ID 703 | Seq ID 1849 | Seq ID 2995 | | | | |
| Seq ID 704 | Seq ID 1850 | Seq ID 2996 | | | | |
| Seq ID 705 | Seq ID 1851 | Seq ID 2997 | | | | |
| Seq ID 706 | Seq ID 1852 | Seq ID 2998 | | | | |
| Seq ID 707 | Seq ID 1853 | Seq ID 2999 | | | | |
| Seq ID 708 | Seq ID 1854 | Seq ID 3000 | X | | | X |
| Seq ID 709 | Seq ID 1855 | Seq ID 3001 | | | | |
| Seq ID 710 | Seq ID 1856 | Seq ID 3002 | X | | | X |
| Seq ID 711 | Seq ID 1857 | Seq ID 3003 | | | | |
| Seq ID 712 | Seq ID 1858 | Seq ID 3004 | | | | |
| Seq ID 713 | Seq ID 1859 | Seq ID 3005 | | | | |
| Seq ID 714 | Seq ID 1860 | Seq ID 3006 | | | | |
| Seq ID 715 | Seq ID 1861 | Seq ID 3007 | | | | |
| Seq ID 716 | Seq ID 1862 | Seq ID 3008 | X | | | |
| Seq ID 717 | Seq ID 1863 | Seq ID 3009 | | | | |
| Seq ID 718 | Seq ID 1864 | Seq ID 3010 | | | | |
| Seq ID 719 | Seq ID 1865 | Seq ID 3011 | | | | |
| Seq ID 720 | Seq ID 1866 | Seq ID 3012 | X | | | |
| Seq ID 721 | Seq ID 1867 | Seq ID 3013 | | | | |
| Seq ID 722 | Seq ID 1868 | Seq ID 3014 | | | | |
| Seq ID 723 | Seq ID 1869 | Seq ID 3015 | | | | |
| Seq ID 724 | Seq ID 1870 | Seq ID 3016 | X | | | |
| Seq ID 725 | Seq ID 1871 | Seq ID 3017 | | | | |
| Seq ID 726 | Seq ID 1872 | Seq ID 3018 | | | | |
| Seq ID 727 | Seq ID 1873 | Seq ID 3019 | | | | |
| Seq ID 728 | Seq ID 1874 | Seq ID 3020 | | | | |
| Seq ID 729 | Seq ID 1875 | Seq ID 3021 | | | | |
| Seq ID 730 | Seq ID 1876 | Seq ID 3022 | | | | |
| Seq ID 731 | Seq ID 1877 | Seq ID 3023 | X | | | |
| Seq ID 732 | Seq ID 1878 | Seq ID 3024 | X | | | |
| Seq ID 733 | Seq ID 1879 | Seq ID 3025 | | | | |
| Seq ID 734 | Seq ID 1880 | Seq ID 3026 | | | | |
| Seq ID 735 | Seq ID 1881 | Seq ID 3027 | | | | |
| Seq ID 736 | Seq ID 1882 | Seq ID 3028 | | | | |
| Seq ID 737 | Seq ID 1883 | Seq ID 3029 | | | | |
| Seq ID 738 | Seq ID 1884 | Seq ID 3030 | | | | |
| Seq ID 739 | Seq ID 1885 | Seq ID 3031 | | | | |
| Seq ID 740 | Seq ID 1886 | Seq ID 3032 | | | | |
| Seq ID 741 | Seq ID 1887 | Seq ID 3033 | | | | |
| Seq ID 742 | Seq ID 1888 | Seq ID 3034 | | | | |
| Seq ID 743 | Seq ID 1889 | Seq ID 3035 | | | | |
| Seq ID 744 | Seq ID 1890 | Seq ID 3036 | X | | | X |
| Seq ID 745 | Seq ID 1891 | Seq ID 3037 | | | | |
| Seq ID 746 | Seq ID 1892 | Seq ID 3038 | | | | |
| Seq ID 747 | Seq ID 1893 | Seq ID 3039 | | | | |
| Seq ID 748 | Seq ID 1894 | Seq ID 3040 | | | | |
| Seq ID 749 | Seq ID 1895 | Seq ID 3041 | | | | |
| Seq ID 750 | Seq ID 1896 | Seq ID 3042 | | | | |
| Seq ID 751 | Seq ID 1897 | Seq ID 3043 | | | | |
| Seq ID 752 | Seq ID 1898 | Seq ID 3044 | | | | |
| Seq ID 753 | Seq ID 1899 | Seq ID 3045 | | | | |
| Seq ID 754 | Seq ID 1900 | Seq ID 3046 | | | | |
| Seq ID 755 | Seq ID 1901 | Seq ID 3047 | | | | |
| Seq ID 756 | Seq ID 1902 | Seq ID 3048 | | | | |
| Seq ID 757 | Seq ID 1903 | Seq ID 3049 | | | | |
| Seq ID 758 | Seq ID 1904 | Seq ID 3050 | | | | |
| Seq ID 759 | Seq ID 1905 | Seq ID 3051 | | | | |
| Seq ID 760 | Seq ID 1906 | Seq ID 3052 | | | | |
| Seq ID 761 | Seq ID 1907 | Seq ID 3053 | | | | |
| Seq ID 762 | Seq ID 1908 | Seq ID 3054 | | | | |
| Seq ID 763 | Seq ID 1909 | Seq ID 3055 | | | | |
| Seq ID 764 | Seq ID 1910 | Seq ID 3056 | | | | |
| Seq ID 765 | Seq ID 1911 | Seq ID 3057 | | | | |
| Seq ID 766 | Seq ID 1912 | Seq ID 3058 | | | | |
| Seq ID 767 | Seq ID 1913 | Seq ID 3059 | | | | |
| Seq ID 768 | Seq ID 1914 | Seq ID 3060 | | | | |
| Seq ID 769 | Seq ID 1915 | Seq ID 3061 | | | | |
| Seq ID 770 | Seq ID 1916 | Seq ID 3062 | | | | |
| Seq ID 771 | Seq ID 1917 | Seq ID 3063 | | | | |
| Seq ID 772 | Seq ID 1918 | Seq ID 3064 | | | | |
| Seq ID 773 | Seq ID 1919 | Seq ID 3065 | X | | | |
| Seq ID 774 | Seq ID 1920 | Seq ID 3066 | X | | | |
| Seq ID 775 | Seq ID 1921 | Seq ID 3067 | X | | | X |
| Seq ID 776 | Seq ID 1922 | Seq ID 3068 | X | | | |
| Seq ID 777 | Seq ID 1923 | Seq ID 3069 | X | | | X |
| Seq ID 778 | Seq ID 1924 | Seq ID 3070 | | | | |
| Seq ID 779 | Seq ID 1925 | Seq ID 3071 | | | | X |
| Seq ID 780 | Seq ID 1926 | Seq ID 3072 | | | | |
| Seq ID 781 | Seq ID 1927 | Seq ID 3073 | | | | |
| Seq ID 782 | Seq ID 1928 | Seq ID 3074 | | | | |
| Seq ID 783 | Seq ID 1929 | Seq ID 3075 | | | | |
| Seq ID 784 | Seq ID 1930 | Seq ID 3076 | | | | |
| Seq ID 785 | Seq ID 1931 | Seq ID 3077 | | | | |
| Seq ID 786 | Seq ID 1932 | Seq ID 3078 | | | | |
| Seq ID 787 | Seq ID 1933 | Seq ID 3079 | | | | |
| Seq ID 788 | Seq ID 1934 | Seq ID 3080 | X | | | |
| Seq ID 789 | Seq ID 1935 | Seq ID 3081 | | | | |
| Seq ID 790 | Seq ID 1936 | Seq ID 3082 | | | | |
| Seq ID 791 | Seq ID 1937 | Seq ID 3083 | | | | |
| Seq ID 792 | Seq ID 1938 | Seq ID 3084 | | X | | |
| Seq ID 793 | Seq ID 1939 | Seq ID 3085 | X | | | X |
| Seq ID 794 | Seq ID 1940 | Seq ID 3086 | | | | |
| Seq ID 795 | Seq ID 1941 | Seq ID 3087 | | | | |
| Seq ID 796 | Seq ID 1942 | Seq ID 3088 | | | | |
| Seq ID 797 | Seq ID 1943 | Seq ID 3089 | | | | |
| Seq ID 798 | Seq ID 1944 | Seq ID 3090 | | | | |
| Seq ID 799 | Seq ID 1945 | Seq ID 3091 | | | | |
| Seq ID 800 | Seq ID 1946 | Seq ID 3092 | | | | |
| Seq ID 801 | Seq ID 1947 | Seq ID 3093 | | | | |
| Seq ID 802 | Seq ID 1948 | Seq ID 3094 | | | | |
| Seq ID 803 | Seq ID 1949 | Seq ID 3095 | | | | |
| Seq ID 804 | Seq ID 1950 | Seq ID 3096 | | | | |
| Seq ID 805 | Seq ID 1951 | Seq ID 3097 | | | | |
| Seq ID 806 | Seq ID 1952 | Seq ID 3098 | | | | |
| Seq ID 807 | Seq ID 1953 | Seq ID 3099 | | | | |
| Seq ID 808 | Seq ID 1954 | Seq ID 3100 | | | | |
| Seq ID 809 | Seq ID 1955 | Seq ID 3101 | | | | |
| Seq ID 810 | Seq ID 1956 | Seq ID 3102 | | | | |
| Seq ID 811 | Seq ID 1957 | Seq ID 3103 | | | | |
| Seq ID 812 | Seq ID 1958 | Seq ID 3104 | | | | |
| Seq ID 813 | Seq ID 1959 | Seq ID 3105 | | | | |
| Seq ID 814 | Seq ID 1960 | Seq ID 3106 | | | | |
| Seq ID 815 | Seq ID 1961 | Seq ID 3107 | | | | |
| Seq ID 816 | Seq ID 1962 | Seq ID 3108 | | | | |
| Seq ID 817 | Seq ID 1963 | Seq ID 3109 | | | | |
| Seq ID 818 | Seq ID 1964 | Seq ID 3110 | | | | |
| Seq ID 819 | Seq ID 1965 | Seq ID 3111 | | | | |
| Seq ID 820 | Seq ID 1966 | Seq ID 3112 | | | | |
| Seq ID 821 | Seq ID 1967 | Seq ID 3113 | X | | | X |
| Seq ID 822 | Seq ID 1968 | Seq ID 3114 | | | | |
| Seq ID 823 | Seq ID 1969 | Seq ID 3115 | | | | |
| Seq ID 824 | Seq ID 1970 | Seq ID 3116 | | | | |
| Seq ID 825 | Seq ID 1971 | Seq ID 3117 | | | | X |
| Seq ID 826 | Seq ID 1972 | Seq ID 3118 | | | | |
| Seq ID 827 | Seq ID 1973 | Seq ID 3119 | | | | |
| Seq ID 828 | Seq ID 1974 | Seq ID 3120 | | | | |
| Seq ID 829 | Seq ID 1975 | Seq ID 3121 | | | | |
| Seq ID 830 | Seq ID 1976 | Seq ID 3122 | | | | |
| Seq ID 831 | Seq ID 1977 | Seq ID 3123 | | | | |
| Seq ID 832 | Seq ID 1978 | Seq ID 3124 | | | | |
| Seq ID 833 | Seq ID 1979 | Seq ID 3125 | | | | |
| Seq ID 834 | Seq ID 1980 | Seq ID 3126 | | | | |
| Seq ID 835 | Seq ID 1981 | Seq ID 3127 | | | | |
| Seq ID 836 | Seq ID 1982 | Seq ID 3128 | | | | |
| Seq ID 837 | Seq ID 1983 | Seq ID 3129 | | X | | |
| Seq ID 838 | Seq ID 1984 | Seq ID 3130 | | | | |
| Seq ID 839 | Seq ID 1985 | Seq ID 3131 | | | | |
| Seq ID 840 | Seq ID 1986 | Seq ID 3132 | | | | |
| Seq ID 841 | Seq ID 1987 | Seq ID 3133 | | | | |
| Seq ID 842 | Seq ID 1988 | Seq ID 3134 | X | | | |
| Seq ID 843 | Seq ID 1989 | Seq ID 3135 | | | | |
| Seq ID 844 | Seq ID 1990 | Seq ID 3136 | | | | |
| Seq ID 845 | Seq ID 1991 | Seq ID 3137 | | | | |
| Seq ID 846 | Seq ID 1992 | Seq ID 3138 | | | | |
| Seq ID 847 | Seq ID 1993 | Seq ID 3139 | | | | |
| Seq ID 848 | Seq ID 1994 | Seq ID 3140 | | | | |
| Seq ID 849 | Seq ID 1995 | Seq ID 3141 | | | | |
| Seq ID 850 | Seq ID 1996 | Seq ID 3142 | | | | |
| Seq ID 851 | Seq ID 1997 | Seq ID 3143 | | | | |
| Seq ID 852 | Seq ID 1998 | Seq ID 3144 | | | | |
| Seq ID 853 | Seq ID 1999 | Seq ID 3145 | | | | |
| Seq ID 854 | Seq ID 2000 | Seq ID 3146 | | | | |
| Seq ID 855 | Seq ID 2001 | Seq ID 3147 | | | | |
| Seq ID 856 | Seq ID 2002 | Seq ID 3148 | | | | |
| Seq ID 857 | Seq ID 2003 | Seq ID 3149 | | | | |
| Seq ID 858 | Seq ID 2004 | Seq ID 3150 | | | | |
| Seq ID 859 | Seq ID 2005 | Seq ID 3151 | X | | | |
| Seq ID 860 | Seq ID 2006 | Seq ID 3152 | | | | |
| Seq ID 861 | Seq ID 2007 | Seq ID 3153 | | | | |
| Seq ID 862 | Seq ID 2008 | Seq ID 3154 | | | | |
| Seq ID 863 | Seq ID 2009 | Seq ID 3155 | | | | |
| Seq ID 864 | Seq ID 2010 | Seq ID 3156 | | | | |
| Seq ID 865 | Seq ID 2011 | Seq ID 3157 | | | | |
| Seq ID 866 | Seq ID 2012 | Seq ID 3158 | X | | | |
| Seq ID 867 | Seq ID 2013 | Seq ID 3159 | | | | |
| Seq ID 868 | Seq ID 2014 | Seq ID 3160 | | | | |
| Seq ID 869 | Seq ID 2015 | Seq ID 3161 | | | | |
| Seq ID 870 | Seq ID 2016 | Seq ID 3162 | | | | |
| Seq ID 871 | Seq ID 2017 | Seq ID 3163 | | | | |
| Seq ID 872 | Seq ID 2018 | Seq ID 3164 | | | | |
| Seq ID 873 | Seq ID 2019 | Seq ID 3165 | | | | |
| Seq ID 874 | Seq ID 2020 | Seq ID 3166 | | | | |
| Seq ID 875 | Seq ID 2021 | Seq ID 3167 | | | | |
| Seq ID 876 | Seq ID 2022 | Seq ID 3168 | | | | |
| Seq ID 877 | Seq ID 2023 | Seq ID 3169 | | | | |
| Seq ID 878 | Seq ID 2024 | Seq ID 3170 | X | | | X |
| Seq ID 879 | Seq ID 2025 | Seq ID 3171 | | | | |
| Seq ID 880 | Seq ID 2026 | Seq ID 3172 | | | | |
| Seq ID 881 | Seq ID 2027 | Seq ID 3173 | | | | |
| Seq ID 882 | Seq ID 2028 | Seq ID 3174 | | | | |
| Seq ID 883 | Seq ID 2029 | Seq ID 3175 | | | | |
| Seq ID 884 | Seq ID 2030 | Seq ID 3176 | | | | |
| Seq ID 885 | Seq ID 2031 | Seq ID 3177 | | | | |
| Seq ID 886 | Seq ID 2032 | Seq ID 3178 | | | | |
| Seq ID 887 | Seq ID 2033 | Seq ID 3179 | | | | |
| Seq ID 888 | Seq ID 2034 | Seq ID 3180 | X | | | |
| Seq ID 889 | Seq ID 2035 | Seq ID 3181 | | | | |
| Seq ID 890 | Seq ID 2036 | Seq ID 3182 | | | | |
| Seq ID 891 | Seq ID 2037 | Seq ID 3183 | X | | | |
| Seq ID 892 | Seq ID 2038 | Seq ID 3184 | | | | |
| Seq ID 893 | Seq ID 2039 | Seq ID 3185 | | | | |
| Seq ID 894 | Seq ID 2040 | Seq ID 3186 | | | | |
| Seq ID 895 | Seq ID 2041 | Seq ID 3187 | | | | |
| Seq ID 896 | Seq ID 2042 | Seq ID 3188 | | | | |
| Seq ID 897 | Seq ID 2043 | Seq ID 3189 | | | | |
| Seq ID 898 | Seq ID 2044 | Seq ID 3190 | | | | |
| Seq ID 899 | Seq ID 2045 | Seq ID 3191 | | | | |
| Seq ID 900 | Seq ID 2046 | Seq ID 3192 | | | | |
| Seq ID 901 | Seq ID 2047 | Seq ID 3193 | | | | |
| Seq ID 902 | Seq ID 2048 | Seq ID 3194 | | | | |
| Seq ID 903 | Seq ID 2049 | Seq ID 3195 | | | | |
| Seq ID 904 | Seq ID 2050 | Seq ID 3196 | | | | |
| Seq ID 905 | Seq ID 2051 | Seq ID 3197 | | | | |
| Seq ID 906 | Seq ID 2052 | Seq ID 3198 | | | | |
| Seq ID 907 | Seq ID 2053 | Seq ID 3199 | | | | |
| Seq ID 908 | Seq ID 2054 | Seq ID 3200 | | | | |
| Seq ID 909 | Seq ID 2055 | Seq ID 3201 | | | | |
| Seq ID 910 | Seq ID 2056 | Seq ID 3202 | | | | |
| Seq ID 911 | Seq ID 2057 | Seq ID 3203 | | | | |
| Seq ID 912 | Seq ID 2058 | Seq ID 3204 | | | | |
| Seq ID 913 | Seq ID 2059 | Seq ID 3205 | | | | |
| Seq ID 914 | Seq ID 2060 | Seq ID 3206 | | | | |
| Seq ID 915 | Seq ID 2061 | Seq ID 3207 | | | | |
| Seq ID 916 | Seq ID 2062 | Seq ID 3208 | | | | |
| Seq ID 917 | Seq ID 2063 | Seq ID 3209 | | | | |
| Seq ID 918 | Seq ID 2064 | Seq ID 3210 | | | | |
| Seq ID 919 | Seq ID 2065 | Seq ID 3211 | | | | |
| Seq ID 920 | Seq ID 2066 | Seq ID 3212 | | | | |
| Seq ID 921 | Seq ID 2067 | Seq ID 3213 | | | | |
| Seq ID 922 | Seq ID 2068 | Seq ID 3214 | | | | |
| Seq ID 923 | Seq ID 2069 | Seq ID 3215 | | | | |
| Seq ID 924 | Seq ID 2070 | Seq ID 3216 | | | | |
| Seq ID 925 | Seq ID 2071 | Seq ID 3217 | | | | |
| Seq ID 926 | Seq ID 2072 | Seq ID 3218 | X | | | |
| Seq ID 927 | Seq ID 2073 | Seq ID 3219 | | | | |
| Seq ID 928 | Seq ID 2074 | Seq ID 3220 | | | | |
| Seq ID 929 | Seq ID 2075 | Seq ID 3221 | | | | |
| Seq ID 930 | Seq ID 2076 | Seq ID 3222 | | | | |
| Seq ID 931 | Seq ID 2077 | Seq ID 3223 | | | | |
| Seq ID 932 | Seq ID 2078 | Seq ID 3224 | | | | |
| Seq ID 933 | Seq ID 2079 | Seq ID 3225 | | | | |
| Seq ID 934 | Seq ID 2080 | Seq ID 3226 | | | | |
| Seq ID 935 | Seq ID 2081 | Seq ID 3227 | | | | |
| Seq ID 936 | Seq ID 2082 | Seq ID 3228 | | | | |
| Seq ID 937 | Seq ID 2083 | Seq ID 3229 | | | | |
| Seq ID 938 | Seq ID 2084 | Seq ID 3230 | | | | |
| Seq ID 939 | Seq ID 2085 | Seq ID 3231 | | | | |
| Seq ID 940 | Seq ID 2086 | Seq ID 3232 | | | | |
| Seq ID 941 | Seq ID 2087 | Seq ID 3233 | | | | |
| Seq ID 942 | Seq ID 2088 | Seq ID 3234 | | | | |
| Seq ID 943 | Seq ID 2089 | Seq ID 3235 | | | | |
| Seq ID 944 | Seq ID 2090 | Seq ID 3236 | | | | |
| Seq ID 945 | Seq ID 2091 | Seq ID 3237 | | | | |
| Seq ID 946 | Seq ID 2092 | Seq ID 3238 | | | | |
| Seq ID 947 | Seq ID 2093 | Seq ID 3239 | | | | |
| Seq ID 948 | Seq ID 2094 | Seq ID 3240 | | | | |
| Seq ID 949 | Seq ID 2095 | Seq ID 3241 | | | | |
| Seq ID 950 | Seq ID 2096 | Seq ID 3242 | | | | |
| Seq ID 951 | Seq ID 2097 | Seq ID 3243 | | | | |
| Seq ID 952 | Seq ID 2098 | Seq ID 3244 | | | | |
| Seq ID 953 | Seq ID 2099 | Seq ID 3245 | | | | |
| Seq ID 954 | Seq ID 2100 | Seq ID 3246 | X | | | |
| Seq ID 955 | Seq ID 2101 | Seq ID 3247 | | | | |
| Seq ID 956 | Seq ID 2102 | Seq ID 3248 | | | | |
| Seq ID 957 | Seq ID 2103 | Seq ID 3249 | | | | |
| Seq ID 958 | Seq ID 2104 | Seq ID 3250 | | | | |
| Seq ID 959 | Seq ID 2105 | Seq ID 3251 | | | | |
| Seq ID 960 | Seq ID 2106 | Seq ID 3252 | | | | |
| Seq ID 961 | Seq ID 2107 | Seq ID 3253 | | | | |
| Seq ID 962 | Seq ID 2108 | Seq ID 3254 | | | | |
| Seq ID 963 | Seq ID 2109 | Seq ID 3255 | X | | | |
| Seq ID 964 | Seq ID 2110 | Seq ID 3256 | | | | |
| Seq ID 965 | Seq ID 2111 | Seq ID 3257 | | | | |
| Seq ID 966 | Seq ID 2112 | Seq ID 3258 | | | | |
| Seq ID 967 | Seq ID 2113 | Seq ID 3259 | | | | |
| Seq ID 968 | Seq ID 2114 | Seq ID 3260 | | | | |
| Seq ID 969 | Seq ID 2115 | Seq ID 3261 | | | | |
| Seq ID 970 | Seq ID 2116 | Seq ID 3262 | | | | |
| Seq ID 971 | Seq ID 2117 | Seq ID 3263 | | | | |
| Seq ID 972 | Seq ID 2118 | Seq ID 3264 | | | | |
| Seq ID 973 | Seq ID 2119 | Seq ID 3265 | X | | | |
| Seq ID 974 | Seq ID 2120 | Seq ID 3266 | | | | |
| Seq ID 975 | Seq ID 2121 | Seq ID 3267 | | | | |
| Seq ID 976 | Seq ID 2122 | Seq ID 3268 | | | | |
| Seq ID 977 | Seq ID 2123 | Seq ID 3269 | | | | |
| Seq ID 978 | Seq ID 2124 | Seq ID 3270 | | | | |
| Seq ID 979 | Seq ID 2125 | Seq ID 3271 | | | | |
| Seq ID 980 | Seq ID 2126 | Seq ID 3272 | | | | |
| Seq ID 981 | Seq ID 2127 | Seq ID 3273 | | | | |
| Seq ID 982 | Seq ID 2128 | Seq ID 3274 | X | | | |
| Seq ID 983 | Seq ID 2129 | Seq ID 3275 | X | | | X |
| Seq ID 984 | Seq ID 2130 | Seq ID 3276 | X | | | |
| Seq ID 985 | Seq ID 2131 | Seq ID 3277 | | | | |
| Seq ID 986 | Seq ID 2132 | Seq ID 3278 | | | | |
| Seq ID 987 | Seq ID 2133 | Seq ID 3279 | | | | |
| Seq ID 988 | Seq ID 2134 | Seq ID 3280 | | | | |
| Seq ID 989 | Seq ID 2135 | Seq ID 3281 | | | | |
| Seq ID 990 | Seq ID 2136 | Seq ID 3282 | | | | |
| Seq ID 991 | Seq ID 2137 | Seq ID 3283 | | | | |
| Seq ID 992 | Seq ID 2138 | Seq ID 3284 | | | | |
| Seq ID 993 | Seq ID 2139 | Seq ID 3285 | | | | |
| Seq ID 994 | Seq ID 2140 | Seq ID 3286 | X | | | |
| Seq ID 995 | Seq ID 2141 | Seq ID 3287 | | | | |
| Seq ID 996 | Seq ID 2142 | Seq ID 3288 | X | | X | |
| Seq ID 997 | Seq ID 2143 | Seq ID 3289 | | | | |
| Seq ID 998 | Seq ID 2144 | Seq ID 3290 | | | | |
| Seq ID 999 | Seq ID 2145 | Seq ID 3291 | | | | |
| Seq ID 1000 | Seq ID 2146 | Seq ID 3292 | | | | |
| Seq ID 1001 | Seq ID 2147 | Seq ID 3293 | | | | |
| Seq ID 1002 | Seq ID 2148 | Seq ID 3294 | | | | |
| Seq ID 1003 | Seq ID 2149 | Seq ID 3295 | | | | |
| Seq ID 1004 | Seq ID 2150 | Seq ID 3296 | X | | | |
| Seq ID 1005 | Seq ID 2151 | Seq ID 3297 | | | | |
| Seq ID 1006 | Seq ID 2152 | Seq ID 3298 | | | | |
| Seq ID 1007 | Seq ID 2153 | Seq ID 3299 | | | | |
| Seq ID 1008 | Seq ID 2154 | Seq ID 3300 | | | | |
| Seq ID 1009 | Seq ID 2155 | Seq ID 3301 | | | | |
| Seq ID 1010 | Seq ID 2156 | Seq ID 3302 | | | | |
| Seq ID 1011 | Seq ID 2157 | Seq ID 3303 | | | | |
| Seq ID 1012 | Seq ID 2158 | Seq ID 3304 | | | | |
| Seq ID 1013 | Seq ID 2159 | Seq ID 3305 | | | | |
| Seq ID 1014 | Seq ID 2160 | Seq ID 3306 | | | | |
| Seq ID 1015 | Seq ID 2161 | Seq ID 3307 | | | | |
| Seq ID 1016 | Seq ID 2162 | Seq ID 3308 | | | | |
| Seq ID 1017 | Seq ID 2163 | Seq ID 3309 | | | | |
| Seq ID 1018 | Seq ID 2164 | Seq ID 3310 | | | | |
| Seq ID 1019 | Seq ID 2165 | Seq ID 3311 | X | | | |
| Seq ID 1020 | Seq ID 2166 | Seq ID 3312 | | | | |
| Seq ID 1021 | Seq ID 2167 | Seq ID 3313 | | | | |
| Seq ID 1022 | Seq ID 2168 | Seq ID 3314 | | | | |
| Seq ID 1023 | Seq ID 2169 | Seq ID 3315 | | | | |
| Seq ID 1024 | Seq ID 2170 | Seq ID 3316 | | | | |
| Seq ID 1025 | Seq ID 2171 | Seq ID 3317 | | | | |
| Seq ID 1026 | Seq ID 2172 | Seq ID 3318 | X | | | |
| Seq ID 1027 | Seq ID 2173 | Seq ID 3319 | | | | |
| Seq ID 1028 | Seq ID 2174 | Seq ID 3320 | | | X | |
| Seq ID 1029 | Seq ID 2175 | Seq ID 3321 | | | | |
| Seq ID 1030 | Seq ID 2176 | Seq ID 3322 | | | | |
| Seq ID 1031 | Seq ID 2177 | Seq ID 3323 | | | | |
| Seq ID 1032 | Seq ID 2178 | Seq ID 3324 | | | | |
| Seq ID 1033 | Seq ID 2179 | Seq ID 3325 | | | | |
| Seq ID 1034 | Seq ID 2180 | Seq ID 3326 | | | | |
| Seq ID 1035 | Seq ID 2181 | Seq ID 3327 | | | | |
| Seq ID 1036 | Seq ID 2182 | Seq ID 3328 | | | | |
| Seq ID 1037 | Seq ID 2183 | Seq ID 3329 | | | | |
| Seq ID 1038 | Seq ID 2184 | Seq ID 3330 | | | | |
| Seq ID 1039 | Seq ID 2185 | Seq ID 3331 | | | | |
| Seq ID 1040 | Seq ID 2186 | Seq ID 3332 | | | | |
| Seq ID 1041 | Seq ID 2187 | Seq ID 3333 | | | | |
| Seq ID 1042 | Seq ID 2188 | Seq ID 3334 | | | | |
| Seq ID 1043 | Seq ID 2189 | Seq ID 3335 | | | | |
| Seq ID 1044 | Seq ID 2190 | Seq ID 3336 | | | | |
| Seq ID 1045 | Seq ID 2191 | Seq ID 3337 | | | | |
| Seq ID 1046 | Seq ID 2192 | Seq ID 3338 | | | | |
| Seq ID 1047 | Seq ID 2193 | Seq ID 3339 | | | | |
| Seq ID 1048 | Seq ID 2194 | Seq ID 3340 | | | | |
| Seq ID 1049 | Seq ID 2195 | Seq ID 3341 | | | | |
| Seq ID 1050 | Seq ID 2196 | Seq ID 3342 | | | | |
| Seq ID 1051 | Seq ID 2197 | Seq ID 3343 | | | | |
| Seq ID 1052 | Seq ID 2198 | Seq ID 3344 | X | | | |
| Seq ID 1053 | Seq ID 2199 | Seq ID 3345 | | | | |
| Seq ID 1054 | Seq ID 2200 | Seq ID 3346 | | | | |
| Seq ID 1055 | Seq ID 2201 | Seq ID 3347 | | | | |
| Seq ID 1056 | Seq ID 2202 | Seq ID 3348 | | | | |
| Seq ID 1057 | Seq ID 2203 | Seq ID 3349 | | | | |
| Seq ID 1058 | Seq ID 2204 | Seq ID 3350 | | | | |
| Seq ID 1059 | Seq ID 2205 | Seq ID 3351 | | | | |
| Seq ID 1060 | Seq ID 2206 | Seq ID 3352 | X | | | |
| Seq ID 1061 | Seq ID 2207 | Seq ID 3353 | | | | |
| Seq ID 1062 | Seq ID 2208 | Seq ID 3354 | | | | |
| Seq ID 1063 | Seq ID 2209 | Seq ID 3355 | | | | |
| Seq ID 1064 | Seq ID 2210 | Seq ID 3356 | X | | | |
| Seq ID 1065 | Seq ID 2211 | Seq ID 3357 | | | | |
| Seq ID 1066 | Seq ID 2212 | Seq ID 3358 | X | | | |
| Seq ID 1067 | Seq ID 2213 | Seq ID 3359 | | | | |
| Seq ID 1068 | Seq ID 2214 | Seq ID 3360 | | | | |
| Seq ID 1069 | Seq ID 2215 | Seq ID 3361 | | | | |
| Seq ID 1070 | Seq ID 2216 | Seq ID 3362 | | | | |
| Seq ID 1071 | Seq ID 2217 | Seq ID 3363 | | | | |
| Seq ID 1072 | Seq ID 2218 | Seq ID 3364 | | | | |
| Seq ID 1073 | Seq ID 2219 | Seq ID 3365 | | | | |
| Seq ID 1074 | Seq ID 2220 | Seq ID 3366 | | | | |
| Seq ID 1075 | Seq ID 2221 | Seq ID 3367 | | | | |
| Seq ID 1076 | Seq ID 2222 | Seq ID 3368 | X | | | |
| Seq ID 1077 | Seq ID 2223 | Seq ID 3369 | | | | |
| Seq ID 1078 | Seq ID 2224 | Seq ID 3370 | | | | |
| Seq ID 1079 | Seq ID 2225 | Seq ID 3371 | | | | |
| Seq ID 1080 | Seq ID 2226 | Seq ID 3372 | | | | |
| Seq ID 1081 | Seq ID 2227 | Seq ID 3373 | X | | | |
| Seq ID 1082 | Seq ID 2228 | Seq ID 3374 | | | | |
| Seq ID 1083 | Seq ID 2229 | Seq ID 3375 | | | | |
| Seq ID 1084 | Seq ID 2230 | Seq ID 3376 | | | | |
| Seq ID 1085 | Seq ID 2231 | Seq ID 3377 | | | | |
| Seq ID 1086 | Seq ID 2232 | Seq ID 3378 | | | | |
| Seq ID 1087 | Seq ID 2233 | Seq ID 3379 | | | | |
| Seq ID 1088 | Seq ID 2234 | Seq ID 3380 | | | | |
| Seq ID 1089 | Seq ID 2235 | Seq ID 3381 | | | | |
| Seq ID 1090 | Seq ID 2236 | Seq ID 3382 | | | | |
| Seq ID 1091 | Seq ID 2237 | Seq ID 3383 | | | | |
| Seq ID 1092 | Seq ID 2238 | Seq ID 3384 | | | | |
| Seq ID 1093 | Seq ID 2239 | Seq ID 3385 | | | | |
| Seq ID 1094 | Seq ID 2240 | Seq ID 3386 | | | | |
| Seq ID 1095 | Seq ID 2241 | Seq ID 3387 | X | | | |
| Seq ID 1096 | Seq ID 2242 | Seq ID 3388 | | | | |
| Seq ID 1097 | Seq ID 2243 | Seq ID 3389 | | | | |
| Seq ID 1098 | Seq ID 2244 | Seq ID 3390 | | | | |
| Seq ID 1099 | Seq ID 2245 | Seq ID 3391 | | | | |
| Seq ID 1100 | Seq ID 2246 | Seq ID 3392 | | | | |
| Seq ID 1101 | Seq ID 2247 | Seq ID 3393 | | | | |
| Seq ID 1102 | Seq ID 2248 | Seq ID 3394 | | | | |
| Seq ID 1103 | Seq ID 2249 | Seq ID 3395 | X | | | |
| Seq ID 1104 | Seq ID 2250 | Seq ID 3396 | | | | |
| Seq ID 1105 | Seq ID 2251 | Seq ID 3397 | | | | |
| Seq ID 1106 | Seq ID 2252 | Seq ID 3398 | | | | |
| Seq ID 1107 | Seq ID 2253 | Seq ID 3399 | | | | |
| Seq ID 1108 | Seq ID 2254 | Seq ID 3400 | | | | |
| Seq ID 1109 | Seq ID 2255 | Seq ID 3401 | | | | |
| Seq ID 1110 | Seq ID 2256 | Seq ID 3402 | | | | |
| Seq ID 1111 | Seq ID 2257 | Seq ID 3403 | | | | |
| Seq ID 1112 | Seq ID 2258 | Seq ID 3404 | | | | |
| Seq ID 1113 | Seq ID 2259 | Seq ID 3405 | | | | |
| Seq ID 1114 | Seq ID 2260 | Seq ID 3406 | | | | |
| Seq ID 1115 | Seq ID 2261 | Seq ID 3407 | | | | |
| Seq ID 1116 | Seq ID 2262 | Seq ID 3408 | | | | |
| Seq ID 1117 | Seq ID 2263 | Seq ID 3409 | | | | |
| Seq ID 1118 | Seq ID 2264 | Seq ID 3410 | | | | |
| Seq ID 1119 | Seq ID 2265 | Seq ID 3411 | | | | |
| Seq ID 1120 | Seq ID 2266 | Seq ID 3412 | | | | |
| Seq ID 1121 | Seq ID 2267 | Seq ID 3413 | | | | |
| Seq ID 1122 | Seq ID 2268 | Seq ID 3414 | | | | |
| Seq ID 1123 | Seq ID 2269 | Seq ID 3415 | | | | |
| Seq ID 1124 | Seq ID 2270 | Seq ID 3416 | | | | |
| Seq ID 1125 | Seq ID 2271 | Seq ID 3417 | | | | |
| Seq ID 1126 | Seq ID 2272 | Seq ID 3418 | | | | |
| Seq ID 1127 | Seq ID 2273 | Seq ID 3419 | | | | |
| Seq ID 1128 | Seq ID 2274 | Seq ID 3420 | | | | |
| Seq ID 1129 | Seq ID 2275 | Seq ID 3421 | | | | |
| Seq ID 1130 | Seq ID 2276 | Seq ID 3422 | | | | |
| Seq ID 1131 | Seq ID 2277 | Seq ID 3423 | | | | |
| Seq ID 1132 | Seq ID 2278 | Seq ID 3424 | | | | |
| Seq ID 1133 | Seq ID 2279 | Seq ID 3425 | | | | |
| Seq ID 1134 | Seq ID 2280 | Seq ID 3426 | | | | |
| Seq ID 1135 | Seq ID 2281 | Seq ID 3427 | | | | |
| Seq ID 1136 | Seq ID 2282 | Seq ID 3428 | | | | |
| Seq ID 1137 | Seq ID 2283 | Seq ID 3429 | | | | |
| Seq ID 1138 | Seq ID 2284 | Seq ID 3430 | | | | |
| Seq ID 1139 | Seq ID 2285 | Seq ID 3431 | | | | |
| Seq ID 1140 | Seq ID 2286 | Seq ID 3432 | | | | |
| Seq ID 1141 | Seq ID 2287 | Seq ID 3433 | | | | |
| Seq ID 1142 | Seq ID 2288 | Seq ID 3434 | | | | |
| Seq ID 1143 | Seq ID 2289 | Seq ID 3435 | | | | |
| Seq ID 1144 | Seq ID 2290 | Seq ID 3436 | | | | |
| Seq ID 1145 | Seq ID 2291 | Seq ID 3437 | | | | |
| Seq ID 1146 | Seq ID 2292 | Seq ID 3438 | | | | |

TABLE 7

| Colon | Nonparametric | | Global | | | | | |
|---|---|---|---|---|---|---|---|---|
| Nucleotide ID | Exp | Order | 5 | 4 | 3 | 2 | 1 | 0 |
| Seq ID 002 | 1 | 1 | 0 | 0 | 0 | 1 | 4 | 6 |
| Seq ID 008 | 3 | 2 | 0 | 0 | 3 | 3 | 1 | 4 |
| Seq ID 009 | 2 | 3 | 0 | 0 | 1 | 6 | 0 | 4 |
| Seq ID 013 | 2 | 2 | 0 | 0 | 0 | 3 | 2 | 6 |
| Seq ID 014 | 3 | 2 | 0 | 0 | 2 | 2 | 1 | 6 |
| Seq ID 019 | 3 | 3 | 0 | 1 | 2 | 1 | 3 | 4 |
| Seq ID 023 | 2 | 3 | 0 | 1 | 0 | 4 | 2 | 4 |
| Seq ID 025 | 3 | 3 | 0 | 3 | 2 | 0 | 2 | 4 |
| Seq ID 028 | 3 | 3 | 0 | 0 | 7 | 1 | 0 | 3 |
| Seq ID 046 | 2 | 3 | 0 | 0 | 0 | 4 | 3 | 4 |
| Seq ID 052 | 5 | 4 | 3 | 0 | 0 | 0 | 5 | 3 |
| Seq ID 072 | 1 | 3 | 0 | 0 | 1 | 1 | 2 | 7 |
| Seq ID 078 | 3 | 4 | 0 | 2 | 1 | 1 | 2 | 5 |
| Seq ID 079 | 4 | 4 | 0 | 3 | 1 | 0 | 6 | 1 |
| Seq ID 086 | 3 | 3 | 0 | 0 | 4 | 0 | 1 | 6 |
| Seq ID 116 | 3 | 3 | 0 | 0 | 4 | 0 | 2 | 5 |
| Seq ID 129 | 3 | 3 | 0 | 0 | 3 | 1 | 1 | 6 |
| Seq ID 142 | 2 | 4 | 0 | 0 | 3 | 5 | 0 | 3 |
| Seq ID 160 | 3 | 3 | 0 | 1 | 2 | 0 | 2 | 6 |
| Seq ID 174 | 2 | 3 | 0 | 0 | 1 | 5 | 0 | 5 |
| Seq ID 178 | 2 | 2 | 0 | 0 | 3 | 1 | 1 | 6 |
| Seq ID 198 | 3 | 3 | 0 | 0 | 2 | 0 | 7 | 2 |
| Seq ID 203 | 3 | 3 | 0 | 0 | 3 | 0 | 6 | 2 |
| Seq ID 212 | 2 | 3 | 0 | 0 | 0 | 3 | 2 | 6 |
| Seq ID 235 | 3 | 4 | 0 | 1 | 5 | 2 | 0 | 3 |
| Seq ID 269 | 4 | 4 | 0 | 3 | 0 | 0 | 0 | 8 |
| Seq ID 270 | 3 | 2 | 0 | 0 | 3 | 1 | 3 | 4 |
| Seq ID 274 | 2 | 3 | 0 | 0 | 0 | 5 | 0 | 6 |
| Seq ID 282 | 3 | 4 | 0 | 3 | 3 | 2 | 0 | 3 |
| Seq ID 286 | 3 | 3 | 0 | 0 | 4 | 0 | 4 | 3 |
| Seq ID 288 | 3 | 4 | 0 | 2 | 3 | 2 | 1 | 3 |
| Seq ID 292 | 3 | 3 | 1 | 0 | 0 | 1 | 2 | 7 |
| Seq ID 305 | 3 | 4 | 0 | 1 | 1 | 2 | 3 | 4 |
| Seq ID 312 | 1 | 1 | 0 | 0 | 1 | 1 | 5 | 4 |
| Seq ID 316 | 3 | 3 | 0 | 0 | 2 | 0 | 1 | 8 |
| Seq ID 347 | 2 | 1 | 0 | 0 | 0 | 1 | 4 | 6 |
| Seq ID 348 | 3 | 3 | 0 | 0 | 2 | 1 | 3 | 5 |
| Seq ID 354 | 3 | 3 | 0 | 0 | 3 | 4 | 1 | 3 |
| Seq ID 355 | 3 | 4 | 0 | 1 | 5 | 1 | 1 | 3 |
| Seq ID 357 | 3 | 4 | 0 | 1 | 4 | 4 | 0 | 2 |
| Seq ID 360 | 3 | 3 | 0 | 0 | 2 | 1 | 4 | 4 |
| Seq ID 371 | 2 | 3 | 0 | 0 | 1 | 6 | 1 | 3 |
| Seq ID 372 | 3 | 3 | 0 | 1 | 2 | 2 | 1 | 5 |
| Seq ID 404 | 0 | 0 | 0 | 1 | 0 | 0 | 0 | 10 |
| Seq ID 433 | 3 | 3 | 0 | 0 | 3 | 1 | 0 | 7 |
| Seq ID 435 | 4 | 4 | 0 | 1 | 2 | 2 | 0 | 6 |
| Seq ID 437 | 1 | 1 | 0 | 0 | 0 | 0 | 4 | 7 |
| Seq ID 463 | 2 | 3 | 0 | 0 | 0 | 4 | 3 | 4 |
| Seq ID 469 | 2 | 3 | 0 | 0 | 2 | 1 | 2 | 6 |
| Seq ID 471 | 3 | 3 | 0 | 2 | 1 | 4 | 1 | 3 |
| Seq ID 477 | 2 | 2 | 0 | 0 | 1 | 0 | 1 | 9 |
| Seq ID 479 | 3 | 3 | 0 | 0 | 6 | 1 | 0 | 4 |
| Seq ID 486 | 3 | 3 | 0 | 0 | 5 | 0 | 0 | 6 |
| Seq ID 500 | 3 | 3 | 0 | 0 | 4 | 0 | 4 | 3 |
| Seq ID 507 | 2 | 2 | 0 | 0 | 1 | 7 | 0 | 3 |
| Seq ID 509 | 3 | 3 | 0 | 0 | 3 | 0 | 5 | 3 |
| Seq ID 525 | 2 | 2 | 0 | 0 | 0 | 2 | 2 | 7 |
| Seq ID 534 | 3 | 3 | 0 | 3 | 1 | 1 | 3 | 3 |
| Seq ID 543 | 1 | 2 | 0 | 0 | 0 | 0 | 3 | 8 |
| Seq ID 553 | 3 | 3 | 0 | 0 | 3 | 2 | 2 | 4 |
| Seq ID 562 | 2 | 2 | 0 | 0 | 1 | 3 | 2 | 5 |
| Seq ID 565 | 4 | 4 | 0 | 2 | 3 | 0 | 1 | 5 |
| Seq ID 586 | 1 | 2 | 0 | 0 | 2 | 2 | 1 | 6 |
| Seq ID 592 | 3 | 2 | 0 | 1 | 4 | 1 | 2 | 3 |
| Seq ID 613 | 2 | 2 | 0 | 0 | 1 | 6 | 0 | 4 |
| Seq ID 651 | 3 | 2 | 0 | 0 | 1 | 0 | 1 | 9 |
| Seq ID 657 | 3 | 3 | 0 | 0 | 4 | 0 | 4 | 3 |
| Seq ID 658 | 1 | 2 | 0 | 0 | 1 | 4 | 0 | 6 |
| Seq ID 668 | 2 | 3 | 0 | 0 | 0 | 1 | 5 | 5 |
| Seq ID 686 | 3 | 3 | 0 | 1 | 5 | 0 | 2 | 3 |
| Seq ID 688 | 1 | 1 | 0 | 0 | 0 | 1 | 5 | 5 |
| Seq ID 708 | 3 | 3 | 0 | 0 | 3 | 0 | 3 | 5 |
| Seq ID 710 | 1 | 1 | 0 | 0 | 0 | 0 | 9 | 2 |
| Seq ID 716 | 2 | 2 | 0 | 1 | 0 | 4 | 3 | 3 |
| Seq ID 720 | 3 | 3 | 0 | 3 | 1 | 1 | 4 | 2 |
| Seq ID 724 | 0 | 2 | 0 | 2 | 0 | 0 | 1 | 8 |
| Seq ID 731 | 1 | 2 | 0 | 0 | 0 | 2 | 1 | 8 |
| Seq ID 732 | 3 | 4 | 0 | 1 | 3 | 0 | 3 | 4 |
| Seq ID 744 | 1 | 0 | 0 | 0 | 3 | 0 | 1 | 7 |
| Seq ID 773 | 2 | 3 | 1 | 0 | 0 | 3 | 5 | 2 |
| Seq ID 774 | 3 | 3 | 0 | 1 | 4 | 1 | 1 | 4 |
| Seq ID 775 | 3 | 3 | 0 | 0 | 2 | 3 | 3 | 3 |
| Seq ID 776 | 3 | 4 | 0 | 2 | 3 | 1 | 2 | 3 |
| Seq ID 777 | 3 | 4 | 0 | 2 | 5 | 0 | 0 | 4 |
| Seq ID 788 | 3 | 4 | 0 | 1 | 4 | 0 | 0 | 6 |
| Seq ID 793 | 3 | 3 | 0 | 0 | 6 | 0 | 1 | 4 |
| Seq ID 821 | 1 | 1 | 0 | 0 | 0 | 0 | 5 | 6 |
| Seq ID 842 | 2 | 2 | 0 | 1 | 1 | 2 | 1 | 6 |
| Seq ID 859 | 3 | 3 | 0 | 0 | 2 | 3 | 1 | 5 |
| Seq ID 866 | 2 | 3 | 0 | 0 | 0 | 6 | 0 | 5 |
| Seq ID 878 | 3 | 3 | 0 | 0 | 5 | 0 | 4 | 2 |
| Seq ID 888 | 2 | 3 | 0 | 0 | 1 | 1 | 0 | 9 |
| Seq ID 891 | 4 | 4 | 0 | 4 | 2 | 1 | 0 | 4 |
| Seq ID 926 | 2 | 1 | 0 | 0 | 2 | 1 | 0 | 8 |
| Seq ID 954 | 4 | 4 | 0 | 3 | 1 | 1 | 1 | 5 |
| Seq ID 963 | 1 | 0 | 0 | 0 | 0 | 0 | 4 | 7 |
| Seq ID 973 | 2 | 3 | 0 | 0 | 3 | 1 | 1 | 6 |
| Seq ID 982 | 2 | 2 | 0 | 0 | 0 | 2 | 4 | 5 |
| Seq ID 983 | 3 | 3 | 0 | 0 | 6 | 3 | 0 | 2 |
| Seq ID 984 | 2 | 3 | 0 | 0 | 1 | 4 | 0 | 6 |
| Seq ID 994 | 3 | 4 | 0 | 5 | 0 | 1 | 0 | 5 |
| Seq ID 996 | 3 | 4 | 0 | 1 | 1 | 1 | 2 | 6 |
| Seq ID 1004 | 2 | 3 | 0 | 0 | 2 | 3 | 1 | 5 |
| Seq ID 1019 | 2 | 4 | 0 | 1 | 1 | 5 | 3 | 1 |
| Seq ID 1026 | 2 | 3 | 0 | 0 | 2 | 2 | 2 | 5 |
| Seq ID 1052 | 3 | 2 | 0 | 0 | 1 | 0 | 6 | 4 |
| Seq ID 1060 | 2 | 4 | 0 | 2 | 1 | 1 | 1 | 6 |
| Seq ID 1064 | 1 | 2 | 0 | 0 | 0 | 0 | 5 | 6 |
| Seq ID 1066 | 0 | 0 | 0 | 0 | 0 | 3 | 0 | 8 |
| Seq ID 1076 | 2 | 2 | 0 | 1 | 0 | 2 | 0 | 8 |
| Seq ID 1081 | 2 | 2 | 0 | 0 | 1 | 5 | 0 | 5 |
| Seq ID 1095 | 2 | 3 | 0 | 0 | 3 | 2 | 4 | 2 |
| Seq ID 1103 | 3 | 3 | 0 | 0 | 6 | 0 | 1 | 4 |

TABLE 8

| Lung | Nonparametric | | Global | | | | | |
|---|---|---|---|---|---|---|---|---|
| Nucleotide ID | Exp | Order | 5 | 4 | 3 | 2 | 1 | 0 |
| Seq ID 034 | 2 | 2 | 1 | 3 | 0 | 1 | 4 | 1 |
| Seq ID 310 | 2 | 3 | 0 | 0 | 0 | 0 | 5 | 5 |
| Seq ID 368 | 4 | 4 | 1 | 2 | 0 | 0 | 4 | 3 |
| Seq ID 371 | 3 | 3 | 0 | 0 | 0 | 9 | 0 | 1 |
| Seq ID 444 | 1 | 1 | 0 | 0 | 0 | 1 | 0 | 9 |
| Seq ID 649 | 1 | 0 | 0 | 0 | 0 | 0 | 0 | 10 |
| Seq ID 792 | 1 | 0 | 2 | 1 | 0 | 0 | 4 | 3 |
| Seq ID 837 | 0 | 0 | 1 | 0 | 1 | 0 | 0 | 8 |

TABLE 9

| Pancreas | Nonparametric | | Global | | | | | |
|---|---|---|---|---|---|---|---|---|
| Nucleotide ID | Exp | Order | 5 | 4 | 3 | 2 | 1 | 0 |
| Seq ID 078 | 4 | 4 | 0 | 3 | 2 | 0 | 0 | 0 |
| Seq ID 089 | 4 | 4 | 4 | 0 | 0 | 0 | 0 | 1 |
| Seq ID 129 | 5 | 5 | 1 | 4 | 0 | 0 | 0 | 0 |
| Seq ID 292 | 5 | 5 | 4 | 0 | 1 | 0 | 0 | 0 |
| Seq ID 559 | 4 | 5 | 4 | 0 | 0 | 0 | 0 | 1 |
| Seq ID 614 | 5 | 4 | 1 | 3 | 0 | 1 | 0 | 0 |
| Seq ID 996 | 5 | 5 | 0 | 4 | 0 | 0 | 0 | 1 |
| Seq ID 1028 | 5 | 5 | 2 | 0 | 0 | 1 | 1 | 1 |

TABLE 10

| Ovary | Nonparameteric | | Global | | | | | |
|---|---|---|---|---|---|---|---|---|
| Nucleotide ID | Exp | Order | 5 | 4 | 3 | 2 | 1 | 0 |
| Seq ID 014 | 4 | 4 | 2 | 1 | 1 | 1 | 0 | 1 |
| Seq ID 046 | 4 | 4 | 1 | 0 | 4 | 1 | 0 | 0 |
| Seq ID 062 | 3 | 4 | 0 | 2 | 0 | 0 | 4 | 0 |
| Seq ID 198 | 4 | 4 | 2 | 1 | 3 | 0 | 0 | 0 |
| Seq ID 270 | 4 | 4 | 2 | 0 | 3 | 1 | 0 | 0 |
| Seq ID 282 | 4 | 4 | 0 | 2 | 3 | 1 | 0 | 0 |
| Seq ID 311 | 0 | 0 | 1 | 0 | 0 | 1 | 0 | 4 |
| Seq ID 355 | 4 | 4 | 0 | 1 | 4 | 1 | 0 | 0 |
| Seq ID 360 | 3 | 3 | 0 | 0 | 3 | 0 | 3 | 0 |
| Seq ID 486 | 4 | 4 | 0 | 3 | 2 | 1 | 0 | 0 |
| Seq ID 500 | 3 | 3 | 0 | 1 | 4 | 0 | 1 | 0 |
| Seq ID 507 | 5 | 5 | 0 | 3 | 2 | 1 | 0 | 0 |
| Seq ID 509 | 3 | 3 | 0 | 0 | 5 | 1 | 0 | 0 |
| Seq ID 559 | 5 | 5 | 2 | 0 | 0 | 1 | 3 | 0 |
| Seq ID 581 | 3 | 3 | 0 | 0 | 4 | 1 | 1 | 0 |
| Seq ID 592 | 3 | 2 | 0 | 0 | 4 | 1 | 1 | 0 |
| Seq ID 651 | 4 | 3 | 1 | 1 | 3 | 0 | 0 | 1 |
| Seq ID 657 | 3 | 3 | 1 | 0 | 3 | 0 | 2 | 0 |
| Seq ID 668 | 4 | 4 | 2 | 0 | 3 | 1 | 0 | 0 |
| Seq ID 675 | 4 | 5 | 2 | 0 | 0 | 1 | 1 | 2 |
| Seq ID 708 | 4 | 4 | 0 | 1 | 4 | 1 | 0 | 0 |
| Seq ID 710 | 4 | 5 | 0 | 0 | 3 | 2 | 1 | 0 |
| Seq ID 744 | 4 | 4 | 1 | 1 | 3 | 0 | 1 | 0 |
| Seq ID 775 | 4 | 4 | 0 | 1 | 5 | 0 | 0 | 0 |
| Seq ID 777 | 3 | 4 | 0 | 2 | 2 | 0 | 2 | 0 |
| Seq ID 779 | 5 | 2 | 0 | 4 | 0 | 2 | 0 | 0 |
| Seq ID 793 | 3 | 3 | 0 | 1 | 4 | 0 | 0 | 1 |
| Seq ID 821 | 4 | 4 | 1 | 3 | 2 | 0 | 0 | 0 |
| Seq ID 825 | 3 | 3 | 0 | 1 | 3 | 2 | 0 | 0 |
| Seq ID 878 | 3 | 3 | 1 | 1 | 3 | 0 | 1 | 0 |
| Seq ID 983 | 3 | 3 | 0 | 1 | 3 | 2 | 0 | 0 |

TABLE 11
Fold Expression for SEQ ID 142

| | Sample 1 | Sample 2 | Sample 3 | Sample 4 | Sample 5 | Sample 6 | Sample 7 | Sample 8 | Sample 9 | Sample 10 |
|---|---|---|---|---|---|---|---|---|---|---|
| Fold expression | 3.49 | 2.7 | 6.72 | 2.93 | 4.93 | 10.31 | 6.84 | 4.16 | 2.7 | 4.27 |

1. An array comprising a plurality of nucleic acid molecules selected from the group consisting of: the nucleotide sequences set forth in SEQ ID NOs: 1-1146, 3439-3445, and 3452-3457, their complements and hybridizing fragments thereof.

2-22. (canceled)

23. A method of associating a plurality of molecules selected from the group consisting of nucleic acid molecules, protein molecules and fragments thereof comprising the steps of:
(a) querying a sequence database for the presence of transmembrane molecules using a transmembrane protein topology prediction program to provide a transmembrane selection set;
(b) comparing homology of the molecules of the transmembrane selection set with array associated molecules; and
(c) excluding those molecules from the transmembrane selection set exhibiting substantial homology with one or more array associated molecules to provide a transmembrane signature set comprising a plurality of molecules.

24-41. (canceled)

42. A method for treating a hyperproliferative disease in an animal, comprising administering to an animal in need of treatment a composition comprising a binding molecule which specifically binds to a polypeptide, variant, or fragment thereof, wherein said polypeptide, variant, or fragment thereof is selected from the group consisting of:
(i) a colon tumor-associated polypeptide;
(ii) a lung tumor-associated polypeptide;
(iii) a pancreatic tumor-associated polypeptide; and
(iv) an ovarian tumor-associated polypeptide.

43. A method for treating a hyperproliferative disorder in an animal, comprising administering to an animal in need of treatment a composition comprising a binding molecule which specifically binds to a polypeptide, variant, or fragment thereof, which is at least 70% identical to a colon tumor-associated polypeptide selected from the group consisting of:
(i) SEQ ID NO:1288;
(ii) SEQ ID NO:3446;
(iii) SEQ ID NO:3447;
(iv) SEQ ID NO:3448;
(v) SEQ ID NO:3449;
(vi) SEQ ID NO:3450;
(vii) SEQ ID NO:3451;
(viii) SEQ ID NO:3452;
(ix) SEQ ID NO:3458;
(x) SEQ ID NO:3459;
(xi) SEQ ID NO:3460;
(xii) SEQ ID NO:3461; and
(xiii) SEQ ID NO:3462.

44-46. (canceled)

47. The method of claim 46, wherein said polypeptide, variant, or fragment thereof, is at least 90% identical to said colon tumor-associated polypeptide.

48. The method of claim 47, wherein said polypeptide, variant, or fragment thereof, is at least 95% identical to said colon tumor-associated polypeptide.

49. The method of claim 48, wherein said polypeptide, variant, or fragment thereof, is 100% identical to said colon tumor-associated polypeptide.

50. The method of claim 42, wherein said polypeptide, variant, or fragment thereof, is selected from the group consisting of:
(i) amino acids 193-218 of SEQ ID NO:3446; and
(ii) amino acids 349-626 of SEQ ID NO:3446.

51. The method of claim 42, wherein said polypeptide, variant, or fragment thereof, is selected from the group consisting of:
(i) amino acids 1-129 of SEQ ID NO:3447;
(ii) amino acids 1-23 of SEQ ID NO:3447;
(iii) amino acids 5-223 of SEQ ID NO:3447;
(iv) amino acids 5-131 of SEQ ID NO:3447;
(v) amino acids 5-510 of SEQ ID NO:3447;
(vi) amino acids 12-220 of SEQ ID NO:3447;
(vii) amino acids 20-227 of SEQ ID NO:3447;
(viii) amino acids 25-145 of SEQ ID NO:3447;
(ix) amino acids 28-232 of SEQ ID NO:3447;
(x) amino acids 29-391 of SEQ ID NO:3447;
(xi) amino acids 30-457 of SEQ ID NO:3447;
(xii) amino acids 30-427 of SEQ ID NO:3447;
(xiii) amino acids 34-216 of SEQ ID NO:3447;
(xiv) amino acids 35-457 of SEQ ID NO:3447;
(xv) amino acids 37-59 of SEQ ID NO:3447;
(xvi) amino acids 38-140 of SEQ ID NO:3447;
(xvii) amino acids 38-212 of SEQ ID NO:3447;
(xviii) amino acids 62-458 of SEQ ID NO:3447;
(xix) amino acids 66-303 of SEQ ID NO:3447;
(xx) amino acids 72-94 of SEQ ID NO:3447;
(xxi) amino acids 97-250 of SEQ ID NO:3447;
(xxii) amino acids 105-207 of SEQ ID NO:3447;
(xxiii) amino acids 106-259 of SEQ ID NO:3447;
(xxiv) amino acids 106-458 of SEQ ID NO:3447;
(xxv) amino acids 109-131 of SEQ ID NO:3447;
(xxvi) amino acids 147-429 of SEQ ID NO:3447;
(xxvii) amino acids 152-174 of SEQ ID NO:3447;
(xxviii) amino acids 176-289 of SEQ ID NO:3447;
(xxix) amino acids 197-417 of SEQ ID NO:3447;
(xxx) amino acids 197-422 of SEQ ID NO:3447;
(xxxi) amino acids 198-220 of SEQ ID NO:3447;
(xxxii) amino acids 202-239 of SEQ ID NO:3447;
(xxxiii) amino acids 207-279 of SEQ ID NO:3447;
(xxxiv) amino acids 212-407 of SEQ ID NO:3447;
(xxxv) amino acids 217-271 of SEQ ID NO:3447;
(xxxvi) amino acids 220-256 of SEQ ID NO:3447;
(xxxvii) amino acids 221-297 of SEQ ID NO:3447;
(xxxviii) amino acids 243-401 of SEQ ID NO:3447;
(xxxix) amino acids 247-321 of SEQ ID NO:3447;
(xl) amino acids 247-263 of SEQ ID NO:3447;
(xli) amino acids 271-364 of SEQ ID NO:3447;
(xlii) amino acids 271-338 of SEQ ID NO:3447;
(xliii) amino acids 278-371 of SEQ ID NO:3447;
(xliv) amino acids 292-301 of SEQ ID NO:3447;
(xlv) amino acids 312-373 of SEQ ID NO:3447;
(xlvi) amino acids 321-481 of SEQ ID NO:3447;
(xlvii) amino acids 369-470 of SEQ ID NO:3447;
(xlviii) amino acids 379-395 of SEQ ID NO:3447;
(xlix) amino acids 387-402 of SEQ ID NO:3447;
(l) amino acids 406-428 of SEQ ID NO:3447; and
(li) amino acids 418-464 of SEQ ID NO:3447.

52. The method of claim 42, wherein said polypeptide, variant, or fragment thereof, is selected from the group consisting of:
(i) amino acids 1-43 of SEQ ID NO:3448; and
(ii) amino acids 29-51 of SEQ ID NO:3448.

53. The method of claim 42, wherein said polypeptide, variant, or fragment thereof, is selected from the group consisting of:
(i) amino acids 1-21 of SEQ ID NO:3449;
(ii) amino acids 41-287 of SEQ ID NO:3449;
(iii) amino acids 25-47 of SEQ ID NO:3449;
(iv) amino acids 59-78 of SEQ ID NO:3449;
(v) amino acids 98-120 of SEQ ID NO:3449;
(vi) amino acids 141-163 of SEQ ID NO:3449;
(vii) amino acids 200-222 of SEQ ID NO:3449;
(viii) amino acids 243-265 of SEQ ID NO:3449; and
(ix) amino acids 270-289 of SEQ ID NO:3449.

54. The method of claim 42, wherein said polypeptide, variant, or fragment thereof, is selected from the group consisting of:
(i) amino acids 1-39 of SEQ ID NO:3450;
(ii) amino acids 41-290 of SEQ ID NO:3450;
(iii) amino acids 26-48 of SEQ ID NO:3450;
(iv) amino acids 61-83 of SEQ ID NO:3450;
(v) amino acids 98-120 of SEQ ID NO:3450;
(vi) amino acids 144-166 of SEQ ID NO:3450;
(vii) amino acids 199-221 of SEQ ID NO:3450;
(viii) amino acids 241-263 of SEQ ID NO:3450; and
(ix) amino acids 273-292 of SEQ ID NO:3450.

55. The method of claim 42, wherein said polypeptide, variant, or fragment thereof, is selected from the group consisting of:
(i) amino acids 125-139 of SEQ ID NO:3451;
(ii) amino acids 1-123 of SEQ ID NO:3451;
(iii) amino acids 1-83 of SEQ ID NO:3451;
(iv) amino acids 3-72 of SEQ ID NO:3451;
(v) amino acids 4-77 of SEQ ID NO:3451;
(vi) amino acids 7-109 of SEQ ID NO:3451;
(vii) amino acids 8-119 of SEQ ID NO:3451;
(viii) amino acids 14-108 of SEQ ID NO:3451;
(ix) amino acids 17-107 of SEQ ID NO:3451;
(x) amino acids 24-99 of SEQ ID NO:3451;
(xi) amino acids 25-137 of SEQ ID NO:3451;
(xii) amino acids 25-124 of SEQ ID NO:3451;
(xiii) amino acids 27-87 of SEQ ID NO:3451;
(xiv) amino acids 30-119 of SEQ ID NO:3451;
(xv) amino acids 30-50 of SEQ ID NO:3451;
(xvi) amino acids 32-58 of SEQ ID NO:3451;
(xvii) amino acids 41-112 of SEQ ID NO:3451;
(xviii) amino acids 44-119 of SEQ ID NO:3451;
(xix) amino acids 44-123 of SEQ ID NO:3451;
(xx) amino acids 53-108 of SEQ ID NO:3451;
(xxi) amino acids 60-128 of SEQ ID NO:3451;
(xxii) amino acids 63-115 of SEQ ID NO:3451;
(xxiii) amino acids 63-84 of SEQ ID NO:3451;
(xxiv) amino acids 63-102 of SEQ ID NO:3451;
(xxv) amino acids 65-94 of SEQ ID NO:3451;
(xxvi) amino acids 67-140 of SEQ ID NO:3451;
(xxvii) amino acids 69-113 of SEQ ID NO:3451;
(xxviii) amino acids 69-128 of SEQ ID NO:3451;
(xxix) amino acids 95-117 of SEQ ID NO:3451; and
(xxx) amino acids 110-124 of SEQ ID NO:3451.

56. The method of claim 42, wherein said polypeptide, variant, or fragment thereof, is selected from the group consisting of:
(i) amino acids 39-285 of SEQ ID NO:3452;
(ii) amino acids 1-49 of SEQ ID NO:3452;
(iii) amino acids 31-53 of SEQ ID NO:3452;
(iv) amino acids 58-80 of SEQ ID NO:3452;
(v) amino acids 100-122 of SEQ ID NO:3452;
(vi) amino acids 143-165 of SEQ ID NO:3452;
(vii) amino acids 201-223 of SEQ ID NO:3452;
(viii) amino acids 236-258 of SEQ ID NO:3452; and
(ix) amino acids 268-287 of SEQ ID NO:3452.

57. The method of claim 42, wherein said polypeptide, variant, or fragment thereof is selected from the group consisting of:
(i) amino acids 1-627 of SEQ ID NO:3446;
(ii) amino acids 1-36 of SEQ ID NO:3447;
(iii) amino acids 95-108 of SEQ ID NO:3447;
(iv) amino acids 175-197 of SEQ ID NO:3447;
(v) amino acids 429-437 of SEQ ID NO:3447;
(vi) amino acids 1-28 of SEQ ID NO:3448;
(vii) amino acids 1-24 of SEQ ID NO:3449;
(viii) amino acids 79-97 of SEQ ID NO:3449;
(ix) amino acids 164-199 of SEQ ID NO:3449;
(x) amino acids 166-169 of SEQ ID NO:3449;
(xi) amino acids 1-25 of SEQ ID NO:3450;
(xii) amino acids 84-87 of SEQ ID NO:3450;
(xiii) amino acids 167-198 of SEQ ID NO:3450;
(xiv) amino acids 264-272 of SEQ ID NO:3450;
(xv) amino acids 1-30 of SEQ ID NO:3452;
(xvi) amino acids 81-99 of SEQ ID NO:3452;
(xvii) amino acids 166-200 of SEQ ID NO:3452; and
(xviii) amino acids 259-267 of SEQ ID NO:3452.

58. The method of claim 42, wherein said polypeptide, variant, or fragment thereof, is selected from the group consisting of:
(i) amino acids 1154-1311 of SEQ ID NO:3458;
(ii) amino acids 1310-1425 of SEQ ID NO:3458;
(iii) amino acids 1428-1610 of SEQ ID NO:3458;
(iv) amino acids 1834-1866 of SEQ ID NO:3458;
(v) amino acids 1873-1914 of SEQ ID NO:3458;
(vi) amino acids 2080-2117 of SEQ ID NO:3458;
(vii) amino acids 2129-2151 of SEQ ID NO:3458;
(viii) amino acids 40-344 of SEQ ID NO:3458;
(ix) amino acids 66-122 of SEQ ID NO:3458;
(x) amino acids 69-273 of SEQ ID NO:3458;
(xi) amino acids 145-311 of SEQ ID NO:3458;
(xii) amino acids 205-328 of SEQ ID NO:3458;
(xiii) amino acids 223-304 of SEQ ID NO:3458;
(xiv) amino acids 358-385 of SEQ ID NO:3458;
(xv) amino acids 372-470 of SEQ ID NO:3458;
(xvi) amino acids 715-763 of SEQ ID NO:3458;
(xvii) amino acids 895-967 of SEQ ID NO:3458;
(xviii) amino acids 909-940 of SEQ ID NO:3458;
(xix) amino acids 957-1155 of SEQ ID NO:3458;
(xx) amino acids 1118-1264 of SEQ ID NO:3458;
(xxi) amino acids 1350-1577 of SEQ ID NO:3458;
(xxii) amino acids 1365-1422 of SEQ ID NO:3458;
(xxiii) ‘amino acids 1469-1661 of SEQ ID NO:3458;
(xxiv) amino acids 1471-1509 of SEQ ID NO:3458;
(xxv) amino acids 1641-1665 of SEQ ID NO:3458;
(xxvi) amino acids 1652-1750 of SEQ ID NO:3458;
(xxvii) amino acids 1661-1689 of SEQ ID NO:3458;
(xxviii) amino acids 1710-1845 of SEQ ID NO:3458;
(xxix) amino acids 1716-1794 of SEQ ID NO:3458;
(xxx) amino acids 1793-1833 of SEQ ID NO:3458;
(xxxi) amino acids 1799-1854 of SEQ ID NO:3458;
(xxxii) amino acids 1814-1885 of SEQ ID NO:3458;
(xxxiii) amino acids 1817-1865 of SEQ ID NO:3458;
(xxxiv) amino acids 1825-1874 of SEQ ID NO:3458;
(xxxv) amino acids 1825-1885 of SEQ ID NO:3458;
(xxxvi) amino acids 1829-1953 of SEQ ID NO:3458;
(xxxvii) amino acids 1829-1891 of SEQ ID NO:3458;
(xxxviii) amino acids 1833-1877 of SEQ ID NO:3458;
(xxxix) amino acids 1840-1896 of SEQ ID NO:3458;
(xl) amino acids 1844-1865 of SEQ ID NO:3458;
(xli) amino acids 1846-1890 of SEQ ID NO:3458;
(xlii) amino acids 1848-1886 of SEQ ID NO:3458;
(xliii) amino acids 1850-1885 of SEQ ID NO:3458;
(xliv) amino acids 1851-1884 of SEQ ID NO:3458;
(xlv) amino acids 1852-1900 of SEQ ID NO:3458;
(xlvi) amino acids 1854-1902 of SEQ ID NO:3458;
(xlvii) amino acids 1860-1894 of SEQ ID NO:3458;
(xlviii) amino acids 1865-1898 of SEQ ID NO:3458;
(xlix) amino acids 1873-1898 of SEQ ID NO:3458;
(l) amino acids 1873-1902 of SEQ ID NO:3458;
(li) amino acids 1874-1913 of SEQ ID NO:3458;
(lii) amino acids 1879-1902 of SEQ ID NO:3458;
(liii) amino acids 1890-1940 of SEQ ID NO:3458;
(liv) amino acids 1897-1934 of SEQ ID NO:3458;
(lv) amino acids 1968-2082 of SEQ ID NO:3458;
(lvi) amino acids 2058-2117 of SEQ ID NO:3458;
(lvii) amino acids 2072-2096 of SEQ ID NO:3458;
(lviii) amino acids 2080-2102 of SEQ ID NO:3458;
(lix) amino acids 2081-2117 of SEQ ID NO:3458; and
(lx) amino acids 2082-2113 of SEQ ID NO:3458

59. The method of claim 42, wherein said polypeptide, variant, or fragment thereof, is selected from the group consisting of:
(i) amino acids 1153-1255 of SEQ ID NO:3459;
(ii) amino acids 23-163 of SEQ ID NO:3459;
(iii) amino acids 40-344 of SEQ ID NO:3459;
(iv) amino acids 66-122 of SEQ ID NO:3459;
(v) amino acids 69-273 of SEQ ID NO:3459;
(vi) amino acids 145-311 of SEQ ID NO:3459;
(vii) amino acids 205-328 of SEQ ID NO:3459;
(viii) amino acids 223-304 of SEQ ID NO:3459;
(ix) amino acids 358-385 of SEQ ID NO:3459;
(x) amino acids 372-470 of SEQ ID NO:3459;
(xi) amino acids 594-702 of SEQ ID NO:3459;
(xii) amino acids 715-763 of SEQ ID NO:3459;
(xiii) amino acids 743-857 of SEQ ID NO:3459;
(xiv) amino acids 831-968 of SEQ ID NO:3459;
(xv) amino acids 894-966 of SEQ ID NO:3459;
(xvi) amino acids 908-939 of SEQ ID NO:3459;
(xvii) amino acids 956-1154 of SEQ ID NO:3459

60. The method of claim 42, wherein said polypeptide, variant, or fragment thereof, is selected from the group consisting of:
(i) amino acids 1154-1311 of SEQ ID NO:3460;
(ii) amino acids 1310-1425 of SEQ ID NO:3460;
(iii) amino acids 1428-1610 of SEQ ID NO:3460;
(iv) amino acids 40-344 of SEQ ID NO:3460;
(v) amino acids 66-122 of SEQ ID NO:3460;
(vi) amino acids 69-273 of SEQ ID NO:3460;
(vii) amino acids 145-311 of SEQ ID NO:3460;
(viii) amino acids 205-328 of SEQ ID NO:3460;
(ix) amino acids 223-304 of SEQ ID NO:3460;
(x) amino acids 358-385 of SEQ ID NO:3460;
(xi) amino acids 372-470 of SEQ ID NO:3460;
(xii) amino acids 594-702 of SEQ ID NO:3460;
(xiii) amino acids 715-763 of SEQ ID NO:3460;
(xiv) amino acids 895-967 of SEQ ID NO:3460;
(xv) amino acids 909-940 of SEQ ID NO:3460;
(xvi) amino acids 957-1155 of SEQ ID NO:3460;
(xvii) amino acids 1118-1264 of SEQ ID NO:3460;
(xviii) amino acids 1350-1577 of SEQ ID NO:3460;
(xix) amino acids 1363-1378 of SEQ ID NO:3460;
(xx) amino acids 1365-1422 of SEQ ID NO:3460;
(xxi) amino acids 1377-1409 of SEQ ID NO:3460;
(xxii) amino acids 1469-1661 of SEQ ID NO:3460;
(xxiii) amino acids 1471-1509 of SEQ ID NO:3460;
(xxiv) amino acids 1641-1665 of SEQ ID NO:3460;
(xxv) amino acids 1652-1750 of SEQ ID NO:3460;
(xxvi) amino acids 1661-1689 of SEQ ID NO:3460;
(xxvii) amino acids 1716-1794 of SEQ ID NO:3460;
(xxviii) amino acids 1785-1826 of SEQ ID NO:3460;
(xxix) amino acids 1790-1820 of SEQ ID NO:3460

61. The method of claim 42, wherein said polypeptide, variant, or fragment thereof, is selected from the group consisting of:
(i) amino acids 161-318 of SEQ ID NO:3461;
(ii) amino acids 317-432 of SEQ ID NO:3461;
(iii) amino acids 435-617 of SEQ ID NO:3461;
(iv) amino acids 841-873 of SEQ ID NO:3461;
(v) amino acids 880-921 of SEQ ID NO:3461;
(vi) amino acids 1087-1124 of SEQ ID NO:3461;
(vii) amino acids 1136-1158 of SEQ ID NO:3461;
(viii) amino acids 125-271 of SEQ ID NO:3461;
(ix) amino acids 214-252 of SEQ ID NO:3461;
(x) amino acids 357-584 of SEQ ID NO:3461;
(xi) amino acids 372-429 of SEQ ID NO:3461;
(xii) amino acids 476-668 of SEQ ID NO:3461;
(xiii) amino acids 478-516 of SEQ ID NO:3461;
(xiv) amino acids 480-510 of SEQ ID NO:3461;
(xv) amino acids 648-672 of SEQ ID NO:3461;
(xvi) amino acids 659-757 of SEQ ID NO:3461;
(xvii) amino acids 668-696 of SEQ ID NO:3461;
(xviii) amino acids 717-852 of SEQ ID NO:3461;
(xix) amino acids 723-801 of SEQ ID NO:3461;
(xx) amino acids 800-840 of SEQ ID NO:3461;
(xxi) amino acids 806-861 of SEQ ID NO:3461;
(xxii) amino acids 821-892 of SEQ ID NO:3461;
(xxiii) amino acids 824-872 of SEQ ID NO:3461;
(xxiv) amino acids 832-881 of SEQ ID NO:3461;
(xxv) amino acids 832-892 of SEQ ID NO:3461;
(xxvi) amino acids 836-960 of SEQ ID NO:3461;
(xxvii) amino acids 836-898 of SEQ ID NO:3461;
(xxviii) amino acids 840-884 of SEQ ID NO:3461;
(xxix) amino acids 847-903 of SEQ ID NO:3461;
(xxx) amino acids 851-872 of SEQ ID NO:3461;
(xxxi) amino acids 853-897 of SEQ ID NO:3461;
(xxxii) amino acids 855-893 of SEQ ID NO:3461;
(xxxiii) amino acids 857-892 of SEQ ID NO:3461;
(xxxiv) amino acids 858-891 of SEQ ID NO:3461;
(xxxv) amino acids 859-907 of SEQ ID NO:3461;
(xxxvi) amino acids 861-909 of SEQ ID NO:3461;
(xxxvii) amino acids 867-901 of SEQ ID NO:3461;
(xxxviii) amino acids 872-905 of SEQ ID NO:3461;
(xxxix) amino acids 880-909 of SEQ ID NO:3461;
(xl) amino acids 880-905 of SEQ ID NO:3461;
(xli) amino acids 881-920 of SEQ ID NO:3461;
(xlii) amino acids 886-909 of SEQ ID NO:3461;
(xliii) amino acids 897-947 of SEQ ID NO:3461;
(xliv) amino acids 904-941 of SEQ ID NO:3461;
(xlv) amino acids 975-1089 of SEQ ID NO:3461;
(xlvi) amino acids 1021-1089 of SEQ ID NO:3461;
(xlvii) amino acids 1065-1124 of SEQ ID NO:3461;
(xlviii) amino acids 1079-1103 of SEQ ID NO:3461;
(xlix) amino acids 1087-1109 of SEQ ID NO:3461;
(l) amino acids 1088-1124 of SEQ ID NO:3461; and
(li) amino acids 1089-1120 of SEQ ID NO:3461

62. The method of claim 42, wherein said polypeptide, variant, or fragment thereof, is selected from the group consisting of:
(i) amino acids 12-34 of SEQ ID NO:3462;
(ii) amino acids 110-267 of SEQ ID NO:3462;
(iii) amino acids 266-381 of SEQ ID NO:3462;
(iv) amino acids 384-566 of SEQ ID NO:3462;
(v) amino acids 790-822 of SEQ ID NO:3462;
(vi) amino acids 829-870 of SEQ ID NO:3462;
(vii) amino acids 1036-1073 of SEQ ID NO:3462;
(viii) amino acids 1085-1107 of SEQ ID NO:3462;
(ix) amino acids 74-220 of SEQ ID NO:3462;
(x) amino acids 163-201 of SEQ ID NO:3462;
(xi) amino acids 306-533 of SEQ ID NO:3462;
(xii) amino acids 321-378 of SEQ ID NO:3462;
(xiii) amino acids 425-617 of SEQ ID NO:3462;
(xiv) amino acids 427-465 of SEQ ID NO:3462;
(xv) amino acids 429-459 of SEQ ID NO:3462;
(xvi) amino acids 597-621 of SEQ ID NO:3462;
(xvii) amino acids 608-706 of SEQ ID NO:3462;
(xviii) amino acids 617-645 of SEQ ID NO:3462;
(xix) amino acids 666-801 of SEQ ID NO:3462;
(xx) amino acids 672-750 of SEQ ID NO:3462;
(xxi) amino acids 749-789 of SEQ ID NO:3462;
(xxii) amino acids 755-810 of SEQ ID NO:3462;
(xxiii) amino acids 770-841 of SEQ ID NO:3462;
(xxiv) amino acids 773-821 of SEQ ID NO:3462;
(xxv) amino acids 781-830 of SEQ ID NO:3462;
(xxvi) amino acids 781-841 of SEQ ID NO:3462;
(xxvii) amino acids 785-909 of SEQ ID NO:3462;
(xxviii) amino acids 785-847 of SEQ ID NO:3462;
(xxix) amino acids 789-833 of SEQ ID NO:3462;
(xxx) amino acids 796-852 of SEQ ID NO:3462;
(xxxi) amino acids 800-821 of SEQ ID NO:3462;
(xxxii) amino acids 802-846 of SEQ ID NO:3462;
(xxxiii) amino acids 804-842 of SEQ ID NO:3462;
(xxxiv) amino acids 806-841 of SEQ ID NO:3462;
(xxxv) amino acids 807-840 of SEQ ID NO:3462;
(xxxvi) amino acids 808-856 of SEQ ID NO:3462;
(xxxvii) amino acids 810-858 of SEQ ID NO:3462;
(xxxviii) amino acids 816-850 of SEQ ID NO:3462;
(xxxix) amino acids 821-854 of SEQ ID NO:3462;
(xl) amino acids 829-858 of SEQ ID NO:3462;
(xli) amino acids 829-854 of SEQ ID NO:3462;
(xlii) amino acids 830-869 of SEQ ID NO:3462;
(xliii) amino acids 835-858 of SEQ ID NO:3462;
(xliv) amino acids 846-896 of SEQ ID NO:3462;
(xlv) amino acids 853-890 of SEQ ID NO:3462;
(xlvi) amino acids 924-1038 of SEQ ID NO:3462;
(xlvii) amino acids 970-1038 of SEQ ID NO:3462;
(xlviii) amino acids 1014-1073 of SEQ ID NO:3462;
(xlix) amino acids 1028-1052 of SEQ ID NO:3462;
(l) amino acids 1036-1058 of SEQ ID NO:3462;
(li) amino acids 1037-1073 of SEQ ID NO:3462; and
(lii) amino acids 1038-1069 of SEQ ID NO:3462.

63. The method of claim 42, wherein said polypeptide, variant, or fragment thereof is selected from the group consisting of:
(i) amino acids 1-2128 of SEQ ID NO:3458;
(ii) amino acids 1-1255 of SEQ ID NO:3459;
(iii) amino acids 1-1827 of SEQ ID NO:3460;
(iv) amino acids 1-1135 of SEQ ID NO:3461; and
(v) amino acids 35-1084 of SEQ ID NO:3462.

64-113. (canceled)

114. A method of detecting abnormal hyperproliferative cell growth in a patient comprising:
(a) obtaining a biological sample from the patient;
(b) contacting said sample with a binding molecule which specifically binds to a polypeptide, variant, or fragment thereof, which is at least 70% identical to a colon tumor-associated polypeptide selected from the group consisting of:
(i) SEQ ID NO:1288
(ii) SEQ ID NO:3446;
(iii) SEQ ID NO:3447;
(iv) SEQ ID NO:3448;
(v) SEQ ID NO:3449;
(vi) SEQ ID NO:3450;
(vii) SEQ ID NO:3451;
(viii) SEQ ID NO:3452;
(ix) SEQ ID NO:3458;
(x) SEQ ID NO:3459;
(xi) SEQ ID NO:3460;
(xii) SEQ ID NO:3461; and
(xiii) SEQ ID NO:3462;
(c) assaying the expression level of said colon tumor-associated polypeptide in said sample.

115-124. (canceled)

125. A method of diagnosing a hyperproliferative disease or disorder in a patient, comprising:
(a) administering to said patient a sufficient amount of a detectably labeled binding molecule which specifically binds to a polypeptide, variant, or fragment thereof, which is at least 70% identical to a colon tumor-associated polypeptide selected from the group consisting of:
(i) SEQ ID NO:1288
(ii) SEQ ID NO:3446;
(iii) SEQ ID NO:3447;
(iv) SEQ ID NO:3448;
(v) SEQ ID NO:3449;
(vi) SEQ ID NO:3450;
(vii) SEQ ID NO:3451;
(viii) SEQ ID NO:3452;
(ix) SEQ ID NO:3458;
(x) SEQ ID NO:3459;
(xi) SEQ ID NO:3460;
(xii) SEQ ID NO:3461; and
(xiii) SEQ ID NO:3462;
(b) waiting for a time interval following said administration to allow said binding molecule to contact said polypeptide, variant, or fragment thereof; and
(c) detecting the amount of said binding molecule bound to said polypeptide, variant of fragment thereof in said patient.

126-193. (canceled)

194. A polynucleotide, variant, or fragment thereof, which is at least 85% identical to a nucleic acid molecule selected from the group consisting of SEQ ID NO: 142, SEQ ID NOs: 3439-3445 and SEQ ID NOs: 3453-3457.

195. The polynucleotide of claim 194 which is at least 90% identical to a nucleic acid molecule selected from the group consisting of SEQ ID NO: 142, SEQ ID NOs: 3439-3445 and SEQ ID NOs: 3453-3457.

196. The polynucleotide of claim 195 which is at least 95% identical to a nucleic acid molecule selected from the group consisting of SEQ ID NO: 142, SEQ ID NOs: 3439-3445 and SEQ ID NOs: 3453-3457.

197. The polynucleotide of claim 196 which is at least 100% identical to a nucleic acid molecule selected from the group consisting of SEQ ID NO: 142, SEQ ID NOs: 3439-3445 and SEQ ID NOs: 3453-3457.

198. A polypeptide, variant, or fragment thereof, which is at least 85% identical to a nucleic acid molecule selected from the group consisting of SEQ ID NO: 1228, SEQ ID NOs: 3446-3452 and SEQ ID NOs: 3458-3462.

199. The polypeptide of claim 198 which is at least 90% identical to a nucleic acid molecule selected from the group consisting of SEQ ID NO: 1228, SEQ ID NOs: 3446-3452 and SEQ ID NOs: 3458-3462.

200. The polynucleotide of claim 199 which is at least 95% identical to a nucleic acid molecule selected from the group consisting of SEQ ID NO: 1228, SEQ ID NOs: 3446-3452 and SEQ ID NOs: 3458-3462.

201. The polynucleotide of claim 200 which is at least 100% identical to a nucleic acid molecule selected from the group consisting of SEQ ID NO: 1228, SEQ ID NOs: 3446-3452 and SEQ ID NOs: 3458-3462.
